(19) United States
(12) Patent Application Publication
(10) Pub. No.: US 2017/0327553 A1
(43) Pub. Date: Nov. 16, 2017

(54) COMPOSITIONS AND METHODS FOR LONG ACTING PROTEINS

(71) Applicants: Janet DAWSON KING, Bennwil (CH); Joy GHOSH, Boston, MA (US); Barbara NUESSLEIN-HILDESHEIM, Eimeldigen (DE)

(72) Inventors: Janet DAWSON KING, Bennwil (CH); Joy GHOSH, Boston, MA (US); Barbara NUESSLEIN-HILDESHEIM, Eimeldigen (DE)

(73) Assignee: Novartis AG, Basel (CH)

(21) Appl. No.: 15/321,065

(22) PCT Filed: Jun. 24, 2015
(86) PCT No.: PCT/IB2015/054737

§ 371 Date: Dec. 21, 2016

Related U.S. Application Data
(60) Provisional application No. 62/016,962, filed on Jun. 25, 2014.

(51) Int. Cl. C07K 14/525; C07K 16/24

(52) U.S. Cl. CPC C07K 14/525; C07K 16/241; C07K 2319/33; C07K 2317/72

(57) ABSTRACT

The present invention provides peptide tags that can be linked to a therapeutic molecule in order to decrease the clearance of the therapeutic molecule from the synovial joint, thereby increasing its intra-articular half-life.

### BACKGROUND OF THE INVENTION

Synovial joint diseases, including arthritis, have an inflammatory component that leads to limited mobility in the joint or pain and stiffness with movement. Thus there is a need for joint therapy that can be delivered less frequently, yet still provide the same treatment benefit seen with weekly, monthly or bi-monthly treatment with these agents.

The synovial joint, or diarthrosis, is one of the most common joints in mammals. It is also the joint that can be moved the most. This joint gains movement through the contacting point with the surrounding bones, which is common among most of the other joints. There are structural and functional differences that distinguish the synovial joints from all of the rest of the joints. The main difference between the synovial joints and others is the presence of the capsules around the surface of the synovial joint, along with the presence of the lubricating fluid. High levels of hyaluronic acid is present in the synovial fluid of synovial joints. The present invention describes peptide tags that bind hyaluronic acid in the synovial joints enabling the molecules to which they are linked to have longer half-life, longer intra-articular retention and a longer duration of action in synovial joint diseases and injuries.

The present invention provides peptide tags that can be linked to a therapeutic molecule in order to decrease the clearance of the therapeutic molecule from the synovial joint, thereby increasing its intra-articular half-life. For example, peptide tagged molecules are described herein with increased duration of efficacy in the synovial joints relative to an untagged molecule, which clinically will lead to less frequent intra-articular injections and improved patient treatment.

### SUMMARY OF THE INVENTION

The present invention relates to peptide tags, as described herein, that bind hyaluronan (HA) in a synovial joint. In certain aspects the invention relates to a peptide tag, as described herein, that bind hyaluronan (HA) in a synovial joint with a K_{D} of less than or equal to 9.0 uM. For example, the peptide tag can bind HA with a KD of less than or equal to 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 9.0 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. The invention also relates to an isolated peptide tag that binds, or is capable of binding, HA comprising the sequence of SEQ ID NO: 32, SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207, or SEQ ID NO: 220.

The present invention also relates to a peptide tagged molecule comprising one or more peptide tags linked to a protein or nucleic acid, where the peptide tag comprises the sequence of SEQ ID NO: 32, SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207, or SEQ ID NO: 220. Where a peptide tag is linked to a protein, the tag can be linked to an amino acid of such protein. Where the peptide tag is linked to a nucleic acid, the tag can be linked to a nucleotide of such nucleic acid. In certain aspects is it contemplated that the peptide tag is linked to the N-terminus and/or C-terminus of a protein molecule or at the 5′ and/or 3′ end of a nucleic acid. In addition, the peptide tag may be linked directly to the protein or nucleic acid, or the peptide tag may be linked indirectly to the protein or nucleic acid via a linker. It is contemplated that the peptide tagged molecules described herein may be useful as a medicament.

In certain aspects of the invention, the peptide tagged molecule comprises a peptide tag linked to protein, for example, an antibody, or antigen binding fragment, a therapeutic protein, a protein receptor, or a designed-ankyrin repeat protein (DARPin). In certain aspects of the invention the peptide tagged molecule comprises a peptide tag linked to an aptamer. It is contemplated that the peptide tagged molecule binds TNFα, VEGF, C5, Factor P, Factor D, EPO, EPOR, IL-1β, IL-17A, IL-6, IL-18, IL-8, bFGF, MCP-1, IL-6R, CD20, FGFR2, CD132, IGF-1 and/or PDGF-BB.

The present invention also relates to a peptide tagged molecule comprising an isolated antibody or antigen binding fragment that binds TNFα and comprises heavy chain CDR1, 2, and 3 sequences of SEQ ID NOs: 108, 109 and 110, respectively and light chain CDR1, 2, and 3 sequences of SEQ ID NOs: 117, 118 and 119, respectively.

The present invention also relates to a peptide tagged molecule comprising an isolated antibody or antigen binding fragment that binds TNFα comprises heavy chain CDR1, CDR2, and CDR3 sequences of SEQ ID NOS: 208, 209, and 210, respectively and light chain CDR1, CDR2, and CDR3 sequences of SEQ ID NOs 213, 214 and 215, respectively.

The present invention also relates to a peptide tagged molecule comprising an isolated antibody or antigen binding fragment further comprising a variable heavy chain domain and a variable light chain domain having the sequences of SEQ ID NO: 111 and SEQ ID NO: 120, respectively. In certain aspects, the invention relates to a peptide tagged molecule comprising an isolated antibody or antigen binding fragment having a heavy chain and a light chain sequence of SEQ ID NO: 113 and SEQ ID NO: 122, respectively. More specifically, the peptide tagged molecule comprises, respectively, the tagged heavy chain sequence and light chain sequence of SEQ ID NOs: 115 and 122.

The present invention also relates to a peptide tagged molecule comprising an isolated antibody or antigen binding fragment further comprising a variable heavy chain domain and a variable light chain domain having the sequences of SEQ ID NO: 211 and SEQ ID NO: 216, respectively. In certain aspects, the invention relates to a peptide tagged molecule comprising an isolated antibody or antigen binding fragment having a heavy chain and a light chain sequence of SEQ ID NO: 212 and SEQ ID NO: 217, respectively. More specifically, the peptide tagged molecule comprises, respectively, the tagged heavy chain sequence and light chain sequence of SEQ ID NOs: 218 and 219.

The present invention also relates to a peptide tag or peptide tagged molecule as described in Tables 1, 2, 4, 4b, or 5. More specifically, in certain aspects the peptide tagged molecule is NVS1, NVS2, NVS3, NVS36, NVS37, NVS70T, NVS71T, NVS72T, NVS73T, NVS74T, NVS75T, NVS76T, NVS77T, NVS78T, NVS80T, NVS81T, NVS82T, NVS83T, NVS84T, NVS1b, NVS1c, NVS1d, NVS1e, NVS1f, NVS1g, NVS1h, NVS1j, mAb1 or mAb2.

The invention also relates to compositions comprising the peptide tag, for example a peptide tag having the sequence of SEQ ID NO: 32, SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207, or SEQ ID NO: 220. The invention further relates to peptide tagged molecules as described herein, specifically peptide tagged molecules comprising a peptide tag having the sequence of SEQ ID NO: 32, SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207, or SEQ ID NO: 220. In certain aspects the compositions described herein further comprise a pharmaceutically acceptable excipient, diluent or carrier. It is also contemplated that the compositions may be formulated for joint delivery (e.g., intra-articular). In certain aspects the compositions for joint delivery may comprise a peptide tag that binds HA with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 9.0 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain aspects the composition includes 12 mg or less of the peptide tagged molecule. In a further aspect, the composition is formulated to deliver 12 mg/joint or less of a peptide tagged molecule per dose. In certain aspects the compositions described herein comprise 6 mg/50 ul or less of a peptide tagged molecule. In certain aspects of the invention it is contemplated that the composition includes 12 mg or less of the peptide tag.

Another aspect of the invention provides for a nucleic acid molecule encoding a peptide tag comprising a sequence of SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207, or SEQ ID NO: 220. More specifically, the nucleic acid molecule may encode the peptide tag HA10.1, HA10.2, HA11, HA11.1, NVS-X, NVS-Y, NVS-AX, or NVS-AY, or NVS-Z. Further aspects of the invention provide for a nucleic acid molecule encoding peptide tagged molecule as described Tables 1, 2, 4, 4b, or 5. In certain aspects the nucleic acid molecule may encode NVS1, NVS2, NVS3, NVS36, NVS37, NVS70T, NVS71T, NVS72T, NVS73T, NVS74T, NVS75T, NVS76T, NVS77T, NVS78T, NVS80T, NVS81T, NVS82T, NVS83T, NVS84T, NVS1b, NVS1c, NVS1d, NVS1e, NVS1f, NVS1g, NVS1h, NVS1j, mAb1 or mAb2. In certain specific aspects the nucleic acid comprises the sequence SEQ ID NO: 10, 20, 22, 24, 26, 28, and/or 30.

The present invention relates to expression vectors comprising the nucleic acids described herein. More specifically, for example, the expression vectors may comprise nucleic acids as described in Tables 1 and 2. In certain aspects the invention further provide a host cell comprising one or more expression vectors as described herein, wherein the host cell may be used for the production of a peptide tag having a sequence of SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207, or SEQ ID NO: 220. Alternatively, a host cell comprising one or more expression vectors as described herein may be used for the production of a peptide tagged molecule as described in Tables 1, 2, 4, 4b, or 5. In certain aspects it is contemplated that the host cell is a mammalian cell.

It is contemplated that the host cells described herein are useful for producing the peptide tags and peptide tagged molecules of the invention. Thus, the invention further relates to a process for producing a peptide tag and/or a peptide tagged molecule as described herein, for example a peptide tag or peptide tagged molecule as described in Tables 1, 2, 4, 4b, or 5. It is contemplated that the process further includes a step of culturing the host cell under appropriate conditions for the production of a peptide tag or peptide tagged molecule, and further isolating the peptide tag or peptide tagged molecule.

The invention still further relates to compositions comprising the peptide tag or peptide tagged molecules described herein. It is also contemplated that the peptide tag, peptide tagged molecules and/or compositions may be useful for therapy, more specifically for joint therapy. In addition, the peptide tag, peptide tagged molecules and/or compositions may be useful for treating a condition or disorder associated with joint disease or injury in a subject. In certain aspects, the joint disease may be synovial joint disease (e.g., inflammatory arthritides, osteoarthritis). Inflammatory arthritides are inflammatory diseases affecting the synovial joints and related structures. Presentation may include monoarticular, oligoarticular or polyarticular involvement, and may be acute or chronic. In certain aspects, the disease associated with inflammatory arthritides may be inflammatory connective tissue disease (e.g., rheumatoid arthritis, symstemic lupus erythematosus), crystal induced inflammatory arthritis (e.g., gout, pseudo-gout), seronegative spondyloarthropathies (ankylosing spondylitis, psoriatic arthritis), or infectious arthritis (e.g., gonorrhea, tuberculosis, osteomyelitis). In certain aspects, the joint injury may be an acromioclavicular joint injury, an elbow joint injury, a pivot joint injury (e.g., atlanto-axial joint, proximal radioulnar joint, distal radioulnar joint), a condyloid joint injury, a saddle joint injury (e.g., carpometacarpal or trapeziometacarpal joint of thumb), a ball and socket joint injury (e.g., shoulder and hip joints), a knee joint injury, a hinge joint injury, or an interphalangeal joint injury.

In certain specific aspects of the invention compositions comprising a peptide tagged molecules comprising an anti-TNFα antibody or antigen binding fragment thereof may be useful for treating a TNFα-mediated disorder in a subject. In certain aspects, the TNFα-mediated disorder may be diseases affecting synovial joints, in particular the inflammatory arthritides and osteoarthritis. In certain specific aspects, the composition useful for treating TNFα mediated disorders comprises an anti-TNFα antibody or antigen binding fragment comprising heavy chain CDR1, 2, and 3 sequences of SEQ ID NOs: 108, 109 and 110, respectively and light chain CDR1, 2, and 3 sequences of SEQ ID NOs: 117, 118 and 119, respectively.

In certain specific aspects, the composition useful for treating TNFα mediated disorders comprises an anti-TNFα antibody or antigen binding fragment comprising heavy chain CDR1, 2, and 3 sequences of SEQ ID NOs: 208, 209 and 210, respectively and light chain CDR1, 2, and 3 sequences of SEQ ID NOs: 213, 214 and 215, respectively.

The invention also relates to a method of treating a condition or disorder associated with synovial joint diseases and injuries in a subject, wherein the method comprises administering to the subject a composition comprising the peptide tag and/or peptide tagged molecule described herein. In certain specific aspects the method comprises administering a composition comprising a peptide tag or peptide tagged molecule, wherein the peptide tag binds HA with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In certain specific aspects the peptide tag binds HA with a KD of less than or equal to 8.0 uM. In certain specific aspects the peptide tag binds HA with a KD of less than or equal to 7.2 uM. In certain specific aspects the peptide tag binds HA with a KD of less than or equal to 5.5 uM.

In certain aspects, the condition or disorder associated with synovial joint disease is rheumatoid arthritis, systemic lupus erythematosus, gout, pseudo-gout, ankylosing spondylitis, psoriatic arthritis, gonorrhea, tuberculosis, osteomyelitis, or osteoarthritis.

The invention further relates to a method of treating a condition or disorder associated with synovial joint disease or injury in a subject, wherein the method comprises administering to the subject a composition comprising a peptide tag and/or peptide tagged molecule as described herein. In certain specific aspects the method comprises administering a composition comprising a peptide tag or peptide tagged molecule, wherein the peptide tag binds HA with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain aspects, the condition or disorder associated with arthritis is rheumatoid arthritis, psoriatic arthritis, infectious arthritis, or osteoarthritis.

The invention further relates to a method of treating a TNFα-mediated disorder in a subject, wherein the method comprises the step of administering to the subject a composition comprising a peptide tag that binds HA with a KD of less than or equal to 9.0 uM linked to an anti-TNFα antibody or antigen binding fragment thereof. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain aspects the method relates to treating a TNFα-mediated disorder in the joint of a subject. The invention still further relates to a method of treating a TNFα-mediated disorder in a subject, wherein the method comprises the step of administering to the subject a composition comprising a peptide tag comprising a sequence of SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207 or SEQ ID NO: 220 linked to an anti-TNFα antibody or antigen binding fragment thereof. It is contemplated that the anti-TNFα antibody or antigen binding fragment thereof comprises heavy chain CDR1, 2, and 3 sequences of SEQ ID NOs: 108, 109 and 110, respectively and light chain CDR1, 2, and 3 sequences of SEQ ID NOs: 117, 118 and 119, respectively. The anti-TNFα antibody or antigen binding fragment thereof may also comprise heavy chain CDR1, 2, and 3 sequences of SEQ ID NOs: 208, 209 and 210, respectively and light chain CDR1, 2, and 3 sequences of SEQ ID NOs: 213, 214 and 215, respectively

In certain specific aspects, the TNFα-mediated disorder is rheumatoid arthritis, systemic lupus erythematosus, gout, pseudo-gout, ankylosing spondylitis, psoriatic arthritis, gonorrhea, tuberculosis, osteomyelitis or osteoarthritis.

The invention also relates to a method of increasing half-life, mean residence time, or terminal concentration of molecule in the joint or decreasing clearance of a molecule from the joint comprising the step of administering a composition comprising a peptide tagged molecule to the joint of the subject, wherein the peptide tag binds HA with a KD of less than or equal to 9.0 uM. For example, the peptide tag can bind HA with a Kd of less than or equal to 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 9.0 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM.

The invention also relates to methods of increasing the intra-articular half-life of a molecule comprising the step of linking the molecule to a peptide tag that binds HA with a KD of less than or equal to 9.0 uM. In certain aspects the invention relates to methods of increasing the intra-articular mean residence time of a molecule comprising the step of linking the molecule to a peptide tag that binds HA with a KD of less than or equal to 9.0 uM. In certain aspects the invention relates to methods of increasing the intra-articular terminal concentration of a molecule comprising the step of linking the molecule to a peptide tag that binds HA with a KD of less than or equal to 9.0 uM. In certain aspects the invention relates to methods of decreasing the intra-articular clearance of a molecule comprising the step of linking the molecule to a peptide tag that binds HA with a KD of less than or equal to 9.0 uM. In each of the foregoing methods, the peptide tag binds HA with a KD of less than or equal to 9.0 uM, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect, the peptide tag binds HA with a KD of less than or equal to 9.0 uM. In one aspect, the peptide tag binds HA with a KD of less than or equal to 8.0 uM. In one aspect, the peptide tag binds HA with a KD of less than or equal to 7.2 uM. In one aspect, the peptide tag binds HA with a KD of less than or equal to 5.5 uM. In one aspect, the peptide tag comprises the sequence of SEQ ID NO: 32, SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207, or SEQ ID NO: 220.

The invention further relates to a method of producing a composition for intra-articular delivery comprising the step of linking a peptide tag that binds HA with a KD of less than or equal to 9.0 uM to a molecule that binds a target in the joint. For example, the peptide tag can bind HA with a KD of less than or equal to 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. The invention still further relates to a method of making a peptide tagged molecule comprising a sequence of SEQ ID NO: 32, SEQ ID NO: 33, SEQ ID NO: 34, SEQ ID NO: 35, SEQ ID NO: 36, SEQ ID NO: 204, SEQ ID NO:205, SEQ ID NO:206 or SEQ ID NO:207 or SEQ ID NO: 220 is linked to a molecule, for example, a protein or nucleic acid. In certain aspects it is contemplated that linking the peptide tag to a molecule creates a peptide tagged molecule, that when administered to the joint, has a decreased intra-articular clearance, increased intra-articular mean residence time, and/or increased intra-articular terminal concentration compared to the molecule without the tag.

### Definitions

Unless defined otherwise, all technical and scientific terms used herein have the same meaning as commonly understood by those of ordinary skill in the art to which this invention pertains.

The term “synovial joint” as used herein refers to a joint between two bones that includes an articular capsule forming a synovial cavity typically containing synovial fluid (although it is contemplated that a joint having an articular capsule absent synovial fluid (e.g., where the fluid may have been removed surgically) is still considered a synovial joint). The term “intra-articular” or “intra-articular space” refers to the space (whether or not containing synovial fluid) confined by the articular capsule. Accordingly, an intra-articular administration of a molecule described herein means administration within the intra-articular space.

The term “antibody” as used herein means a whole antibody. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding fragment” of an antibody, as used herein, refers to one or more fragments of an antibody that retain the ability to specifically bind to a given antigen (e.g., tumor necrosis factor: TNF). Antigen binding functions of an antibody can be performed by fragments of an intact antibody. Examples of binding fragments encompassed within the term antigen binding fragment of an antibody include, but are not limited to, a Fab fragment, a monovalent fragment consisting of the VL, VH, CL and CH1 domains; a F(ab)₂ fragment, a bivalent fragment comprising two Fab fragments linked by a disulfide bridge at the hinge region; an Fd fragment consisting of the VH and CH1 domains; an Fv fragment consisting of the VL and VH domains of a single arm of an antibody (scFv); a single domain antibody (dAb) fragment (Ward et al., 1989 Nature 341:544-546), which consists of a VH domain or a VL domain; and an isolated complementarity determining region (CDR).

Furthermore, although the two domains of the Fv fragment, VL and VH, are coded for by separate genes, they can be joined, using recombinant methods, by an artificial peptide linker that enables them to be made as a single protein chain in which the VL and VH regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies may include one or more antigen binding fragments of an antibody. These antigen binding fragments are obtained using conventional techniques known to those of skill in the art, and the fragments are screened for utility in the same manner as are intact antibodies.

Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies).

Antigen binding fragments can be incorporated into single chain molecules comprising a pair of tandem Fv segments (VH-CH1-VH-CH1) which, together with complementary light chain polypeptides, form a pair of antigen binding regions (Zapata et al., 1995 Protein Eng. 8(10):1057-1062; and U.S. Pat. No. 5,641,870).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “complement C5 protein” or “C5” are used interchangeably, and refers to the complement component 5 protein in different species. For example, human C5 has the sequence as set in SEQ ID NO: 99 (see Table 2b). Human C5 is known in the art and can be obtained from Quidel (Cat. Number A403).

The term “conditions or disorders associated with joint disease or synovial joint disease” refers to any number of conditions or diseases in which the synovial joints are affected. This includes acute and chronic joint diseases, for example, inflammatory connective tissue diseases (e.g., rheumatoid arthritis, systemic lupus erythematosus); crystal induced inflammatory arthritis (e.g., gout, pseudo-gout); seronegative spondyloarthropathies (ankylosing spondylitis, psoriatic arthritis); and infectious arthritis (e.g., gonorrhea, tuberculosis, osteomyelitis).

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some embodiments, the term “conservative sequence modifications” or “conservative modifications” are used to refer to amino acid modifications that do not significantly affect or alter the binding characteristics of the antibody containing the amino acid sequence.

As used herein, the term “DARPin” (an acronym for designed ankyrin repeat proteins) refers to an antibody mimetic protein typically exhibiting highly specific and high-affinity target protein binding. They are typically genetically engineered and derived from natural ankyrin proteins and consist of at least three, usually four or five repeat motifs of these proteins. Their molecular mass is about 14 or 18 kDa (kilodaltons) for four- or five-repeat DARPins, respectively. Examples of DARPins can be found, for example in U.S. Pat. No. 7,417,130.

The term “dose” refers to the quantity of peptide tag, peptide tagged molecule, protein or nucleic acid administered to a subject all at one time (unit dose), or in two or more administrations over a defined time interval. For example, dose can refer to the quantity of protein (e.g., a peptide tagged molecule, for example, a peptide tagged protein comprising an anti-TNFα antigen binding fragment and a peptide tag the binds HA) administered to a subject over the course of three weeks or one, two, three or more months (e.g., by a single administration, or by two or more administrations). The interval between doses can be any desired amount of time and is referred to as the “dosing interval”. The term “pharmaceutically effective” when referring to a dose means sufficient amount of the protein (e.g.: antibody or antigen binding fragment), peptide tag or other pharmaceutically active agent to provide the desired effect. The amount that is “effective” will vary from subject to subject, depending on the age and general condition of the individual, the particular drug or pharmaceutically active agent and the like. Thus, it is not always possible to specify an exact “effective” amount applicable for all patients. However, an appropriate “effective” dose in any individual case may be determined by one of ordinary skill in the art using routine experimentation.

The terms “Epo protein” or “Epo antigen” or “EPO” or “Epo” are used interchangeably, and refer to the erythropoietin protein in different species. For example, human EPO has the sequence as set out in Table 2b: SEQ ID NO: 98. The protein sequences for human, cynomolgus, mouse, rat, and rabbit Epo are publicly available. Human EPO can also be hyperglycosylated.

The terms “Epo Receptor” or “EPOR” are used interchangeably, and refer to the erythropoietin receptor protein, and refer to the erythropoietin receptor protein in different species. EPOR has been described by Winkelmann J. C., Penny L. A., Deaven L. L., Forget B. G., Jenkins R. B. Blood 76:24-30(1990).

The term “Factor D protein” or “Factor D antigen” or “Factor D” are used interchangeably, and refers to the Factor D protein in different species. The sequence of Human Factor D has been described by Johnson et al. (FEBS Lett. 1984 Jan. 30; 166(2):347-51). Antibodies to Factor D are known in the art and described in U.S. Pat. No. 8,273,352.

The term “Factor P protein” or “Factor P antigen” or “Factor P” are used interchangeably, and refers to the Factor P protein in different species. For example, human Factor P has the sequence as set out in Table 2b: SEQ ID NO: 100. Human Factor P can be obtained from Complement Tech, Tyler, Tex. Cynomolgus Factor P can be purified from cynomolgus serum (protocol adapted from Nakano et al., (1986) J Immunol Methods 90:77-83). Factor P is also know in the art as “Properdin”.

The term “FGFR2” refers to fibroblast growth factor receptor 2 in different species. FGFR2 has been described by Dionne C. A., Crumley G. R., Bellot F., Kaplow J. M., Searfoss G., Ruta M., Burgess W. H., Jaye M., Schlessinger J. EMBO J. 9:2685-2692(1990).

The term “hyaluronan” or “hyaluronic acid” or “HA” refers a large polymeric glycosamine containing repeating disaccharide units of N-acetyl glucosamine and glucuronic acid that occurs in extracellular matrix and on cell surfaces. Hyaluronan, is further described in J. Necas, L. Bartosikova, P. Brauner, J. Kolar, Veterinarni Medicina, 53, 2008 (8): 397-411.

The term “hyaladherin” or “hyaluronan binding proteins” or “HA binding proteins” refers to a protein or a family of proteins that bind Hyaluronan. Examples of HA binding proteins are known in the art (Day, et al. 2002 J Bio. Chem 277:7, 4585 and Yang, et al. 1994, EMBO J 13:2, 286-296) (e.g.: Link, CD44, RHAMM, Aggrecan, Versican, bacterial HA synthase, collagen VI, and TSG-6). Many HA binding proteins, and peptide fragments, contain a common structural domain of ˜100 amino acids in length involved in HA binding; the structural domain is referred to as a “LINK Domain” (Yang, et al. 1994, EMBO J 13:2, 286-296 and Mahoney, et al. 2001, J Bio. Chem 276:25, 22764-22771). For example, the LINK Domain of TSG-6, an HA binding protein, includes amino acid residues 36-128 of the human TSG-6 sequence (SEQ ID NO: 30).

The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies of the invention may include amino acid residues not encoded by human sequences (e.g., mutations introduced by random or site-specific mutagenesis in vitro or by somatic mutation in vivo).

The term “human monoclonal antibody” refers to antibodies displaying a single binding specificity which have variable regions in which both the framework and CDR regions are derived from human sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994. Other examples of human engineering technology include, but are not limited to Xoma technology disclosed in U.S. Pat. No. 5,766,886.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (Ringbou ed., 2003)).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available on the world wide web at gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the two nucleic acid sequences.

The term “isolated antibody” refers to an antibody that is substantially free of other antibodies or other proteins having different antigenic specificities (e.g., an isolated antibody that specifically binds VEGF is substantially free of antibodies that specifically bind antigens other than VEGF). An isolated antibody that specifically binds VEGF may, however, have cross-reactivity to other antigens. Moreover, an isolated antibody may be substantially free of other cellular material and/or chemicals, for example, an antibody isolated from a cell supernatant.

The term “IL-1β” refers to refers to the Interleukin-1 beta protein a cytokine that is encoded in humans by the IL1B gene. For example, human IL-1β has the sequence as set out in Table 2b: SEQ ID NO: 102.

The terms “IL-10” or “IL10” are used interchangeably, and refer to the interleukin-10 protein, and refer to the interleukin-10 protein in different species. IL10 has been described by Vieira P., de Waal-Malefyt R., Dang M.-N., Johnson K. E., Kastelein R., Fiorentino D. F., Devries J. E., Roncarolo M.-G., Mosmann T. R., Moore K. W. Proc. Natl. Acad. Sci. U.S.A. 88:1172-1176(1991).

The term “IL-17A” refers to Interleukin 17A, is a 155-amino acid protein that is a disulfide-linked, homodimeric, secreted glycoprotein with a molecular mass of 35 kDa (Kolls J K, Lindén A 2004, Immunity 21:467-76).

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors.

The term “linked” or “linking” refers to the attachment of a peptide tag, such as, for example, the peptide tags that bind HA listed in Table 1 and 2, to a molecule, for example a protein or a nucleic acid. Attachment of the peptide tag to a protein or nucleic acid molecule, can occur, for example, at the amino or carboxy terminus of the molecule. The peptide tag can also be attached to both the amino and carboxy termini of the molecule. The peptide tag can also be attached to one or more amino acids or nucleic acids within the protein or nucleic acid molecule, respectively. In addition, “linked” can also refer to the association of two or more peptide tags to each other and/or the association of two or more peptide tags to distinct sites on a molecule. Linking of the peptide tag to a molecule may be accomplished by several methods know in the art, including, but not limited to, expression of the peptide tag(s) and molecule as a fusion protein, linkage of two or more peptide tags via a “peptide linker” between tags and/or molecule, or by chemically joining peptide tags to a molecule after translation, either directly to each other, or through a linker by disulfide bonds, etc.

The term “peptide linker” refers to an amino acid sequence that functions to covalently join the peptide tag to a molecule. The peptide linker may be covalently attached to one or both of the amino or carboxy termini of a peptide tag and/or a protein or nucleic acid molecule. The peptide linker may also be conjugated to an amino acid or nucleic acid within the sequence of a protein or nucleic acid molecule, respectively. It is contemplated that peptide linkers may be, for example, about 2 to 25 residues in length.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “clearance” refers to is the volume of a substance (e.g.: matrix, tissue, plasma, or other substance such as a drug or such as a peptide tagged molecule) cleared per unit time (Shargel, L and Yu, ABC: Applied Biopharmaceutics & Pharmacokinetics, 4^{th} Edition (1999)). “Intra-articular clearance” refers to clearance of a substance such as a peptide tagged molecule from the joint.

The term “operably linked” refers to a functional relationship between two or more polynucleotide (e.g., DNA) segments. Typically, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The term “PDGF-BB” refers to platelet-derived growth factor subunit B, this protein has been as described by Josephs S. F., Ratner L., Clarke M. F., Westin E. H., Reitz M. S., Wong-Staal F. Science 225:636-639(1984).

The term “peptide tag” or “protein tag”, are used interchangeably to refer to a short protein sequence, peptide fragment, or peptidomimetic, that binds molecules found in various synovial joint compartments including: synovial cavity, articular capsule, articular cartilage, articular discs or menisci, articular fat pads, tendons, accessory ligaments, or bursae. For example, the intra-articular molecules bound by the peptide tag may include extracellular matrix components, proteoglycans, collagen, elastin, fibronectin; and carbohydrate containing molecules including hyaluronic acid, glycosaminoglycans and other extracellular proteoglycans. Specific examples of peptide tags include, for example, peptide tags that bind HA (i.e.: HA-binding peptide tags). Peptide tags of the invention, including peptide tags that bind HA may increase intra-articular half-life (T_{1/2} or t_{1/2}), and/or increase mean intra-articular mean residence time, and/or decrease intra-articular clearance rate, and/or increase the dosing interval of a peptide tagged molecule (e.g.: protein or nucleic acid) as compared to the same molecule not linked to a peptide tag, (i.e.: an untagged molecule).

Peptide tags can be linked to form a multimer by several methods known in the art, including, but not limited to, expression of the protein tags as a fusion protein, linkage of two or more protein tags via a peptide linker between tags, or by chemically joining peptide tags after translation, either directly to each other, or through a linker by disulfide bonds, etc. The term “peptide tagged molecule” refers to a molecule that is linked to one or more peptide tags of the invention. The molecule may be, but is not limited to, a protein or nucleic acid. The term “tagged antibody” or “peptide tagged antibody” refers to an antibody, or antigen binding fragment thereof, that is linked to one or more protein tags of the invention. The term “peptide tagged antigen binding fragment” refers to an antigen binding fragment that is linked to one or more protein tags of the invention.

The term “half-life”, as used herein, refers to the time required for the concentration of a drug to fall by one-half (Rowland M and Towzer T N: Clinical Pharmacokinetics. Concepts and Applications. Third edition (1995) and Bonate P L and Howard D R (Eds): Pharmacokinetics in Drug Development, Volume 1 (2004)).

As used herein, the term “mean residence time” or “MRT” is the average time that the drug (e.g.: a peptide tagged molecule) resides in the body, including in a specific organ or tissue (e.g., synovial joint).

As used herein, the term “Ctrough” refers to the lowest concentration of drug measured in a matrix or tissue throughout the dosing interval, most often occurring immediately prior to repeat dose administration.

As used herein, the term “protein” refers to any organic compounds made of amino acids arranged in one or more linear chains and folded into a globular form. The amino acids in a polymer chain are joined together by the peptide bonds between the carboxyl and amino groups of adjacent amino acid residues. The term “protein” further includes, without limitation, peptides, single chain polypeptide or any complex molecules consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation and the like, incorporation of non-natural amino acids, and amino acid modification for chemical conjugation with another molecule.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates (e.g.: mammals and non-mammals) such as, non-human primates (e.g.: cynomolgus monkey), sheep, dog, cow, chickens, amphibians, and reptiles. Except when noted, the terms “patient” or “subject” are used herein interchangeably. As used herein, the terms “cyno” or “cynomolgus” refer to the cynomolgus monkey (Macaca fascicularis).

The term “terminal concentration” refers to the concentration of the peptide tag, peptide tagged molecule, etc. that is measured at the end of the experiment or study. An “increase in terminal drug concentration” refers to an at least 25% increase in terminal concentration of the peptide tagged molecule.

As used herein, the term “treating” or “treatment” of any conditions or disorders associated with synovial joint diseases, e.g., osteoarthritis, conditions or disorders associated with inflammatory connective tissue diseases, conditions or disorders associated with crystal induced inflammatory arthritis, conditions or disorders associated with seronegative spondyloarthropathies and/or conditions or disorders associated with infectious arthritis refers in one aspect, to ameliorating the disease or disorder (i.e., slowing or arresting or reducing the development of the disease or at least one of the clinical symptoms thereof). In another aspect “treating” or “treatment” refers to alleviating or ameliorating at least one physical parameter including those which may not be discernible by the patient. In yet another aspect, “treating” or “treatment” refers to modulating the disease or disorder, either physically, (e.g., stabilization of a discernible symptom), physiologically, (e.g., stabilization of a physical parameter), or both. In yet another aspect, “treating” or “treatment” refers to preventing or delaying the onset or development or progression of the disease or disorder. “Prevention” as it relates to indications described herein, including, conditions or disorders associated with osteoarthritis, conditions or disorders associated with inflammatory connective tissue diseases, conditions or disorders associated with crystal induced inflammatory arthritis, and/or conditions or disorders associated with seronegative spondyloarthropathies, and/or conditions or disorders associated with infectious arthritis means any action that prevents or slows a worsening in joint function, joint anatomy, osteoarthritis parameter, inflammatory connective tissue disease parameter, crystal induced inflammatory arthritis parameter, seronegative spondyloarthropathies parameter and/or infectious arthritis parameter, as described below, in a patient at risk for said worsening. More specifically, “treatment” of conditions or disorders associated with osteoarthritis, conditions or disorders associated with inflammatory connective tissue diseases, conditions or disorders associated with crystal induced inflammatory arthritis, seronegative spondyloarthropathies, and/or conditions or disorders associated with infectious arthritis means any action that results in, or is contemplated to result in, the improvement or preservation of joint function and/or joint anatomy. Methods for assessing treatment and/or prevention of disease are known in the art and described herein below.

The term “TNFα” refers to tumor necrosis factor alpha (also known as, cachectin), a naturally occurring mammalian cytokine produced by numerous cell types, including monocytes and macrophages in response to endotoxin or other stimuli. TNFα is a major mediator of inflammatory, immunological, and pathophysiological reactions (Grell, M., et al. (1995) Cell, 83: 793-802). Soluble TNFα is formed by the cleavage of a precursor transmembrane protein (Kriegler, et al. (1988) Cell 53: 45-53), and the secreted 17 kDa polypeptides assemble to soluble homotrimer complexes (Smith, et al. (1987), J. Biol. Chem. 262: 6951-6954; for reviews of TNFα, see Butler, et al. (1986), Nature 320:584; Old (1986), Science 230: 630). The sequence for human TNFα is described in Table 1 and has the sequence of SEQ ID NO: 101.

The term “TSG-6” refers to Tumor Necrosis Factor-Inducible Gene 6. TSG-6 is a member of an HA binding protein family and contains a LINK Domain. (Lee et al. J Cell Bio (1992) 116:2, 545-57). The LINK Domain from TSG-6 is also referred to herein as the “TSG-6 LINK Domain”.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “VEGF” refers to the 165-amino acid vascular endothelial cell growth factor, and related 121-, 189-, and 206-amino acid vascular endothelial cell growth factors, as described by Leung et al., Science 246:1306 (1989), and Houck et al., Mol. Endocrin. 5:1806 (1991) together with the naturally occurring allelic and processed forms of those growth factors. The sequence for human VEGF is described in Table 2b and has a sequence of SEQ ID NO: 97.

The term “VEGF-mediated disorder” refers to any disorder, the onset, progression or the persistence of the symptoms or disease states of which requires the participation of VEGF. Exemplary VEGF-mediated disorders include, but are not limited to, age-related macular degeneration, neovascular glaucoma, diabetic retinopathy, macular edema, diabetic macular edema, pathologic myopia, retinal vein occlusions, retinopathy of prematurity, abnormal vascular proliferation associated with phakomatoses, edema (such as that associated with brain tumors), Meigs' syndrome, rheumatoid arthritis, psoriasis and atherosclerosis.

As used herein, the term “therapeutic protein” refers to a protein that is useful to treat, prevent or ameliorate a disease, condition or disorder.

As used herein, the term “protein receptor” refers to a protein that is a cellular receptor and binds a ligand.

### BRIEF DESCRIPTION OF THE DRAWINGS

FIG. 1. shows the terminal concentrations in synovial fluid (1A), synovial tissue (1B), and cartilage (1C) 2, 6, and 24 hrs post-intra-articular injections of a Fab fused with an HA binding peptide tag (NVS73T) and its unmodified version (NVS73) as measured by mass spectrometry. The terminal concentrations of the Fab fused with an HA binding peptide tag are significantly higher than the unmodified Fab at every time point and in every tissue/fluid analyzed. Plotted based on data calculated from standard curves that were plotted in ng of drug vs mass spectrometric signal.

FIG. 2. shows the effect of an intra-articular injection of 3, 10 or 30 ug rhTNFα on knee swelling in the rat between 1 and 24 hours. The results are expressed as the ratio of the left knee diameter in mm/right knee diameter in mm. Data are the mean±sem of n=5 rats per group.

FIG. 3. illustrates the differences in the knee swelling ratios in rat knees injected with 30 ug rhTNFα which were previously injected with either an anti-TNFα Fab (NVS73) or a hyaluronic acid binding peptide tagged anti-TNFα Fab (NVS73T). The results are expressed as the ratio of the left knee diameter in mm/right knee diameter in mm. Columns represent the mean±sem of n=5 rats. *p<0.05 and **p<0.01 ANOVA, followed by Dunnett's test for multiple comparisons. NS—not significant vs control rats.

FIG. 4. shows the terminal concentrations in rat cartilage 2, 6, and 24 hrs post-intra-articular injections of a Fab fused with an HA binding peptide tag (NVS73T) and its unmodified version (NVS73). The terminal concentrations of the Fab fused with an HA binding peptide tag are significantly higher than the unmodified Fab at every time point analyzed.

FIG. 5. illustrates the percentage inhibition in the knee swelling in rat knees injected with 30 μg rhTNFα in animals previously receiving either adalimumab (Humira®) or hyaluronic acid tagged adalimumab on the heavy or light chain. The results are expressed as the percentage inhibition of the area under the curve (AUC) vs the swelling response in vehicle (PBS injected rats). Columns represent the mean±sem of n=5 rats. *p<0.05 and **p<0.01 ANOVA, followed by Dunnett's test for multiple comparisons. NS—not significant vs control rats.

FIG. 6. shows the terminal concentrations in synovial fluid (6A), synovial tissue (6B), and cartilage (6C) 2, 6, and 24 hrs post-intra-articular injections of a Fab fused with an HA binding peptide tag (NVS73T) and its unmodified version (NVS73) as measured by mass spectrometry. The terminal concentrations of the Fab fused with an HA binding peptide tag are significantly higher than the unmodified Fab at every time point and in every tissue/fluid analyzed. Plotted based on data calculated from standard curves that were plotted in ng/ml of drug vs mass spectrometric signal.

### DETAILED DESCRIPTION

The present invention is based, in part, on the discovery of peptide tags that increase the half-life and/or mean residence time of proteins or nucleic acids in the synovial joints. In certain aspects the invention peptide tags increase the half-life and/or mean residence time of antibodies and antigen binding fragments, therapeutic proteins, protein receptors, DARPins and/or aptamers in the synovial joints. The invention also relates to the discovery of long acting antibody molecules that specifically bind intra-articular proteins (e.g.: HA and/or TNFα) and exhibit an increased half-life and/or mean residence time in the synovial joints. The invention relates to both full IgG format antibodies as well as antigen binding fragments, such as Fab fragments, linked to a protein tag.

### Peptide Tags

Many factors may affect a protein's half-life in vivo. For example, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the serum half-life of antibodies, antigen binding fragments, or antibody mimetics. For example, by attaching polysialic acid (PSA), hydroxyethyl starch (HES), albumin-binding ligands, and carbohydrate shields; by genetic fusion to proteins binding to serum proteins, such as albumin, IgG, FcRn, and transferrin; by coupling (genetically or chemically) to other binding moieties that bind to serum proteins, such as nanobodies, Fabs, DARPins, avimers, affibodies, and anticalins; by genetic fusion to albumin or a domain of albumin, albumin-binding proteins, an antibody Fc region; or by incorporation into nanocarriers, slow release formulations, or medical devices.

The present invention provides peptide tags that specifically bind hyaluronan in the synovial joints. Hyaluronan is present in the body in various sizes in many organs in tissues. For example, the synovial fluid and the human eye contain the highest concentrations of hyaluronan concentrations with 0.14-0.338 mg/ml and 1.42-3.6 mg/ml respectively, while other tissues/fluids contain much lower concentrations of hyaluronan such as serum in which hyaluronan concentrations are 0.00001-0.0001 mg/ml (Laurent and Fraser, 1986 Ciba Found Symp. 1986; 124:9-29.).

The present invention is based on the surprising discovery of peptide tags that bind HA in the synovial joints and are suitable for extending the half-life of a protein or nucleic acid in the synovial joints, increasing the terminal concentration of a protein or nucleic acid in the synovial joints, decreasing the intra-articular clearance of a protein or nucleic acid in the synovial joints, and/or increasing mean residence time of a protein or nucleic acid in the synovial joints. In certain aspects of the invention the peptide tag binds HA in the synovial joint with a KD of less than or equal to 9.0 uM, less than or equal to 8.5 uM, less than or equal to 8.0 uM, less than or equal to 7.5 uM, less than or equal to 7.0 uM, less than or equal to 6.5 uM, less than or equal to 6.0 uM, less than or equal to 5.5 uM, less than or equal to 5.0 uM, less than or equal to 4.5 uM, less than or equal to 4.0 uM, less than or equal to 3.5 uM, less than or equal to 3.0 uM, less than or equal to 2.5 uM, less than or equal to 2.0 uM, less than or equal to 1.5 uM, less than or equal to 1.0 uM, less than or equal to 0.5 uM, or less than or equal to 100 nM. In more specific aspects, for example, the peptide tag binds HA in the synovial joints with a KD of less than or equal to 8.0 uM, less than or equal to 7.2 uM, less than or equal to 6.0 uM, or less than or equal to 5.5 uM. In some aspects of the invention the peptide tag that binds HA has a LINK domain. In certain other aspects of the invention the LINK domain is a TSG-6 LINK domain. Still other aspects of the invention are based on the discovery of modified versions of the peptide tag that also resist proteolytic cleavage and/or glycosylation. More specifically the invention may include a peptide tag that binds, or is capable of binding, HA comprising a sequence of SEQ ID NO: 32, 33, 34, 35, 36, SEQ ID NO: 204, SEQ ID NO:205, SEQ ID NO:206 or SEQ ID NO:207. It is contemplated that the peptide tag comprising a sequence of SEQ ID NO: 32, 33, 34, 35, 36, 204, 205, 206, or 207 binds, or is capable of binding, HA in the synovial joint of a subject. It is contemplated that the peptide tag may be any one of the peptide tags listed in Table 1. More specifically, the peptide tag may be HA10, HA10.1, HA10.2, HA11, HA11.1, NVS-X, NVS-Y, NVS-AX, NVS-AY.

In certain aspects, the peptide tag can have a sequence comprising 30, 35, 40, 45, 50, 55, 60, 65, 70, 75, 80, 85, 90, 91, 92, 93, 94, 95, 96, 97 or 98 consecutive amino acids of SEQ ID NOs: 32, 33, 34, 35, 36, 204, 205, 206, or 207. In certain other aspects, it is contemplated that a peptide tag is a truncated variant of a peptide tag comprising a sequence of SEQ ID NO: 32, 33, 34, 35, 36, 204, 205, 206, or 207. Amino acids may be cleaved from the N-terminus, C-terminus or both of the peptide tag comprising a sequence of SEQ ID NO: 32, 33, 34, 35, 36, 204, 205, 206, or 207 to produce a truncated variant of the peptide tags HA10, HA10.1, HA10.2, HA11, HA11.1, NVS-X, NVS-Y, NVS-AX, or NVS-AY. It is further contemplated that the sequence may cleaved from the N-terminus of SEQ ID NO: 32, 33, 34, 35, 36, 204, 205, 206, or 207 and up to (but not including) the first N-terminal cysteine. It is further contemplated that the sequence may cleaved from the C-terminus of SEQ ID NO: 32, 33, 34, 35, 36, 204, 205, 206, or 207 and up to (but not including) the first C-terminal cysteine. It is further contemplated that the sequence may cleaved from both the N-terminus and the C-terminus of SEQ ID NO: 32, 33, 34, 35, 36, 204, 205, 206, or 207 and up to (but not including) the first N-terminal cysteine and up to (but not including) the first C-terminal cysteine. For example, with respect to SEQ ID NO: 32, one of skill in the art could remove up to 22 amino acids from the N-terminal end (bold) and/or up to six amino acids from the C-terminal end (underline):

(SEQ ID NO: 32)
GVYHREARSGKYKLTYAEAKAVCEFEGGHLATYKQLEAARKIGFHVCAAG
WMAKGRVGYPIVKPGPNCGFGKTGIIDYGIRLNRSERWDAYCYNPHAK

The peptide tag of the invention can be linked to a molecule to extend the intra-articular half-life of the molecule, for example the molecule may be a protein or nucleic acid. Specific examples of proteins and nucleic acids that can be modified by the protein tags described herein include, but are not limited to, antibodies, antigen binding fragments, therapeutic proteins, protein receptors, DARPins, and/or aptamers, as well as multivalent combinations proteins and nucleic acids. In certain aspects, these proteins and nucleic acids bind a target protein in the synovial joint, for example, TNFα, VEGF, C5, Factor P, Factor D, EPO, EPOR, IL-1β, IL-17A, IL-6, IL-10, IL-18, IL-8, bFGF, MCP-1, FGFR2, CD132, IL6R, CD20, IGF-1 and/or PDGF (including PDGF-BB). Without being bound to any particular theory, the peptide tags of the invention, when linked to a protein or nucleic acid that binds a target protein in the synovial joint, decrease intra-articular clearance, increase the mean residence time, increase half-life (T_{1/2}), and/or increase terminal drug concentration of the tagged molecule (e.g.: protein or nucleic acid) in the synovial joint relative to the untagged molecule.

The invention also relates to the surprising finding that linking a peptide tag that binds, or is capable of binding HA in the synovial joint to a molecule (e.g.: a protein or nucleic acid) significantly improves the biophysical properties of the peptide tagged molecule compared to the molecule without the tag. It is contemplated the biophysical properties of the peptide tagged molecule improve a statistically significant amount (i.e.: p<0.05) compared to the molecule without a peptide tag, including, but not limited to improved solubility, improved isoelectric point (pI) and/or improved binding affinity of the peptide tagged molecule to its target relative to an untagged version of the molecule. In specific aspects the invention relates to a method of increasing the solubility of a molecule comprising the step of linking the molecule to a peptide tag that binds HA in the synovial joint. In specific aspects the invention relates to a method of increasing the pI of a molecule comprising the step of linking the molecule to a peptide tag that binds HA in the synovial joint. In certain aspects the linking a peptide tag to a molecule increases the pI up to 3 fold compared to the untagged molecule. In other aspects the pI of a peptide tagged molecule increases up to 2.8, 2.5, 2.0, 1.75, 1.5, 1.0, or 0.5 fold as compared to the untagged molecule.

In specific aspects the invention relates to a method of increasing the binding affinity of a molecule to its target comprising the step of linking the molecule to a peptide tag that binds HA in the synovial joint. In certain specific aspects the linking a peptide tag to a molecule improves the binding affinity of the molecule for the primary target by 135 fold, 130 fold, 120 fold, 110 fold, 100 fold, 90 fold, 80 fold, 75 fold, 50 fold, 40 fold, 30 fold, 20 fold, 15 fold 10 fold, 7.5 fold, 5 fold, 4 fold, 2 fold, 1.75 fold. It is contemplated that the peptide tagged molecule binds HA in the synovial joint with a KD of less than or equal to 9.0 uM, 8.0 uM, 6.0 uM, or 5.5 uM. It is further contemplated that the peptide tag comprising a sequence of SEQ ID NO: 32, 33, 34, 35, 36, 204, 205, 206 or 207 improves the biophysical properties of a molecule to which it is linked by a statistically significant amount when compared to the molecule without the tag. It is still further contemplates that multiple peptide tags may be used in any of the methods described herein to improve the binding affinity for HA in the synovial joint, more specifically for example a peptide tagged molecule comprising more than one peptide tag binds HA with a KD of less than or equal to 1.0 uM, 0.9 uM, 0.8 uM, 0.7 uM, 0.6 uM, 0.5 uM, 0.4 uM, 0.3 uM, 0.2 uM, or 0.1 uM.

In certain aspects of the invention it is contemplated that a single peptide tag is linked to a molecule, for example a protein or nucleic acid molecule. In other aspects of the invention it is contemplated that two, three, four or more peptide tags may be linked to the protein or nucleic acid. It is contemplated that the peptide tag is linked either to the carboxy-terminus or the amino-terminus of the protein. It is also contemplated that the peptide tag may be linked to the heavy chain or light chain of an antibody, or antigen binding fragment thereof, or alternatively linked to both chains. It is contemplated that the peptide tag may be linked to the 5′ and/or 3′ of the nucleic acid molecule. Multiple tags may be concatenated and/or linked to multiple protein chains (e.g.: linked to heavy and light chains). It is also contemplated that the protein tags and/or proteins and/or nucleic acids may be chemically joined after translation, either directly to each other, or through disulfide bond linkage, peptide linkers, etc. Peptide linkers and methods of linking protein tags to proteins (e.g.: antibodies and antigen binding fragments) or nucleic acids are known in the art and described herein.

### Peptide Tagged Molecules

Another aspect of the invention includes peptide tagged molecules. In certain aspects of the invention, the peptide tagged molecules may comprise a peptide tag that binds, or is capable of binding, HA. In certain aspects the peptide tagged molecule comprises a peptide tag that binds HA in the synovial joint with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. In certain specific aspects, the peptide tag may comprise a sequence of SEQ ID NO: 32, 33, 34, 35, 36, 204, 205, 206, or 207. It is also contemplated that the peptide tag is linked to a molecule that is a protein or a molecule that is a nucleic acid. Examples of molecules that can be linked to protein tags are described herein.

### Protein Molecules

The present invention provides proteins that can be linked to peptide tags of the invention. In certain aspects of the invention the protein may be an isolated antibody, or antigen binding fragment thereof (e.g.: Fab, scFv, Fc Trap, etc.), a protein that is a therapeutic protein (e.g. EPO, Insulin, cytokines, etc.), a protein receptor (e.g.: EPO receptor, FGFR2, etc), or DARPins. In certain aspects of the invention the protein binds, or is capable of binding, TNFα, VEGF, C5, Factor P, Factor D, EPO, EPOR, IL-1β, IL-17A, IL-6, IL-18, IL-8, bFGF, MCP-1, FGFR2, CD132, IL6R, CD20, IGF-1, and/or PDGF (including PDGF-BB). It is further contemplated that the protein binding occurs in the synovial joint.

One aspect of the invention includes proteins that bind TNFα. Numerous TNF binding proteins are known in the art and described herein, see for example Table 1. In certain aspects, the anti-TNFα binding proteins may have the sequences of NVS73. In certain specific aspects, for example, the invention also provides antibodies and antigen binding fragments that specifically bind TNFα. TNFα antibodies and antigen binding fragments of the invention include, but are not limited to the antibodies and fragments, isolated and described in Table 1 and the examples. Other anti-TNFα antibodies, TNFα antagonists, and TNFα receptor antagonists that can be linked to the protein tags described herein and used in the methods described herein include, for example: infliximab (Remicade®), entanercept (Embrel®), golimumab (Simponi®), and adalimumab (Humira®) and anti-TNFα antibodies and fragments as described in 20130230886.

A particular aspect of the invention provides antibodies that specifically bind a TNFα protein, wherein the antibodies comprise a VH domain comprising an amino acid sequence of SEQ ID NO: 111, or SEQ ID NO: 211. The present invention also provides antibodies that specifically bind a TNFα protein wherein the antibodies, antigen binding fragments comprise a heavy chain having an amino acid sequence of SEQ ID NO: 113. The present invention also provides antibodies that specifically bind a TNFα protein wherein the antibodies, antigen binding fragments having a peptide tagged heavy chain comprising an amino acid sequence of SEQ ID NO: 113 or 115, or SEQ ID NO: 212 or 218. The present invention also provides antibodies that specifically bind to a TNFα protein (e.g., human, cynomolgus, rat and/or mouse TNFα), wherein the antibodies comprise a VH CDR having an amino acid sequence of any one of the VH CDRs listed in Table 1, infra. In particular, the invention provides antibodies that specifically bind to a TNFα protein, wherein the antibodies comprise (or alternatively, consist of) one, two, three, or more VH CDRs having an amino acid sequence of any of the VH CDRs listed in Table 1, infra.

The present invention provides antibodies that specifically bind to a TNFα protein, said antibodies comprising a VL domain having an amino acid sequence of SEQ ID NO:120, or SEQ ID NO: 216. The present invention also provides antibodies that specifically bind a TNFα protein wherein the antibodies, antigen binding fragments comprise a light chain having an amino acid sequence of SEQ ID NO: 122, or SEQ ID NO: 217 or 219. The present invention also provides antibodies that specifically bind to a TNFα protein, said antibodies comprising a VL CDR having an amino acid sequence of any one of the VL CDRs listed in Table 1, infra. In particular, the invention provides antibodies that specifically bind to a TNFα protein, said antibodies comprising (or alternatively, consisting of) one, two, three or more VL CDRs having an amino acid sequence of any of the VL CDRs listed in Table 1, infra.

Alternate aspects of the invention provide additional proteins that can be linked to the peptide tags described herein. In certain aspects, the protein is an antibody or antigen binding fragment that binds VEGF (e.g., Ranibizumab), C5 (e.g., Eculizumab), Factor P, Factor D, EPO, EPOR, IL-1β (e.g., Gevokizumab), IL-17A (e.g., Ixekizumab), IL-6 (e.g., Siltuximab), IL-18, IL-8, bFGF, MCP-1 (e.g., Carlumab), FGFR2, CD132, IL-6R (e.g., Atlizumab, tocilizumab), CD20 (e.g., Ocrelizumab), IGF-1, and/or PDGF (including PDGF-BB). In certain aspects the protein may be a therapeutic protein such as erythropoietin, Insulin, human growth factor, interleukin-10, complement factor H, CD35, CD46, CD55, CD59, complement factor I, complement receptor 1-related (CRRY), nerve growth factor, angiostatin, pigment epithelium-derived factor, endostatin, ciliary neurotrophic factor, complement factor 1 inhibitor, complement factor like-1, complement factor I or the like. In other aspects, the protein may be a receptor such as EPOR. Additional examples of proteins that can be linked to peptide tags are provided in Table 2, 4 and 4b. More specifically, the proteins may be NVS70, NVS71, NVS72, NVS74, NVS75, NVS76, NVS77, NVS78 or NVS90.

Other proteins of the invention include amino acids that have been mutated, yet have at least 60, 70, 80, 85, 90, 95, 96, 97, 98 or 99 percent identity to the sequences described in Table 1, 2, 4b or 5b. In some embodiments, it includes mutant amino acid sequences wherein no more than 1, 2, 3, 4 or 5 amino acids have been mutated in the sequence described in Table 1, 2, 4b or 5.

The present invention also provides nucleic acid sequences that encode the protein molecules described herein. Such nucleic acid sequences can be optimized for expression in mammalian cells.

### Nucleic Acid Molecules

The present invention provides nucleic acids that can be linked to peptide tags of the invention. In certain aspects the nucleic acid that is linked to a peptide tag may be an mRNA or an RNAi agent, a ribozyme or an antisense oligonucleotide. More specifically, RNAi agents linked to the peptide tag may be an siRNA, shRNA, microRNA (i.e.: miRNA), anti-microRNA oligonucleotide, aptamer, or the like. In certain specific aspects, the nucleic acid molecule may be an aptamer. In particular, the aptamer may bind PDGF-BB. More specifically, the nucleic acid may be TNFα.

TABLE 1
Examples of peptide tagged anti-TNFa molecules and component sequences:
including, the untagged anti-TNFa molecule (NVS73), linkers and peptide tags.

| NVS73 and NVS73T | | SEQUENCE (OR SEQ ID NO) |
|---|---|---|
| SEQ ID NO: 108 | HCDR1 | GFTISRSYWIC |
| SEQ ID NO: 109 | HCDR2 | CIYGDNDITPLYANWAKG |
| SEQ ID NO: 110 | HCDR3 | LGYADYAYDL |
| SEQ ID NO: 111 | VH | EVQLVESGGGSVQPGGSLRLSCTASGFTISRSYWICWVRQA |
| | | PGKGLEWVGCIYGDNDITPLYANWAKGRFTISRDTSKNTVY |
| | | LQMNSLRAEDTATYYCARLGYADYAYDLWGQGTTVTVSS |
| SEQ ID NO: 112 | DNA of VH | GAGGTCCAGCTGGTGGAGAGCGGAGGAGGAAGCGTCCA |
| | SEQ ID NO: 111 | GCCTGGAGGCAGCCTGAGACTGAGCTGCACCGCCAGCGG |
| | | CTTCACCATCAGCAGGAGCTACTGGATCTGCTGGGTGAGG |
| | | CAGGCTCCTGGCAAGGGACTCGAGTGGGTGGGCTGCATC |
| | | TACGGCGACAACGACATCACCCCCCTCTACGCCAACTGGG |
| | | CTAAGGGCAGGTTCACCATTAGCAGGGACACCAGCAAGA |
| | | ACACCGTGTACCTCCAGATGAACAGCCTGAGGGCCGAGG |
| | | ATACCGCCACCTACTATTGCGCCAGGCTGGGCTACGCCGA |
| | | TTACGCCTATGACCTCTGGGGCCAGGGCACCACAGTGACC |
| | | GTCAGCTCA |
| SEQ ID NO: 113 | Heavy Chain | EVQLVESGGGSVQPGGSLRLSCTASGFTISRSYWICWVRQA |
| | | PGKGLEWVGCIYGDNDITPLYANWAKGRFTISRDTSKNTVY |
| | | LQMNSLRAEDTATYYCARLGYADYAYDLWGQGTTVTVSSA |
| | | STKGPSVFPLAPSSKSTSGGTAALGCLVKDYFPEPVTVSWNS |
| | | GALTSGVHTFPAVLQSSGLYSLSSVVTVPSSSLGTQTYICNVN |
| | | HKPSNTKVDKRVEPKSC |
| SEQ ID NO: 114 | DNA of Heavy | GAGGTCCAGCTGGTGGAGAGCGGAGGAGGAAGCGTCCA |
| | Chain SEQ ID | GCCTGGAGGCAGCCTGAGACTGAGCTGCACCGCCAGCGG |
| | NO: 113 | CTTCACCATCAGCAGGAGCTACTGGATCTGCTGGGTGAGG |
| | | CAGGCTCCTGGCAAGGGACTCGAGTGGGTGGGCTGCATC |
| | | TACGGCGACAACGACATCACCCCCCTCTACGCCAACTGGG |
| | | CTAAGGGCAGGTTCACCATTAGCAGGGACACCAGCAAGA |
| | | ACACCGTGTACCTCCAGATGAACAGCCTGAGGGCCGAGG |
| | | ATACCGCCACCTACTATTGCGCCAGGCTGGGCTACGCCGA |
| | | TTACGCCTATGACCTCTGGGGCCAGGGCACCACAGTGACC |
| | | GTCAGCTCAGCCTCCACCAAGGGACCTTCCGTGTTCCCCCT |
| | | GGCCCCTAGCTCCAAGTCCACCAGCGGAGGAACAGCCGCT |
| | | CTGGGCTGTCTGGTGAAGGACTACTTCCCCGAGCCTGTGA |
| | | CCGTGTCCTGGAATTCCGGCGCCCTCACAAGCGGAGTGCA |
| | | TACCTTCCCCGCCGTGCTGCAAAGCTCCGGACTGTACTCCC |
| | | TCTCCAGCGTGGTGACAGTGCCTTCCAGCAGCCTCGGCAC |
| | | CCAGACCTACATCTGCAACGTGAACCACAAGCCCTCCAAT |
| | | ACCAAGGTGGACAAGAGGGTCGAGCCTAAAAGCTGT |
| SEQ ID NO : 115 | Heavy Chain + | EVQLVESGGGSVQPGGSLRLSCTASGFTISRSYWICWVRQA |
| | Linker + | PGKGLEWVGCIYGDNDITPLYANWAKGRFTISRDTSKNTVY |
| | protein tag | LQMNSLRAEDTATYYCARLGYADYAYDLWGQGTTVTVSSA |
| | (SEQ ID NO: | STKGPSVFPLAPSSKSTSGGTAALGCLVKDYFPEPVTVSWNS |
| | 113 + SEQ ID | GALTSGVHTFPAVLQSSGLYSLSSVVTVPSSSLGTQTYICNVN |
| | NO: 31 + SEQ | HKPSNTKVDKRVEPKSCGSGGGGVYHREAQSGKYKLTYAEA |
| | ID NO: 33) | KAVCEFEGGHLATYKQLEAARKIGFHVCAAGWMAKGRVGY |
| | | PIVKPGPNCGFGKTGIIDYGIRLNRSERWDAYCYNPHA |
| SEQ ID NO: 116 | DNA of Heavy | GAGGTCCAGCTGGTGGAGAGCGGAGGAGGAAGCGTCCA |
| | Chain + Linker + | GCCTGGAGGCAGCCTGAGACTGAGCTGCACCGCCAGCGG |
| | protein tag | CTTCACCATCAGCAGGAGCTACTGGATCTGCTGGGTGAGG |
| | SEQ ID NO: | CAGGCTCCTGGCAAGGGACTCGAGTGGGTGGGCTGCATC |
| | 115 | TACGGCGACAACGACATCACCCCCCTCTACGCCAACTGGG |
| | | CTAAGGGCAGGTTCACCATTAGCAGGGACACCAGCAAGA |
| | | ACACCGTGTACCTCCAGATGAACAGCCTGAGGGCCGAGG |
| | | ATACCGCCACCTACTATTGCGCCAGGCTGGGCTACGCCGA |
| | | TTACGCCTATGACCTCTGGGGCCAGGGCACCACAGTGACC |
| | | GTCAGCTCAGCCTCCACCAAGGGACCTTCCGTGTTCCCCCT |
| | | GGCCCCTAGCTCCAAGTCCACCAGCGGAGGAACAGCCGCT |
| | | CTGGGCTGTCTGGTGAAGGACTACTTCCCCGAGCCTGTGA |
| | | CCGTGTCCTGGAATTCCGGCGCCCTCACAAGCGGAGTGCA |
| | | TACCTTCCCCGCCGTGCTGCAAAGCTCCGGACTGTACTCCC |
| | | TCTCCAGCGTGGTGACAGTGCCTTCCAGCAGCCTCGGCAC |
| | | CCAGACCTACATCTGCAACGTGAACCACAAGCCCTCCAAT |
| | | ACCAAGGTGGACAAGAGGGTCGAGCCTAAAAGCTGTGGA |
| | | TCCGGAGGAGGCGGCGTGTATCATAGAGAGGCCCAGTCC |
| | | GGCAAGTACAAGCTGACCTACGCCGAAGCCAAGGCCGTG |
| | | TGTGAGTTCGAGGGCGGACACCTGGCTACCTACAAACAGC |
| | | TCGAAGCCGCTAGGAAGATCGGATTCCACGTGTGCGCCGC |
| | | CGGATGGATGGCCAAAGGCAGAGTGGGCTACCCCATTGT |
| | | CAAGCCCGGACCCAACTGCGGATTCGGCAAGACCGGCATC |
| | | ATCGACTACGGCATCAGGCTCAACAGGTCCGAGAGATGG |
| | | GACGCTTACTGCTACAATCCCCACGCC |
| SEQ ID NO: 117 | LCDR1 | QSSQSVYGNIWMA |
| SEQ ID NO: 118 | LCDR2 | QASKLAS |
| SEQ ID NO: 119 | LCDR3 | QGNFNTGDRYA |
| SEQ ID NO: 120 | VL | EIVMTQSPSTLSASVGDRVIITCQSSQSVYGNIWMAWYQQ |
| | | KPGRAPKLLIYQASKLASGVPSRFSGSGSGAEFTLTISSLQPD |
| | | DFATYYCQGNFNTGDRYAFGQGTKLTVLKR |
| SEQ ID NO: 121 | DNA of VL | GAGATCGTCATGACCCAGAGCCCCAGCACACTCAGCGCCT |
| | SEQ ID NO: 120 | CCGTGGGAGACAGGGTGATCATCACCTGCCAGTCCTCCCA |
| | | GTCCGTGTACGGCAACATCTGGATGGCCTGGTACCAGCAG |
| | | AAGCCCGGCAGAGCCCCCAAGCTGCTGATCTACCAGGCCA |
| | | GCAAGCTCGCCTCCGGAGTGCCCAGCAGATTTTCCGGCTC |
| | | CGGATCCGGAGCCGAGTTCACACTGACCATCAGCAGCCTG |
| | | CAGCCCGATGACTTCGCCACCTACTATTGCCAGGGCAACTT |
| | | CAACACCGGCGACAGGTACGCCTTTGGCCAGGGCACCAA |
| | | GCTGACCGTCCTCAAGCGT |
| SEQ ID NO: 122 | Light Chain | EIVMTQSPSTLSASVGDRVIITCQSSQSVYGNIWMAWYQQ |
| | | KPGRAPKLLIYQASKLASGVPSRFSGSGSGAEFTLTISSLQPD |
| | | DFATYYCQGNFNTGDRYAFGQGTKLTVLKRTVAAPSVFIFPP |
| | | SDEQLKSGTASVVCLLNNFYPREAKVQWKVDNALQSGNSQ |
| | | ESVTEQDSKDSTYSLSSTLTLSKADYEKHKVYACEVTHQGLSS |
| | | PVTKSFNRGEC |
| SEQ ID NO: 123 | DNA of Light | GAGATCGTCATGACCCAGAGCCCCAGCACACTCAGCGCCT |
| | Chain SEQ ID | CCGTGGGAGACAGGGTGATCATCACCTGCCAGTCCTCCCA |
| | NO: 122 | GTCCGTGTACGGCAACATCTGGATGGCCTGGTACCAGCAG |
| | | AAGCCCGGCAGAGCCCCCAAGCTGCTGATCTACCAGGCCA |
| | | GCAAGCTCGCCTCCGGAGTGCCCAGCAGATTTTCCGGCTC |
| | | CGGATCCGGAGCCGAGTTCACACTGACCATCAGCAGCCTG |
| | | CAGCCCGATGACTTCGCCACCTACTATTGCCAGGGCAACTT |
| | | CAACACCGGCGACAGGTACGCCTTTGGCCAGGGCACCAA |
| | | GCTGACCGTCCTCAAGCGTACGGTGGCTGCTCCCAGCGTC |
| | | TTCATCTTCCCCCCCAGCGATGAGCAGCTCAAGAGCGGCA |
| | | CAGCCTCCGTGGTGTGCCTCCTGAACAACTTCTACCCTAGG |
| | | GAGGCCAAGGTGCAATGGAAGGTGGACAACGCCCTGCAG |
| | | AGCGGCAACAGCCAGGAGTCCGTGACCGAGCAGGACTCC |
| | | AAGGACAGCACCTACAGCCTGAGCAGCACACTCACCCTGA |
| | | GCAAAGCCGACTACGAGAAGCACAAGGTCTACGCCTGCG |
| | | AGGTGACCCATCAGGGCCTGTCCAGCCCCGTGACCAAGAG |
| | | CTTCAACAGAGGCGAGTGC |
| NVS4 | | SEQUENCE (OR SEQ ID NO: #) |
| SEQ ID NO: 1 (Kabat) | HCDR1 | DYYYMT |
| SEQ ID NO: 2 (Kabat) | HCDR2 | FIDPDDDPYYATWAKG |
| SEQ ID NO: 3 (Kabat) | HCDR3 | GDHNSGWGLDI |
| SEQ ID NO: 4 (Chothia) | HCDR1 | GFSLTDYY |
| SEQ ID NO: 5 (Chothia) | HCDR2 | DPDDD |
| SEQ ID NO: 6 (Chothia) | HCDR3 | GDHNSGWGLDI |
| SEQ ID NO: 7 | VH | EVQLVESGGGLVQPGGSLRLSCTASGFSLTDYYYMTWVRQA |
| | | PGKGLEWVGFIDPDDDPYYATWAKGRFTISRDNSKNTLYLQ |
| | | MNSLRAEDTAVYYCAGGDHNSGWGLDIWGQGTLVTVSS |
| SEQ ID NO: 8 | DNA of VH | GAGGTGCAGCTGGTGGAATCAGGCGGCGGACTGGTGCAG |
| | SEQ ID NO: 7 | CCTGGCGGTAGCCTGAGACTGAGCTGCACCGCTAGTGGCT |
| | | TTAGCCTGACCGACTACTACTATATGACCTGGGTCAGACAG |
| | | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGCGATCACAATAGCGGCTGGGGCCTGG |
| | | ATATCTGGGGTCAGGGCACCCTGGTCACCGTGTCTAGC |
| SEQ ID NO: 9 | Heavy Chain | EVQLVESGGGLVQPGGSLRLSCTASGFSLTDYYYMTWVRQA |
| | | PGKGLEWVGFIDPDDDPYYATWAKGRFTISRDNSKNTLYLQ |
| | | MNSLRAEDTAVYYCAGGDHNSGWGLDIWGQGTLVTVSSAS |
| | | TKGPSVFPLAPSSKSTSGGTAALGCLVKDYFPEPVTVSWNSGA |
| | | LTSGVHTFPAVLOSSGLYSLSSVVTVPSSSLGTQTYICNVNHKP |
| | | SNTKVDKRVEPKSC |
| SEQ ID NO: 10 | DNA of Heavy | GAGGTGCAATTGGTTGAATCTGGGGGCGGACTGGTGCAGC |
| | Chain SEQ ID | CCGGTGGATCTTTGCGCCTGTCCTGTACAGCTTCTGGCTTCT |
| | NO: 9 | CCTTGACCGACTACTATTACATGACTTGGGTTCGCCAAGCC |
| | | CCAGGCAAAGGGCTTGAATGGGTGGGGTTCATTGACCCCG |
| | | ACGATGATCCTTACTACGCCACATGGGCAAAGGGCCGGTTT |
| | | ACTATCAGCCGGGATAATTCCAAAAACACATTGTATTTGCA |
| | | AATGAACTCACTGAGAGCAGAAGATACGGCTGTGTACTAT |
| | | TGCGCAGGCGGCGATCATAACTCCGGCTGGGGCCTGGACA |
| | | TCTGGGGGCAGGGGACCCTGGTGACAGTCAGCTCAGCCTC |
| | | AACGAAGGGGCCCAGCGTGTTTCCTTTGGCCCCAAGCAGC |
| | | AAGTCCACGTCCGGTGGGACTGCAGCTCTTGGTTGTCTGGT |
| | | CAAGGATTATTTCCCAGAACCCGTGACCGTGTCTTGGAACA |
| | | GTGGTGCATTGACATCAGGAGTGCATACATTCCCAGCTGTG |
| | | CTGCAGAGCTCTGGCCTGTATAGCCTTTCCTCTGTTGTCACG |
| | | GTGCCCAGCTCCAGCCTGGGGACGCAGACCTATATTTGTAA |
| | | CGTGAACCATAAACCCTCCAACACCAAGGTTGATAAAAGA |
| | | GTGGAGCCCAAGTCTTGT |
| SEQ ID NO: 11 (Kabat) | LCDR1 | QASEIIHSWLA |
| SEQ ID NO: 12 (Kabat) | LCDR2 | LASTLAS |
| SEQ ID NO: 13 (Kabat) | LCDR3 | QNVYLASTNGAN |
| SEQ ID NO: 14 (Chothia) | LCDR1 | SEIIHSW |
| SEQ ID NO: 15 (Chothia) | LCDR2 | LAS |
| SEQ ID NO: 16 (Chothia) | LCDR3 | VYLASTNGA |
| SEQ ID NO: 17 | VL | EIVMTQSPSTLSASVGDRVIITCQASEIIHSWLAWYQQKPGKA |
| | | PKLLIYLASTLASGVPSRFSGSGSGAEFTLTISSLQPDDFATYYC |
| | | QNVYLASTNGANFGQGTKLTVLK |
| SEQ ID NO: 18 | DNA of VL SEQ | GAGATCGTGATGACTCAGTCACCTAGCACCCTGAGCGCTA |
| | ID NO: 17 | GTGTGGGCGATAGAGTGATTATCACCTGTCAGGCTAGTGA |
| | | AATTATTCACTCCTGGCTGGCCTGGTATCAGCAGAAGCCCG |
| | | GTAAAGCCCCTAAGCTGCTGATCTACCTGGCCTCTACCCTG |
| | | GCTAGTGGCGTGCCCTCTAGGTTTAGCGGTAGCGGTAGTG |
| | | GCGCCGAGTTCACCCTGACTATCTCTAGCCTGCAGCCCGAC |
| | | GACTTCGCTACCTACTACTGTCAGAACGTCTACCTGGCTAG |
| | | TACTAACGGCGCTAACTTCGGTCAGGGCACTAAGCTGACC |
| | | GTGCTGAAG |
| SEQ ID NO: 19 | Light Chain | EIVMTQSPSTLSASVGDRVIITCQASEIIHSWLAWYQQKPGKA |
| | | PKLLIYLASTLASGVPSRFSGSGSGAEFTLTISSLQPDDFATYYC |
| | | QNVYLASTNGANFGQGTKLTVLKRTVAAPSVFIFPPSDEQLKS |
| | | GTASVVCLLNNFYPREAKVQWKVDNALQSGNSQESVTEQDS |
| | | KDSTYSLSSTLTLSKADYEKHKVYACEVTHQGLSSPVTKSFNRG |
| | | EC |
| SEQ ID NO: 20 | DNA of Light | GAGATCGTGATGACTCAGTCACCTAGCACCCTGAGCGCTA |
| | Chain SEQ ID | GTGTGGGCGATAGAGTGATTATCACCTGTCAGGCTAGTGA |
| | NO: 19 | AATTATTCACTCCTGGCTGGCCTGGTATCAGCAGAAGCCCG |
| | | GTAAAGCCCCTAAGCTGCTGATCTACCTGGCCTCTACCCTG |
| | | GCTAGTGGCGTGCCCTCTAGGTTTAGCGGTAGCGGTAGTG |
| | | GCGCCGAGTTCACCCTGACTATCTCTAGCCTGCAGCCCGAC |
| | | GACTTCGCTACCTACTACTGTCAGAACGTCTACCTGGCTAG |
| | | TACTAACGGCGCTAACTTCGGTCAGGGCACTAAGCTGACC |
| | | GTGCTGAAGCGGACCGTGGCCGCTCCTAGTGTGTTTATCTT |
| | | CCCACCTAGCGACGAGCAGCTGAAGTCAGGCACCGCTAGT |
| | | GTCGTGTGCCTGCTGAACAACTTCTACCCTAGAGAAGCTAA |
| | | GGTGCAGTGGAAAGTGGATAACGCCCTGCAGTCAGGTAAT |
| | | AGTCAGGAATCAGTCACCGAGCAGGACTCTAAGGATAGCA |
| | | CCTATAGCCTGTCTAGCACACTGACCCTGTCTAAGGCCGAC |
| | | TACGAGAAGCACAAGGTCTACGCCTGCGAAGTGACTCACC |
| | | AGGGACTGTCTAGCCCCGTGACTAAGTCCTTTAATAGAGGC |
| | | GAGTGC |
| NVS1 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 21 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREARSGKYKLTYAEAKAVC |
| | NO: 32) | EFEGGHLATYKQLEAARKIGFHVCAAGWMAKGRVGYPIVKP |
| | | GPNCGFGKTGIIDYGIRLNRSERWDAYCYNPHA |
| SEQ ID NO: 22 | DNA of Heavy | GAGGTGCAGCTGGTGGAATCAGGCGGCGGACTGGTGCAG |
| | Chain + Linker + | CCTGGCGGTAGCCTGAGACTGAGCTGCACCGCTAGTGGCT |
| | protein tag | TTAGCCTGACCGACTACTACTATATGACCTGGGTCAGACAG |
| | SEQ ID NO: 21 | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGCGATCACAATAGCGGCTGGGGCCTGG |
| | | ATATCTGGGGTCAGGGCACCCTGGTCACCGTGTCTAGCGCC |
| | | TCTACTAAGGGACCTAGCGTGTTCCCCCTGGCCCCTAGCTC |
| | | TAAGTCTACTAGCGGCGGCACCGCCGCTCTGGGCTGCCTG |
| | | GTCAAGGACTACTTCCCCGAGCCCGTGACCGTCAGCTGGA |
| | | ATAGCGGCGCTCTGACTAGCGGAGTGCACACCTTCCCCGCC |
| | | GTGCTGCAGTCTAGCGGCCTGTATAGCCTGTCTAGCGTCGT |
| | | GACCGTGCCTAGCTCTAGCCTGGGCACTCAGACCTATATCT |
| | | GTAACGTGAACCACAAGCCCTCTAACACTAAGGTGGACAA |
| | | GCGGGTGGAACCTAAGTCCTGCGGTAGCGGCGGAGGCGG |
| | | AGTCTATCACAGAGAGGCTAGATCAGGCAAGTATAAGCTG |
| | | ACCTACGCCGAGGCTAAGGCCGTGTGCGAGTTCGAGGGCG |
| | | GTCACCTGGCTACCTATAAGCAGCTGGAAGCCGCTAGAAA |
| | | GATCGGCTTTCACGTGTGCGCCGCTGGCTGGATGGCTAAG |
| | | GGTAGAGTGGGCTACCCTATCGTGAAGCCTGGCCCTAACT |
| | | GCGGCTTCGGTAAAACCGGAATTATCGACTACGGGATTAG |
| | | GCTGAATAGATCAGAGCGCTGGGACGCCTACTGCTATAAC |
| | | CCTCACGCT |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL SEQ | 18 |
| | ID NO: 17 | |
| SEQ ID NO: 19 | Light Chain | 19 |
| SEQ ID NO: 20 | DNA of Light | 20 |
| | Chain SEQ ID | |
| | NO: 19 | |
| NVS2 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 23 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREAQSGKYKLTYAEAKAVC |
| | NO: 33) | EFEGGHLATYKQLEAARKIGFHVCAAGWMAKGRVGYPIVKP |
| | | GPNCGFGKTGIIDYGIRLNRSERWDAYCYNPHA |
| SEQ ID NO: 24 | DNA of Heavy | GAGGTGCAGCTGGTGGAATCAGGCGGCGGACTGGTGCAG |
| | Chain + Linker + | CCTGGCGGTAGCCTGAGACTGAGCTGCACCGCTAGTGGCT |
| | protein tag | TTAGCCTGACCGACTACTACTATATGACCTGGGTCAGACAG |
| | SEQ ID NO: 23 | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGTGATCACAATAGCGGCTGGGGCCTGG |
| | | ATATCTGGGGTCAAGGCACCCTGGTCACCGTGTCTAGCGCC |
| | | TCTACTAAGGGCCCCTCAGTGTTCCCCCTGGCCCCTAGCTCT |
| | | AAGTCTACTAGCGGCGGCACCGCCGCTCTGGGCTGCCTGG |
| | | TCAAGGACTACTTCCCCGAGCCCGTGACCGTCAGCTGGAAT |
| | | AGCGGCGCTCTGACTAGCGGAGTGCACACCTTCCCCGCCGT |
| | | GCTGCAGTCTAGCGGCCTGTATAGCCTGTCTAGCGTCGTGA |
| | | CCGTGCCTAGCTCTAGCCTGGGCACTCAGACCTATATCTGT |
| | | AACGTGAACCACAAGCCCTCTAACACTAAGGTGGACAAGC |
| | | GGGTGGAACCTAAGTCCTGCGGTAGCGGCGGAGGCGGAG |
| | | TCTATCACAGAGAGGCTCAGTCAGGCAAGTATAAGCTGAC |
| | | CTACGCCGAGGCTAAGGCCGTGTGCGAGTTCGAGGGCGGT |
| | | CACCTGGCTACCTATAAGCAGCTGGAAGCCGCTAGAAAGA |
| | | TCGGCTTTCACGTGTGCGCCGCTGGCTGGATGGCTAAGGG |
| | | TAGAGTGGGCTACCCTATCGTGAAGCCTGGCCCTAACTGCG |
| | | GCTTCGGTAAAACCGGAATTATCGACTACGGGATTAGGCT |
| | | GAATAGATCAGAGCGCTGGGACGCCTACTGCTATAACCCTC |
| | | ACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL SEQ | 18 |
| | ID NO: 18 | |
| SEQ ID NO: 19 | Light Chain | 19 |
| SEQ ID NO: 20 | DNA of Light | 10 |
| | Chain SEQ ID | |
| | NO: 20 | |
| NVS3 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 25 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREAASGKYKLTYAEAKAVC |
| | NO: 34) | EFEGGHLATYKQLEAARKIGFHVCAAGWMAKGRVGYPIVKP |
| | | GPNCGFGKTGIIDYGIRLNRSERWDAYCYNPHA |
| SEQ ID NO: 26 | DNA of Heavy | GAGGTGCAGCTGGTGGAATCAGGCGGCGGACTGGTGCAG |
| | Chain + Linker + | CCTGGCGGTAGCCTGAGACTGAGCTGCACCGCTAGTGGCT |
| | protein tag | TTAGCCTGACCGACTACTACTATATGACCTGGGTCAGACAG |
| | SEQ ID NO: 25 | GCCCCTGGTAAAGGCCTGGAGTGGGTCGGCTTTATCGACC |
| | | CCGACGACGACCCCTACTACGCTACCTGGGCTAAGGGCCG |
| | | GTTCACTATCTCTAGGGATAACTCTAAGAACACCCTGTACCT |
| | | GCAGATGAATAGCCTGAGAGCCGAGGACACCGCCGTCTAC |
| | | TACTGCGCCGGCGGTGATCACAATAGCGGCTGGGGCCTGG |
| | | ATATCTGGGGTCAAGGCACCCTGGTCACCGTGTCTAGCGCC |
| | | TCTACTAAGGGCCCCTCAGTGTTCCCCCTGGCCCCTAGCTCT |
| | | AAGTCTACTAGCGGCGGCACCGCCGCTCTGGGCTGCCTGG |
| | | TCAAGGACTACTTCCCCGAGCCCGTGACCGTCAGCTGGAAT |
| | | AGCGGCGCTCTGACTAGCGGAGTGCACACCTTCCCCGCCGT |
| | | GCTGCAGTCTAGCGGCCTGTATAGCCTGTCTAGCGTCGTGA |
| | | CCGTGCCTAGCTCTAGCCTGGGCACTCAGACCTATATCTGT |
| | | AACGTGAACCACAAGCCCTCTAACACTAAGGTGGACAAGC |
| | | GGGTGGAACCTAAGTCCTGCGGTAGCGGCGGAGGCGGAG |
| | | TCTATCACAGAGAGGCTGCTAGCGGTAAATACAAGCTGAC |
| | | CTACGCCGAGGCTAAGGCCGTGTGCGAGTTCGAGGGCGGT |
| | | CACCTGGCTACCTATAAGCAGCTGGAAGCCGCTAGAAAGA |
| | | TCGGCTTTCACGTGTGCGCCGCTGGCTGGATGGCTAAGGG |
| | | TAGAGTGGGCTACCCTATCGTGAAGCCTGGCCCTAACTGCG |
| | | GCTTCGGTAAAACCGGAATTATCGACTACGGGATTAGGCT |
| | | GAATAGATCAGAGCGCTGGGACGCCTACTGCTATAACCCTC |
| | | ACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL SEQ | 18 |
| | ID NO: 18 | |
| SEQ ID NO: 19 | Light Chain | 19 |
| SEQ ID NO: 20 | DNA of Light | 20 |
| | Chain SEQ ID | |
| | NO: 19 | |
| NVS36 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 7 |
| SEQ ID NO: 8 | DNA of VH | 8 |
| | SEQ ID NO: 7 | |
| SEQ ID NO: 9 | Heavy Chain | 9 |
| SEQ ID NO: 27 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGACGVYHREAQSGKYKLTYAEAKA |
| | NO: 35) | VCEFEGGHLATYKQLECARKIGFHVCAAGWMAKGRVGYPIV |
| | | KPGPNCGFGKTGIIDYGIRLNRSERWDAYCYNPHA |
| SEQ ID NO: 28 | DNA of Heavy | GAAGTGCAGCTGGTGGAAAGCGGCGGAGGCCTGGTGCAG |
| | Chain + Linker + | CCTGGCGGATCTCTGAGACTGAGCTGTACCGCCAGCGGCTT |
| | protein tag | CAGCCTGACCGACTACTACTACATGACCTGGGTCCGACAGG |
| | SEQ ID NO: 27 | CCCCTGGCAAGGGACTGGAATGGGTCGGATTCATCGACCC |
| | | CGACGACGACCCCTACTACGCCACATGGGCCAAGGGCCGG |
| | | TTCACCATCAGCCGGGACAACAGCAAGAACACCCTGTACCT |
| | | GCAGATGAACAGCCTGCGGGCCGAGGACACCGCCGTGTAC |
| | | TATTGTGCCGGCGGAGATCACAACAGCGGCTGGGGCCTGG |
| | | ATATCTGGGGACAGGGAACACTGGTCACCGTGTCTAGCGC |
| | | CAGCACCAAGGGCCCTAGCGTGTTCCCTCTGGCCCCTAGCA |
| | | GCAAGAGCACATCTGGCGGAACAGCCGCCCTGGGCTGCCT |
| | | GGTCAAGGACTACTTTCCCGAGCCCGTGACCGTGTCCTGGA |
| | | ACTCTGGCGCTCTGACAAGCGGCGTGCACACCTTTCCAGCC |
| | | GTGCTGCAGAGCAGCGGCCTGTACTCTCTGAGCAGCGTGG |
| | | TCACAGTGCCCAGCTCTAGCCTGGGAACCCAGACCTACATC |
| | | TGCAACGTGAACCACAAGCCCAGCAACACCAAGGTGGACA |
| | | AGCGGGTGGAACCCAAGAGCTGCGGATCCGGCGGAGGCG |
| | | CCTGTGGCGTGTATCACAGGGAGGCCCAGAGCGGCAAGTA |
| | | CAAGCTCACCTACGCCGAGGCCAAGGCCGTGTGCGAATTC |
| | | GAGGGCGGCCACCTGGCCACCTACAAGCAGCTGGAGTGCG |
| | | CCAGGAAGATCGGCTTCCACGTGTGTGCCGCCGGCTGGAT |
| | | GGCCAAAGGCAGAGTGGGCTACCCCATCGTGAAACCCGGC |
| | | CCCAACTGCGGCTTCGGCAAGACAGGCATCATCGACTACG |
| | | GCATCAGGCTGAACAGGAGCGAGAGGTGGGACGCCTACT |
| | | GCTACAACCCCCACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL SEQ | 18 |
| | ID NO: 18 | |
| SEQ ID NO: 19 | Light Chain | 19 |
| SEQ ID NO: 20 | DNA of Light | 20 |
| | Chain SEQ ID | |
| | NO: 19 | |
| NVS37 | | |
| SEQ ID NO: 1 (Kabat) | HCDR1 | 1 |
| SEQ ID NO: 2 (Kabat) | HCDR2 | 2 |
| SEQ ID NO: 3 (Kabat) | HCDR3 | 3 |
| SEQ ID NO: 4 (Chothia) | HCDR1 | 4 |
| SEQ ID NO: 5 (Chothia) | HCDR2 | 5 |
| SEQ ID NO: 6 (Chothia) | HCDR3 | 6 |
| SEQ ID NO: 7 | VH | 6 |
| SEQ ID NO: 8 | DNA of VH | 8 |
| | SEQ ID NO: 7 | |
| SEQ ID NO: 9 | Heavy Chain | 9 |
| SEQ ID NO: 29 | Heavy Chain + | EVQLVESGGGLVQPGGSLRLSCTASGFSLTDYYYMTWVRQA |
| | Linker + | PGKGLEWVGFIDPDDDPYYATWAKGRFTISRDNSKNTLYLQ |
| | protein tag | MNSLRAEDTAVYYCAGGDHNSGWGLDIWGQGTLVTVSSAS |
| | (SEQ ID NO: 9 + | TKGPSVFPLAPSSKSTSGGTAALGCLVKDYFPEPVTVSWNSGA |
| | SEQ ID NO: | LTSGVHTFPAVLQSSGLYSLSSVVTVPSSSLGTQTYICNVNHKP |
| | 31 + SEQ ID | SNTKVDKRVEPKSCGSGGGGVYHREAQSGKYKLTYAEAKAVC |
| | NO: 36) | EFEGGHLCTYKQLEAARKIGFHVCAAGWMAKGRVGYPIVKP |
| | | GPNCGFGKTGIIDYGIRLNRSERWDAYCCNPHA |
| SEQ ID NO: 30 | DNA of Heavy | GAAGTGCAGCTGGTGGAAAGCGGCGGAGGCCTGGTGCAG |
| | Chain + Linker + | CCTGGCGGATCTCTGAGACTGAGCTGTACCGCCAGCGGCTT |
| | protein tag | CAGCCTGACCGACTACTACTACATGACCTGGGTCCGACAGG |
| | SEQ ID NO: 29 | CCCCTGGCAAGGGACTGGAATGGGTCGGATTCATCGACCC |
| | | CGACGACGACCCCTACTACGCCACATGGGCCAAGGGCCGG |
| | | TTCACCATCAGCCGGGACAACAGCAAGAACACCCTGTACCT |
| | | GCAGATGAACAGCCTGCGGGCCGAGGACACCGCCGTGTAC |
| | | TATTGTGCCGGCGGAGATCACAACAGCGGCTGGGGCCTGG |
| | | ATATCTGGGGACAGGGAACACTGGTCACCGTGTCTAGCGC |
| | | CAGCACCAAGGGCCCTAGCGTGTTCCCTCTGGCCCCTAGCA |
| | | GCAAGAGCACATCTGGCGGAACAGCCGCCCTGGGCTGCCT |
| | | GGTCAAGGACTACTTTCCCGAGCCCGTGACCGTGTCCTGGA |
| | | ACTCTGGCGCTCTGACAAGCGGCGTGCACACCTTTCCAGCC |
| | | GTGCTGCAGAGCAGCGGCCTGTACTCTCTGAGCAGCGTGG |
| | | TCACAGTGCCCAGCTCTAGCCTGGGAACCCAGACCTACATC |
| | | TGCAACGTGAACCACAAGCCCAGCAACACCAAGGTGGACA |
| | | AGCGGGTGGAACCCAAGAGCTGCGGATCCGGCGGCGGCG |
| | | GAGTGTATCACAGAGAGGCCCAGAGCGGCAAGTACAAGCT |
| | | GACCTACGCCGAGGCCAAGGCCGTGTGTGAGTTCGAGGGC |
| | | GGCCACCTGTGCACCTACAAGCAGCTGGAGGCCGCCAGGA |
| | | AGATCGGCTTCCACGTGTGTGCCGCCGGCTGGATGGCTAA |
| | | AGGCAGGGTGGGCTACCCCATTGTGAAGCCCGGCCCCAAT |
| | | TGCGGCTTCGGCAAGACCGGCATCATCGACTACGGCATCA |
| | | GGCTGAACAGGAGCGAGAGGTGGGACGCCTACTGCTGCA |
| | | ACCCCCACGCC |
| SEQ ID NO: 11 (Kabat) | LCDR1 | 11 |
| SEQ ID NO: 12 (Kabat) | LCDR2 | 12 |
| SEQ ID NO: 13 (Kabat) | LCDR3 | 13 |
| SEQ ID NO: 14 (Chothia) | LCDR1 | 14 |
| SEQ ID NO: 15 (Chothia) | LCDR2 | 15 |
| SEQ ID NO: 16 (Chothia) | LCDR3 | 16 |
| SEQ ID NO: 17 | VL | 17 |
| SEQ ID NO: 18 | DNA of VL SEQ | 18 |
| | ID NO: 18 | |
| SEQ ID NO: 19 | Light Chain | 19 |
| SEQ ID NO: 20 | DNA of Light | 20 |
| | Chain SEQ ID | |
| | NO: 19 | |
| Tag and Linker Sequences | | |
| SEQ ID NO: 31 | Linker | GSGGG |
| SEQ ID NO: 124 | Linker | GSGG |
| SEQ ID NO: 32 | Protein tag 1 | GVYHREARSGKYKLTYAEAKAVCEFEGGHLATYKQLEAARKIG |
| | (HA10) | FHVCAAGWMAKGRVGYPIVKPGPNCGFGKTGIIDYGIRLNRS |
| | | ERWDAYCYNPHAK |
| SEQ ID NO: 33 | Protein tag 2 | GVYHREAQSGKYKLTYAEAKAVCEFEGGHLATYKQLEAARKIG |
| | (HA10.1) | FHVCAAGWMAKGRVGYPIVKPGPNCGFGKTGIIDYGIRLNRS |
| | | ERWDAYCYNPHA |
| SEQ ID NO: 34 | Protein tag 3 | GVYHREAASGKYKLTYAEAKAVCEFEGGHLATYKQLEAARKIG |
| | (HA10.2) | FHVCAAGWMAKGRVGYPIVKPGPNCGFGKTGIIDYGIRLNRS |
| | | ERWDAYCYNPHA |
| SEQ ID NO: 35 | Protein tag 4 | ACGVYHREAQSGKYKLTYAEAKAVCEFEGGHLATYKQLECAR |
| | (HA11) | KIGFHVCAAGWMAKGRVGYPIVKPGPNCGFGKTGIIDYGIRL |
| | | NRSERWDAYCYNPHA |
| SEQ ID NO: 36 | Protein tag 5 | GVYHREAQSGKYKLTYAEAKAVCEFEGGHLCTYKQLEAARKIG |
| | (HA11.1) | FHVCAAGWMAKGRVGYPIVKPGPNCGFGKTGIIDYGIRLNRS |
| | | ERWDAYCCNPHA |
| SEQ ID NO: 103 | DNA of SEQ ID | GGAGTCTATCACAGAGAGGCTAGATCAGGCAAGTATAAGC |
| | NO: 32 (HA10) | TGACCTACGCCGAGGCTAAGGCCGTGTGCGAGTTCGAGGG |
| | | CGGTCACCTGGCTACCTATAAGCAGCTGGAAGCCGCTAGA |
| | | AAGATCGGCTTTCACGTGTGCGCCGCTGGCTGGATGGCTA |
| | | AGGGTAGAGTGGGCTACCCTATCGTGAAGCCTGGCCCTAA |
| | | CTGCGGCTTCGGTAAAACCGGAATTATCGACTACGGGATTA |
| | | GGCTGAATAGATCAGAGCGCTGGGACGCCTACTGCTATAA |
| | | CCCTCACGCTAAG |
| SEQ ID NO: 104 | DNA of SEQ ID | GGAGTCTATCACAGAGAGGCTCAGTCAGGCAAGTATAAGC |
| | NO: 33 | TGACCTACGCCGAGGCTAAGGCCGTGTGCGAGTTCGAGGG |
| | (HA10.1) | CGGTCACCTGGCTACCTATAAGCAGCTGGAAGCCGCTAGA |
| | | AAGATCGGCTTTCACGTGTGCGCCGCTGGCTGGATGGCTA |
| | | AGGGTAGAGTGGGCTACCCTATCGTGAAGCCTGGCCCTAA |
| | | CTGCGGCTTCGGTAAAACCGGAATTATCGACTACGGGATTA |
| | | GGCTGAATAGATCAGAGCGCTGGGACGCCTACTGCTATAA |
| | | CCCTCACGCC |
| SEQ ID NO: 105 | DNA of SEQ ID | GGAGTCTATCACAGAGAGGCTGCTAGCGGTAAATACAAGC |
| | NO: 34 (HA | TGACCTACGCCGAGGCTAAGGCCGTGTGCGAGTTCGAGGG |
| | 10.2) | CGGTCACCTGGCTACCTATAAGCAGCTGGAAGCCGCTAGA |
| | | AAGATCGGCTTTCACGTGTGCGCCGCTGGCTGGATGGCTA |
| | | AGGGTAGAGTGGGCTACCCTATCGTGAAGCCTGGCCCTAA |
| | | CTGCGGCTTCGGTAAAACCGGAATTATCGACTACGGGATTA |
| | | GGCTGAATAGATCAGAGCGCTGGGACGCCTACTGCTATAA |
| | | CCCTCACGCC |
| SEQ ID NO: 106 | DNA of SEQ ID | GGCGCCTGTGGCGTGTATCACAGGGAGGCCCAGAGCGGC |
| | NO: 35 (HA | AAGTACAAGCTCACCTACGCCGAGGCCAAGGCCGTGTGCG |
| | 11) | AATTCGAGGGCGGCCACCTGGCCACCTACAAGCAGCTGGA |
| | | GTGCGCCAGGAAGATCGGCTTCCACGTGTGTGCCGCCGGC |
| | | TGGATGGCCAAAGGCAGAGTGGGCTACCCCATCGTGAAAC |
| | | CCGGCCCCAACTGCGGCTTCGGCAAGACAGGCATCATCGA |
| | | CTACGGCATCAGGCTGAACAGGAGCGAGAGGTGGGACGC |
| | | CTACTGCTACAACCCCCACGCC |
| SEQ ID NO: 107 | DNA of SEQ ID | GGAGTGTATCACAGAGAGGCCCAGAGCGGCAAGTACAAG |
| | NO: 36 (HA | CTGACCTACGCCGAGGCCAAGGCCGTGTGTGAGTTCGAGG |
| | 11.1) | GCGGCCACCTGTGCACCTACAAGCAGCTGGAGGCCGCCAG |
| | | GAAGATCGGCTTCCACGTGTGTGCCGCCGGCTGGATGGCT |
| | | AAAGGCAGGGTGGGCTACCCCATTGTGAAGCCCGGCCCCA |
| | | ATTGCGGCTTCGGCAAGACCGGCATCATCGACTACGGCATC |
| | | AGGCTGAACAGGAGCGAGAGGTGGGACGCCTACTGCTGC |
| | | AACCCCCACGCC |
| SEQ ID NO: 204 | NVS-X | GVYHREAISGKYYLTYAEAKAVCEFEGGHLATYKQLLA |
| | | AQKIGFHVCAAGWMAKGRVGYPIVKPGPNCGFGKTGII |
| | | DYGIRLNRSERWDAYCYNPHA |
| SEQ ID NO: 205 | NVS-Y | GVYHREAISGKYYLTYAEAKAVCEFEGGHLATYKQLQA |
| | | AQKIGFHVCAAGWMAKGRVGYPIVKPGPNCGFGKTGII |
| | | DYGIRLNRSERWDAYCYNPHA |
| SEQ ID NO: 206 | NVS-AX | ACGVYHREAISGKYYLTYAEAKAVCEFEGGHLATYKQL |
| | | LAAQKIGFHVCAAGWMAKGRVGYPIVKPGPNCGFGKT |
| | | GIIDYGIRLNRSERWDAYCYNPHA |
| SEQ ID NO: 207 | NVS-AY | ACGVYHREAISGKYYLTYAEAKAVCEFEGGHLATYKQL |
| | | QAAQKIGFHVCAAGWMAKGRVGYPIVKPGPNCGFGKT |
| | | GIIDYGIRLNRSERWDAYCYNPHA |
| SEQ ID NO: 220 | NVS-Z | ACGVYHREAQSGKYYLTYAEAKAVCEFEGGHLATYKQ |
| | | LLCAQKIGFHVCAAGWMAKGRVGYPIVKPGPNCGFGK |
| | | TGIIDYGIRLNRSERWDAYCYNPHA |
| mAb1 | | |
| SEQ ID NO: 208 (Kabat) | HCDR1 | GFTFDDYAMH |
| SEQ ID NO: 209 (Kabat) | HCDR2 | AITWNSGHIDYADSVEG |
| SEQ ID NO: 210 (Kabat) | HCDR3 | VSYLSTASSLDY |
| SEQ ID NO: 211 | VH | EVQLVESGGGLVQPGRSLRLSCAASGFTFDDYAMHW |
| | | VRQAPGKGLEVVVSAITWNSGHIDYADSVEGRFTISRDN |
| | | AKNSLYLQMNSLRAEDTAVYYCAKVSYLSTASSLDYW |
| | | GQGTLVTVSS |
| SEQ ID NO: 212 | Heavy Chain | MKHLWFFLLLVAAPRVVVLSEVQLVESGGGLVQPGRSL |
| | | RLSCAASGFTFDDYAMHWVRQAPGKGLEWVSAITWN |
| | | SGHIDYADSVEGRFTISRDNAKNSLYLQMNSLRAEDTA |
| | | VYYCAKVSYLSTASSLDYWGQGTLVTVSSASTKGPSV |
| | | FPLAPSSKSTSGGTAALGCLVKDYFPEPVTVSWNSGAL |
| | | TSGVHTFPAVLQSSGLYSLSSVVTVPSSSLGTQTYICN |
| | | VNHKPSNTKVDKKVEPKSCDKTHTCPPCPAPELLGGP |
| | | SVFLFPPKPKDTLMISRTPEVTCVVVDVSHEDPEVKFN |
| | | WYVDGVEVHNAKTKPREEQYNSTYRVVSVLTVLHQD |
| | | WLNGKEYKCKVSNKALPAPIEKTISKAKGQPREPQVYT |
| | | LPPSRDELTKNQVSLTCLVKGFYPSDIAVEWESNGQPE |
| | | NNYKTTPPVLDSDGSFFLYSKLTVDKSRWQQGNVFSC |
| | | SVMHEALHNHYTQKSLSLSPGGSGGGACGVYHREAQ |
| | | SGKYYLTYAEAKAVCEFEGGHLATYKQLLCAQKIGFHV |
| | | CAAGWMAKGRVGYPIVKPGPNCGFGKTGIIDYGIRLNR |
| | | SERWDAYCYNPHA |
| SEQ ID NO: 213 (Kabat) | LCDR1 | RASQGIRNYLA |
| SEQ ID NO: 214 (Kabat) | LCDR2 | AASTLQS |
| SEQ ID NO: 215 (Kabat) | LCDR3 | QRYNRAPYT |
| SEQ ID NO: 216 | VL | DIQMTQSPSSLSASVGDRVTITCRASQGIRNYLAWYQQ |
| | | KPGKAPKLLIYAASTLQSGVPSRFSGSGSGTDFTLTISS |
| | | LQPEDVATYYCQRYNRAPYTFGQGTKVEIK |
| SEQ ID NO: 217 | Light Chain | MVLQTQVFISLLLWISGAYGDIQMTQSPSSLSASVGDR |
| | | VTITCRASQGIRNYLAWYQQKPGKAPKLLIYAASTLQSG |
| | | VPSRFSGSGSGTDFTLTISSLQPEDVATYYCQRYNRAP |
| | | YTFGQGTKVEIKRTVAAPSVFIFPPSDEQLKSGTASVVC |
| | | LLNNFYPREAKVQWKVDNALQSGNSQESVTEQDSKDS |
| | | TYSLSSTLTLSKADYEKHKVYACEVTHQGLSSPVTKSF |
| | | NRGEC |
| mAb2 | | |
| SEQ ID NO: 208 (Kabat) | HCDR1 | GFTFDDYAMH |
| SEQ ID NO: 209 (Kabat) | HCDR2 | AITWNSGHIDYADSVEG |
| SEQ ID NO: 210 (Kabat) | HCDR3 | VSYLSTASSLDY |
| SEQ ID NO: 211 | VH | EVQLVESGGGLVQPGRSLRLSCAASGFTFDDYAMHW |
| | | VRQAPGKGLEVVVSAITWNSGHIDYADSVEGRFTISRDN |
| | | AKNSLYLQMNSLRAEDTAVYYCAKVSYLSTASSLDYW |
| | | GQGTLVTVSS |
| SEQ ID NO: 218 | Heavy Chain | MKHLWFFLLLVAAPRVVVLSEVQLVESGGGLVQPGRSL |
| | | RLSCAASGFTFDDYAMHWVRQAPGKGLEWVSAITWN |
| | | SGHIDYADSVEGRFTISRDNAKNSLYLQMNSLRAEDTA |
| | | VYYCAKVSYLSTASSLDYWGQGTLVTVSSASTKGPSV |
| | | FPLAPSSKSTSGGTAALGCLVKDYFPEPVTVSWNSGAL |
| | | TSGVHTFPAVLQSSGLYSLSSVVTVPSSSLGTQTYICN |
| | | VNHKPSNTKVDKKVEPKSCDKTHTCPPCPAPELLGGP |
| | | SVFLFPPKPKDTLMISRTPEVTCVVVDVSHEDPEVKFN |
| | | WYVDGVEVHNAKTKPREEQYNSTYRVVSVLTVLHQD |
| | | WLNGKEYKCKVSNKALPAPIEKTISKAKGQPREPQVYT |
| | | LPPSRDELTKNQVSLTCLVKGFYPSDIAVEWESNGQPE |
| | | NNYKTTPPVLDSDGSFFLYSKLTVDKSRWQQGNVFSC |
| | | SVMHEALHNHYTQKSLSLSPG |
| SEQ ID NO: 213 (Kabat) | LCDR1 | RASQGIRNYLA |
| SEQ ID NO: 214 (Kabat) | LCDR2 | AASTLQS |
| SEQ ID NO: 215 (Kabat) | LCDR3 | QRYNRAPYT |
| SEQ ID NO: 216 | VL | DIQMTQSPSSLSASVGDRVTITCRASQGIRNYLAWYQQ |
| | | KPGKAPKLLIYAASTLQSGVPSRFSGSGSGTDFTLTISS |
| | | LQPEDVATYYCQRYNRAPYTFGQGTKVEIK |
| SEQ ID NO: 219 | Light Chain | MVLQTQVFISLLLWISGAYGDIQMTQSPSSLSASVGDR |
| | | VTITCRASQGIRNYLAWYQQKPGKAPKLLIYAASTLQSG |
| | | VPSRFSGSGSGTDFTLTISSLQPEDVATYYCQRYNRAP |
| | | YTFGQGTKVEIKRTVAAPSVFIFPPSDEQLKSGTASVVC |
| | | LLNNFYPREAKVQWKVDNALQSGNSQESVTEQDSKDS |
| | | TYSLSSTLTLSKADYEKHKVYACEVTHQGLSSPVTKSF |
| | | NRGECGSGGGACGVYHREAQSGKYYLTYAEAKAVCE |
| | | FEGGHLATYKQLLCAQKIGFHVCAAGWMAKGRVGYPI |
| | | VKPGPNCGFGKTGIIDYGIRLNRSERWDAYCYNPHA |

TABLE 2
Examples of additional peptide tagged molecules (e.g.: NVS70T, NVS71T, NVS72T and
NVS75T), untagged molecules (e.g.: NVS70, NVS71, NVS72 and NVS75) and component sequences.

| NVS70 and NVS70T | | |
|---|---|---|
| SEQ ID NO: 37 | HCDR1 | SYAIS |
| SEQ ID NO: 38 | HCDR2 | GIGPFFGTANYAQKFQG |
| SEQ ID NO: 39 | HCDR3 | DTPYFDY |
| SEQ ID NO: 40 | VH | EVQLVQSGAEVKKPGSSVKVSCKASGGTFSSYAISWVRQAPG |
| | | QGLEWMGGIGPFFGTANYAQKFQGRVTITADESTSTAYMEL |
| | | SSLRSEDTAVYYCARDTPYFDYWGQGTLVTVSS |
| SEQ ID NO: 41 | DNA of VH | GAGGTGCAATTGGTTCAGTCTGGCGCGGAAGTGAAAAAAC |
| | SEQ ID NO: 40 | CGGGCAGCAGCGTGAAAGTGAGCTGCAAAGCCTCCGGAG |
| | | GCACTTTTTCTTCTTATGCCATTTCTTGGGTGCGCCAAGCCC |
| | | CTGGGCAGGGTCTCGAGTGGATGGGCGGTATCGGTCCGTT |
| | | TTTTGGCACTGCGAATTACGCGCAGAAGTTTCAGGGCCGG |
| | | GTGACCATTACCGCGGATGAAAGCACCAGCACCGCGTATA |
| | | TGGAACTGAGCAGCCTGCGTAGCGAAGATACGGCCGTGTA |
| | | TTATTGCGCGCGTGATACTCCTTATTTTGATTATTGGGGCCA |
| | | AGGCACCCTGGTGACGGTTAGCTCA |
| SEQ ID NO: 42 | Heavy Chain | EVQLVQSGAEVKKPGSSVKVSCKASGGTFSSYAISWVRQAPG |
| | | QGLEWMGGIGPFFGTANYAQKFQGRVTITADESTSTAYMEL |
| | | SSLRSEDTAVYYCARDTPYFDYWGQGTLVTVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSC |
| SEQ ID NO: 43 | DNA of Heavy | GAGGTGCAATTGGTCCAAAGCGGCGCTGAGGTCAAGAAG |
| | Chain SEQ ID | CCTGGCAGCAGCGTGAAGGTCTCCTGCAAGGCCAGCGGCG |
| | NO: 42 | GCACATTCTCCAGCTATGCTATCAGCTGGGTCAGACAAGCC |
| | | CCCGGCCAAGGACTGGAATGGATGGGAGGAATCGGCCCTT |
| | | TCTTCGGAACCGCCAACTACGCCCAGAAGTTTCAGGGAAG |
| | | GGTGACCATCACCGCCGATGAGAGCACATCCACAGCCTAT |
| | | ATGGAGCTCTCCAGCCTGAGATCCGAAGACACCGCCGTCTA |
| | | CTACTGCGCTAGGGACACCCCCTACTTCGACTATTGGGGCC |
| | | AGGGCACACTCGTGACCGTGAGCTCAGCCAGCACCAAAGG |
| | | CCCTAGCGTCTTCCCCCTGGCTCCTTCCAGCAAGAGCACAA |
| | | GCGGAGGAACAGCTGCTCTCGGCTGCCTGGTCAAGGACTA |
| | | CTTCCCCGAGCCTGTCACAGTGTCCTGGAATAGCGGAGCCC |
| | | TGACCAGCGGCGTGCATACATTCCCCGCTGTGCTCCAGAGC |
| | | TCCGGCCTCTACAGCCTCAGCTCCGTGGTCACCGTCCCTAG |
| | | CTCCTCCCTGGGCACACAGACCTACATCTGCAACGTCAACC |
| | | ACAAGCCCTCCAACACCAAGGTGGACAAGAGGGTGGAGCC |
| | | CAAAAGCTGT |
| SEQ ID NO: 44 | Heavy Chain + | EVQLVQSGAEVKKPGSSVKVSCKASGGTFSSYAISWVRQAPG |
| | Linker + | QGLEWMGGIGPFFGTANYAQKFQGRVTITADESTSTAYMEL |
| | protein tag | SSLRSEDTAVYYCARDTPYFDYWGQGTLVTVSSASTKGPSVFP |
| | (SEQ ID NO: | LAPSSKSTSGGTAALGCLVKDYFPEPVTVSWNSGALTSGVHTF |
| | 42 + SEQ ID | PAVLQSSGLYSLSSVVTVPSSSLGTQTYICNVNHKPSNTKVDKR |
| | NO: 31 + SEQ | VEPKSCGSGGGGVYHREAQSGKYKLTYAEAKAVCEFEGGHLA |
| | ID NO: 34) | TYKQLEAARKIGFHVCAAGWMAKGRVGYPIVKPGPNCGFGK |
| | | TGIIDYGIRLNRSERWDAYCYNPH |
| | | A |
| SEQ ID NO: 45 | DNA of Heavy | GAGGTGCAATTGGTCCAAAGCGGCGCTGAGGTCAAGAAG |
| | Chain + Linker + | CCTGGCAGCAGCGTGAAGGTCTCCTGCAAGGCCAGCGGCG |
| | protein tag | GCACATTCTCCAGCTATGCTATCAGCTGGGTCAGACAAGCC |
| | SEQ ID NO: 44 | CCCGGCCAAGGACTGGAATGGATGGGAGGAATCGGCCCTT |
| | | TCTTCGGAACCGCCAACTACGCCCAGAAGTTTCAGGGAAG |
| | | GGTGACCATCACCGCCGATGAGAGCACATCCACAGCCTAT |
| | | ATGGAGCTCTCCAGCCTGAGATCCGAAGACACCGCCGTCTA |
| | | CTACTGCGCTAGGGACACCCCCTACTTCGACTATTGGGGCC |
| | | AGGGCACACTCGTGACCGTGAGCTCAGCCAGCACCAAAGG |
| | | CCCTAGCGTCTTCCCCCTGGCTCCTTCCAGCAAGAGCACAA |
| | | GCGGAGGAACAGCTGCTCTCGGCTGCCTGGTCAAGGACTA |
| | | CTTCCCCGAGCCTGTCACAGTGTCCTGGAATAGCGGAGCCC |
| | | TGACCAGCGGCGTGCATACATTCCCCGCTGTGCTCCAGAGC |
| | | TCCGGCCTCTACAGCCTCAGCTCCGTGGTCACCGTCCCTAG |
| | | CTCCTCCCTGGGCACACAGACCTACATCTGCAACGTCAACC |
| | | ACAAGCCCTCCAACACCAAGGTGGACAAGAGGGTGGAGCC |
| | | CAAAAGCTGTGGATCCGGAGGAGGCGGCGTGTATCATAGA |
| | | GAGGCCCAGTCCGGCAAGTACAAGCTGACCTACGCCGAAG |
| | | CCAAGGCCGTGTGTGAGTTCGAGGGCGGACACCTGGCTAC |
| | | CTACAAACAGCTCGAAGCCGCTAGGAAGATCGGATTCCAC |
| | | GTGTGCGCCGCCGGATGGATGGCCAAAGGCAGAGTGGGC |
| | | TACCCCATTGTCAAGCCCGGACCCAACTGCGGATTCGGCAA |
| | | GACCGGCATCATCGACTACGGCATCAGGCTCAACAGGTCC |
| | | GAGAGATGGGACGCTTACTGCTACAATCCCCACGCC |
| SEQ ID NO: 46 | LCDR1 | SGDSIPNYYVY |
| SEQ ID NO: 47 | LCDR2 | DDSNRPS |
| SEQ ID NO: 48 | LCDR3 | QSFDSSLNAEV |
| SEQ ID NO: 49 | VL | SYELTQPLSVSVALGQTARITCSGDSIPNYYVYWYQQKPGQAP |
| | | VLVIYDDSNRPSGIPERFSGSNSGNTATLTISRAQAGDEADYYC |
| | | QSFDSSLNAEVFGGGTKLTVL |
| SEQ ID NO: 50 | DNA of VL SEQ | TCCTATGAACTCACACAGCCCCTGAGCGTGAGCGTGGCCCT |
| | ID NO: 49 | GGGCCAGACCGCCCGGATCACCTGCTCCGGCGACAGCATC |
| | | CCCAACTACTACGTGTACTGGTACCAGCAGAAGCCCGGCCA |
| | | GGCCCCCGTGCTGGTGATCTACGACGACAGCAACCGGCCC |
| | | AGCGGCATCCCCGAGCGGTTCAGCGGCAGCAACAGCGGCA |
| | | ACACCGCCACCCTGACCATTTCCAGAGCACAGGCAGGCGA |
| | | CGAGGCCGACTACTACTGCCAGAGCTTCGACAGCAGCCTG |
| | | AACGCCGAGGTGTTCGGCGGAGGGACCAAGTTAACCGTCC |
| | | TA |
| SEQ ID NO: 51 | Light Chain | SYELTQPLSVSVALGQTARITCSGDSIPNYYVYWYQQKPGQAP |
| | | VLVIYDDSNRPSGIPERFSGSNSGNTATLTISRAQAGDEADYYC |
| | | QSFDSSLNAEVFGGGTKLTVLGQPKAAPSVTLFPPSSEELQAN |
| | | KATLVCLISDFYPGAVTVAWKADSSPVKAGVETTTPSKQSNN |
| | | KYAASSYLSLTPEQWKSHRSYSCQVTHEGSTVEKTVAPTECS |
| SEQ ID NO: 52 | DNA of Light | AGCTACGAGCTGACCCAGCCCCTGAGCGTGAGCGTGGCCC |
| | Chain SEQ ID | TGGGCCAGACCGCCAGGATCACCTGCAGCGGCGACAGCAT |
| | NO: 51 | CCCCAACTACTACGTGTACTGGTATCAGCAGAAGCCCGGCC |
| | | AGGCCCCCGTGCTGGTGATCTACGACGACAGCAACAGGCC |
| | | CAGCGGCATCCCCGAGAGGTTCAGCGGCAGCAACAGCGGC |
| | | AACACCGCCACCCTGACCATCAGCAGAGCCCAGGCCGGCG |
| | | ACGAGGCCGACTACTACTGCCAGAGCTTCGACAGCTCACTG |
| | | AACGCCGAGGTGTTCGGCGGAGGGACCAAGCTGACCGTG |
| | | CTGGGCCAGCCTAAGGCTGCCCCCAGCGTGACCCTGTTCCC |
| | | CCCCAGCAGCGAGGAGCTGCAGGCCAACAAGGCCACCCTG |
| | | GTGTGCCTGATCAGCGACTTCTACCCAGGCGCCGTGACCGT |
| | | GGCCTGGAAGGCCGACAGCAGCCCCGTGAAGGCCGGCGT |
| | | GGAGACCACCACCCCCAGCAAGCAGAGCAACAACAAGTAC |
| | | GCCGCCAGCAGCTACCTGAGCCTGACCCCCGAGCAGTGGA |
| | | AGAGCCACAGGTCCTACAGCTGCCAGGTGACCCACGAGGG |
| | | CAGCACCGTGGAAAAGACCGTGGCCCCAACCGAGTGCAGC |
| NVS71 and NVS71T | | |
| SEQ ID NO: 53 (Kabat) | HCDR1 | SYAIS |
| SEQ ID NO: 54 (Kabat) | HCDR2 | RIIPIFGTANYAQKFQG |
| SEQ ID NO: 55 (Kabat) | HCDR3 | HGGYSFDS |
| SEQ ID NO: 56 (Chothia) | HCDR1 | GGTFNSY |
| SEQ ID NO: 57 (Chothia) | HCDR2 | IPIFGT |
| SEQ ID NO: 58 (Chothia) | HCDR3 | HGGYSFDS |
| SEQ ID NO: 59 | VH | EVQLVQSGAEVKKPGSSVKVSCKASGGTFNSYAISWVRQAPG |
| | | QGLEWMGRIIPIFGTANYAQKFQGRVTITADESTSTAYMELSS |
| | | LRSEDTAVYYCARHGGYSFDSWGQGTLVTVSS |
| SEQ ID NO: 60 | DNA of VH | GAGGTGCAGCTGGTGCAGAGCGGAGCCGAAGTGAAGAAA |
| | SEQ ID NO: 59 | CCCGGCAGCAGCGTGAAGGTGTCCTGCAAGGCCAGCGGC |
| | | GGCACCTTCAACAGCTACGCCATCAGCTGGGTGCGCCAGG |
| | | CTCCTGGACAGGGCCTGGAATGGATGGGCCGGATCATCCC |
| | | CATCTTCGGCACCGCCAACTACGCCCAGAAATTCCAGGGCA |
| | | GAGTGACCATCACCGCCGACGAGAGCACCAGCACCGCCTA |
| | | CATGGAACTGAGCAGCCTGAGAAGCGAGGACACCGCCGT |
| | | GTACTACTGTGCCCGGCACGGCGGCTACAGCTTCGATAGCT |
| | | GGGGCCAGGGCACCCTGGTGACCGTGAGCTCA |
| SEQ ID NO: 61 | Heavy Chain | EVQLVQSGAEVKKPGSSVKVSCKASGGTFSSYAISWVRQAPG |
| | | QGLEWMGRIIPIFGTANYAQKFQGRVTITADESTSTAYMELSS |
| | | LRSEDTAVYYCARHGGYSFDSWGQGTLVTVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSC |
| SEQ ID NO: 62 | DNA of Heavy | GAGGTGCAGCTGGTGCAGAGCGGAGCCGAAGTGAAGAAA |
| | Chain SEQ ID | CCCGGCAGCAGCGTGAAGGTGTCCTGCAAGGCCAGCGGC |
| | NO: 61 | GGCACCTTCAACAGCTACGCCATCAGCTGGGTGCGCCAGG |
| | | CTCCTGGACAGGGCCTGGAATGGATGGGCCGGATCATCCC |
| | | CATCTTCGGCACCGCCAACTACGCCCAGAAATTCCAGGGCA |
| | | GAGTGACCATCACCGCCGACGAGAGCACCAGCACCGCCTA |
| | | CATGGAACTGAGCAGCCTGAGAAGCGAGGACACCGCCGT |
| | | GTACTACTGTGCCCGGCACGGCGGCTACAGCTTCGATAGCT |
| | | GGGGCCAGGGCACCCTGGTGACCGTGAGCTCAGCCTCCAC |
| | | CAAGGGTCCATCGGTCTTCCCCCTGGCACCCTCCTCCAAGA |
| | | GCACCTCTGGGGGCACAGCGGCCCTGGGCTGCCTGGTCAA |
| | | GGACTACTTCCCCGAACCGGTGACGGTGTCGTGGAACTCA |
| | | GGCGCCCTGACCAGCGGCGTGCACACCTTCCCGGCTGTCCT |
| | | ACAGTCCTCAGGACTCTACTCCCTCAGCAGCGTGGTGACCG |
| | | TGCCCTCCAGCAGCTTGGGCACCCAGACCTACATCTGCAAC |
| | | GTGAATCACAAGCCCAGCAACACCAAGGTGGACAAGAGAG |
| | | TTGAGCCCAAATCTTGT |
| SEQ ID NO: 63 | Heavy Chain + | EVQLVQSGAEVKKPGSSVKVSCKASGGTFSSYAISWVRQAPG |
| | Linker + | QGLEWMGRIIPIFGTANYAQKFQGRVTITADESTSTAYMELSS |
| | protein tag | LRSEDTAVYYCARHGGYSFDSWGQGTLVTVSSASTKGPSVFP |
| | (SEQ ID NO: | LAPSSKSTSGGTAALGCLVKDYFPEPVTVSWNSGALTSGVHTF |
| | 61 + SEQ ID | PAVLQSSGLYSLSSVVTVPSSSLGTQTYICNVNHKPSNTKVDKR |
| | NO: 31 + SEQ | VEPKSCGSGGGGVYHREAQSGKYKLTYAEAKAVCEFEGGHLA |
| | ID NO: 33) | TYKQLEAARKIGFHVCAAGWMAKGRVGYPIVKPGPNCGFGK |
| | | TGIIDYGIRLNRSERWDAYCYNPHA |
| SEQ ID NO: 64 | DNA of Heavy | GAGGTGCAATTGGTGCAGAGCGGAGCTGAGGTGAAGAAG |
| | Chain + Linker + | CCCGGCAGCTCCGTCAAGGTGAGCTGCAAAGCCTCCGGAG |
| | protein tag | GCACCTTTTCCTCCTACGCTATCTCCTGGGTGAGGCAAGCC |
| | SEQ ID NO: 63 | CCCGGACAAGGACTGGAGTGGATGGGCAGGATCATCCCCA |
| | | TCTTCGGAACCGCCAACTACGCCCAGAAATTCCAGGGCAG |
| | | GGTGACCATCACCGCCGACGAAAGCACCAGCACCGCCTAC |
| | | ATGGAGCTCTCCAGCCTGAGGAGCGAGGACACCGCTGTGT |
| | | ACTACTGCGCCAGACACGGCGGCTACTATTTCGACAGCTGG |
| | | GGCCAGGGCACACTGGTGACCGTGAGCTCAGCAAGCACCA |
| | | AAGGACCCTCCGTCTTTCCTCTGGCCCCCAGCAGCAAGTCC |
| | | ACAAGCGGAGGAACCGCTGCCCTGGGATGTCTCGTGAAGG |
| | | ACTACTTCCCTGAGCCCGTGACAGTGTCCTGGAATAGCGGC |
| | | GCCCTGACAAGCGGCGTGCACACATTTCCCGCCGTCCTGCA |
| | | AAGCTCCGGCCTCTATAGCCTGAGCTCCGTCGTGACAGTCC |
| | | CCTCCAGCTCCCTGGGAACCCAGACCTACATCTGCAACGTC |
| | | AACCACAAGCCCAGCAACACAAAGGTGGACAAGAGGGTC |
| | | GAGCCTAAGAGCTGTGGATCCGGCGGCGGAGGAGTGTAC |
| | | CATAGGGAGGCCCAGAGCGGAAAGTACAAGCTGACCTATG |
| | | CCGAGGCTAAGGCCGTCTGCGAATTCGAGGGCGGCCATCT |
| | | GGCCACCTACAAGCAACTGGAGGCCGCTAGGAAGATCGGC |
| | | TTCCACGTCTGCGCCGCTGGATGGATGGCCAAGGGCAGAG |
| | | TGGGCTATCCCATCGTGAAGCCCGGCCCCAACTGCGGCTTC |
| | | GGAAAGACAGGCATCATCGACTACGGCATCAGGCTCAACA |
| | | GGAGCGAGAGGTGGGACGCTTACTGCTACAACCCCCATGC |
| | | C |
| SEQ ID NO: 65 (Kabat) | LCDR1 | SGDNLGSKYVD |
| SEQ ID NO: 66 (Kabat) | LCDR2 | SDNNRPS |
| SEQ ID NO: 67 (Kabat) | LCDR3 | QTYTSGNNYL |
| SEQ ID NO: 68 (Chothia) | LCDR1 | DNLGSKY |
| SEQ ID NO: 69 (Chothia) | LCDR2 | SDN |
| SEQ ID NO: 70 (Chothia) | LCDR3 | YTSGNNYL |
| SEQ ID NO: 71 | VL | SYELTQPPSVSVAPGQTARISCSGDNLGSKYVDWYQQKPGQ |
| | | APVLVIYSDNNRPSGIPERFSGSNSGNTATLTISGTQAEDEADY |
| | | YCQTYTSGNNYLVFGGGTKLTVL |
| SEQ ID NO: 72 | DNA of VL SEQ | AGCTACGAGCTGACTCAGCCCCCTTCTGTGTCTGTGGCCCC |
| | ID NO: 71 | TGGCCAGACCGCCAGAATCAGCTGCAGCGGCGACAACCTG |
| | | GGCAGCAAATACGTGGACTGGTATCAGCAGAAGCCCGGCC |
| | | AGGCTCCCGTGCTGGTGATCTACAGCGACAACAACCGGCC |
| | | CAGCGGCATCCCTGAGCGGTTCAGCGGCAGCAACAGCGGC |
| | | AATACCGCCACCCTGACCATCAGCGGCACCCAGGCCGAGG |
| | | ACGAGGCCGACTACTACTGCCAGACCTACACCAGCGGCAA |
| | | CAACTACCTGGTGTTCGGAGGCGGAACAAAGTTAACCGTC |
| | | CTA |
| SEQ ID NO: 73 | Light Chain | SYELTQPPSVSVAPGQTARISCSGDNLGSKYVDWYQQKPGQ |
| | | APVLVIYSDNNRPSGIPERFSGSNSGNTATLTISGTQAEDEADY |
| | | YCQTYTSGNNYLVFGGGTKLTVLGQPKAAPSVTLFPPSSEELQ |
| | | ANKATLVCLISDFYPGAVTVAWKADSSPVKAGVETTTPSKQS |
| | | NNKYAASSYLSLTPEQWKSHRSYSCQVTHEGSTVEKTVAPTEC |
| | | S |
| SEQ ID NO: 74 | DNA of Light | AGCTACGAGCTGACTCAGCCCCCTTCTGTGTCTGTGGCCCC |
| | Chain SEQ ID | TGGCCAGACCGCCAGAATCAGCTGCAGCGGCGACAACCTG |
| | NO: 73 | GGCAGCAAATACGTGGACTGGTATCAGCAGAAGCCCGGCC |
| | | AGGCTCCCGTGCTGGTGATCTACAGCGACAACAACCGGCC |
| | | CAGCGGCATCCCTGAGCGGTTCAGCGGCAGCAACAGCGGC |
| | | AATACCGCCACCCTGACCATCAGCGGCACCCAGGCCGAGG |
| | | ACGAGGCCGACTACTACTGCCAGACCTACACCAGCGGCAA |
| | | CAACTACCTGGTGTTCGGAGGCGGAACAAAGTTAACCGTC |
| | | CTAGGTCAGCCCAAGGCTGCCCCCTCGGTCACTCTGTTCCC |
| | | GCCCTCCTCTGAGGAGCTTCAAGCCAACAAGGCCACACTG |
| | | GTGTGTCTCATAAGTGACTTCTACCCGGGAGCCGTGACAGT |
| | | GGCCTGGAAGGCAGATAGCAGCCCCGTCAAGGCGGGAGT |
| | | GGAGACCACCACACCCTCCAAACAAAGCAACAACAAGTAC |
| | | GCGGCCAGCAGCTATCTGAGCCTGACGCCTGAGCAGTGGA |
| | | AGTCCCACAGAAGCTACAGCTGCCAGGTCACGCATGAAGG |
| | | GAGCACCGTGGAGAAGACAGTGGCCCCTACAGAATGTTCA |
| NVS72 and NVS72T | | |
| SEQ ID NO: 75 (Kabat) | HCDR1 | SYWIG |
| SEQ ID NO: 76 (Kabat) | HCDR2 | WIDPYRSEIRYSPSFQG |
| SEQ ID NO: 77 (Kabat) | HCDR3 | VSSEPFDS |
| SEQ ID NO: 78 (Chothia) | HCDR1 | GYSFTSY |
| SEQ ID NO: 79 (Chothia) | HCDR2 | DPYRSE |
| SEQ ID NO: 80 (Chothia) | HCDR3 | VSSEPFDS |
| SEQ ID NO: 81 | VH | EVQLVQSGAEVKKPGESLKISCKGSGYSFTSYWIGWVRQMPG |
| | | KGLEWMGWIDPYRSEIRYSPSFQGQVTISADKSISTAYLQWSS |
| | | LKASDTAMYYCARVSSEPFDSWGQGTLVTVSS |
| SEQ ID NO: 82 | DNA of VH | GAGGTCCAATTGGTCCAATCCGGAGCCGAAGTCAAGAAAC |
| | SEQ ID NO: 81 | CCGGCGAGTCCCTCAAAATCAGCTGCAAGGGCTCCGGCTA |
| | | CTCCTTCACCAGCTACTGGATCGGATGGGTGAGGCAGATG |
| | | CCCGGCAAAGGCCTCGAGTGGATGGGCTGGATCGACCCCT |
| | | ATAGGTCCGAGATTAGGTACAGCCCCTCCTTCCAGGGCCAG |
| | | GTCACCATCTCCGCCGACAAGAGCATCAGCACCGCCTACCT |
| | | CCAATGGTCCTCCCTCAAGGCCTCCGATACCGCCATGTATT |
| | | ACTGCGCCAGGGTCAGCAGCGAGCCCTTTGACAGCTGGGG |
| | | CCAGGGAACCCTCGTGACCGTCAGCTCA |
| SEQ ID NO: 83 | Heavy Chain | EVQLVQSGAEVKKPGESLKISCKGSGYSFTSYWIGWVRQMPG |
| | | KGLEWMGWIDPYRSEIRYSPSFQGQVTISADKSISTAYLQWSS |
| | | LKASDTAMYYCARVSSEPFDSWGQGTLVTVSSASTKGPSVFP |
| | | LAPSSKSTSGGTAALGCLVKDYFPEPVTVSWNSGALTSGVHTF |
| | | PAVLQSSGLYSLSSVVTVPSSSLGTQTYICNVNHKPSNTKVDKR |
| | | VEPKSC |
| SEQ ID NO: 84 | DNA of Heavy | GAGGTCCAATTGGTCCAATCCGGAGCCGAAGTCAAGAAAC |
| | Chain SEQ ID | CCGGCGAGTCCCTCAAAATCAGCTGCAAGGGCTCCGGCTA |
| | NO: 83 | CTCCTTCACCAGCTACTGGATCGGATGGGTGAGGCAGATG |
| | | CCCGGCAAAGGCCTCGAGTGGATGGGCTGGATCGACCCCT |
| | | ATAGGTCCGAGATTAGGTACAGCCCCTCCTTCCAGGGCCAG |
| | | GTCACCATCTCCGCCGACAAGAGCATCAGCACCGCCTACCT |
| | | CCAATGGTCCTCCCTCAAGGCCTCCGATACCGCCATGTATT |
| | | ACTGCGCCAGGGTCAGCAGCGAGCCCTTTGACAGCTGGGG |
| | | CCAGGGAACCCTCGTGACCGTCAGCTCAGCCAGCACCAAA |
| | | GGACCTAGCGTGTTCCCCCTCGCTCCCTCCTCCAAGAGCAC |
| | | ATCCGGCGGAACCGCTGCTCTGGGATGTCTCGTCAAGGAC |
| | | TACTTCCCCGAGCCCGTGACCGTGAGCTGGAATAGCGGCG |
| | | CCCTGACCTCCGGAGTCCACACATTCCCCGCTGTCCTGCAG |
| | | AGCAGCGGCCTGTATAGCCTGTCCTCCGTCGTGACCGTCCC |
| | | TAGCAGCTCCCTGGGAACCCAGACCTACATCTGCAACGTCA |
| | | ACCACAAGCCTAGCAACACCAAGGTGGACAAGAGGGTGG |
| | | AGCCCAAATCCTGC |
| SEQ ID NO: 85 | Heavy Chain + | EVQLVQSGAEVKKPGESLKISCKGSGYSFTSYWIGWVRQMPG |
| | Linker + | KGLEWMGWIDPYRSEIRYSPSFQGQVTISADKSISTAYLQWSS |
| | protein tag | LKASDTAMYYCARVSSEPFDSWGQGTLVTVSSASTKGPSVFP |
| | (SEQ ID NO: | LAPSSKSTSGGTAALGCLVKDYFPEPVTVSWNSGALTSGVHTF |
| | 83 + SEQ ID | PAVLQSSGLYSLSSVVTVPSSSLGTQTYICNVNHKPSNTKVDKR |
| | NO: 31 + SEQ | VEPKSCGSGGGGVYHREAQSGKYKLTYAEAKAVCEFEGGHLA |
| | ID NO: 33) | TYKQLEAARKIGFHVCAAGWMAKGRVGYPIVKPGPNCGFGK |
| | | TGIIDYGIRLNRSERWDAYCYNPHA |
| SEQ ID NO: 86 | DNA of Heavy | GAGGTCCAATTGGTCCAATCCGGAGCCGAAGTCAAGAAAC |
| | Chain + Linker + | CCGGCGAGTCCCTCAAAATCAGCTGCAAGGGCTCCGGCTA |
| | protein tag | CTCCTTCACCAGCTACTGGATCGGATGGGTGAGGCAGATG |
| | SEQ ID NO: 85 | CCCGGCAAAGGCCTCGAGTGGATGGGCTGGATCGACCCCT |
| | | ATAGGTCCGAGATTAGGTACAGCCCCTCCTTCCAGGGCCAG |
| | | GTCACCATCTCCGCCGACAAGAGCATCAGCACCGCCTACCT |
| | | CCAATGGTCCTCCCTCAAGGCCTCCGATACCGCCATGTATT |
| | | ACTGCGCCAGGGTCAGCAGCGAGCCCTTTGACAGCTGGGG |
| | | CCAGGGAACCCTCGTGACCGTCAGCTCAGCCAGCACCAAA |
| | | GGACCTAGCGTGTTCCCCCTCGCTCCCTCCTCCAAGAGCAC |
| | | ATCCGGCGGAACCGCTGCTCTGGGATGTCTCGTCAAGGAC |
| | | TACTTCCCCGAGCCCGTGACCGTGAGCTGGAATAGCGGCG |
| | | CCCTGACCTCCGGAGTCCACACATTCCCCGCTGTCCTGCAG |
| | | AGCAGCGGCCTGTATAGCCTGTCCTCCGTCGTGACCGTCCC |
| | | TAGCAGCTCCCTGGGAACCCAGACCTACATCTGCAACGTCA |
| | | ACCACAAGCCTAGCAACACCAAGGTGGACAAGAGGGTGG |
| | | AGCCCAAATCCTGCGGATCCGGAGGAGGCGGCGTGTATCA |
| | | CAGAGAGGCCCAGAGCGGCAAGTACAAGCTCACATACGCT |
| | | GAGGCCAAAGCCGTGTGCGAATTCGAGGGCGGACATCTG |
| | | GCCACATATAAGCAGCTGGAGGCCGCCAGGAAGATCGGCT |
| | | TCCACGTGTGCGCTGCCGGCTGGATGGCCAAAGGCAGAGT |
| | | GGGCTACCCTATCGTCAAGCCCGGCCCCAACTGCGGCTTTG |
| | | GCAAGACCGGCATCATCGACTACGGCATCAGGCTCAACAG |
| | | GTCCGAAAGGTGGGATGCCTACTGCTACAATCCCCACGCC |
| SEQ ID NO: 87 (Kabat) | LCDR1 | SGDKLGDHYAY |
| SEQ ID NO: 88 (Kabat) | LCDR2 | DDSKRPS |
| SEQ ID NO: 89 (Kabat) | LCDR3 | ATWTFEGDYV |
| SEQ ID NO: 90 (Chothia) | LCDR1 | DKLGDHY |
| SEQ ID NO: 91 (Chothia) | LCDR2 | DDS |
| SEQ ID NO: 92 (Chothia) | LCDR3 | WTFEGDY |
| SEQ ID NO: 93 | VL | SYVLTQPPSVSVAPGKTARITCSGDKLGDHYAYWYQQKPGQ |
| | | APVLVIYDDSKRPSGIPERFSGSNSGNTATLTISRVEAGDEADY |
| | | YCATWTFEGDYVFGGGTKLTVL |
| SEQ ID NO: 94 | DNA of VL SEQ | TCCTACGTCCTGACACAACCTCCCAGCGTGAGCGTCGCTCC |
| | ID NO: 93 | TGGCAAGACAGCCAGAATCACCTGCAGCGGCGACAAGCTG |
| | | GGCGACCACTACGCCTACTGGTATCAGCAGAAACCCGGCC |
| | | AAGCTCCCGTGCTGGTGATCTATGACGACAGCAAGAGACC |
| | | CTCCGGCATCCCTGAGAGATTCAGCGGAAGCAACTCCGGC |
| | | AACACCGCCACCCTGACCATCAGCAGGGTCGAAGCCGGCG |
| | | ATGAGGCCGACTACTACTGCGCCACCTGGACCTTTGAGGG |
| | | CGACTACGTGTTCGGAGGCGGCACCAAGTTAACCGTCCTA |
| SEQ ID NO: 95 | Light Chain | SYVLTQPPSVSVAPGKTARITCSGDKLGDHYAYWYQQKPGQ |
| | | APVLVIYDDSKRPSGIPERFSGSNSGNTATLTISRVEAGDEADY |
| | | YCATWTFEGDYVFGGGTKLTVLGQPKAAPSVTLFPPSSEELQA |
| | | NKATLVCLISDFYPGAVTVAWKADSSPVKAGVETTTPSKQSN |
| | | NKYAASSYLSLTPEQWKSHRSYSCQVTHEGSTVEKTVAPTECS |
| SEQ ID NO: 96 | DNA of Light | TCCTACGTCCTGACACAACCTCCCAGCGTGAGCGTCGCTCC |
| | Chain SEQ ID | TGGCAAGACAGCCAGAATCACCTGCAGCGGCGACAAGCTG |
| | NO: 95 | GGCGACCACTACGCCTACTGGTATCAGCAGAAACCCGGCC |
| | | AAGCTCCCGTGCTGGTGATCTATGACGACAGCAAGAGACC |
| | | CTCCGGCATCCCTGAGAGATTCAGCGGAAGCAACTCCGGC |
| | | AACACCGCCACCCTGACCATCAGCAGGGTCGAAGCCGGCG |
| | | ATGAGGCCGACTACTACTGCGCCACCTGGACCTTTGAGGG |
| | | CGACTACGTGTTCGGAGGCGGCACCAAGTTAACCGTCCTA |
| | | GGACAGCCTAAGGCCGCTCCCTCCGTGACACTGTTTCCCCC |
| | | TAGCAGCGAGGAGCTGCAGGCCAACAAGGCCACCCTCGTG |
| | | TGCCTCATCTCCGACTTCTACCCTGGCGCCGTCACAGTCGCC |
| | | TGGAAAGCCGACAGCTCCCCCGTCAAAGCTGGCGTGGAGA |
| | | CCACCACCCCCAGCAAGCAGAGCAACAACAAGTACGCCGC |
| | | CTCCTCCTATCTGAGCCTGACCCCCGAGCAGTGGAAGAGCC |
| | | ACAGGAGCTACTCCTGCCAGGTGACACACGAGGGCAGCAC |
| | | CGTCGAGAAGACCGTCGCTCCCACCGAGTGCAGC |
| NVS73 and NVS73T | | |
| SEQ ID NO: 108 | HCDR1 | GFTISRSYWIC |
| SEQ ID NO: 109 | HCDR2 | CIYGDNDITPLYANWAKG |
| SEQ ID NO: 110 | HCDR3 | LGYADYAYDL |
| SEQ ID NO: 111 | VH | EVQLVESGGGSVQPGGSLRLSCTASGFTISRSYWICWVRQAP |
| | | GKGLEWVGCIYGDNDITPLYANWAKGRFTISRDTSKNTVYLQ |
| | | MNSLRAEDTATYYCARLGYADYAYDLWGQGTTVTVSS |
| SEQ ID NO: 112 | DNA of VH | GAGGTCCAGCTGGTGGAGAGCGGAGGAGGAAGCGTCCAG |
| | 111 | TCACCATCAGCAGGAGCTACTGGATCTGCTGGGTGAGGCA |
| | | GGCTCCTGGCAAGGGACTCGAGTGGGTGGGCTGCATCTAC |
| | | GGCGACAACGACATCACCCCCCTCTACGCCAACTGGGCTAA |
| | | GGGCAGGTTCACCATTAGCAGGGACACCAGCAAGAACACC |
| | | GTGTACCTCCAGATGAACAGCCTGAGGGCCGAGGATACCG |
| | | CCACCTACTATTGCGCCAGGCTGGGCTACGCCGATTACGCC |
| | | TATGACCTCTGGGGCCAGGGCACCACAGTGACCGTCAGCT |
| | | CA |
| SEQ ID NO: 113 | Heavy Chain | EVQLVESGGGSVQPGGSLRLSCTASGFTISRSYWICWVRQAP |
| | | GKGLEWVGCIYGDNDITPLYANWAKGRFTISRDTSKNTVYLQ |
| | | MNSLRAEDTATYYCARLGYADYAYDLWGQGTTVTVSSASTK |
| | | GPSVFPLAPSSKSTSGGTAALGCLVKDYFPEPVTVSWNSGALT |
| | | SGVHTFPAVLQSSGLYSLSSVVTVPSSSLGTQTYICNVNHKPSN |
| | | TKVDKRVEPKSC |
| SEQ ID NO: 114 | DNA of Heavy | GAGGTCCAGCTGGTGGAGAGCGGAGGAGGAAGCGTCCAG |
| | Chain SEQ ID | CCTGGAGGCAGCCTGAGACTGAGCTGCACCGCCAGCGGCT |
| | NO: 113 | TCACCATCAGCAGGAGCTACTGGATCTGCTGGGTGAGGCA |
| | | GGCTCCTGGCAAGGGACTCGAGTGGGTGGGCTGCATCTAC |
| | | GGCGACAACGACATCACCCCCCTCTACGCCAACTGGGCTAA |
| | | GGGCAGGTTCACCATTAGCAGGGACACCAGCAAGAACACC |
| | | GTGTACCTCCAGATGAACAGCCTGAGGGCCGAGGATACCG |
| | | CCACCTACTATTGCGCCAGGCTGGGCTACGCCGATTACGCC |
| | | TATGACCTCTGGGGCCAGGGCACCACAGTGACCGTCAGCT |
| | | CAGCCTCCACCAAGGGACCTTCCGTGTTCCCCCTGGCCCCT |
| | | AGCTCCAAGTCCACCAGCGGAGGAACAGCCGCTCTGGGCT |
| | | GTCTGGTGAAGGACTACTTCCCCGAGCCTGTGACCGTGTCC |
| | | TGGAATTCCGGCGCCCTCACAAGCGGAGTGCATACCTTCCC |
| | | CGCCGTGCTGCAAAGCTCCGGACTGTACTCCCTCTCCAGCG |
| | | TGGTGACAGTGCCTTCCAGCAGCCTCGGCACCCAGACCTAC |
| | | ATCTGCAACGTGAACCACAAGCCCTCCAATACCAAGGTGG |
| | | ACAAGAGGGTCGAGCCTAAAAGCTGT |
| SEQ ID NO: 115 | Heavy Chain + | EVQLVESGGGSVQPGGSLRLSCTASGFTISRSYWICWVRQAP |
| | Linker + | GKGLEWVGCIYGDNDITPLYANWAKGRFTISRDTSKNTVYLQ |
| | protein tag | MNSLRAEDTATYYCARLGYADYAYDLWGQGTTVTVSSASTK |
| | (SEQ ID NO: | GPSVFPLAPSSKSTSGGTAALGCLVKDYFPEPVTVSWNSGALT |
| | 113 + SEQ ID | SGVHTFPAVLQSSGLYSLSSVVTVPSSSLGTQTYICNVNHKPSN |
| | NO: 31 + SEQ | TKVDKRVEPKSCGSGGGGVYHREAQSGKYKLTYAEAKAVCEF |
| | ID NO: 33) | EGGHLATYKQLEAARKIGFHVCAAGWMAKGRVGYPIVKPGP |
| | | NCGFGKTGIIDYGIRLNRSERWDAYCYNPHA |
| SEQ ID NO: 116 | DNA of Heavy | GAGGTCCAGCTGGTGGAGAGCGGAGGAGGAAGCGTCCAG |
| | Chain + Linker + | CCTGGAGGCAGCCTGAGACTGAGCTGCACCGCCAGCGGCT |
| | protein tag | TCACCATCAGCAGGAGCTACTGGATCTGCTGGGTGAGGCA |
| | SEQ ID NO: | GGCTCCTGGCAAGGGACTCGAGTGGGTGGGCTGCATCTAC |
| | 115 | GGCGACAACGACATCACCCCCCTCTACGCCAACTGGGCTAA |
| | | GGGCAGGTTCACCATTAGCAGGGACACCAGCAAGAACACC |
| | | GTGTACCTCCAGATGAACAGCCTGAGGGCCGAGGATACCG |
| | | CCACCTACTATTGCGCCAGGCTGGGCTACGCCGATTACGCC |
| | | TATGACCTCTGGGGCCAGGGCACCACAGTGACCGTCAGCT |
| | | CAGCCTCCACCAAGGGACCTTCCGTGTTCCCCCTGGCCCCT |
| | | AGCTCCAAGTCCACCAGCGGAGGAACAGCCGCTCTGGGCT |
| | | GTCTGGTGAAGGACTACTTCCCCGAGCCTGTGACCGTGTCC |
| | | TGGAATTCCGGCGCCCTCACAAGCGGAGTGCATACCTTCCC |
| | | CGCCGTGCTGCAAAGCTCCGGACTGTACTCCCTCTCCAGCG |
| | | TGGTGACAGTGCCTTCCAGCAGCCTCGGCACCCAGACCTAC |
| | | ATCTGCAACGTGAACCACAAGCCCTCCAATACCAAGGTGG |
| | | ACAAGAGGGTCGAGCCTAAAAGCTGTGGATCCGGAGGAG |
| | | GCGGCGTGTATCATAGAGAGGCCCAGTCCGGCAAGTACAA |
| | | GCTGACCTACGCCGAAGCCAAGGCCGTGTGTGAGTTCGAG |
| | | GGCGGACACCTGGCTACCTACAAACAGCTCGAAGCCGCTA |
| | | GGAAGATCGGATTCCACGTGTGCGCCGCCGGATGGATGGC |
| | | CAAAGGCAGAGTGGGCTACCCCATTGTCAAGCCCGGACCC |
| | | AACTGCGGATTCGGCAAGACCGGCATCATCGACTACGGCA |
| | | TCAGGCTCAACAGGTCCGAGAGATGGGACGCTTACTGCTA |
| | | CAATCCCCACGCC |
| SEQ ID NO: 117 | LCDR1 | QSSQSVYGNIWMA |
| SEQ ID NO: 118 | LCDR2 | QASKLAS |
| SEQ ID NO: 119 | LCDR3 | QGNFNTGDRYA |
| SEQ ID NO: 120 | VL | EIVMTQSPSTLSASVGDRVIITCQSSQSVYGNIWMAWYQQK |
| | | PGRAPKLLIYQASKLASGVPSRFSGSGSGAEFTLTISSLQPDDFA |
| | | TYYCQGNFNTGDRYAFGQGTKLTVLKR |
| SEQ ID NO: 121 | DNA of VL SEQ | GAGATCGTCATGACCCAGAGCCCCAGCACACTCAGCGCCTC |
| | ID NO: 120 | CGTGGGAGACAGGGTGATCATCACCTGCCAGTCCTCCCAG |
| | | TCCGTGTACGGCAACATCTGGATGGCCTGGTACCAGCAGA |
| | | AGCCCGGCAGAGCCCCCAAGCTGCTGATCTACCAGGCCAG |
| | | CAAGCTCGCCTCCGGAGTGCCCAGCAGATTTTCCGGCTCCG |
| | | GATCCGGAGCCGAGTTCACACTGACCATCAGCAGCCTGCA |
| | | GCCCGATGACTTCGCCACCTACTATTGCCAGGGCAACTTCA |
| | | ACACCGGCGACAGGTACGCCTTTGGCCAGGGCACCAAGCT |
| | | GACCGTCCTCAAGCGT |
| SEQ ID NO: 122 | Light Chain | EIVMTQSPSTLSASVGDRVIITCQSSQSVYGNIWMAWYQQK |
| | | PGRAPKLLIYQASKLASGVPSRFSGSGSGAEFTLTISSLQPDDFA |
| | | TYYCQGNFNTGDRYAFGQGTKLTVLKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTKSF |
| | | NRGEC |
| SEQ ID NO: 123 | DNA of Light | GAGATCGTCATGACCCAGAGCCCCAGCACACTCAGCGCCTC |
| | Chain SEQ ID | CGTGGGAGACAGGGTGATCATCACCTGCCAGTCCTCCCAG |
| | NO: 122 | TCCGTGTACGGCAACATCTGGATGGCCTGGTACCAGCAGA |
| | | AGCCCGGCAGAGCCCCCAAGCTGCTGATCTACCAGGCCAG |
| | | CAAGCTCGCCTCCGGAGTGCCCAGCAGATTTTCCGGCTCCG |
| | | GATCCGGAGCCGAGTTCACACTGACCATCAGCAGCCTGCA |
| | | GCCCGATGACTTCGCCACCTACTATTGCCAGGGCAACTTCA |
| | | ACACCGGCGACAGGTACGCCTTTGGCCAGGGCACCAAGCT |
| | | GACCGTCCTCAAGCGTACGGTGGCTGCTCCCAGCGTCTTCA |
| | | TCTTCCCCCCCAGCGATGAGCAGCTCAAGAGCGGCACAGC |
| | | CTCCGTGGTGTGCCTCCTGAACAACTTCTACCCTAGGGAGG |
| | | CCAAGGTGCAATGGAAGGTGGACAACGCCCTGCAGAGCG |
| | | GCAACAGCCAGGAGTCCGTGACCGAGCAGGACTCCAAGG |
| | | ACAGCACCTACAGCCTGAGCAGCACACTCACCCTGAGCAAA |
| | | GCCGACTACGAGAAGCACAAGGTCTACGCCTGCGAGGTGA |
| | | CCCATCAGGGCCTGTCCAGCCCCGTGACCAAGAGCTTCAAC |
| | | AGAGGCGAGTGC |
| NVS75 and NVS75T | | |
| SEQ ID NO: 189 | HCDR1 | GFTFSVYGMN |
| SEQ ID NO: 190 | HCDR2 | IIWYDGDNQYYADSVKG |
| SEQ ID NO: 191 | HCDR3 | DLRTGPFDY |
| SEQ ID NO: 192 | VH | QVQLVESGGGVVQPGRSLRLSCAASGFTFSVYGMNWVRQA |
| | | PGKGLEWVAIIWYDGDNQYYADSVKGRFTISRDNSKNTLYLQ |
| | | MNGLRAEDTAVYYCARDLRTGPFDYWGQGTLVTVSS |
| SEQ ID NO: 193 | DNA of VH | CAGGTGCAGCTGGTGGAATCTGGCGGCGGAGTGGTGCAG |
| | SEQ ID NO: 192 | CCTGGCAGAAGCCTGAGACTGAGCTGTGCCGCCAGCGGCT |
| | | TCACCTTCAGCGTGTACGGCATGAACTGGGTGCGCCAGGC |
| | | CCCTGGCAAAGGCCTGGAATGGGTGGCCATCATTTGGTAC |
| | | GACGGCGACAACCAGTACTACGCCGACAGCGTGAAGGGCC |
| | | GGTTCACCATCAGCCGGGACAACAGCAAGAACACCCTGTA |
| | | CCTGCAGATGAACGGCCTGCGGGCCGAGGATACCGCCGTG |
| | | TACTACTGCGCCAGGGACCTGAGAACAGGCCCCTTCGATTA |
| | | TTGGGGCCAGGGCACCCTCGTGACCGTGTCTAGC |
| SEQ ID NO: 194 | Heavy Chain | QVQLVESGGGVVQPGRSLRLSCAASGFTFSVYGMNWVRQA |
| | | PGKGLEWVAIIWYDGDNQYYADSVKGRFTISRDNSKNTLYLQ |
| | | MNGLRAEDTAVYYCARDLRTGPFDYWGQGTLVTVSSASTKG |
| | | PSVFPLAPSSKSTSGGTAALGCLVKDYFPEPVTVSWNSGALTS |
| | | GVHTFPAVLQSSGLYSLSSVVTVPSSSLGTQTYICNVNHKPSNT |
| | | KVDKRVEPKSC |
| SEQ ID NO: 195 | DNA of Heavy | CAGGTGCAGCTGGTGGAATCTGGCGGCGGAGTGGTGCAG |
| | Chain ID NO: | CCTGGCAGAAGCCTGAGACTGAGCTGTGCCGCCAGCGGCT |
| | 194 | TCACCTTCAGCGTGTACGGCATGAACTGGGTGCGCCAGGC |
| | | CCCTGGCAAAGGCCTGGAATGGGTGGCCATCATTTGGTAC |
| | | GACGGCGACAACCAGTACTACGCCGACAGCGTGAAGGGCC |
| | | GGTTCACCATCAGCCGGGACAACAGCAAGAACACCCTGTA |
| | | CCTGCAGATGAACGGCCTGCGGGCCGAGGATACCGCCGTG |
| | | TACTACTGCGCCAGGGACCTGAGAACAGGCCCCTTCGATTA |
| | | TTGGGGCCAGGGCACCCTCGTGACCGTGTCTAGCGCCTCTA |
| | | CAAAGGGCCCCAGCGTGTTCCCTCTGGCCCCTAGCAGCAA |
| | | GTCTACCAGCGGAGGAACAGCCGCCCTGGGCTGCCTCGTG |
| | | AAGGACTACTTTCCCGAGCCCGTGACAGTGTCCTGGAACTC |
| | | TGGCGCCCTGACAAGCGGCGTGCACACCTTTCCAGCCGTGC |
| | | TGCAGAGCAGCGGCCTGTACTCTCTGAGCAGCGTCGTGAC |
| | | TGTGCCCAGCAGCTCTCTGGGCACCCAGACCTACATCTGCA |
| | | ACGTGAACCACAAGCCCAGCAACACCAAGGTGGACAAGCG |
| | | GGTGGAACCCAAGAGCTGT |
| SEQ ID NO: 196 | Heavy Chain + | QVQLVESGGGVVQPGRSLRLSCAASGFTFSVYGMNWVRQA |
| | Linker + | PGKGLEWVAIIWYDGDNQYYADSVKGRFTISRDNSKNTLYLQ |
| | protein tag | MNGLRAEDTAVYYCARDLRTGPFDYWGQGTLVTVSSASTKG |
| | (SEQ ID NO: | PSVFPLAPSSKSTSGGTAALGCLVKDYFPEPVTVSWNSGALTS |
| | 194 + SEQ ID | GVHTFPAVLQSSGLYSLSSVVTVPSSSLGTQTYICNVNHKPSNT |
| | NO: 31 + SEQ | KVDKRVEPKSCGSGGGGVYHREAQSGKYKLTYAEAKAVCEFE |
| | ID NO: 33) | GGHLATYKQLEAARKIGFHVCAAGWMAKGRVGYPIVKPGPN |
| | | CGFGKTGIIDYGIRLNRSERWDAYCYNPHA |
| SEQ ID NO: 197 | DNA of Heavy | CAGGTGCAGCTGGTGGAATCTGGCGGCGGAGTGGTGCAG |
| | Chain SEQ ID | CCTGGCAGAAGCCTGAGACTGAGCTGTGCCGCCAGCGGCT |
| | NO: 196 | TCACCTTCAGCGTGTACGGCATGAACTGGGTGCGCCAGGC |
| | | CCCTGGCAAAGGCCTGGAATGGGTGGCCATCATTTGGTAC |
| | | GACGGCGACAACCAGTACTACGCCGACAGCGTGAAGGGCC |
| | | GGTTCACCATCAGCCGGGACAACAGCAAGAACACCCTGTA |
| | | CCTGCAGATGAACGGCCTGCGGGCCGAGGATACCGCCGTG |
| | | TACTACTGCGCCAGGGACCTGAGAACAGGCCCCTTCGATTA |
| | | TTGGGGCCAGGGCACCCTCGTGACCGTGTCTAGCGCCTCTA |
| | | CAAAGGGCCCCAGCGTGTTCCCTCTGGCCCCTAGCAGCAA |
| | | GTCTACCAGCGGAGGAACAGCCGCCCTGGGCTGCCTCGTG |
| | | AAGGACTACTTTCCCGAGCCCGTGACAGTGTCCTGGAACTC |
| | | TGGCGCCCTGACAAGCGGCGTGCACACCTTTCCAGCCGTGC |
| | | TGCAGAGCAGCGGCCTGTACTCTCTGAGCAGCGTCGTGAC |
| | | TGTGCCCAGCAGCTCTCTGGGCACCCAGACCTACATCTGCA |
| | | ACGTGAACCACAAGCCCAGCAACACCAAGGTGGACAAGCG |
| | | GGTGGAACCCAAGAGCTGT |
| SEQ ID NO: 198 | LCDR1 | RASQSIGSSLH |
| SEQ ID NO: 199 | LCDR2 | YASQSFS |
| SEQ ID NO: 200 | LCDR3 | HQSSSLPFT |
| SEQ ID NO: 201 | VL | EIVLTQSPDFQSVTPKEKVTITCRASQSIGSSLHWYQQKPDQS |
| | | PKLLIKYASQSFSGVPSRFSGSGSGTDFTLTINSLEAEDAAAYYC |
| | | HQSSSLPFTFGPGTKVDIKR |
| SEQ ID NO: 202 | DNA of VL SEQ | GAGATCGTGCTGACCCAGAGCCCCGACTTTCAGAGCGTGA |
| | ID NO: 201 | CCCCCAAAGAAAAAGTGACCATCACCTGTCGGGCCAGCCA |
| | | GAGCATCGGCTCTAGCCTGCACTGGTATCAGCAGAAGCCC |
| | | GACCAGTCCCCCAAGCTGCTGATTAAGTACGCCAGCCAGTC |
| | | CTTCAGCGGCGTGCCCAGCAGATTTTCTGGCAGCGGCTCCG |
| | | GCACCGACTTCACCCTGACCATCAACAGCCTGGAAGCCGA |
| | | GGACGCCGCTGCCTACTACTGTCACCAGAGCAGCAGCCTG |
| | | CCCTTCACCTTTGGCCCTGGCACCAAGGTGGACATCAAGCG |
| | | G |
| SEQ ID NO: 202 | Light Chain | EIVLTQSPDFQSVTPKEKVTITCRASQSIGSSLHWYQQKPDQS |
| | | PKLLIKYASQSFSGVPSRFSGSGSGTDFTLTINSLEAEDAAAYYC |
| | | HQSSSLPFTFGPGTKVDIKRTVAAPSVFIFPPSDEQLKSGTASV |
| | | VCLLNNFYPREAKVQWKVDNALQSGNSQESVTEQDSKDSTY |
| | | SLSSTLTLSKADYEKHKVYACEVTHQGLSSPVTKSFNRGEC |
| SEQ ID NO: 203 | DNA of Light | GAGATCGTGCTGACCCAGAGCCCCGACTTTCAGAGCGTGA |
| | Chain SEQ ID | CCCCCAAAGAAAAAGTGACCATCACCTGTCGGGCCAGCCA |
| | NO: 202 | GAGCATCGGCTCTAGCCTGCACTGGTATCAGCAGAAGCCC |
| | | GACCAGTCCCCCAAGCTGCTGATTAAGTACGCCAGCCAGTC |
| | | CTTCAGCGGCGTGCCCAGCAGATTTTCTGGCAGCGGCTCCG |
| | | GCACCGACTTCACCCTGACCATCAACAGCCTGGAAGCCGA |
| | | GGACGCCGCTGCCTACTACTGTCACCAGAGCAGCAGCCTG |
| | | CCCTTCACCTTTGGCCCTGGCACCAAGGTGGACATCAAGCG |
| | | GACAGTGGCCGCTCCCTCCGTGTTCATCTTCCCACCTAGCG |
| | | ACGAGCAGCTGAAGTCTGGCACAGCCAGCGTCGTGTGCCT |
| | | GCTGAACAACTTCTACCCCCGCGAGGCCAAGGTGCAGTGG |
| | | AAAGTGGACAACGCCCTGCAGAGCGGCAACAGCCAGGAA |
| | | AGCGTGACCGAGCAGGACAGCAAGGACTCCACCTACAGCC |
| | | TGAGCAGCACCCTGACACTGAGCAAGGCCGACTACGAGAA |
| | | GCACAAGGTGTACGCCTGCGAAGTGACCCACCAGGGCCTG |
| | | TCTAGCCCCGTGACCAAGAGCTTCAACCGGGGCGAGTGC |

TABLE 2b
Sequence of Intra-Articular Proteins

| | |
|---|---|
| Human VEGF | SEQ ID NO: 97 |
| | APMAEGGGQNHHEVVKFMDVYQRSYCHPIETLVDIFQEYPDEIEYIFKP |
| | SCVPLMRCGGCCNDEGLECVPTEESNITMQIMRIKPHQGQHIGEMSFLQH |
| | NKCECRPKKDRARQEKKSVRGKGKGQKRKRKKSRYKSWSVYVGARCCLMP |
| | WSLPGPHPCGPCSERRKHLFVQDPQTCKCSCKNTDSRCKARQLELNERTC |
| | RCDKPRR |
| Human EPO | SEQ ID NO: 98 |
| | APPRLICDSRVLERYLLEAKEAENITTGCAEHCSLNENITVPDTKVNFYAWKRMEVGQQA |
| | VEVWQGLALLSEAVLRGQALLVNSSQPWEPLQLHVDKAVSGLRSLTTLLRALGAQKEAIS |
| | PPDAASAAPLRTITADTFRKLFRVYSNFLRGKLKLYTGEACRTGDR |
| Human C5 | SEQ ID NO: 99 |
| | QEQTYVISAPKIFRVGASENIVIQVYGYTEAFDATISIKSYP |
| | DKKFSYSSGHVHLSSENKFQNSAILTIQPKQLPGGQNPVSYVYLEVVSKHFSKSKRMPIT |
| | YDNGFLFIHTDKPVYTPDQSVKVRVYSLNDDLKPAKRETVLTFIDPEGSEVDMVEEIDHI |
| | GIISFPDFKIPSNPRYGMWTIKAKYKEDFSTTGTAYFEVKEYVLPHFSVSIEPEYNFIGY |
| | KNFKNFEITIKARYFYNKVVTEADVYITFGIREDLKDDQKEMMQTAMQNTMLINGIAQVT |
| | FDSETAVKELSYYSLEDLNNKYLYIAVTVIESTGGFSEEAEIPGIKYVLSPYKLNLVATP |
| | LFLKPGIPYPIKVQVKDSLDQLVGGVPVTLNAQTIDVNQETSDLDPSKSVTRVDDGVASF |
| | VLNLPSGVTVLEFNVKTDAPDLPEENQAREGYRAIAYSSLSQSYLYIDWTDNHKALLVGE |
| | HLNIIVTPKSPYIDKITHYNYLILSKGKIIHFGTREKFSDASYQSINIPVTQNMVPSSRL |
| | LVYYIVTGEQTAELVSDSVWLNIEEKCGNQLQVHLSPDADAYSPGQTVSLNMATGMDSWV |
| | ALAAVDSAVYGVQRGAKKPLERVFQFLEKSDLGCGAGGGLNNANVFHLAGLTFLTNANAD |
| | DSQENDEPCKEILRPRRTLQKKIEEIAAKYKHSVVKKCCYDGACVNNDETCEQRAARISL |
| | GPRCIKAFTECCVVASQLRANISHKDMQLGRLHMKTLLPVSKPEIRSYFPESWLWEVHLV |
| | PRRKQLQFALPDSLTTWEIQGVGISNTGICVADTVKAKVFKDVFLEMNIPYSVVRGEQIQ |
| | LKGTVYNYRTSGMQFCVKMSAVEGICTSESPVIDHQGTKSSKCVRQKVEGSSSHLVTFTV |
| | LPLEIGLHNINFSLETWFGKEILVKTLRVVPEGVKRESYSGVTLDPRGIYGTISRRKEFP |
| | YRIPLDLVPKTEIKRILSVKGLLVGEILSAVLSQEGINILTHLPKGSAEAELMSVVPVFY |
| | VFHYLETGNHWNIFHSDPLIEKQKLKKKLKEGMLSIMSYRNADYSYSVWKGGSASTWLTA |
| | FALRVLGQVNKYVEQNQNSICNSLLWLVENYQLDNGSFKENSQYQPIKLQGTLPVEAREN |
| | SLYLTAFTVIGIRKAFDICPLVKIDTALIKADNFLLENTLPAQSTFTLAISAYALSLGDK |
| | THPQFRSIVSALKREALVKGNPPIYRFWKDNLQHKDSSVPNTGTARMVETTAYALLTSLN |
| | LKDINYVNPVIKWLSEEQRYGGGFYSTQDTINAIEGLTEYSLLVKQLRLSMDIDVSYKHK |
| | GALHNYKMTDKNFLGRPVEVLLNDDLIVSTGFGSGLATVHVTTVVHKTSTSEEVCSFYLK |
| | IDTQDIEASHYRGYGNSDYKRIVACASYKPSREESSSGSSHAVMDISLPTGISANEEDLK |
| | ALVEGVDQLFTDYQIKDGHVILQLNSIPSSDFLCVRFRIFELFEVGFLSPATFTVYEYHR |
| | PDKQCTMFYSTSNIKIQKVCEGAACKCVEADCGQMQEELDLTISAETRKQTACKPEIAYA |
| | YKVSITSITVENVFVKYKATLLDIYKTGEAVAEKDSEITFIKKVTCTNAELVKGRQYLIM |
| | GKEALQIKYNFSFRYIYPLDSLTWIEYWPRDTTCSSCQAFLANLDEFAEDIFLNGC |
| Human Factor P | SEQ ID NO: 100 |
| | DPVLCFTQYEESSGKCKGLLGGGVSVEDCCLNTAFAYQKRSGGLCQPCRSPRWSLWSTWA |
| | PCSVTCSEGSQLRYRRCVGWNGQCSGKVAPGTLEWQLQACEDQQCCPEMGGWSGWGPWEP |
| | CSVTCSKGTRTRRRACNHPAPKCGGHCPGQAQESEACDTQQVCPTHGAWATWGPWTPCSA |
| | SCHGGPHEPKETRSRKCSAPEPSQKPPGKPCPGLAYEQRRCTGLPPCPVAGGWGPWGPVS |
| | PCPVTCGLGQTMEQRTCNHPVPQHGGPFCAGDATRTHICNTAVPCPVDGEWDSWGEWSPC |
| | IRRNMKSISCQE1PGQQSRGRTCRGRKFDGHRCAGQQQDIRHCYSIQHCPLKGSWSEWST |
| | WGLCMPPCGPNPTRARQRLCTPLLPKYPPTVSMVEGQGEKNVTFWGRPLPRCEELQGQKL |
| | VVEEKRPCLHVPACKDPEEEEL |
| Human TNFα | SEQ ID NO: 101 |
| | VRSSSRTPSDKPVAHVVANPQAEGQLQWLNRRANALLANGVELRDNQLVVPSEGLYLIYS |
| | QVLFKGQGCPSTHVLLTHTISRIAVSYQTKVNLLSAIKSPCQRETPEGAEAKPWYEPIYL |
| | GGVFQLEKGDRLSAEINRPDYLDFAESGQVYFGIIAL |
| Human IL-1 β | SEQ ID NO: 102 |
| | MAEVPELASEMMAYYSGNEDDLFFEADGPKQMKCSFQDLDLCPLDGGIQLRISDHHYSKG |
| | FRQAASVVVAMDKLRKMLVPCPQTFQENDLSTFFPFIFEEEPIFFDTWDNEAYVHDAPVR |
| | SLNCTLRDSQQKSLVMSGPYELKALHLQGQDMEQQVVFSMSFVQGEESNDKIPVALGLKE |
| | KNLYLSCVLKDDKPTLQLESVDPKNYPKKKMEKRFVFNKIEINNKLEFESAQFPNWYIST |
| | SQAENMPVFLGGTKGGQDITDFTMQFVSS |

### Peptide Linkers

In certain aspects of the invention the protein tags maybe linked to a molecule by a linker. More specifically, the protein tags maybe linked to a protein or a nucleic acid, by a peptide linker (e.g., a (Glyₙ-Serₙ)ₙ or (Serₙ-Glyₙ)ₙ linker) with an optimized length and/or amino acid composition. It is known that peptide linker length can greatly affect how the connected proteins fold and interact. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

The peptide linker sequence may be at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues in length. The peptide linker sequence may be comprised of a naturally, or non-naturally, occurring amino acids. In some aspects, the linker is a glycine polymer. In some aspects, the amino acids glycine and serine comprise the amino acids within the linker sequence. In certain aspects, the linker region comprises sets of glycine repeats (GlySerGly₃)ₙ, where n is a positive integer equal to or greater than 1. More specifically, the linker sequence may be GlySerGlyGlyGly (SEQ ID NO: 31). Alternatively, the linker sequence may be GlySerGlyGly (SEQ ID NO: 124). In certain other aspects, the linker region orientation comprises sets of glycine repeats (SerGly₃)ₙ, where n is a positive integer equal to or greater than 1.

The peptide linkers may also include, but are not limited to, (Gly₄ Ser)₄ or (Gly₄Ser)₃. The amino acid residues Glu and Lys can be interspersed within the Gly-Ser peptide linkers for better solubility. In certain aspects, the peptide linkers may include multiple repeats of (Gly₃Ser), (Gly₂Ser) or (GlySer). In certain aspects, the peptide linkers may include multiple repeats of (SerGly₃), (SerGly₂) or (SerGly). In other aspects, the peptide linkers may include combinations and multiples of (Gly₃Ser)+(Gly₄Ser)+(GlySer). In still other aspects, Ser can be replaced with Ala e.g., (Gly₄Ala) or (Gly₃Ala). In yet other aspects, the linker comprises the motif (GluAlaAlaAlaLys)ₙ, where n is a positive integer equal to or greater than 1. In certain aspects, peptide linkers may also include cleavable linkers.

Peptide linkers can be of varying lengths. In particular, a peptide linker is from about 5 to about 50 amino acids in length; from about 10 to about 40 amino acids in length; from about 15 to about 30 amino acids in length; or from about 15 to about 20 amino acids in length. Variation in peptide linker length may retain or enhance activity, giving rise to superior efficacy in activity studies. Peptide linkers can be introduced into polypeptide and protein sequences using techniques known in the art. For example, PCR mutagenesis can be used. Modifications can be confirmed by DNA sequence analysis. Plasmid DNA can be used to transform host cells for stable production of the polypeptides produced.

Peptide linkers, peptide tags and proteins (e.g.: antibodies or antigen binding fragments) or nucleic acids, or a combination thereof, can be encoded in the same vector and expressed and assembled in the same host cell. Alternatively, each peptide linker, protein tag and protein or nucleic acid can be generated separately and then conjugated to one another. Peptide linkers, peptide tags and proteins or nucleic acids can be prepared by conjugating the constituent components, using methods known in the art. Site-specific conjugation can be achieved using sortase-mediated enzymatic conjugation (Mao H, Hart S A, Schink A, Pollok B A. J Am Chem Soc. 2004 Mar. 10; 126(9):2670-1). A variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohexane-1-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

Engineered and Modified Molecules with Extended Half Life

Production of Peptide Tagged Molecules

The present invention provides peptide tags that can be recombinantly fused (i.e.: linked) or chemically conjugated (including both covalent and non-covalent conjugations) to other molecules, for example other proteins or nucleic acids. In certain aspects one, two, three, four or more peptide tags may be recombinantly fused, linked or chemically conjugated to a protein or nucleic acid. In certain aspects the peptide tag binds HA. In other aspects, the peptide tag binds HA and comprises a LINK Domain. In other aspects, the peptide tag binds HA and comprises a TSG-6 LINK Domain. More specifically, it is contemplated that the peptide tag may be HA10 (SEQ ID NO: 32), HA10.1 (SEQ ID NO: 33), HA10.2 (SEQ ID NO: 34), HA11 (SEQ ID NO: 35) HA11.1 (SEQ ID NO: 36), NVS-X (SEQ ID NO: 204), NVS-Y (SEQ ID NO: 205), NVS-AX (SEQ ID NO: 206), or NVS-AY (SEQ ID NO: 207). In addition, the protein may be any of the proteins, antibodies or antigen binding fragments described herein, including, but not limited to, proteins, antibodies and antigen binding fragments as described above and in Tables 1, 2, 2b, 4b and 5, as well as US20120014958, WO2012015608, WO2012149246, U.S. Pat. No. 8,273,352, WO1998045331, US2012100153, and WO2002016436.

In certain specific aspects, the invention provides peptide tagged molecules comprising antibodies, or antigen binding fragments, and a peptide tag. In particular, the invention provides peptide tagged molecules comprising an antigen-binding fragment of an antibody described herein (e.g., a Fab fragment, Fd fragment, Fv fragment, (Fab′)2 fragment, a VH domain, a VH CDR, a VL domain or a VL CDR) and a peptide tag. Methods for linking, fusing or conjugating proteins, polypeptides, or peptides to an antibody or an antigen binding fragment are known in the art and may be performed using standard molecular biology techniques known to those of skill in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341; Hermanson (2008) Bioconjugate Techniques (2^{nd} edition). Elsevier, Inc.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (e.g., antibodies or fragments thereof with higher affinities and lower dissociation rates) and/or to alter the activity of a peptide tag or protein (e.g., peptide tags and/or proteins with higher affinities and lower dissociation rate). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313, (Pluckthun, 2012), (Wittrup, 2001), (Levin and Weiss, 2006). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to a therapeutic target in a synovial joint, (e.g: the protein TNFα) may be recombined with one or more components, motifs, sections, parts, domains, fragments, etc. of one or more heterologous molecules and/or peptide tags that bind HA.

Moreover, the antibodies, or antigen binding fragments, and/or peptide tags can be fused to marker sequences, such as a peptide to facilitate purification. For example, the marker amino acid sequence is a hexa-histidine peptide, such as the marker provided in a pQE vector (QIAGEN®, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other tags useful for purification include, but are not limited to, the hemagglutinin tag, which corresponds to an epitope derived from the influenza hemagglutinin protein (Wilson et al., 1984, Cell 37:767), and the “flag” tag.

In other embodiments, antibodies, or antigen binding fragments, and/or peptide tags may be conjugated to a diagnostic or detectable agent. Such antibodies and/or peptide tags can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidinlbiotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I,), carbon (14C), sulfur (35S), tritium (3H), indium (115In, 113In, 112In, and 111In,), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and non-radioactive paramagnetic metal ions.

Antibodies, or antigen binding fragments, and peptide tags may also be attached to solid supports, which are particularly useful for immunoassays or purification of the target antigen. Such solid supports include, but are not limited to, gas, cellulose, polyacrylamide, nylon, polystyrene, polyvinyl chloride or polypropylene.

Binding of the peptide tags or peptide tagged molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-ligand complexes of particular interest by employing a labeled reagent (e.g., an antibody) specific for the complex of interest.

Anti-TNFα Antibodies and Antigen Binding Fragments Linked to Peptide Tags

The invention also provides for the peptide tags to be linked to anti-TNFα antibodies, or antigen binding fragments, thereby extending the intra-articular half-life of the anti-TNFα antibodies, or antigen binding fragments.

In certain aspects the peptide tag is a peptide tag that binds HA, which is linked to a anti-TNFα antibody. In one aspect, the peptide tagged molecule comprises a peptide tag that binds HA in the synovial joint with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In one aspect the peptide tag binds HA with a KD of less than or equal to 8.0 uM. In one aspect the peptide tag binds HA with a KD of less than or equal to 7.2 uM. In one aspect the peptide tag binds HA with a KD of less than or equal to 5.5 uM. The peptide tag that binds HA can be a LINK Domain, a TSG-6 LINK Domain, or a specific peptide tag with a sequence of SEQ ID NO: 32, 33, 34, 35, 36, 204, 205, 206 or 207. In certain aspects, the peptide tag is linked to a TNFα binding antibody, or antigen binding fragment (e.g.: such as a Fab) comprising the heavy chain CDRs having the sequence of SEQ ID NOs: 108, 109 and 110, respectively. In other aspects, a peptide tag is linked to a TNFα binding antibody, or antigen binding fragment comprising the light chain CDRs having the sequence of SEQ ID NOs: 117, 118 and 119, respectively. More specifically, a peptide tag is linked to a TNF binding antibody, or antigen binding fragment comprising the heavy chain CDRs having the sequence of SEQ ID NOs: 108, 109 and 110, respectively and the light chain CDRs having the sequence of SEQ ID NOs: 117, 118 and 119, respectively. In still other aspects, a peptide tag is linked to a TNFα binding antibody, or antigen binding fragment comprising the variable heavy chain having the sequence of SEQ ID NOs: 111. In still other aspects, a peptide tag is linked to a TNFα binding antibody, or antigen binding fragment thereof comprising the variable light chain having the sequence of SEQ ID NOs: 120. In further aspects, a peptide tag is linked to a VEGF binding antibody, or antigen binding fragment comprising the variable heavy chain and variable light chain having the sequence of SEQ ID NOs: 111 and 120, respectively. In still other aspects, a peptide tag is linked to a TNFα binding antibody, or antigen binding fragment comprising the heavy chain having the sequence of SEQ ID NOs: 115. In still other aspects, a peptide tag is linked to a TNFα binding antibody, or antigen binding fragment comprising the light chain having the sequence of SEQ ID NOs: 122. In further aspects, a peptide tag is linked to a TNFα binding antibody, or antigen binding fragment comprising the heavy chain and light chain having the sequence of SEQ ID NOs: 115 and 122, respectively. In further aspects, a peptide tag is linked to a TNFα binding antibody, or antigen binding fragment comprising the heavy chain and light chain having the sequence of SEQ ID NOs: 115 and 122, respectively.

In certain aspects a TNFα binding antibody, or antigen binding fragment comprising the heavy chain CDRs having the sequence of SEQ ID NOs: 108, 109 and 110, respectively and the light chain CDRs having the sequence of SEQ ID NOs: 117, 118 and 119, respectively, may have a peptide tag linked to the light chain, the heavy chain and/or have multiple tags on one chain or both chains. More specifically, the peptide tagged TNFα binding antibody, or antigen binding fragment may have heavy chain and light chain with a sequence of SEQ ID NO: 115 & 122, respectively.

It is contemplated that a peptide tag with a sequence of SEQ ID NO: 32, 33, 34, 35, 36, 204, 205, 206 or 207 may be linked to infliximab (Remicade®), entanercept (Embrel®), golimumab (Simponi®), and adalimumab (Humira®).

Other Antibodies or Antigen Binding Fragments Linked to Peptide Tags

The invention also provides for the peptide tags comprising a sequence of SEQ ID NO: 32, 33, 34, 35, 36, 204, 205, 206, or 207 to be linked to antibodies or antigen binding fragments that bind TNFα, VEGF, C5, Factor P, Factor D, EPO, EPOR, IL-1β, IL-17A, IL-6, IL-18, IL-8, bFGF, MCP-1, FGFR2, CD132, IL-6R, CD20, IGF-1, and/or PDGF (including PDGF-BB), thereby extending the intra-articular half-life of the antibodies, or antigen binding fragments. In certain aspects, a peptide tag having a sequence of SEQ ID NO: 32, 33, 34, 35, 36, 204, 205, 206, or 207 is linked to a C5 binding antibody, or antigen binding fragment (e.g.: such as a Fab) comprising the heavy chain CDRs having the sequence of SEQ ID NOs: 37, 38 and 39, respectively. In other aspects, the peptide tag is linked to a C5 binding antibody, or antigen binding fragment comprising the light chain CDRs having the sequence of SEQ ID NOs: 46, 47 and 48, respectively. More specifically, the peptide tag is linked to a C5 binding antibody, or antigen binding fragment comprising the heavy chain CDRs having the sequence of SEQ ID NOs: 37, 38 and 39 respectively and the light chain CDRs having the sequence of SEQ ID NOs: 46, 47 and 48 respectively. In still other aspects, the peptide tag linked to a C5 binding antibody, or antigen binding fragment comprising the variable heavy chain having the sequence of SEQ ID NOs: 40. In still other aspects, the peptide tag linked to a C5 binding antibody, or antigen binding fragment comprising the variable light chain having the sequence of SEQ ID NOs: 49. In further aspects, the peptide tag is linked to a C5 binding antibody, or antigen binding fragment comprising the variable heavy chain and variable light chain having the sequence of SEQ ID NOs: 40 and 49, respectively. In certain aspects, the heavy chain linked to a peptide tag may have the sequence of SEQ ID NO: 44. More specifically, the C5 binding antibody, or antigen binding fragment, linked to a peptide tag has a peptide tagged heavy chain and light chain with a sequence of SEQ ID NO: 44 & 51, respectively.

In certain aspects, a peptide tag having a sequence of SEQ ID NO: 32, 33, 34, 35, 36, 204, 205, 206, or 207 is linked to an Epo binding antibody, or antigen binding fragment (e.g.: such as a Fab) comprising the heavy chain CDRs having the sequence of SEQ ID NOs: 75, 76 and 77, respectively. In other aspects, the peptide tag is linked to a Epo binding antibody, or antigen binding fragment comprising the light chain CDRs having the sequence of SEQ ID NOs: 86, 87 and 88, respectively. More specifically, the peptide tag is linked to a Epo binding antibody, or antigen binding fragment comprising the heavy chain CDRs having the sequence of SEQ ID NOs: 75, 76 and 77, respectively and the light chain CDRs having the sequence of SEQ ID NOs: 86, 87 and 88, respectively. In still other aspects, the peptide tag linked to a Epo binding antibody, or antigen binding fragment comprising the variable heavy chain having the sequence of SEQ ID NOs: 81. In still other aspects, the peptide tag linked to a Epo binding antibody, or antigen binding fragment comprising the variable light chain having the sequence of SEQ ID NOs: 92. In further aspects, the peptide tag is linked to a Epo binding antibody, or antigen binding fragment comprising the variable heavy chain and variable light chain having the sequence of SEQ ID NOs: 81 and 92, respectively. In certain aspects, the heavy chain linked to a peptide tag may have the sequence of SEQ ID NO: 85. More specifically, the Epo binding antibody, or antigen binding fragment, linked to a peptide tag has a peptide tagged heavy chain and light chain with a sequence of SEQ ID NO: 85 & 95, respectively.

In certain aspects, a peptide tag having a sequence of SEQ ID NO: 32, 33, 34, 35, 36, 204, 205, 206, or 207 is linked to a Factor P binding antibody, or antigen binding fragment (e.g.: such as a Fab) comprising the heavy chain CDRs having the sequence of SEQ ID NOs: 53, 54 and 55, respectively. In other aspects, the peptide tag is linked to a Factor P binding antibody, or antigen binding fragment comprising the light chain CDRs having the sequence of SEQ ID NOs: 65, 66 and 67, respectively. More specifically, the peptide tag is linked to a Factor P binding antibody, or antigen binding fragment comprising the heavy chain CDRs having the sequence of SEQ ID NOs: 53, 54 and 55, respectively and the light chain CDRs having the sequence of SEQ ID NOs: 65, 66 and 67, respectively. In still other aspects, the peptide tag linked to a Factor P binding antibody, or antigen binding fragment comprising the variable heavy chain having the sequence of SEQ ID NOs: 59. In still other aspects, the peptide tag linked to a Factor P binding antibody, or antigen binding fragment comprising the variable light chain having the sequence of SEQ ID NOs: 71. In further aspects, the peptide tag is linked to a Factor P binding antibody, or antigen binding fragment comprising the variable heavy chain and variable light chain having the sequence of SEQ ID NOs: 59 and 71, respectively. In certain aspects, the heavy chain linked to a peptide tag may have the sequence of SEQ ID NO: 63. More specifically, the Factor P binding antibody, or antigen binding fragment, linked to a peptide tag has a peptide tagged heavy chain and light chain with a sequence of SEQ ID NO: 63 & 73, respectively.

In certain aspects, a peptide tag having a sequence of SEQ ID NO: 32, 33, 34, 35, 36, 204, 205, 206, or 207 is linked to a VEGF binding antibody, or antigen binding fragment (e.g.: such as a Fab) comprising the heavy chain CDRs having the sequence of SEQ ID NOs: 1, 2 and 3, respectively. In other aspects, the peptide tag is linked to a VEGF binding antibody, or antigen binding fragment comprising the light chain CDRs having the sequence of SEQ ID NOs: 11, 12 and 13, respectively. More specifically, the peptide tag is linked to a VEGF binding antibody, or antigen binding fragment comprising the heavy chain CDRs having the sequence of SEQ ID NOs: 1, 2 and 3, respectively and the light chain CDRs having the sequence of SEQ ID NOs: 11, 12 and 13, respectively. In still other aspects, the peptide tag linked to a VEGF binding antibody, or antigen binding fragment comprising the variable heavy chain having the sequence of SEQ ID NOs: 7. In still other aspects, the peptide tag linked to a VEGF binding antibody, or antigen binding fragment comprising the variable light chain having the sequence of SEQ ID NOs: 17. In further aspects, the peptide tag is linked to a VEGF binding antibody, or antigen binding fragment comprising the variable heavy chain and variable light chain having the sequence of SEQ ID NOs: 7 and 17, respectively. In certain aspects, the heavy chain linked to a peptide tag may have the sequence of SEQ ID NO: 9. More specifically, the VEGF binding antibody, or antigen binding fragment, linked to a peptide tag has a peptide tagged heavy chain and light chain with a sequence of SEQ ID NO: 9 & 19, respectively.

In certain aspects, a peptide tag having a sequence of SEQ ID NO: 32, 33, 34, 35, 36, 204, 205, 206, or 207 is linked to a IL-1β binding antibody, or antigen binding fragment (e.g.: such as a Fab) comprising the heavy chain CDRs having the sequence of SEQ ID NOs: 189, 190 and 191, respectively. In other aspects, the peptide tag is linked to a IL-1β binding antibody, or antigen binding fragment comprising the light chain CDRs having the sequence of SEQ ID NOs: 198, 199 and 200, respectively. More specifically, the peptide tag is linked to a IL-1β binding antibody, or antigen binding fragment comprising the heavy chain CDRs having the sequence of SEQ ID NOs: 189, 190 and 191, respectively and the light chain CDRs having the sequence of SEQ ID NOs: 198, 199 and 200, respectively. In still other aspects, the peptide tag linked to a IL-1β binding antibody, or antigen binding fragment comprising the variable heavy chain having the sequence of SEQ ID NOs: 193. In still other aspects, the peptide tag linked to a IL-1β binding antibody, or antigen binding fragment comprising the variable light chain having the sequence of SEQ ID NOs: 201. In further aspects, the peptide tag is linked to a IL-1β binding antibody, or antigen binding fragment comprising the variable heavy chain and variable light chain having the sequence of SEQ ID NOs: 193 and 201, respectively. In certain aspects, the heavy chain linked to a peptide tag may have the sequence of SEQ ID NO: 194. More specifically, the IL-1β binding antibody, or antigen binding fragment, linked to a peptide tag has a peptide tagged heavy chain and light chain with a sequence of SEQ ID NO: 196 & 202, respectively.

In certain aspects, a peptide tag having a sequence of SEQ ID NO: 32, 33, 34, 35, 36, 204, 205, 206, or 207 is linked to an antibody or antigen binding fragment that binds C5, Epo or Factor P as described in WO2010/015608, or WO2012/149246 and herein incorporated by reference.

### Homologous Proteins

The invention also provides proteins and peptide tags that are homologous to the sequences described herein. More specifically, the present invention provides for a protein comprising amino acid sequences that are homologous to the sequences described in Table 1, 2, 4, 4b, and 5 and the protein or peptide tag binds to the respective intra-articular target, and retains the desired functional properties of those proteins and peptide tags described in Table 1, 2, 4, 4b, 5 and the examples.

For example, the invention provides for anti-TNFα antibodies or antigen binding fragments and peptide tags that are homologous to the sequences described herein. More specifically, the invention provides an antibody, or an antigen binding fragment thereof, comprising a heavy chain variable domain and a light chain variable domain, wherein the heavy chain variable domain comprises an amino acid sequence that is at least 80%, 90%, 95%, 96%, 97%, 98% or 99% identical to the amino acid sequence of SEQ ID NOs: 111; the light chain variable domain comprises an amino acid sequence that is at least 80%, 90%, 95%, 96%, 97%, 98% or 99% A identical to the amino acid sequence of SEQ ID NOs: 120; and the antibody specifically binds to TNFα. In certain aspects of the invention the heavy and light chain sequences further comprise HCDR1, HCDR2, HCDR3, LCDR1, LCDR2, and LCDR3 sequences as defined by Kabat, for example SEQ ID NOs: 108, 109, 110, 117, 118, and 119, respectively.

In other embodiments, the VH and/or VL amino acid sequences may be greater than or equal to 80%, 90%, 95%, 96%, 97%, 98% or 99% A identical to the sequences set forth in Tables 1 and 2. In other embodiments, the VH and/or VL amino acid sequences may be identical except for an amino acid substitution in no more than 1, 2, 3, 4 or 5 amino acid positions. An antibody having VH and VL regions having <100% sequence identity to the VH and VL regions of those described in Tables 1 and 2 can be obtained by mutagenesis (e.g., site-directed or PCR-mediated mutagenesis) of nucleic acid molecules described in Tables 1 and 2 (e.g.: for example, nucleic acid molecules encoding SEQ ID NOs: 111 and SEQ ID NOs: 120, respectively) followed by testing of the encoded altered antibody for retained function using the functional assays described herein and in US20120014958.

In other embodiments, the full length heavy chain and/or full length light chain amino acid sequences may be greater than or equal to 80%, 90%, 95%, 96%, 97%, 98% or 99% identical to the sequences set forth in Tables 1 and 2. An antibody having a heavy chain and light chain having high (i.e., 80% or greater) identity to the heavy chains and light chains described in Tables 1 and 2 (e.g.: the heavy chains of any of SEQ ID NOs: 113 or 115 and light chain of SEQ ID NO: 122) can be obtained by mutagenesis (e.g., site-directed or PCR-mediated mutagenesis) of nucleic acid molecules encoding such polypeptides, followed by testing of the encoded altered antibody for retained function using the functional assays described herein.

In other embodiments, the full length heavy chain and/or full length light chain nucleotide sequences may be greater than or equal to 80%, 90%, 95%, 96%, 97%, 98% or 99% identical to the sequences set forth in Table 1 and Table 2.

In other embodiments, the variable regions of heavy chain and/or the variable regions of light chain nucleotide sequences may be greater than or equal to 80%, 90%, 95%, 96%, 97%, 98% or 99% identical to the sequences set forth in Table 1 and Table 2. It is contemplated that the variability may be in the CDR or framework regions.

In addition, the present invention also provides for a peptide tag comprising amino acid sequences that are homologous to the sequences described in Table 1, and the peptide tag binds to HA and retains the desired functional properties of those peptide tags described herein. More specifically, the amino acid sequences of the peptide tags may be greater than or equal to 80%, 90%, 95%, 96%, 97%, 98% or 99% identical to the sequences set forth in Table 1 and retain the desired functional properties of those the peptide tags described herein.

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

Additionally or alternatively, the protein sequences of the present invention can further be used as a “query sequence” to perform a search against public databases to, for example, identify related sequences. For example, such searches can be performed using the BLAST program (version 2.0) of Altschul, et al., 1990 J. Mol. Biol. 215:403-10.

Proteins with Conservative Modifications

Further included within the scope of the invention are isolated peptide tags and peptide tagged molecules, with conservative modifications. More specifically, the invention is related to peptide tags and peptide tagged molecules with conservative modification to the peptide tags and peptide tagged molecules of Table 1. Also included within the scope of the invention are isolated antibodies, or antigen binding fragments, with conservative modifications. In certain aspects, the peptide tagged antibody of the invention has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibody retains the desired functional properties of the antibodies of the invention. For example, the invention provides a peptide tag linked to a TNFα-binding isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequence is SEQ ID NO: 108, and conservative modifications thereof; the heavy chain variable region CDR2 amino acid sequence is SEQ ID NO: 109, and conservative modifications thereof; the heavy chain variable region CDR3 amino acid sequence is SEQ ID NO: 110, and conservative modifications thereof; the light chain variable regions CDR1 amino acid sequence is SEQ ID NO: 117, and conservative modifications thereof; the light chain variable regions CDR2 amino acid sequence is SEQ ID NO: 118, and conservative modifications thereof; the light chain variable regions of CDR3 amino acid sequence is SEQ ID NO: 119, and conservative modifications thereof; and the antibody or antigen binding fragment thereof specifically binds to TNFα.

In other embodiments, the antibody of the invention is optimized for expression in a mammalian cell and has a full length heavy chain sequence and a full length light chain sequence, wherein one or more of these sequences have specified amino acid sequences based on the antibodies described herein or conservative modifications thereof, and wherein the antibodies retain the desired functional properties of the TNFα binding antibodies of the invention. Accordingly, the invention provides an isolated antibody optimized for expression in a mammalian cell comprising, for example, a variable heavy chain and a variable light chain wherein the variable heavy chain comprises the amino acid sequence of SEQ ID NOs: 111, and conservative modifications thereof; and the variable light chain comprises and amino acid sequence of SEQ ID NOs: 120, and conservative modifications thereof; and the antibody specifically binds to TNFα. The invention further provides an isolated antibody linked to a peptide tag and optimized for expression in a mammalian cell comprising, for example, a variable heavy chain and a variable light chain and a peptide tag wherein the variable heavy chain comprises the amino acid sequence of SEQ ID NOs: 111, and conservative modifications thereof; and the variable light chain comprises an amino acid sequence of SEQ ID NOs: 120, and conservative modifications thereof; and the peptide tag comprises an amino acid sequence selected from SEQ ID NOs: 32, 33, 34, 35, 36, 204, 205, 206, or 207, and the antibody specifically binds to TNFα and the peptide tag specifically binds to HA. The invention provides an isolated antibody optimized for expression in a mammalian cell consisting of a heavy chain and a light chain and a peptide linker and a peptide tag wherein the heavy chain comprising an amino acid sequence of SEQ ID NOs: 115, and conservative modifications thereof; and the light chain comprising an amino acid sequence of SEQ ID NOs: 122, and conservative modifications thereof; and the peptide tag comprising an amino acid sequence selected from SEQ ID NOs: 32, 33, 34, 35, 36, 204, 205, 206 or 207; and the antibody specifically binds to TNFα and the peptide tag specifically binds to HA.

### Methods of Producing Antibodies & Tags of the Invention

### Nucleic Acids Encoding the Antibodies & Peptide Tags

The invention provides substantially purified nucleic acid molecules which encode the peptide tags, and/or peptide tagged molecules described herein. In certain aspects the invention provides substantially purified nucleic acid molecules which encode peptide tagged proteins, for example, the peptide tagged proteins described in Tables 1, 2, 2b, 4b and 5. More specifically, the invention provides substantially purified nucleic acid molecules which encode NVS1, NVS2, NVS3, NVS4, NVS36, NVS37, NVS70, NVS70T, NVS71, NVS71T, NVS72, NVS72T, NVS72, NVS73T, NVS74, NVS74T, NVS75, NVS75T, NVS76, NVS76T, NVS77, NVS77T, NVS78, NVS78T, NVS79, NVS79T, NVS80, NVS80T, NVS81, NVS81T, NVS82, NVS82T, NVS83, NVS83T, NVS84, NVS84T, NVS1b, NVS1c, NVS1d, NVS1e, NVS1f, NVS1g, NVS1h or NVS1j. Also provided in the invention are nucleic acid molecules which encode at least one peptide tag having a peptide sequence of SEQ ID NO: 32, 33, 34, 35, 36, 204, 205, 206, and/or 207. More specifically, for example, the nucleotide sequence encoding the peptide tag may include the nucleotide sequence of SEQ ID NO: 102, 103, 104, 105 and/or 106.

The invention provides substantially purified nucleic acid molecules which encode the proteins described herein, for example, proteins comprising the anti-TNFα, anti-EPO, anti-05, anti-Factor P, anti-VEGF, anti-IL-6, anti-IL-18, anti-bFGF, anti-MCP-1, anti-IL-8, anti-CD132, anti-IL-6R, anti-CD20, anti-IGF-1, or anti-IL-1β antibodies or antigen binding fragments, peptide tags, and/or peptide tagged molecules described above. More specifically, some of the nucleic acids of the invention comprise the nucleotide sequence encoding the heavy chain variable region shown in SEQ ID NO: 111, and/or the nucleotide sequence encoding the light chain variable region shown in SEQ ID NO: 120. In certain specific embodiments, the nucleic acid molecules are those identified in Table 1 or Table 2. Some other nucleic acid molecules of the invention comprise nucleotide sequences that are substantially identical (e.g., at least 65, 80%, 95%, or 99%) to the nucleotide sequences of those identified in Table 1 or Table 2. When expressed from appropriate expression vectors, polypeptides encoded by these polynucleotides are capable of exhibiting target antigen binding capacity, such as, for example, anti-TNFα, anti-EPO, anti-05, anti-Factor P, anti-VEGF, anti-IL-6, anti-IL-18, anti-bFGF, anti-MCP-1, anti-IL-8, anti-CD132, anti-IL-6R, anti-CD20, anti-IGF-1, or anti-IL-1β antigen binding capacity.

Also provided in the invention are polynucleotides which encode at least one CDR region and usually all three CDR regions from the heavy or light chain of the antibody set forth above. Some other polynucleotides encode all or substantially all of the variable region sequence of the heavy chain and/or the light chain of the antibody set forth above. Because of the degeneracy of the code, a variety of nucleic acid sequences may encode each of the immunoglobulin amino acid sequences.

The nucleic acid molecules of the invention can encode both a variable region and a constant region of the antibody. Some of the nucleic acid sequences of the invention comprise nucleotides encoding a modified heavy chain sequence that is substantially identical (e.g., at least 80%, 90%, or 99%) to the original heavy chain sequence (e.g.: substantially identical to the heavy chain of NVS73). Some other nucleic acid sequences comprising nucleotide encoding a modified light chain sequence that is substantially identical (e.g., at least 80%, 90%, or 99%) to the original light chain sequence (e.g.: substantially identical to the light chain of NVS73).

The polynucleotide sequences can be produced by de novo solid-phase DNA synthesis or by PCR mutagenesis of an existing sequence (e.g., sequences as described in the Examples below) encoding a TNFα antibody or its binding fragment. Direct chemical synthesis of nucleic acids can be accomplished by methods known in the art, such as the phosphotriester method of Narang et al., 1979, Meth. Enzymol. 68:90; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, N. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the peptide tags, proteins, antibodies or antigen binding fragments, or peptide tagged molecules described above, for example peptide tagged antibodies or antigen binding fragments described herein. More specifically, the invention provides an expression vector comprising a nucleic acid encoding a peptide tag having the sequence of SEQ ID NO: 32, 33, 34, 35, 36, 204, 205, 206, and/or 207, or alternatively, an expression vector comprising a nucleic acid encoding a peptide tagged molecule as described herein. In certain aspects the expression vector comprises a nucleic acid encoding any one of the peptide tagged molecules described in Tables 1, 2, 4 or 5, for example, NVS1, NVS2, NVS3, NVS4, NVS36, NVS37, NVS70, NVS70T, NVS71, NVS71T, NVS72, NVS72T, NVS72, NVS73T, NVS74, NVS74T, NVS75, NVS75T, NVS76, NVS76T, NVS77, NVS77T, NVS78, NVS78T, NVS79, NVS79T, NVS80, NVS80T, NVS81, NVS81T, NVS82, NVS82T, NVS83, NVS83T, NVS84, NVS84T, NVS1b, NVS1c, NVS1d, NVS1e, NVS1f, NVS1g, NVS1h or NVS1j.

Various expression vectors can be employed to express the polynucleotides encoding the peptide tags, the proteins, the antibody chains or antigen binding fragments or peptide tagged antibodies or antigen binding fragments. Both viral-based and non-viral expression vectors can be used to produce the antibodies in a mammalian host cell. Non-viral vectors and systems include plasmids, episomal vectors, typically with an expression cassette for expressing a protein or RNA, and human artificial chromosomes (see, e.g., Harrington et al., Nat Genet 15:345, 1997). For example, non-viral vectors useful for expression of the peptide tags or TNF polynucleotides and polypeptides in mammalian (e.g., human) cells include pThioHis A, B & C, pcDNA3.1/His, pEBVHis A, B & C, (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

Methods for generating virus vectors are well known in the art and would allow for the skilled artisan to generate the virus vectors of the invention (See, e.g., U.S. Pat. No. 7,465,583).

The choice of expression vector depends on the intended host cells in which the vector is to be expressed. Typically, the expression vectors contain a promoter and other regulatory sequences (e.g., enhancers) that are operably linked to the polynucleotides encoding a antibody chain or fragment, a peptide tag, or a peptide tagged antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of an antibody chain or fragment, a peptide tag, or a peptide tagged antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence positioned to form a fusion protein with polypeptides encoded by inserted peptide tag, antibody, or peptide tagged antibody sequences. More often, such inserted sequences are linked to a signal sequences before inclusion in the vector. Vectors to be used to receive sequences encoding antibody light and heavy chain variable domains, or peptide tagged antibody domains, sometimes also encode constant regions or parts thereof. Such vectors allow expression of the variable regions as fusion proteins with the constant regions thereby leading to production of intact antibodies or antigen binding fragments. Typically, such constant regions are human.

The host cells for harboring and expressing the peptide tags, antibody chains, or peptide tagged molecules (e.g.: peptide tagged antibody or antigen binding fragments), can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express antibodies, or peptide tagged molecules (e.g.: peptide tagged antibodies or antigen binding fragments), or peptide tags of the invention. Insect cells in combination with baculovirus vectors can also be used.

In some preferred embodiments, mammalian host cells are used to express and produce the peptide tags, peptide tagged molecules, and/or untagged molecules described herein (e.g. the peptide tagged antibodies or antigen binding fragments) of the present invention. For example, they can be either a hybridoma cell line expressing endogenous immunoglobulin genes (e.g., the 1D6.C9 myeloma hybridoma clone as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, are known to those of skill in the art, and include CHO cell lines, various Cos cell lines, HeLa cells, myeloma cell lines, transformed B-cells and hybridomas. The use of mammalian tissue cell culture to express polypeptides is discussed generally in, e.g., Winnacker, FROM GENES TO CLONES, VCH Publishers, N.Y., N.Y., 1987. Expression vectors for mammalian host cells can include expression control sequences, such as an origin of replication, a promoter, and an enhancer (see, e.g., Queen, et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP pall promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts. (See generally Sambrook, et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the peptide tags, the antibody chains or antigen binding fragments, or the peptide tagged antibody chains or antigen binding fragments, can be prepared using expression vectors of the invention which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type. The invention further provides for process for producing the peptide tags and/or peptide tagged molecules described herein, wherein a host cell capable of producing a peptide tag or peptide tagged molecule as described herein is cultured under appropriate conditions for the production of one or more peptide tags and/or peptide tagged molecules. The process may further include isolating the peptide tags and/or peptide tagged molecules of the invention

Expression vectors containing nucleic acid sequences encoding the peptide tags, proteins and/or antibodies or antigen binding fragments peptide tags, of the invention can be used for delivering a gene to the synovial joint. In certain aspects of the invention, the expression vector encodes an antibody is linked to one or more peptide tags of the invention and is suitable for delivery to the synovial joint. In other aspects of the invention, the antibody, or antigen binding fragment, and peptide tags are encoded in one or more expression vectors suitable for delivery to the synovial joint. Methods for delivering a gene product to the synovial joint are known in the art (See, e.g., Evans C H et al. Clinical trial to assess the safety, feasibility, and efficacy of transferring a potentially anti-arthritic cytokine gene to human joints with rheumatoid arthritis. Hum Gene Ther 1996; 7: 1261-1280; and P D Robbins, C H Evans and Y Chernajovsky Review: Gene therapy for arthritis. Gene Therapy (2003) 10, 902-911).

### Generation of Monoclonal Antibodies

Monoclonal antibodies (mAbs) can be produced by a variety of techniques, including conventional monoclonal antibody methodology e.g., the standard somatic cell hybridization technique of Kohler and Milstein, 1975 Nature 256: 495. Many techniques for producing monoclonal antibody can be employed e.g., viral or oncogenic transformation of B lymphocytes. For example, methods of producing anti-TNFα antibodies or antigen binding fragments of the invention are described herein, in the examples, and are known in the art.

Animal systems for preparing hybridomas include the murine, rat and rabbit systems. Hybridoma production in the mouse is an established procedure. Immunization protocols and techniques for isolation of immunized splenocytes for fusion are known in the art. Fusion partners (e.g., murine myeloma cells) and fusion procedures are also known.

Chimeric or humanized antibodies of the present invention can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

In a certain embodiment, the antibodies of the invention are human monoclonal antibodies. Such human monoclonal antibodies directed against TNFα can be generated using transgenic or transchromosomic mice carrying parts of the human immune system rather than the mouse system. These transgenic and transchromosomic mice include mice referred to herein as HuMAb mice and KM mice, respectively, and are collectively referred to herein as “human Ig mice.”

The HuMAb Mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al., 1994 Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al., 1994 supra; reviewed in Lonberg, N., 1994 Handbook of Experimental Pharmacology 113:49-101; Lonberg, N. and Huszar, D., 1995 Intern. Rev. Immunol. 13: 65-93, and Harding, F. and Lonberg, N., 1995 Ann. N. Y. Acad. Sci. 764:536-546). The preparation and use of HuMAb mice, and the genomic modifications carried by such mice, is further described in Taylor, L. et al., 1992 Nucleic Acids Research 20:6287-6295; Chen, J. et al., 1993 International Immunology 5: 647-656; Tuaillon et al., 1993 Proc. Natl. Acad. Sci. USA 94:3720-3724; Choi et al., 1993 Nature Genetics 4:117-123; Chen, J. et al., 1993 EMBO J. 12: 821-830; Tuaillon et al., 1994 J. Immunol. 152:2912-2920; Taylor, L. et al., 1994 International Immunology 579-591; and Fishwild, D. et al., 1996 Nature Biotechnology 14: 845-851, the contents of all of which are hereby specifically incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92/03918, WO 93/12227, WO 94/25585, WO 97/13852, WO 98/24884 and WO 99/45962, all to Lonberg and Kay; and PCT Publication No. WO 01/14424 to Korman et al.

In another embodiment, human antibodies of the invention can be raised using a mouse that carries human immunoglobulin sequences on transgenes and transchromosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise antibodies of the invention. For example, an alternative transgenic system referred to as the Xenomouse (Abgenix, Inc.) can be used. Such mice are described in, e.g., U.S. Pat. Nos. 5,939,598; 6,075,181; 6,114,598; 6, 150,584 and 6,162,963 to Kucherlapati et al.

Moreover, alternative transchromosomic animal systems expressing human immunoglobulin genes are available in the art and can be used to raise TNFα antibodies of the invention. For example, mice carrying both a human heavy chain transchromosome and a human light chain tranchromosome, referred to as “TC mice” can be used; such mice are described in Tomizuka et al., 2000 Proc. Natl. Acad. Sci. USA 97:722-727. Furthermore, cows carrying human heavy and light chain transchromosomes have been described in the art (Kuroiwa et al., 2002 Nature Biotechnology 20:889-894) and can be used to raise TNFα antibodies of the invention.

Human monoclonal antibodies of the invention can also be prepared using phage display methods for screening libraries of human immunoglobulin genes. Such phage display methods for isolating human antibodies are established in the art or described in the examples below. See for example: U.S. Pat. Nos. 5,223,409; 5,403,484; and U.S. Pat. No. 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 5,885,793; 6,521,404; 6,544,731; 6,555,313; 6,582,915 and 6,593,081 to Griffiths et al.

Human monoclonal antibodies of the invention can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Methods of Engineering Altered Proteins & Peptide Tags

As discussed above, the peptide tags, proteins, antibodies and antigen binding fragments shown herein can be used to create new peptide tags, proteins, antibodies and antigen binding fragments by modifying the amino acid sequences described. Thus, in another aspect of the invention, the structural features of a peptide tagged antibody of the invention are used to create structurally related peptide tagged antibodies that retain at least one functional property of the peptide tagged antibodies of the invention, such as, for example, binding to human TNFα and also inhibiting one or more functional properties of TNFα (e.g., inhibit TNFα binding to the TNFα receptor).

For example, one or more CDR regions of the antibodies of the present invention, or mutations thereof, can be combined recombinantly with known framework regions and/or other CDRs to create additional, recombinantly-engineered, antibodies of the invention, as discussed above. Other types of modifications include those described in the previous section. The starting material for the engineering method is one or more of the VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, in another embodiment, the invention provides a method for preparing a peptide tagged anti-TNFα antibody or antigen binding fragment consisting of a heavy chain variable region antibody sequence having a CDR1 sequence of SEQ ID NO: 108, a CDR2 sequence of SEQ ID NO: 109, and/or a CDR3 sequence of SEQ ID NO: 110; and a light chain variable region antibody sequence having a CDR1 sequence of SEQ ID NO: 117 a CDR2 sequence of SEQ ID NO: 118, and/or a CDR3 sequence of SEQ ID NO: 119; altering at least one amino acid residue within the heavy chain variable region antibody sequence and/or the light chain variable region antibody sequence to create at least one altered antibody sequence; and expressing the altered antibody sequence as a protein.

The altered antibody sequence can also be prepared by screening antibody libraries having fixed CDR3 sequences or minimal essential binding determinants as described in US20050255552 and diversity on CDR1 and CDR2 sequences. The screening can be performed according to any screening technology appropriate for screening antibodies from antibody libraries, such as phage display technology.

Standard molecular biology techniques can be used to prepare and express the altered peptide tag or peptide tagged molecule sequence. The peptide tag or peptide tagged molecule encoded by the altered sequence(s) is one that retains one, some or all of the functional properties of the peptide tag or peptide tagged molecule, for example the proteins or peptide tagged antibodies described herein, such as, for example, NVS73.

In certain embodiments of the methods of engineering antibodies or peptide tags of the invention, mutations can be introduced randomly or selectively along all or part of an TNFα antibody coding sequence or peptide tag and the resulting modified TNFα antibodies or peptide tag can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

In certain embodiments of the invention antibodies and peptide tags may be engineered to remove sites of deamidation. Deamidation is known to cause structural and functional changes in a peptide or protein. Deamindation can result in decreased bioactivity, as well as alterations in pharmacokinetics and antigenicity of the protein pharmaceutical. (Anal Chem. 2005 Mar. 1; 77(5):1432-9). In certain other aspects of the invention antibodies and peptide tags can be engineered to add or remove sites of protease cleavage. Examples of peptide tag modifications are described in the examples.

The functional properties of the altered antibodies can be assessed using standard assays available in the art and/or described herein, such as those set forth in the Examples.

### Other Antibody Formats

### Camelid Antibodies

Antibody proteins obtained from members of the camel and dromedary (Camelus bactrianus and Calelus dromaderius) family including new world members such as llama species (Lama paccos, Lama glama and Lama vicugna) have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994).

A region of the camelid antibody which is the small single variable domain identified as VHH can be obtained by genetic engineering to yield a small protein having high affinity for a target, resulting in a low molecular weight antibody-derived protein known as a “camelid nanobody”. See U.S. Pat. No. 5,759,808 issued Jun. 2, 1998; see also Stijlemans, B. et al., 2004 J Biol Chem 279: 1256-1261; Dumoulin, M. et al., 2003 Nature 424: 783-788; Pleschberger, M. et al. 2003 Bioconjugate Chem 14: 440-448; Cortez-Retamozo, V. et al. 2002 Int J Cancer 89: 456-62; and Lauwereys, M. et al. 1998 EMBO J 17: 3512-3520. Engineered libraries of camelid antibodies and antigen binding fragments are commercially available, for example, from Ablynx, Ghent, Belgium. As with other antibodies of non-human origin, an amino acid sequence of a camelid antibody can be altered recombinantly to obtain a sequence that more closely resembles a human sequence, i.e., the nanobody can be “humanized”.

The camelid nanobody has a molecular weight approximately one-tenth that of a human IgG molecule, and the protein has a physical diameter of only a few nanometers. One consequence of the small size is the ability of camelid nanobodies to bind to antigenic sites that are functionally invisible to larger antibody proteins, i.e., camelid nanobodies are useful as reagents detect antigens that are otherwise cryptic using classical immunological techniques, and as possible therapeutic agents. Thus yet another consequence of small size is that a camelid nanobody can inhibit as a result of binding to a specific site in a groove or narrow cleft of a target protein, and hence can serve in a capacity that more closely resembles the function of a classical low molecular weight drug than that of a classical antibody.

The low molecular weight and compact size further result in camelid nanobodies being extremely thermostable, stable to extreme pH and to proteolytic digestion, and poorly antigenic. Another consequence is that camelid nanobodies readily move from the circulatory system into tissues. Nanobodies can further facilitate drug transport across the blood brain barrier. See U.S. patent application 20040161738 published Aug. 19, 2004. Further, these molecules can be fully expressed in prokaryotic cells such as E. coli and are expressed as fusion proteins with bacteriophage and are functional.

Accordingly, a feature of the present invention is a camelid antibody or nanobody having, for example, high affinity for TNFα. In certain embodiments herein, the camelid antibody or nanobody is naturally produced in the camelid animal, i.e., is produced by the camelid following immunization with TNFα or a peptide fragment thereof, using techniques described herein for other antibodies. Alternatively, a camelid nanobody is engineered (i.e., produced by selection, for example) from a library of phage displaying appropriately mutagenized camelid nanobody proteins using panning procedures with an appropriate target. Engineered nanobodies can further be customized by genetic engineering. The camelid nanobodiy can be linked to peptide tags as described herein to extend mean residence time, terminal drug concentration and/or increase dose interval, relative to the untagged camelid nanobody. In a specific aspects, the camelid antibody or nanobody is obtained by grafting the CDRs sequences of the heavy or light chain of the human antibodies of the invention into nanobody or single domain antibody framework sequences, as described for example in PCT/EP93/02214.

### Bi-Specific Molecules and Multivalent Antibodies

In another aspect, the present invention features bi-specific or multi-specific molecules comprising a peptide tag of the invention. More specifically, it is contemplated that the present invention features bi-specific or multi-specific molecules comprising a peptide tag, and more than one protein and/or nucleic acid molecule. For example, a multi-specific molecule may comprise a peptide tag, an antibody, or antigen binding fragment thereof, and a nucleic acid molecule of the invention.

An antibody of the invention, or antigen-binding fragment thereof, can be derivatized or linked to another functional molecule, e.g., another peptide or protein (e.g., another antibody or ligand for a receptor) to generate a bi-specific molecule that binds to at least two different binding sites or target molecules. The antibody of the invention may in fact be derivatized or linked to more than one other functional molecule to generate multi-specific molecules that bind to more than two different binding sites and/or target molecules; such multi-specific molecules are also intended to be encompassed by the term “bi-specific molecule” as used herein. To create a bi-specific molecule of the invention, an antibody of the invention can be functionally linked (e.g., by chemical coupling, genetic fusion, non-covalent association or otherwise) to one or more other binding molecules, such as another antibody, antigen binding fragment, peptide, or binding mimetic, such that a bi-specific molecule results.

Accordingly, the present invention includes bi-specific molecules comprising at least one first binding specificity for TNFα and a second binding specificity for a second target epitope. For example, the second target epitope is another epitope of TNFα different from the first target epitope. Alternatively, the second target epitope is an epitope of an alternate synovial joint molecule. Alternatively, the second target epitope is an epitope of HA.

Additionally, for the invention in which the bi-specific molecule is multi-specific, the molecule can further include a third binding specificity, in addition to the first and second target epitope. Alternatively, the second target epitope is an epitope of an alternate synovial joint molecule.

In one embodiment, a bi-specific molecule can comprise as a binding specificity at least one antibody, or an antigen binding fragment thereof, including, e.g., a Fab, Fab′, F(ab′)2, Fv, or a single chain Fv. The antibody may also be a light chain or heavy chain dimer, or any minimal fragment thereof such as a Fv or a single chain construct as described in Ladner et al. U.S. Pat. No. 4,946,778.

Diabodies are bivalent, bi-specific molecules in which VH and VL domains are expressed on a single polypeptide chain, connected by a linker that is too short to allow for pairing between the two domains on the same chain. The VH and VL domains pair with complementary domains of another chain, thereby creating two antigen binding sites (see e.g., Holliger et al., 1993 Proc. Natl. Acad. Sci. USA 90:6444-6448; Poljak et al., 1994 Structure 2:1121-1123). Diabodies can be produced by expressing two polypeptide chains with either the structure VHA-VLB and VHB-VLA (VH-VL configuration), or VLA-VHB and VLB-VHA (VL-VH configuration) within the same cell. Most of them can be expressed in soluble form in bacteria. Single chain diabodies (scDb) are produced by connecting the two diabody-forming polypeptide chains with linker of approximately 15 amino acid residues (see Holliger and Winter, 1997 Cancer Immunol. Immunother., 45(3-4):128-30; Wu et al., 1996 Immunotechnology, 2(1):21-36). scDb can be expressed in bacteria in soluble, active monomeric form (see Holliger and Winter, 1997 Cancer Immunol. Immunother., 45(34): 128-30; Wu et al., 1996 Immunotechnology, 2(1):21-36; Pluckthun and Pack, 1997 Immunotechnology, 3(2): 83-105; Ridgway et al., 1996 Protein Eng., 9(7):617-21). A diabody can be fused to Fc to generate a “di-diabody” (see Lu et al., 2004 J. Biol. Chem., 279(4):2856-65).

Other antibodies which can be employed in the bi-specific molecules of the invention are murine, chimeric and humanized monoclonal antibodies.

Bi-specific molecules can be prepared by conjugating the constituent binding specificities, using methods known in the art. For example, each binding specificity of the bi-specific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohexane-I-carboxylate (sulfo-SMCC) (see e.g., Karpovsky et al., 1984 J. Exp. Med. 160:1686; Liu, M A et al., 1985 Proc. Natl. Acad. Sci. USA 82:8648). Other methods include those described in Paulus, 1985 Behring Ins. Mitt. No. 78, 118-132; Brennan et al., 1985 Science 229:81-83), and Glennie et al., 1987 J. Immunol. 139: 2367-2375). Conjugating agents are SATA and sulfo-SMCC, both available from Pierce Chemical Co. (Rockford, Ill.).

When the binding specificities are antibodies, they can be conjugated by sulfhydryl bonding of the C-terminus hinge regions of the two heavy chains. In a particularly embodiment, the hinge region is modified to contain an odd number of sulfhydryl residues, for example one, prior to conjugation.

Alternatively, both binding specificities can be encoded in the same vector and expressed and assembled in the same host cell. This method is particularly useful where the bi-specific molecule is a mAb×mAb, mAb×Fab, Fab×F(ab′)2, ligand×Fab, peptide tag×mAb, peptide tag×Fab fusion protein. A bi-specific molecule of the invention can be a single chain molecule comprising one single chain antibody and a binding determinant, or a single chain bi-specific molecule comprising two binding determinants. Bi-specific molecules may comprise at least two single chain molecules. Methods for preparing bi-specific molecules are described for example in U.S. Pat. No. 5,260,203; U.S. Pat. No. 5,455,030; U.S. Pat. No. 4,881,175; U.S. Pat. No. 5,132,405; U.S. Pat. No. 5,091,513; U.S. Pat. No. 5,476,786; U.S. Pat. No. 5,013,653; U.S. Pat. No. 5,258,498; and U.S. Pat. No. 5,482,858.

Binding of the bi-specific, or multivalent, molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

In another aspect, the present invention provides multivalent molecules comprising at least two identical or different antigen-binding portions of the antibodies of the invention binding to TNFα. In a further aspect, the present invention provides multivalent compounds comprising at least two identical or different antigen-binding portions of the peptide tags of the invention binding to HA. The antigen-binding portions can be linked together via protein fusion or covalent or non-covalent linkage. Alternatively, methods of linkage have been described for the multi-specific molecules. Tetravalent compounds can be obtained for example by cross-linking antibodies of the antibodies of the invention with an antibody that binds to the constant regions of the antibodies of the invention, for example the Fc or hinge region.

Trimerizing domain are described for example in Borean patent EP 1 012 28061. Pentamerizing modules are described for example in PCT/EP97/05897.

### Prophylactic and Therapeutic Uses

Many synovial joint diseases, specifically, for example inflammatory arthritides and osteoarthitis, are treated with therapies that require intra-articular injection weekly, bi-weekly, or monthly. The method and frequency of treatment poses a significant health-care burden to doctors and patients. In addition there also a significant risk to patients associated with frequent intra-articular injections, due to the risk of infection. Thus, the ability to administer therapies dosed quarterly or less frequently will provide the best improvements in joint outcomes while reducing the treatment burden and risks associated with frequent intra-articular injections.

Synovial joint diseases including inflammatory arthritides and osteoarthiritis have an inflammatory component that leads to pain, stiffness, swelling, and in some cases permanent joint injury/damage. Clinical trials have demonstrated that these diseases can be treated effectively with weekly, monthly, or bi-monthly intra-articular injections of intra-articular biologic therapies, for example anti-TNFα therapies such as, infliximab (Remicade®), entanercept (Embrel®), golimumab (Simponi®), and adalimumab (Humira®). Despite the efficacy of these therapies, weekly, monthly or bi-monthly treatment is a significant health-care burden for patients and physicians (Oishi et al. (2011). Thus, there is often a need for an intra-articular therapy that can be delivered less frequently, yet still provide the same treatment benefit seen with monthly or bi-monthly treatment. Anti-TNFα therapies are generally safe and well-tolerated by most patients. Thus an anti-TNFα therapy that could be administered less frequently would have a safety benefit due to the reduced number of intra-articular procedures and lower systemic suppression of TNFα.

There is a need for anti-TNFα therapies that have longer duration of action that will result in patients needing injections less frequently than monthly or bi-monthly while still maintaining the efficacy that is achieved with monthly or bi-monthly dosing regimens.

In addition to TNFα, other proangiogenic, inflammatory, or growth factor mediators are involved in the synovial joint diseases, such as, for example, inflammatory arthritides and osteoarthritis. Examples of these proangiogenic, inflammatory, or growth factor mediator molecules include but are not limited to PDGF (Boyer, 2013), angiopoietin (Oliner et al., 2012), S1P (Kaiser, 2013), integrins αvβ3, αvβ5, α5β1 (Kaiser et al., 2013; Patel, 2009a; Patel, 2009b), betacellulin (Anand-Apte et al., 2010), apelin/APJ (Hara et al., 2013), erythropoietin (Watanabe et al., 2005; Aiello, 2005), complement factor D, VEGF, and proteins linked to AMD risk by genetic association studies such as proteins of the complement pathway including C2, factor B, factor H, CFHR3, C3b, C5, C5a, and C3a, and HtrA1, ARMS2, TIMP3, HLA, IL8, CX3CR1, TLR3, TLR4, CETP, LIPC, COL10A1, and TNFRSF10A (Nussenblatt et al., 2013). As therapies are developed that effectively target these molecules and pathways, there will be a need to provide the improvements in visual outcomes while reducing the treatment burden and risks associated with frequent intra-articular injections. Synovial joint diseases that include but are not limited to rheumatoid arthritis, systemic lupus erythematosus, gout, pseudo-gout, ankylosing spondylitis, psoriatic arthritis, gonorrhea, tuberculosis, osteomyelitis, and osteoarthritis may be amenable to treatment with therapies delivered intra-articularly.

The present invention provides peptide tags that can be attached to a therapeutic molecule to slow the clearance of the therapeutic molecule from the synovial joint, thereby increasing its intra-articular half-life. The invention relates to peptide tags and peptide tagged molecules with increased duration of efficacy relative to an untagged molecule, which will lead to less frequent intra-articular injections and improved patient treatment in the clinic.

The peptide tagged molecules described herein can be used as a medicament. In particular the peptide tagged molecules of the invention may be used for treating a condition or disorder associated with synovial joint disease in a subject. For example, peptide tagged antibodies or antigen binding fragments that bind TNFα as described herein, can be used at a therapeutically useful concentration for the treatment of a synovial joint disease or disorder associated with increased TNFα levels and/or activity by administering to a subject in need thereof an effective amount of the tagged antibodies or antigen binding fragments of the invention.

The present invention provides a method of treating conditions or disorders associated with synovial joint disease by administering to a subject in need thereof an effective amount of the peptide tagged molecules of the invention. The present invention provides a method of treating conditions or disorders associated with rheumatoid arthritis by administering to a subject in need thereof an effective amount of the peptide tagged molecules of the invention. The present invention provides a method of treating conditions or disorders associated with systemic lupus erythematosus by administering to a subject in need thereof an effective amount of the peptide tagged molecules of the invention. The invention also provides a method of treating gout by administering to a subject in need thereof an effective amount of the peptide tagged molecules of the invention. The present invention further provides a method of treating pseudo-gout by administering to a subject in need thereof an effective amount of the peptide tagged molecules of the invention. Still further, the present invention provides methods for treating ankylosing spondylitis, by administering to a subject in need thereof an effective amount of the peptide tagged molecules of the invention. The present invention provides methods for treating psoriatic arthritis, by administering to a subject in need thereof an effective amount of the peptide tagged molecules of the invention. The present invention also provides methods for treating gonorrhea, by administering to a subject in need thereof an effective amount of the peptide tagged molecules of the invention. The present invention also provides methods for treating tuberculosis, by administering to a subject in need thereof an effective amount of the peptide tagged molecules of the invention. The present invention further provides methods for treating osteomyelitis, by administering to a subject in need thereof an effective amount of the peptide tagged molecules of the invention. The present invention further provides methods for treating osteoarthritis, by administering to a subject in need thereof an effective amount of the peptide tagged molecules of the invention. Further still, the invention relates to a method of treating a TNFα-mediated disorder by administering to a subject in need thereof an effective amount of the peptide tagged molecules of the invention. It is contemplated that the peptide tagged molecules comprises a peptide tag that binds HA in the synovial joint with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. It is contemplated that the peptide tagged molecules is a peptide tagged antibody or antigen binding fragment as described herein. In one aspect, the peptide tagged molecule comprises a peptide tag that binds HA in the synovial joint with a KD of less than or equal to 8.0 uM. In one aspect, the peptide tagged molecule comprises a peptide tag that binds HA in the synovial joint with a KD of less than or equal to 7.2 uM. In one aspect, the peptide tagged molecule comprises a peptide tag that binds HA in the synovial joint with a KD of less than or equal to 6.0 uM. In one aspect, the peptide tagged molecule comprises a peptide tag that binds HA in the synovial joint with a KD of less than or equal to 5.5 uM. In certain specific aspects, the peptide tag may comprise a sequence of SEQ ID NO: 32, 33, 34, 35, 36, 204, 205, 206, and 207. In a further aspect, the foregoing methods further comprise, prior to the step of administering, the step of diagnosing a subject with such condition or disorder.

In one aspect, the invention relates to a method of treating a TNFα-mediated disorder in a subject that is refractory to anti-TNFα therapy by administering to the subject in need thereof an effective amount of the peptide tagged molecules of the invention. It is contemplated that the peptide tagged molecules comprises a peptide tag that binds HA in the synovial joint with a KD of less than or equal to 9.0 uM. For example, the peptide tag can bind HA with a KD of less than or equal to, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM or 0.5 uM. In certain specific aspects, the peptide tag may comprise a sequence of SEQ ID NO: 32, 33, 34, 35, 36, 204, 205, 206 or 207. As used here, “refractory to anti-TNFα therapy” refers to the inability to achieve a satisfactory physiological response with known anti-TNFα therapy, such as infliximab (Remicade®), entanercept (Embrel®), golimumab (Simponi®), and adalimumab (Humira®). In one embodiment, a patient who is refractory to anti-TNFα therapy experiences a continuing worsening of pain, stiffness, and restricted joint movement despite infliximab (Remicade®), entanercept (Embrel®), golimumab (Simponi®), and adalimumab (Humira®) therapy. In some embodiments, patients refractory to anti-TNFα therapy demonstrate negligible anatomical improvement despite receiving infliximab (Remicade®), entanercept (Embrel®), golimumab (Simponi®), and adalimumab (Humira®) therapy.

The peptide tagged molecules (e.g.: peptide tagged antibodies or antigen binding fragments) of the invention can be used, inter alia, to prevent progression of conditions or disorders associated with synovial joint disease (for example, rheumatoid arthritis, systemic lupus erythematosus, gout, pseudo-gout, ankylosing spondylitis, psoriatic arthritis, gonorrhea, tuberculosis, osteomyelitis, and osteoarthritis), to treat or prevent osteoarthritis, to reduce the frequency of intra-articular injections compared to the frequency of injections needed with current anti-TNFα drugs (e.g., infliximab (Remicade®), entanercept (Embrel®), golimumab (Simponi®), and adalimumab (Humira®)), and to improve diminished joint movement due to synovial joint disease progression. The peptide tagged molecules (e.g.: the peptide tagged antibodies or antigen binding fragments) of the invention can also be used in combination with, for example, other anti-TNF therapies, other anti-PDGF therapies, other anti-complement therapies, or other anti-EPO therapies, or other anti-inflammatory therapies for the treatment of patients with synovial joint disease.

Treatment and/or prevention of synovial joint disease, rheumatoid arthritis, systemic lupus erythematosus, gout, pseudo-gout, ankylosing spondylitis, psoriatic arthritis, gonorrhea, tuberculosis, osteomyelitis, and osteoarthritis, and TNFα-mediated disorder, and other conditions or disorders associated with synovial joint disease can be determined by a rheumatologist or health care professional using clinically relevant measurements of joint function and/or joint anatomy. Treatment of conditions or disorders associated with synovial joint disease means any action (e.g., administration of a peptide tagged anti-TNF antibody described herein) that results in, or is contemplated to result in, the improvement or preservation of joint function and/or joint anatomy. In addition, prevention as it relates to conditions or disorders associated with synovial joint disease means any action (e.g., administration of a peptide tagged anti-TNFα antibody described herein) that prevents or slows a worsening in joint function, joint anatomy, and/or a synovial joint disease parameter, as defined herein, in a patient at risk for said worsening.

Exemplary measures of joint function include range of motion, shock absorbancy, and patient reported satisfaction. Therapies for rheumatoid arthritis (RA) may be assessed according to relative levels of measures to compare efficacy to another therapy or to a placebo, as in the American College of Rheumatology (ACR) 20%, 50%, or 70% (ACR 20 ACR 50 and ACR 70) responses, or by absolute levels of measures, as in disease activity scores (DAS) Thus, treatment of synovial joint disease can be said to be achieved upon improvement in the ACR (Pinals R S, et al. Arthritis Rheum 1981; 24:1308-15) or DAS28 (Fransen J et al. Rheumatology 2004; 43:1252-5) scores up to and including remission (Felson D T, Smolen J S, Wells G, et al. American College of Rheumatology/European League Against Rheumatism provisional definition of remission in rheumatoid arthritis for clinical trials. Ann Rheum Dis. 2011; 70:404). Therapies for osteoarthritis arthritis (OA) may be assessed according to Western Ontario and McMaster Universities Osteoarthritis Index (WOMAC). The WOMAC consists of 24 items divided into 3 subscales: pain, stiffness and physical function.

Undesirable aspects of joint anatomy that may be treated or prevented include, for example, articular cartilage damage, periarticular osteoporosis, synovial inflammation, synovial effusion, joint pain and swelling.

Exemplary means of assessing synovial joint anatomy include physical examination, plain film radiographs, magnetic resonance imaging and ultrasound. Thus, synovial joint disease can be said to be treated in a subject upon a response of ACR20, ACR50, ACR70 or a reduction in DAS28 vs baseline (Arnett F C, Edworthy S M, Bloch D A, McShane D J, Fries J F, Cooper N S, et al. The American Rheumatism Association 1987 revised criteria for the classification of rheumatoid arthritis. Arthritis Rheum 1988; 31:315-24).

Treatment and/or prevention of synovial joint disease such as rheumatoid arthritis, systemic lupus erythematosus, gout, pseudo-gout, ankylosing spondylitis, psoriatic arthritis, gonorrhea, tuberculosis, osteomyelitis, and osteoarthritis can be determined by a rheumatologist or health care professional using clinically relevant measurements of joint function and/or joint anatomy by any of the measures described above. Although the measures described herein don't apply to each and every joint disease herein, one of skill in the art would recognize the clinically relevant measurement of joint function and/or joint anatomy that could be used to treat the given synovial joint disease.

When the therapeutic agents of the present invention are administered together with another agent, the two can be administered sequentially in either order or simultaneously. In some aspects, a tagged antibody or antigen binding fragment of the present invention is administered to a subject who is also receiving therapy with a second agent (e.g., Remicade). In other aspects, the binding molecule is administered in conjunction with surgical treatments.

Suitable agents for combination treatment with a tagged antibody or antigen binding fragment of the invention include agents known in the art that are able to modulate the activities of TNFα, TNFα receptors, other receptor tyrosine kinase inhibitors, or other entities that modulate HIF-1 mediated pathways. Other agents have been reported to inhibit these pathways include infliximab (Remicade®), entanercept (Embrel®), golimumab (Simponi®), and adalimumab (Humira®). Combination treatments with anti-inflammatory agents such as corticosteroids, NSAIDS, and VEGF inhibitors could also be beneficial in the treatment of synovial joint disease, for example, inflammatory arthritides and osteoarthritis.

A combination therapy regimen may be additive, or it may produce synergistic results (e.g., reductions in retinopathy severity more than expected for the combined use of the two agents). In some embodiments, the present invention provides a combination therapy for preventing and/or treating synovial joint diseases, specifically inflammatory arthritides and osteoarthritis, including rheumatoid arthritis, systemic lupus erythematosus, gout, pseudo-gout, ankylosing spondylitis, psoriatic arthritis, gonorrhea, tuberculosis, osteomyelitis as described above, with a tagged antibody or antigen binding fragment of the invention and an anti-angiogenic, such as second anti-TNFα agent. In certain other embodiments, the present invention provides a combination therapy for preventing and/or treating synovial joint diseases, specifically rheumatoid arthritis, systemic lupus erythematosus, gout, pseudo-gout, ankylosing spondylitis, psoriatic arthritis, gonorrhea, tuberculosis, osteomyelitis, osteoarthritis as described above, with a peptide tagged antibody or peptide tagged antigen binding fragment of the invention and an agent that inhibits other synovial joint targets such as VEGF, PDGF, EPO, components of the complement pathway (e.g.: C5, Factor D, Factor P, C3), SDF1, Apelin, Betacellulin, or an anti-inflammatory agent (e.g: steroid).

In one aspect, the invention relates to a method of extending the duration of efficacy of an intra-articularly-administered therapeutic. Extending duration of efficacy (e.g., increasing dosing interval) can be achieved by increasing the intra-articular half-life, decreasing intra-articular clearance, or increasing the intra-articular mean residence time of the therapeutic. Half-life or mean residence time can be increased (and clearance decreased) by linking the therapeutic (e.g., a protein or nucleic acid) to a peptide tag that binds HA. Accordingly, in one aspect, the invention relates to a method of increasing the half-life, mean residence time, and/or decreasing the clearance of a molecule in the synovial joint. In particular the invention relates to a method of increasing the half-life and/or mean residence time, or decreasing the clearance of a protein or nucleic acid in the synovial joint by linking the protein or nucleic acid to a peptide tag described herein.

An increase in dosing interval results from the increased half-life, increased mean residence time, increased terminal concentration, and/or decreased clearance rate of a molecule from the synovial joint. The invention also provides for methods for increasing half-life of molecule in the synovial joint comprising the step of administering, to the synovial joint of the subject, a composition comprising the molecule linked to a peptide tag that binds HA with a KD of less than or equal to 9.0 uM. In certain specific aspects, the method comprises administering a composition comprising the molecule linked to a peptide tag that binds HA with a KD of less than or equal to 8.0 uM. In certain specific aspects, the method comprises administering a composition comprising the molecule linked to a peptide tag that binds HA with a KD of less than or equal to 7.2 uM. In certain specific aspects, the method comprises administering a composition comprising the molecule linked to a peptide tag that binds HA with a KD of less than or equal to 5.5 uM. The invention provides for methods for increasing mean residence time, increasing terminal concentration and/or decreasing clearance of molecule in/from the synovial joint comprising the step of administering, to the synovial joint of the subject, a composition comprising the molecule linked to a peptide tag that binds HA with a KD of less than or equal to 9.0 uM. In certain specific aspects, the method comprises administering a composition comprising the molecule linked to a peptide tag that binds HA with a KD of less than or equal to 8.0 uM. In certain specific aspects, the method comprises administering a composition comprising the molecule linked to a peptide tag that binds HA with a KD of less than or equal to 7.2 uM. In certain specific aspects, the method comprises administering a composition comprising the molecule linked to a peptide tag that binds HA with a KD of less than or equal to 5.5 uM. In certain aspects the peptide tag comprises the sequence of SEQ ID NO: 32, 33, 34, 36, 37, 204, 205, 206, or 207. It is contemplated that the composition comprises a peptide tag that binds HA with a KD of less than or equal to 9.0 uM, 8.0 uM, 7.2 uM, or 5.5 uM linked to a protein or nucleic acid, for example, an antibody or antigen binding fragment, more specifically, for example, an anti-TNFα antibody or antigen binding fragment.

Half-life as described herein, refers to the time required for the concentration of a drug to fall by one-half (Rowland M and Towzer T N: Clinical Pharmacokinetics. Concepts and Applications. Third edition (1995) and Bonate P L and Howard D R (Eds): Pharmacokinetics in Drug Development, Volume 1 (2004)). Details may also be found in Kenneth, A et al: Chemical Stability of Pharmaceuticals: A Handbook for Pharmacists and in Peters et al, Pharmacokinetic analysis: A Practical Approach (1996). Reference is also made to “Pharmacokinetics”, M Gibaldi & D Perron, published by Marcel Dekker, 2 nd Rev. ex edition (1982), which describes pharmacokinetic parameters such as alpha half-life and beta half-life and area under the curve (AUC). Optionally, all pharmacokinetic parameters and values quoted herein are to be read as being values in a human. Optionally, all pharmacokinetic parameters and values quoted herein are to be read as being values in a mouse or rat or Cynomolgus monkey.

In one aspect, at least a 25% increase (e.g. from 5 to 6.25 days) in half-life by binding to HA is contemplated. In another aspect at least a 50% increase (e.g. from 5 to 7.5 days) in half-life is contemplated. In another aspect at least a 75% increase (e.g. from 5 to 8.75 days) in half-life is contemplated. In another aspect, at least a 100% increase (e.g. from 5 to 10 days) in half-life is contemplated. In another aspect, a greater than 100% increase (e.g., 150%, 200%) in half-life is contemplated. In one aspect, linking a peptide tag to a molecule as described herein can increase the intra-articular half-life by at least 1.5 fold, at least 2 fold, at least 2.5 fold, at least 3 fold, at least 3.5 fold, and at least 4 fold or more relative to the intra-articular half-life of the molecule without the tag. Relative increases in intra-articular half-life for an HA-binding peptide tagged molecule compared to an untagged molecule can be determined by administering the molecules by intra-articular injection and measuring the concentrations remaining at various time points using analytical methods known in the art, for example ELISA, mass spectrometry, western blot, radio-immunoassay, or fluorescent labeling. Clearance from the synovial joint of an intra-articularly administered biologic molecule has been shown to fit a first-order exponential decay function (equation 1) (Krohne et al., 2008; Krohne et al., 2012; Bakri et al., 2007b; Bakri et al., 2007a; Gaudreault et al., 2007; Gaudreault et al., 2005).

Ct=Ct=0*e^{-kt} (1)

The rate constant k is:

$$k = \frac{ln 2}{t 1 / 2} \qquad ( 2 )$$

Cₜ is the concentration at time t after intravitreal administration.
Cₜ₌₀ is the concentration at time 0 after intravitreal administration.
T_{1/2} is the intra-articular half-life after intravitreal administration.

The effects of increasing the intra-articular half-life can be modeled using equations (1) and (2).

Methods for pharmacokinetic analysis and determination of mean residence time and/or half-life of a peptide tagged molecule will be familiar to those skilled in the art. In addition, details related to methods for pharmacokinetic analysis and determination of mean residence time of a peptide tagged molecule may be found in Shargel, L and Yu, A B C: Applied Biopharmaceutics & Pharmacokinetics, 4^{th} Edition (1999), Rowland M and Towzer T N: Clinical Pharmacokinetics. Concepts and Applications. Third edition (1995) and Bonate P L and Howard D R (Eds): Pharmacokinetics in Drug Development, Volume 1 (2004), which describes pharmacokinetic parameters such as Mean Residence Time. Mean residence time and AUC can be determined from a curve of matrix or tissue (e.g.: serum) concentration of a drug (e.g.: therapeutic protein, peptide tagged protein, peptide tag, etc.) against time. Phoenix WinNonlin software, eg version 6.1 (available from Pharsight Corp., Cary, N.C., USA) can be used, for example, to analyze and/or model such data. The mean residence time is the average time that the drug resides in the body and encompasses absorption, distribution and elimination processes. MRT represents the time when 63.2% of the dose has been eliminated.

In one aspect, the invention relates to a method of increasing mean residence time of a molecule (such as a protein or nucleic acid) by linking the molecule to a peptide tag as described herein. In one aspect linking a peptide tag to a molecule as described herein can increase the mean residence time of the molecule in the synovial joint by 10% or more. In a further aspect linking a peptide tag to a molecule as described here in can increase the mean residence time of the molecule in the synovial joint by 20%, 30%, 40%, 50%, 60%, 70%, 80%, 90%, or 100% or more.

In a further aspect, the invention relates to a method of decreasing intra-articular clearance of the molecule (such as a protein or nucleic acid) by linking the molecule to a peptide tag as described herein. In one aspect, linking a peptide tag to a molecule as described herein can decrease intra-articular clearance of the molecule in the synovial joint by 10% or more. In a further aspect, linking a peptide tag to a molecule as described herein can decrease intra-articular clearance of the molecule in the synovial joint by 20%, 30%, 40%, 50%, 60%, 70%, 80%, 90%, or 100% or more.

### Pharmaceutical Compositions

Delivery of Peptide Tags & Peptide Tagged Molecules

The invention provides compositions comprising a peptide tag of the invention, for example a peptide tag that binds HA in the synovial joint with a KD of less than or equal to 9.0 uM, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM, or 0.5 uM. In certain specific aspects the peptide tag may comprise the sequence of SEQ ID NO: 32, 33, 34, 35, 36, 204, 205, 206, or 207, formulated together, or separately, with a pharmaceutically acceptable excipient, diluent or carrier. The invention also provides compositions comprising a peptide tagged molecules (e.g.: a peptide tag linked to a protein or a nucleic acid), formulated together, or separately, with a pharmaceutically acceptable excipient, diluent or carrier. In certain aspects the peptide tagged molecule comprises a peptide tag that binds HA in the synovial joint as described above. The invention also provides compositions comprising peptide tagged antibodies, or peptide tagged antigen binding fragments, and/or a peptide tag, formulated together, or separately, with a pharmaceutically acceptable excipient, diluent or carrier. In certain aspects, the invention provides compositions comprising a TNFα antibody, or antigen binding fragment thereof, linked to a peptide tag, formulated together with a pharmaceutically acceptable excipient, diluent or carrier. In more specific aspects, the invention provides compositions comprising the peptide tagged molecule: NVS37. In still more specific aspects, the invention provides compositions comprising the peptide tagged molecule in any of Tables 1, 2, 4, 4b, or 5. The compositions described herein may be formulated together with a pharmaceutically acceptable excipient, diluent or carrier. The compositions can additionally contain one or more other therapeutic agents that are suitable for treating or preventing, for example, conditions or disorders associated with synovial joint disease. Pharmaceutically acceptable carriers enhance or stabilize the composition, or can be used to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that the composition be suitable for administration to the synovial joint, more specifically, the composition may be suitable for intra-articular administration. The pharmaceutically acceptable excipient, diluent or carrier should be suitable for administration to the synovial joint. (e.g., by injection, subconjunctival or topical administration), more specifically, for intra-articular administration. Depending on the route of administration, the active compound (i.e., antibody, bi-specific and multi-specific molecule), may be coated in a material to protect the compound from the action of acids and other natural conditions that may inactivate the compound. The invention also provides for methods of producing a composition for intra-articular delivery wherein the method includes the step of linking a peptide tag that binds HA in the synovial joint with a KD of less than or equal to 9.0 uM, 8.5 uM, 8.0 uM, 7.5 uM, 7.0 uM, 6.5 uM, 6.0 uM, 5.5 uM, 5.0 uM, 4.5 uM, 4.0 uM, 3.5 uM, 3.0 uM, 2.5 uM, 2.0 uM, 1.5 uM, 1.0 uM, or 0.5 uM to a molecule (e.g.: a protein or nucleic acid) that binds or is capable of binding a target in the synovial joint (e.g.: TNFα, Factor P, Factor D, EPO, VEGF, C5, IL-1β, IL-6, IL-18, bFGF, MCP-1, IL-8, CD132, IL-6R, CD20, IGF-1, etc).

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the molecule employed in the pharmaceutical compositions of the invention. The peptide tagged molecules are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors. Dosage level may be selected and/or adjusted to achieve a therapeutic response as determined using one or more of the joint/movement assessments described herein.

A physician or veterinarian can start doses of the peptide tagged molecules of the invention employed in the pharmaceutical composition at levels lower than that required to achieve the desired therapeutic effect and gradually increase the dosage until the desired effect is achieved. In general, effective doses of the compositions of the present invention, for the treatment of a synovial joint disease described herein vary depending upon many different factors, including means of administration, target site, physiological state of the patient, whether the patient is human or an animal, other medications administered, and whether treatment is prophylactic or therapeutic. Treatment dosages need to be titrated to optimize safety and efficacy. Dosage for intra-articular administration with a peptide tagged molecule may range from 0.1 mg/joint to 6 mg/joint per injection. A single dose per joint may be carried out in 2 injections per joint. For example, a single dose of 12 mg/joint may be delivered in 2 injections of 6 mg each, resulting in a total dose of 12 mg. In certain specific aspects, a dose may be 12 mg/joint, 11 mg/joint, 10 mg/joint, 9 mg/joint, 8 mg/joint, 7 mg/joint, 6 mg/joint, 5 mg/joint, 4.5 mg/joint, 4 mg/joint, 3.5 mg/joint, 3 mg/joint, 2.5 mg/joint, 2 mg/joint, 1.5 mg/joint, 1 mg/joint, 0.9 mg/joint, 0.8 mg/joint, 0.7 mg/joint, 0.6 mg/joint, 0.5 mg/joint, 0.4 mg/joint, 0.3 mg/joint, 0.2 mg/joint, or 0.1 mg/joint or lower. Each dose may be carried out in one or more injections per joint. The volume per injection may be between 10 microliters and 50 micoliters, while the volume per dose may be between 10 microliters and 100 micoliters. For example, doses include 0.1 mg/50 ul, 0.2 mg/50 ul, 0.3 mg/50 ul, 0.4 mg/50 ul, 0.5 mg/50 ul, 0.6 mg/50 ul, 0.7 mg/50 ul, 0.8 mg/50 ul, 0.9 mg/50 ul, 1.0 mg/50 ul, 1.1 mg/50 ul, 1.2 mg/50 ul, 1.3 mg/50 ul, 1.4 mg/50 ul, 1.5 mg/50 ul, 1.6 mg/50 ul, 1.7 mg/50 ul, 1.8 mg/50 ul, 1.9 mg/50 ul, 2.0 mg/50 ul, 2.1 mg/50 ul, 2.2 mg/50 ul, 2.3 mg/50 ul, 2.4 mg/50 ul, 2.5 mg/50 ul, 2.6 mg/50 ul, 2.7 mg/50 ul, 2.8 mg/50 ul, 2.9 mg/50 ul, 3.0 mg/50 ul, 3.1 mg/50 ul, 3.2 mg/50 ul, 3.3 mg/50 ul, 3.4 mg/50 ul, 3.5 mg/50 ul, 3.6 mg/50 ul, 3.7 mg/50 ul, 3.8 mg/50 ul, 3.9 mg/50 ul, 4.0 mg/50 ul, 4.1 mg/50 ul, 4.2 mg/50 ul, 4.3 mg/50 ul, 4.4 mg/50 ul, 4.5 mg/50 ul, 4.6 mg/50 ul, 4.7 mg/50 ul, 4.8 mg/50 ul, 4.9 mg/50 ul, 5.0 mg/50 ul, 5.1 mg/50 ul, 5.2 mg/50 ul, 5.3 mg/50 ul, 5.4 mg/50 ul, 5.5 mg/50 ul, 5.6 mg/50 ul, 5.7 mg/50 ul, 5.8 mg/50 ul, 5.9 mg/50 ul, or 6.0 mg/50 ul per joint per injection. An exemplary treatment regime entails IVT administration once per every two weeks or once a month or once every 2 months or once every 3 to 6 months or as needed (PRN). The peptide tagged molecules allow for an increase in dosing intervals which improve the treatment regime of current therapies and is described in further detail below.

A composition of a peptide tag or peptide tagged molecule may be administered on multiple occasions. Intervals between single dosages can be weekly, monthly or yearly. Intervals can also be irregular as indicated by the need for retreatment in the patient, based for example on range of motion or inflammation. In addition alternative dosing intervals can be determined by a physician and administered monthly or as necessary to be efficacious. Efficacy is based physician and patient scores for joint swelling and tenderness, as well as radiographic, ultrasound, or MRI improvement in joint pathology. Dosage and frequency may vary depending on the half-life of the peptide tagged molecule in the patient and levels of the therapeutic target (e.g., TNFα, C5, EPO, Factor P, etc.). Extending the duration of efficacy of a therapeutic molecule administered IVT can be achieved by increasing the intra-articular T_{1/2} and/or increasing its intra-articular mean residence time and/or decreasing clearance. Extending the duration of efficacy can be achieved, for example by linking an HA-binding peptide tag to a molecule to slow its clearance from the vitreous, retina and/or RPE/choroid resulting in an increased intra-articular half-life of the peptide tagged molecule. Relative increases in intra-articular half-life for a peptide tagged molecule that binds HA compared to an untagged molecule can be determined by administering the molecules by intra-articular injection and measuring the concentrations remaining at various time points using analytical methods known in the art, for example ELISA, mass spectrometry, western blot, radio-immunoassay, or fluorescent labeling. Blood concentrations can also be measured and used to calculate the rate of clearance from the synovial joint as described (Evans, C. H. et al., Nat. Rev. Rheumatol. 10, 11-22 (2014)).

In general, molecules (for example, antibodies or fragments) linked to peptide tags of the invention show longer intra-articular half-life than that of untagged molecules. For example, a molecule linked to a peptide tag that binds HA in the synovial joint can have a 25% increase (e.g. from 5 to 6.25 days) in half-life compared to the untagged molecule, a 50% increase (e.g. from 5 to 7.5 days) in half-life compared to the untagged molecule, a 75% increase (e.g. from 5 to 8.75 days) in half-compared to the untagged molecule, or a 100% increase (e.g. from 5 to 10 days) in half-life compared to the untagged molecule. In certain aspects, it is contemplated that half-life of the peptide tagged molecule may increase more than 100% compared to the untagged molecule (e.g.: from 5 to 15, 20 or 30 days; from 1 week to 3 weeks, 4 weeks or more; etc.).

The dosage and frequency of administration can vary depending on whether the treatment is prophylactic or therapeutic and is directly affected by the half-life of the molecule dosed. Administration of the peptide tags or peptide tagged molecules described herein lead to a clinically meaningful improvement of dose and dosing frequency. For example, the peptide tags or peptide tagged molecules can be dosed at lower frequency compared to untagged molecules. Achieving a clinically meaningful improvement in dose and dosing frequency can vary depending on the initial starting dose of a composition. For example, for molecules that are dosed daily, weekly, bi-weekly, monthly or bi-monthly, a clinically meaningful improvement in dosing frequency that could be achieved with the peptide tagged molecule would be, for example, at least a 25%, 30%, 50%, 75%, or 100% increase in the dosing interval. In certain aspects, for example a clinically meaningful improvement of dosing frequency occurs by reducing the dosing frequency from daily to every other day, weekly to every two weeks, or monthly to every six weeks or bimonthly, or longer respectively.

More specifically the peptide tag of the invention may be used to improve the dosing interval of current intra-articular therapies. In certain aspects a peptide tag may be useful for increasing the dosing interval of a molecule by at least 25%. For example, the dosing interval can be increased by 30%, 40%, 50%, 60%, 70%, 75%, 80%, 90%, 100%, or more. The intra-articular dosing interval of a molecule may be increased by linking the molecule to a peptide tag that binds HA in the synovial joint with a KD of less than or equal to 7.5 uM, less than or equal to 7.0 uM, less than or equal to 6.5 uM, less than or equal to 6.0 uM, less than or equal to 5.5 uM, less than or equal to 5.0 uM, less than or equal to 4.5 uM, less than or equal to 4.0 uM, less than or equal to 3.5 uM, less than or equal to 3.0 uM, less than or equal to 2.5 uM, less than or equal to 2.0 uM, less than or equal to 1.5 uM, less than or equal to 1.0 uM, less than or equal to 0.5 uM, or less than or equal to 100 nM. Symptomatic relief with corticosteroids normally lasts up to four weeks only. Linking anti-TNFα antibodies is expected to provide relief for twelve week intervals. For other molecules that require dosed frequencies of every two months, or longer, a clinically meaningful improvement would be increasing the dosing interval by an additional month or longer (i.e. at least 50% increase in dosing interval).

In certain specific aspects the composition is formulated to deliver 12 mg, 11 mg, 10 mg, 9 mg, 8 mg, 7 mg, 6 mg, 5 mg, 4.5 mg, 4 mg, 3.5 mg, 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, or 0.1 mg of the peptide tagged molecule per dose. In certain specific aspects the composition is formulated to deliver 6 mg, 5 mg, 4.5 mg, 4 mg, 3.5 mg, 3 mg, 2.5 mg, 2 mg, 1.5 mg, 1 mg, 0.9 mg, 0.8 mg, 0.7 mg, 0.6 mg, 0.5 mg, 0.4 mg, 0.3 mg, 0.2 mg, 0.1 mg, or 0.05 mg of the peptide tagged molecule per injection. In a particular aspect the composition is formulated to deliver 12 mg of the peptide tagged molecule per dose and/or 6 mg of the peptide tagged molecule per injection. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### EXAMPLES

The Examples herein describe hyaluronan (HA) binding peptide tags that extend the half-life of molecules in the synovial joints, for example the molecules may be proteins or nucleic acids.

### Example 1

TABLE 3
Summary of in vitro and in vivo data for 14 tagged antibodies. The untagged
antibody NVS4 was modified with the sequences shown (linker + peptide
tag) to produce the 14 tagged antibodies tested. (Linker sequence underlined)

| | Sequence of GSGGG | | | >1% injected | |
|---|---|---|---|---|---|
| | linker + peptide tag | | | dose at | Positive |
| | linked to NVS4 | Origin of | HA | 96 hrs in rat | Rabbit |
| NVS ID | (SEQ ID NO:) | peptide tag | binding | PET/CT PK | Efficacy |
| NVS1 | GSGGGGVYHREARSGKYKLTY | Tumor necrosis | Yes | Yes | Yes |
| | AEAKAVCEFEGGHLATYKQLE | factor-inducible | | | |
| | AARKIGFHVCAAGWMAKGR | gene 6 protein | | | |
| | VGYPIVKPGPNCGFGKTGIIDY | (TNFAIP6/TSG6 | | | |
| | GIRLNRSERWDAYCYNPHAK | aa 36-129) | | | |
| | (SEQ ID NO: 127) | | | | |
| NVS16 | GSGGGKQKIKHVVKLKGSGG | Hyaluronan | Yes | Yes | No |
| | GKLKSQLVKRK (SEQ ID NO: | mediated | | | |
| | 128) | motility receptor | | | |
| | | (HMMR aa 401- | | | |
| | | 411, 423-432) | | | |
| NVS17 | GSGGGKNGRYSISRGSGGGR | CD44 antigen | Yes | Yes | No |
| | DGTRYVQKGEYRGSGGGRRR | (CD44 aa 38- | | | |
| | CGQKKK (SEQ ID NO: 129) | 46, 150-162, 292- | | | |
| | | 300) | | | |
| NVS18 | GSGGGVFPYHPRGGRYKLTFA | Hyaluronan and | Yes | Yes | No |
| | EAQRACAEQDGILASAEQLHA | proteoglycan link | | | |
| | AWRDGLDWCNAGWLRDGS | protein 4 | | | |
| | VQYPVNRPREPCGGLGGTGS | (HAPLN4 aa163- | | | |
| | AGGGGDANGGLRNYGYRHN | 267) | | | |
| | AEERYDAFCF (SEQ ID NO: | | | | |
| | 130) | | | | |
| NVS5 | GSGGEVFYVGPARRLTLAGAR | Neurocan core | Yes | Yes | No |
| | AQCRRQGAALASVGQLHLA | protein (NCAN | | | |
| | WHEGLDQCDPGWLADGSVR | Link 2 aa 259- | | | |
| | YPIQTPRRRCGGPAPGVRTVY | 357) | | | |
| | RFANRTGFPSPAERFDAYCFR | | | | |
| | (SEQ ID NO 131) | | | | |
| NVS11 | GSGGLKQKIKHVVKLKDENSQ | Hyaluronan | Yes | Yes | No |
| | LKSEVSKLRSQLVKRKQNGSG | mediated | | | |
| | GAHWQFNALTVRGGGSSTM | motility receptor | | | |
| | MSRSHKTRSHHV (SEQ ID | (HMMR and HA | | | |
| | NO: 132) | phage peptide) | | | |
| NVS8 | GSGGGVFHLRSPLGQYKLTFD | Stabilin-2 (Stab2 | Yes | Yes | No |
| | KAREACANEAATMATYNQLS | aa 2199-2296) | | | |
| | YAQKAKYHLCSAGWLETGRV | | | | |
| | AYPTAFASQNCGSGVVGIVDY | | | | |
| | GPRPNKREMWDVFCYRMKD | | | | |
| | VN (SEQ ID NO: 133) | | | | |
| NVS9 | GSGGGHQNLKQKIKHVVKLK | Hyaluronan | Yes | Yes | No |
| | DENSQLKSEVSKLRSQLAKKK | mediated | | | |
| | QSETKLQ (SEQ ID NO: 134) | motility receptor | | | |
| | | (HMMR aa 516- | | | |
| | | 559) | | | |
| NVS10 | GSGGGGVYHREARSGKYKLTY | Tumor necrosis | Yes | Yes | No |
| | AEAKAVCEFEGGHLATYKQLE | factor-inducible | | | |
| | AARKIGFHVCSAGWLETGRV | gene 6 protein | | | |
| | AYPTAFASQNCGSGVVGIVDY | (TNFAIP6/TSG6) | | | |
| | GIRLQRSERWDAYCYNPHAK | and Stabilin-2 | | | |
| | AHP (SEQ ID NO: 135) | (Stab2) Chimeric | | | |
| NVS7 | GSGGGKVGKSPPVRGSGGGH | HUMAN GHAP | Yes | Yes | No |
| | REARSGKYK (SEQ ID NO: | S4, TSG6 aa 39- | | | |
| | 136) | 48 | | | |
| NVS6 | GSKQKIKHVVKLKGGGSREAR | RHAMM/TSG6 | Yes | Yes | No |
| | SGKYK (SEQ ID NO: 137) | BX7B Link | | | |
| NVS92 | GSGGGKGGNGEPRGDTYRAY | Bone | Yes | Yes | No |
| | GSGGGKGGPQVTRGDVFTM | Sialoprotein and | | | |
| | P (SEQ ID NO: 138) | Vitronectin | | | |
| | Sequence of GSGGG | | | >1% injected | |
| | linker + peptide tag | | Collagen | dose at | Positive |
| | linked to NVS4 | Origin of | II | 96 hrs in rat | Rabbit |
| NVS ID | (SEQ ID NO:) | peptide tag | binding | PET/CT PK | Efficacy |
| NVS67 | Not applicable* | Not applicable* | Yes | Yes | No |
| NVS68 | GSGGGRRANAALKAGELYKSI | Osteopontin/B | Yes | Yes | No |
| | LYG (SEQ ID NO: 139) | (X)7 B | | | |
| NVS69 | GSGGGRRANAALKAGELYKSI | SLRP | Yes | Yes | No |
| | LYG (SEQ ID NO: 140) | | | | |

*NVS67 is an anti-VEGF scFv fused with an anti-collagen II scFv in a tandem manner.

### Generation of Proteins and Nucleic Acids Linked to an HA-Binding Peptide Tag.

To test the ability of the HA-binding peptide tags to extend the half-life of proteins or nucleic acids in the synovial joint, the peptide tags of the invention were linked to numerous antibodies, proteins and nucleic acids which bind a variety of intra-articular protein targets.

### Generation of Peptide Tagged Antibodies and Proteins

Tagged and untagged recombinant antibodies and proteins were expressed by transient transfections of mammalian expression vectors in HEK293 cells and purified using standard affinity resins for example, KappaSelect (Cat #17-5458-01, GE Healthcare Biosciences®) and HisTrap (Cat #17-5255-01, GE Healthcare Biosciences®). Various antibody and protein formats were tested, including: Fabs, IgGs, Fc Traps and proteins. These antibodies and proteins targets several synovial joint targets, for example, C5, Factor P, EPO, EPOR, TNFα, Factor D, IL-1β, IL-17A, FGFR2, or IL-10.

Fabs linked to single peptide tags were generated as described above by linking the HA-binding tag sequence to the C-terminal of the heavy chain of a Fab using a GSGGG linker (e.g.: SEQ ID NO: 31). To generate peptide tagged IgGs (e.g.: IgG fusions that contain HA-binding tag sequences) the HA-binding tag sequence was fused to the C-terminal of the heavy chain or light chain of an IgG using a GSGGG linker (e.g.: SEQ ID NO: 31). To generate peptide tagged proteins than contain an Fc portion, for example, Fc trap protein linked to an HA-binding tag, the HA-binding tag was linked to the C-terminal of the Fc portion of the protein using a GSGGG linker (e.g.: SEQ ID NO: 31). To generate additional peptide tagged proteins, the HA-binding tag was linked to the C-terminus of the protein of interest using a GSGGG linker (e.g.: SEQ ID NO: 31). In all cases described above, production of candidates entails nucleotide synthesis encoding the amino acid of desired proteins followed by expression and purification using mammalian expression systems described above.

The peptide tagged antibodies and peptide antigen binding fragments exemplified herein may also be converted and used in alternate antibody formats. For example, peptide tagged IgGs, can be converted to peptide tagged Fabs or peptide tagged scFvs, or vice versa.

### Generation of Peptide Tagged Nucleic Acids

Nucleic acids including RNA or DNA aptamers can be conjugated an HA-binding peptide as described below. In to a solution of B—3-(2-carboxyethyl)-1-(1-(2-hydrazinyl-4-methylpentanoyl)pyrrolidin-2-yl)-6-(1-hydroxyethyl)-1,4,7,10-tetraoxo-2,5, 8,11-tetraazatridecan-13-oic acid (198 mg, 0.280 mmol) in ACN (Volume: 1.75 mL) at room temperature is added DIPEA (0.098 mL, 0.559 mmol) and a solution of A—(3S,6S)-1-((S)-1-((S)-2-amino-4-methylpentanoyl)pyrrolidin-2-yl)-3-(2-carboxyethyl)-6-((R)-1-hydroxyethyl)-1,4,7,10-tetraoxo-2,5,8,11-tetraazatridecan-13-oic acid (32 mg, 0.056 mmol) in DMSO (Volume: 1.75 mL). The mixture is stirred at room temperature for 1 h and then purified using Sunfire Prep C18 eluting with 10 to 90% ACN-water+0.1% TFA to afford 27 mg pure desired product C-(3S,6S)-3-(2-carboxyethyl)-1-((S)-1-((S)-34-((2,5-dioxopyrrolidin-1-yl)oxy)-2-isobutyl-4,34-dioxo-7,10,13,16,19,22,25,28,31-nonaoxa-3-azatetratriacontan-1-oyl)pyrrolidin-2-yl)-6-((R)-1-hydroxyethyl)-1,4,7,10-tetraoxo-2,5,8,11-tetraazatridecan-13-oic acid. To a solution of D—ARC126-NH₂ (25 mg/ml in NaHCO3 pH-8.5 buffer) (18.63 mg, 230 μl, 1.807 μmol) is added C-(3S,6S)-3-(2-carboxyethyl)-1-((S)-1-((S)-34-((2,5-dioxopyrrolidin-1-yl)oxy)-2-isobutyl-4,34-dioxo-7,10,13,16,19,22,25,28,31-nonaoxa-3-azatetratriacontan-1-oyl)pyrrolidin-2-yl)-6-((R)-1-hydroxyethyl)-1,4,7,10-tetraoxo-2,5,8,11-tetraazatridecan-13-oic acid (100 mg/ml in DMSO) (5.26 mg, 52.6 μl, 4.52 μmol). The reaction is stirred at room temperature for 1.5 hr. The crude is passed through a 3K MW CO Amicon filter column (3K MW cut-off) and simultaneously buffer exchanged to sortase buffer 0.1M Tris pH8.0+CaCl2 0.01M+NaCl 0.15M. To a solution of F—the HA-peptide tag (287 μL, 0.047 μmol) in Tris 0.25M pH 7.4+CaCl2 5 mM and NaCl 150 mM (Volume: 313 μL) is added E (57.4 μL, 0.703 μmol) followed by immobilized Sortase A on beads (87 μL, 0.016 μmol). The mixture is agitated at 20° C. for 2 days. The resultant aptamer-HA binding peptide conjugate was NVS79T.

TABLE 4
Examples of proteins and nucleic acids linked to a peptide
tag that binds HA. The proteins and nucleic acids exemplified
cover various examples of proteins and nucleic acids that
bind different targets in the synovial joint.

| | Synovial | | | |
|---|---|---|---|---|
| NVS | Joint | | | |
| ID | Target | HA Tag | Format | Location of HA tag |
| NVS70 | C5 | None | Fab | None |
| NVS70T | C5 | SEQ ID NO: | Fab | C-terminus of NVS70 |
| | | 33 | | heavy chain |
| NVS71 | Factor | None | Fab | None |
| | P | | | |
| NVS71T | Factor | SEQ ID NO: | Fab | C-terminus of NVS71 |
| | P | 33 | | heavy chain |
| NVS72 | EPO | None | Fab | None |
| NVS72T | EPO | SEQ ID NO: | Fab | C-terminus of NVS72 |
| | | 33 | | heavy chain |
| NVS73 | TNFα | None | Fab | None |
| NVS73T | TNFα | SEQ ID NO: | Fab | C-terminus of NVS73 |
| | | 33 | | heavy chain |
| NVS74 | Factor | None | Fab | None |
| | D | | | |
| NVS74T | Factor | SEQ ID NO: | Fab | C-terminus of NVS74 |
| | D | 33 | | heavy chain |
| NVS75 | IL-1β | None | Fab | None |
| NCS75T | IL-1β | SEQ ID NO: | Fab | C-terminus of NVS75 |
| | | 33 | | heavy chain |
| NVS76 | IL-17A | None | Fab | None |
| NVS76T | IL-17A | SEQ ID NO: | Fab | C-terminus of NVS76 |
| | | 33 | | heavy chain |
| NVS77 | FGFR2 | None | Fab | None |
| NVS77T | FGFR2 | SEQ ID NO: | Fab | C-terminus of NVS77 |
| | | 33 | | heavy chain |
| NVS78 | EPO | None | Fc Trap | None |
| NVS78T | EPO | SEQ ID NO: | Fc Trap | C-terminus of Fc of |
| | | 33 | | NVS78 |
| NVS90 | EPOR | None | Protein | None |
| NVS90T | EPOR | SEQ ID NO: | Protein | C-terminus of NVS90 |
| | | 33 | | |
| NVS79 | PDGF- | None | Aptamer | None |
| | BB | | | |
| NVS79T | PDGF- | SEQ ID NO: | Aptamer | Chemically conjugated |
| | BB | 33 | | to NVS79 |
| NVS91 | IL-10R | None | Protein | None |
| NVS91T | IL-10R | SEQ ID NO: | Protein | C-terminus |
| | | 33 | | |
| mAb1 | TNFα | SEQ ID NO: | Antibody | CH3 |
| | | 220 | | |
| mAb2 | TNFα | SEQ ID NO: | Antibody | K chain |
| | | 220 | | |

TABLE 4b
Sequences of peptide tagged molecules.

| | Synovial | Light | |
|---|---|---|---|
| NVS ID | Joint Target | Chain (or single chain) | Heavy Chain |
| NVS70 | C5 | SEQ ID NO: 51 | SEQ ID NO: 42 |
| NVS70T | C5 | SEQ ID NO: 51 | SEQ ID NO: 44 |
| NVS71 | Factor P | SEQ ID NO: 73 | SEQ ID NO: 61 |
| NVS71T | Factor P | SEQ ID NO: 73 | SEQ ID NO: 63 |
| NVS72 | EPO | SEQ ID NO: 95 | SEQ ID NO: 83 |
| NVS72T | EPO | SEQ ID NO: 95 | SEQ ID NO: 85 |
| NVS73 | TNFα | SEQ ID NO: 122 | SEQ ID NO: 113 |
| NVS73T | TNFα | SEQ ID NO: 122 | SEQ ID NO: 115 |
| NVS74 | Factor D | SEQ ID NO: 142 | SEQ ID NO: 143 |
| | | DIQVTQSPSSLSASVGDRVTIT | QLVQSGPELKKPGASVKVSC |
| | | CITSTDIDDDMNWYQQKPGK | KASGYTFTNYGMNWVRQAP |
| | | VPKLLISGGNTLRPGVPSRFS | GQGLEWMGWINTYTGETTYA |
| | | GSGSGTDFTLTISSLQPEDVA | DDFKGRFVFSLDTSVSTAYLQ |
| | | TYYCLQSDSLPYTFGQGTKVE | ISSLKAEDTAVYYCEREGGVN |
| | | IKRTVAAPSVFIFPPSDEQLKS | NWGQGTLVTVSSASTKGPSV |
| | | GTASVVCLLNNFYPREAKVQ | FPLAPSSKSTSGGTAALGCLV |
| | | WKVDNALQSGNSQESVTEQD | KDYFPEPVTVSWNSGALTSG |
| | | SKDSTYSLSSTLTLSKADYEK | VHTFPAVLQSSGLYSLSSVVT |
| | | HKVYACEVTHQGLSSPVTKSF | VPSSSLGTQTYICNVNHKPSN |
| | | NRGEC | TKVDKRVEPKSC |
| NVS74T | Factor D | SEQ ID NO: 144 | SEQ ID NO: 145 |
| | | DIQVTQSPSSLSASVGDRVTIT | QLVQSGPELKKPGASVKVSC |
| | | CITSTDIDDDMNWYQQKPGK | KASGYTFTNYGMNWVRQAP |
| | | VPKLLISGGNTLRPGVPSRFS | GQGLEWMGWINTYTGETTYA |
| | | GSGSGTDFTLTISSLQPEDVA | DDFKGRFVFSLDTSVSTAYLQ |
| | | TYYCLQSDSLPYTFGQGTKVE | ISSLKAEDTAVYYCEREGGVN |
| | | IKRTVAAPSVFIFPPSDEQLKS | NWGQGTLVTVSSASTKGPSV |
| | | GTASVVCLLNNFYPREAKVQ | FPLAPSSKSTSGGTAALGCLV |
| | | WKVDNALQSGNSQESVTEQD | KDYFPEPVTVSWNSGALTSG |
| | | SKDSTYSLSSTLTLSKADYEK | VHTFPAVLQSSGLYSLSSVVT |
| | | HKVYACEVTHQGLSSPVTKSF | VPSSSLGTQTYICNVNHKPSN |
| | | NRGEC | TKVDKRVEPKSCGSGGGGVY |
| | | | HREAQSGKYKLTYAEAKAVC |
| | | | EFEGGHLATYKQLEAARKIGF |
| | | | HVCAAGWMAKGRVGYPIVKP |
| | | | GPNCGFGKTGIIDYGIRLNRSE |
| | | | RWDAYCYNPHA |
| NVS75 | IL-1β | SEQ ID NO: 194 | SEQ ID NO: 202 |
| NCS75T | IL-1β | SEQ ID NO: 196 | SEQ ID NO: 202 |
| NVS78 | EPOR | SEQ ID NO: 146 | None |
| | | GGGGGPPPNLPDPKFESKAA | |
| | | LLAARGPEELLCFTERLEDLV | |
| | | CFWEEAASAGVGPGNYSFSY | |
| | | QLEDEPWKLCRLHQAPTARG | |
| | | AVRFWCSLPTADTSSFVPLEL | |
| | | RVTAASGAPRYHRVIHINEVVL | |
| | | LDAPVGLVARLADESGHVVLR | |
| | | WLPPPETPMTSHIRYEVDVSA | |
| | | GNGAGSVQRVEILEGRTECVL | |
| | | SNLRGRTRYTFAVRARMAEP | |
| | | SFGGFWSAWSEPVSLLTPSD | |
| | | LDPRIPKVDKKVEPKSCDKTH | |
| | | TCPPCPAPELLGGPSVFLFPP | |
| | | KPKDTLMISRTPEVTCVVVDV | |
| | | SHEDPEVKFNWYVDGVEVHN | |
| | | AKTKPREEQYNSTYRVVSVLT | |
| | | VLHQDWLNGKEYKCRVSNKA | |
| | | LPAPIEKTISKAKGQPREPQVY | |
| | | TLPPSRDELTKNQVSLTCLVK | |
| | | GFYPSDIAVEWESNGQPENN | |
| | | YKTTPPVLDSDGSFFLYSKLT | |
| | | VDKSRWQQGNVFSCSVMHE | |
| | | ALHNHYTQKSLSLSP | |
| NVS78T | EPOR | SEQ ID NO: 147 | None |
| | | GGGGGPPPNLPDPKFESKAA | |
| | | LLAARGPEELLCFTERLEDLV | |
| | | CFWEEAASAGVGPGNYSFSY | |
| | | QLEDEPWKLCRLHQAPTARG | |
| | | AVRFWCSLPTADTSSFVPLEL | |
| | | RVTAASGAPRYHRVIHINEVVL | |
| | | LDAPVGLVARLADESGHVVLR | |
| | | WLPPPETPMTSHIRYEVDVSA | |
| | | GNGAGSVQRVEILEGRTECVL | |
| | | SNLRGRTRYTFAVRARMAEP | |
| | | SFGGFWSAWSEPVSLLTPSD | |
| | | LDPRIPKVDKKVEPKSCDKTH | |
| | | TCPPCPAPELLGGPSVFLFPP | |
| | | KPKDTLMISRTPEVTCVVVDV | |
| | | SHEDPEVKFNWYVDGVEVHN | |
| | | AKTKPREEQYNSTYRVVSVLT | |
| | | VLHQDWLNGKEYKCRVSNKA | |
| | | LPAPIEKTISKAKGQPREPQVY | |
| | | TLPPSRDELTKNQVSLTCLVK | |
| | | GFYPSDIAVEWESNGQPENN | |
| | | YKTTPPVLDSDGSFFLYSKLT | |
| | | VDKSRWQQGNVFSCSVMHE | |
| | | ALHNHYTQKSLSLSPGSGGG | |
| | | GVYHREAQSGKYKLTYAEAK | |
| | | AVCEFEGGHLATYKQLEAARK | |
| | | IGFHVCAAGWMAKGRVGYPI | |
| | | VKPGPNCGFGKTGIIDYGIRLN | |
| | | RSERWDAYCYNPHA | |
| NVS90 | EPOR | SEQ ID NO: 148 | None |
| | | APPRLICDSRVLERYLLEAKEA | |
| | | ENITTGCAEHCSLNENITVPDT | |
| | | KVNFYAWKRMEVGQQAVEV | |
| | | WQGLALLSEAVLRGQALLVNS | |
| | | SQPWEPLQLHVDKAVSGLRS | |
| | | LTTLLRALGAQKEAISPPDAAS | |
| | | AAPLRTITADTFRKLFRVYSNF | |
| | | LRGKLKLYTGEACRTGDR | |
| NVS90T | EPOR | SEQ ID NO: 149 | None |
| | | APPRLICDSRVLERYLLEAKEA | |
| | | ENITTGCAEHCSLNENITVPDT | |
| | | KVNFYAWKRMEVGQQAVEV | |
| | | WQGLALLSEAVLRGQALLVNS | |
| | | SQPWEPLQLHVDKAVSGLRS | |
| | | LTTLLRALGAQKEAISPPDAAS | |
| | | AAPLRTITADTFRKLFRVYSNF | |
| | | LRGKLKLYTGEACRTGDRGS | |
| | | GGGGVYHREAQSGKYYLTYA | |
| | | EAKAVCEFEGGHLATYKQLEA | |
| | | ARKIGFHVCAAGWMAKGRVG | |
| | | YPIVKPGPNCGFGKTGIIDYGI | |
| | | RLNRSERWDAYCYNPHAGSH | |
| | | HHHHH | |
| NVS79 | PDGF-BB | SEQ ID NO: 150 | None |
| | | 5′-(C6-NH2)-dC-dA-dG-dG-dC- | |
| | | fU-dA-fC-mG-HEG-dC-dG-T- | |
| | | dA-mG-dA-mG-dC-dA-fU-fC- | |
| | | mA-HEG-T-dG-dA-T-fC-fC-fU- | |
| | | mG-3′-dT-3′ | |
| | | HEG = hexaethylene glycol | |
| | | phosphoamidite | |
| NVS79T | PDGF-BB | SEQ ID NO: 151 | None |
| | | 5′-(C6-NH2)- dC-dA-dG-dG-dC- | |
| | | fU-dA-fC-mG-HEG-dC-dG-T- | |
| | | dA-mG-dA-mG-dC-dA-fU-fC- | |
| | | mA-HEG-T-dG-dA-T-fC-fC-fU- | |
| | | mG-3′-dT-3′- | |
| | | LPETGGGGGGSGGGGVYHR | |
| | | EAQSGKYYLTYAEAKAVCEFE | |
| | | GGHLATYKQLEAARKIGFHVC | |
| | | AAGWMAKGRVGYPIVKPGPN | |
| | | CGFGKTGIIDYGIRLNRSERW | |
| | | DAYCYNPHAGGSHHHHHH | |
| | | HEG = hexaethylene glycol | |
| | | phosphoamidite | |
| NVS91 | IL-10R | SEQ ID NO: 152 | None |
| | | SPGQGTQSENSCTHFPGNLP | |
| | | NMLRDLRDAFSRVKTFFQMK | |
| | | DQLDNLLLKESLLEDFKGYLG | |
| | | CQALSEMIQFYLEEVMPQAEN | |
| | | QDPDIKAHVNSLGENLKTLRL | |
| | | RLRRCHRFLPCENKSKAVEQ | |
| | | VKNAFNKLQEKGIYKAMSEFD | |
| | | IFINYIEAYMTMKIRN | |
| NVS91T | IL-10R | SEQ ID NO: 153 | None |
| | | SPGQGTQSENSCTHFPGNLP | |
| | | NMLRDLRDAFSRVKTFFQMK | |
| | | DQLDNLLLKESLLEDFKGYLG | |
| | | CQALSEMIQFYLEEVMPQAEN | |
| | | QDPDIKAHVNSLGENLKTLRL | |
| | | RLRRCHRFLPCENKSKAVEQ | |
| | | VKNAFNKLQEKGIYKAMSEFD | |
| | | IFINYIEAYMTMKIRNGSGGGG | |
| | | VYHREAQSGKYKLTYAEAKAV | |
| | | CEFEGGHLATYKQLEAARKIG | |
| | | FHVCAAGWMAKGRVGYPIVK | |
| | | PGPNCGFGKTGI | |
| | | IDYGIRLNRSERWDAYCYNPH | |
| | | AGSGGHHHHHH | |
| mAb1 | TNFα | SEQ ID NO: 217 | SEQ ID NO: 212 |
| mAb2 | TNFα | SEQ ID NO: 219 | SEQ ID NO: 218 |

Underlined sequences indicate additional optional sequence used for cloning (i.e.: GGGGG, SEQ ID NO: 187) or purification methods (e.g.: a hexa-histidine peptide, HHHHHH, SEQ ID NO: 188) described.

TABLE 5
Sequences of VEGF binding proteins linked to a peptide tag that binds HA.

| NVS ID | Single Chain or Heavy chain | Light Chain |
|---|---|---|
| NVS80 | SEQ ID NO: 154 | None |
| | SDTGRPFVEMYSEIPEIIHMTEGRELVIPC | |
| | RVTSPNITVTLKKFPLDTLIPDGKRIIWDSR | |
| | KGIISNATYKEIGLLTCEATVNGHLYKTNY | |
| | LTHRQTNTIIDVVLSPSHGIELSVGEKLVLN | |
| | CTARTELNVGIDFNWEYPSSKHQHKKLVN | |
| | RDLKTQSGSEMKKFLSTLTIDGVTRSDQG | |
| | LYTCAASSGLMTKKNSTFVRVHEKDKTHT | |
| | CPPCPAPEAAGGPSVFLFPPKPKDTLMIS | |
| | RTPEVTCVVVDVSHEDPEVKFNWYVDGV | |
| | EVHNAKTKPREEQYNSTYRVVSVLTVLHQ | |
| | DWLNGKEYKCKVSNKALPAPIEKTISKAK | |
| | GQPREPQVYTLPPSRDELTKNQVSLTCLV | |
| | KGFYPSDIAVEWESNGQPENNYKTTPPVL | |
| | DSDGSFFLYSKLTVDKSRWQQGNVFSCS | |
| | VMHEALHNHYTQKSLSLSPGK | |
| NVS80T | SEQ ID NO: 156 | None |
| | SDTGRPFVEMYSEIPEIIHMTEGRELVIPC | |
| | RVTSPNITVTLKKFPLDTLIPDGKRIIWDSR | |
| | KGFIISNATYKEIGLLTCEATVNGHLYKTNY | |
| | LTHRQTNTIIDVVLSPSHGIELSVGEKLVLN | |
| | CTARTELNVGIDFNWEYPSSKHQHKKLVN | |
| | RDLKTQSGSEMKKFLSTLTIDGVTRSDQG | |
| | LYTCAASSGLMTKKNSTFVRVHEKDKTHT | |
| | CPPCPAPEAAGGPSVFLFPPKPKDTLMIS | |
| | RTPEVTCVVVDVSHEDPEVKFNWYVDGV | |
| | EVHNAKTKPREEQYNSTYRVVSVLTVLHQ | |
| | DWLNGKEYKCKVSNKALPAPIEKTISKAK | |
| | GQPREPQVYTLPPSRDELTKNQVSLTCLV | |
| | KGFYPSDIAVEWESNGQPENNYKTTPPVL | |
| | DSDGSFFLYSKLTVDKSRWQQGNVFSCS | |
| | VMHEALHNHYTQKSLSLSPGGSGGGGVY | |
| | HREAISGKYYLTYAEAKAVCEFEGGHLAT | |
| | YKQLEAAQQIGFHVCAAGWMAKGRVGYP | |
| | IVKPGPNCGFGKTGIIDYGIRLQRSERWD | |
| | AYCYNPHA | |
| NVS81 | SEQ ID NO: 157 | SEQ ID NO: 158 |
| | EVQLVESGGGLVQPGGSLRLSCAASGYT | DIQMTQSPSSLSASVGDRVTITCSASQ |
| | FTNYGMNWVRQAPGKGLEWVGWINTYT | DISNYLNWYQQKPGKAPKVLIYFTSSL |
| | GEPTYAADFKRRFTFSLDTSKSTAYLQMN | HSGVPSRFSGSGSGTDFTLTISSLQP |
| | SLRAEDTAVYYCAKYPHYYGSSHWYFDV | EDFATYYCQQYSTVPVVTFGQGTKVEI |
| | WGQGTLVTVSSASTKGPSVFPLAPSSKS | KRTVAAPSVFIFPPSDEQLKSGTASVV |
| | TSGGTAALGCLVKDYFPEPVTVSWNSGA | CLLNNFYPREAKVQWKVDNALQSGN |
| | LTSGVHTFPAVLQSSGLYSLSSVVTVPSS | SQESVTEQDSKDSTYSLSSTLTLSKA |
| | SLGTQTYICNVNHKPSNTKVDKKVEPKSC | DYEKHKVYACEVTHQGLSSPVTKSFN |
| | DKTHTCPPCPAPELLGGPSVFLFPPKPKD | RGEC |
| | TLMISRTPEVTCVVVDVSHEDPEVKFNWY | |
| | VDGVEVHNAKTKPREEQYNSTYRVVSVL | |
| | TVLHQDWLNGKEYKCKVSNKALPAPIEKT | |
| | ISKAKGQPREPQVYTLPPSREEMTKNQVS | |
| | LTCLVKGFYPSDIAVEWESNGQPENNYKT | |
| | TPPVLDSDGSFFLYSKLTVDKSRWQQGN | |
| | VFSCSVMHEALHNHYTQKSLSLSPGK | |
| NVS81T | SEQ ID NO: 159 | SEQ ID NO: 160 |
| | EVQLVESGGGLVQPGGSLRLSCAASGYT | DIQMTQSPSSLSASVGDRVTITCSASQ |
| | FTNYGMNWVRQAPGKGLEWVGWINTYT | DISNYLNWYQQKPGKAPKVLIYFTSSL |
| | GEPTYAADFKRRFTFSLDTSKSTAYLQMN | HSGVPSRFSGSGSGTDFTLTISSLQP |
| | SLRAEDTAVYYCAKYPHYYGSSHWYFDV | EDFATYYCQQYSTVPVVTFGQGTKVEI |
| | WGQGTLVTVSSASTKGPSVFPLAPSSKS | KRTVAAPSVFIFPPSDEQLKSGTASVV |
| | TSGGTAALGCLVKDYFPEPVTVSWNSGA | CLLNNFYPREAKVQWKVDNALQSGN |
| | LTSGVHTFPAVLQSSGLYSLSSVVTVPSS | SQESVTEQDSKDSTYSLSSTLTLSKA |
| | SLGTQTYICNVNHKPSNTKVDKKVEPKSC | DYEKHKVYACEVTHQGLSSPVTKSFN |
| | DKTHTCPPCPAPELLGGPSVFLFPPKPKD | RGEC |
| | TLMISRTPEVTCVVVDVSHEDPEVKFNWY | |
| | VDGVEVHNAKTKPREEQYNSTYRVVSVL | |
| | TVLHQDWLNGKEYKCKVSNKALPAPIEKT | |
| | ISKAKGQPREPQVYTLPPSREEMTKNQVS | |
| | LTCLVKGFYPSDIAVEWESNGQPENNYKT | |
| | TPPVLDSDGSFFLYSKLTVDKSRWQQGN | |
| | VFSCSVMHEALHNHYTQKSLSLSPGKGS | |
| | GGGGVYHREAQSGKYKLTYAEAKAVCEF | |
| | EGGHLATYKQLEAARKIGFHVCAAGWMA | |
| | KGRVGYPIVKPGPNCGFGKTGIIDYGIRLN | |
| | RSERWDAYCYNPHA | |
| NVS82 | SEQ ID NO: 161 | SEQ ID NO: 162 |
| | EVQLVESGGGLVQPGGSLRLSCTASGFS | EIVMTQSPSTLSASVGDRVIITCQASEII |
| | LTDYYYMTWVRQAPGKGLEWVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCDKT | KADYEKHKVYACEVTHQGLSSPVTKS |
| | HTCPPCPAPEAAGGPSVFLFPPKPKDTLM | FNRGEC |
| | ISRTPEVTCVVVDVSHEDPEVKFNWYVD | |
| | GVEVHNAKTKPREEQYNSTYRVVSVLTVL | |
| | HQDWLNGKEYKCKVSNKALPAPIEKTISK | |
| | AKGQPREPQVYTLPPSREEMTKNQVSLT | |
| | CLVKGFYPSDIAVEWESNGQPENNYKTT | |
| | PPVLDSDGSFFLYSKLTVDKSRWQQGNV | |
| | FSCSVMHEALHNHYTQKSLSLSPGK | |
| NVS82T | SEQ ID NO: 163 | SEQ ID NO: 164 |
| | EVQLVESGGGLVQPGGSLRLSCTASGFS | EIVMTQSPSTLSASVGDRVIITCQASEII |
| | LTDYYYMTWVRQAPGKGLEWVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCDKT | KADYEKHKVYACEVTHQGLSSPVTKS |
| | HTCPPCPAPEAAGGPSVFLFPPKPKDTLM | FNRGECGSGGGGVYHREAQSGKYKL |
| | ISRTPEVTCVVVDVSHEDPEVKFNWYVD | TYAEAKAVCEFEGGHLATYKQLEAAR |
| | GVEVHNAKTKPREEQYNSTYRVVSVLTVL | KIGFHVCAAGWMAKGRVGYPIVKPGP |
| | HQDWLNGKEYKCKVSNKALPAPIEKTISK | NCGFGKTGIIDYGIRLNRSERWDAYC |
| | AKGQPREPQVYTLPPSREEMTKNQVSLT | YNPHA |
| | CLVKGFYPSDIAVEWESNGQPENNYKTT | |
| | PPVLDSDGSFFLYSKLTVDKSRWQQGNV | |
| | FSCSVMHEALHNHYTQKSLSLSPGK | |
| NVS83 | SEQ ID NO: 165 | None |
| | MEIVMTQSPSTLSASVGDRVIITCQASEIIH | |
| | SWLAWYQQKPGKAPKLLIYLASTLASGVP | |
| | SRFSGSGSGAEFTLTISSLQPDDFATYYC | |
| | QNVYLASTNGANFGQGTKLTVLGGGGGG | |
| | SGGGGSGGGGSSGGGSEVQLVESGGGL | |
| | VQPGGSLRLSCTASGFSLTDYYYMTWVR | |
| | QAPGKGLEWVGFIDPDDDPYYATWAKGR | |
| | FTISRDNSKNTLYLQMNSLRAEDTAVYYC | |
| | AGGDHNSGWGLDIWGQGTLVTVSSHHH | |
| | HHH | |
| NVS83T | SEQ ID NO: 166 | None |
| | MEIVMTQSPSTLSASVGDRVIITCQASEIIH | |
| | SWLAWYQQKPGKAPKLLIYLASTLASGVP | |
| | SRFSGSGSGAEFTLTISSLQPDDFATYYC | |
| | QNVYLASTNGANFGQGTKLTVLGGGGGG | |
| | SGGGGSGGGGSSGGGSEVQLVESGGGL | |
| | VQPGGSLRLSCTASGFSLTDYYYMTWVR | |
| | QAPGKGLEWVGFIDPDDDPYYATWAKGR | |
| | FTISRDNSKNTLYLQMNSLRAEDTAVYYC | |
| | AGGDHNSGWGLDIWGQGTLVTVSSGSG | |
| | GGGVYHREAQSGKYKLTYAEAKAVCEFE | |
| | GGHLATYKQLEAARKIGFHVCAAGWMAK | |
| | GRVGYPIVKPGPNCGFGKTGIIDYGIRLNR | |
| | SERWDAYCYNPHAHHHHHH | |
| NVS84 | SEQ ID NO: 167 | None |
| | SDLGKKLLEAARAGQDDEVRILMANGADV | |
| | NTADSTGVVTPLHLAVPWGHLEIVEVLLKY | |
| | GADVNAKDFQGVVTPLHLAAAIGHQEIVEV | |
| | LLKNGADVNAQDKFGKTAFDISIDNGNED | |
| | LAEILQKAAGSLPETGGGSGHHHHHH | |
| NVS84T | SEQ ID NO: 168 | None |
| | SDLGKKLLEAARAGQDDEVRILMANGADV | |
| | NTADSTGVVTPLHLAVPWGHLEIVEVLLKY | |
| | GADVNAKDFQGVVTPLHLAAAIGHQEIVEV | |
| | LLKNGADVNAQDKFGKTAFDISIDNGNED | |
| | LAEILQKAAGSGGGGVYHREAQSGKYKL | |
| | TYAEAKAVCEFEGGHLATYKQLEAARKIG | |
| | FHVCAAGWMAKGRVGYPIVKPGPNCGF | |
| | GKTGIIDYGIRLNRSERWDAYCYNPHAGS | |
| | GGHHHHHH | |
| NVS85 | SEQ ID NO: 169 | None |
| | SDLGKKLLEAARAGQDDEVRILMANGADV | |
| | NAFDWMGVVTPLHLAAHEGHLEIVEVLLK | |
| | NGADVNATDVSGYTPLHLAAADGHLEIVE | |
| | VLLKYGADVNTKDNTGVVTPLHLSADLGRL | |
| | EIVEVLLKYGADVNAQDKFGKTAFDISIDN | |
| | GNEDLAEILQKAAHHHHHH | |
| NVS85T | SEQ ID NO: 170 | None |
| | SDLGKKLLEAARAGQDDEVRILMANGADV | |
| | NAFDWMGVVTPLHLAAHEGHLEIVEVLLK | |
| | NGADVNATDVSGYTPLHLAAADGHLEIVE | |
| | VLLKYGADVNTKDNTGVVTPLHLSADLGRL | |
| | EIVEVLLKYGADVNAQDKFGKTAFDISIDN | |
| | GNEDLAEILQKAAGSGGGGVYHREAQSG | |
| | KYKLTYAEAKAVCEFEGGHLATYKQLEAA | |
| | RKIGFHVCAAGWMAKGRVGYPIVKPGPN | |
| | CGFGKTGIIDYGIRLNRSERWDAYCYNPH | |
| | AGSGGHHHHHH | |
| NVS1b | SEQ ID NO: 171 | SEQ ID NO: 172 |
| | EVQLVESGGGLVQPGGSLRLSCTASGFS | GGGGGEIVMTQSPSTLSASVGDRVIIT |
| | LTDYYYMTWVRQAPGKGLEWVGFIDPDD | CQASEIIHSWLAWYQQKPGKAPKLLIY |
| | DPYYATWAKGRFTISRDNSKNTLYLQMN | LASTLASGVPSRFSGSGSGAEFTLTIS |
| | SLRAEDTAVYYCAGGDHNSGWGLDIWG | SLQPDDFATYYCQNVYLASTNGANFG |
| | QGTLVTVSSASTKGPSVFPLAPSSKSTSG | QGTKLTVLKRTVAAPSVFIFPPSDEQL |
| | GTAALGCLVKDYFPEPVTVSWNSGALTS | KSGTASVVCLLNNFYPREAKVQWKVD |
| | GVHTFPAVLQSSGLYSLSSVVTVPSSSLG | NALQSGNSQESVTEQDSKDSTYSLSS |
| | TQTYICNVNHKPSNTKVDKRVEPKSCGS | TLTLSKADYEKHKVYACEVTHQGLSS |
| | GGGGVYHREAQSGKYKLTYAEAKAVCEF | PVTKSFNRGEC |
| | EGGHLATYKQLEAARKIGFHVCAAGWMA | |
| | KGRVGYPIVKPGPNCGFGKTGIIDYGIRLN | |
| | RSERWDAYCYNPHA | |
| NVS1c | SEQ ID NO: 173 | SEQ ID NO: 174 |
| | VYHREARSGKYKLTYAEAKAVCEFEGGH | VYHREARSGKYKLTYAEAKAVCEFEG |
| | LATYKQLEAARKIGFHVCAAGWMAKGRV | GHLATYKQLEAARKIGFHVCAAGWMA |
| | GYPIVKPGPNCGFGKTGIIDYGIRLNRSER | KGRVGYPIVKPGPNCGFGKTGIIDYGI |
| | WDAYCYNPHAKGGGSEVQLVESGGGLV | RLNRSERWDAYCYNPHAKGGGSEIV |
| | QPGGSLRLSCTASGFSLTDYYYMTWVRQ | MTQSPSTLSASVGDRVIITCQASEIIHS |
| | APGKGLEWVGFIDPDDDPYYATWAKGRF | WLAWYQQKPGKAPKLLIYLASTLASG |
| | TISRDNSKNTLYLQMNSLRAEDTAVYYCA | VPSRFSGSGSGAEFTLTISSLQPDDFA |
| | GGDHNSGWGLDIWGQGTLVTVSSASTK | TYYCQNVYLASTNGANFGQGTKLTVL |
| | GPSVFPLAPSSKSTSGGTAALGCLVKDYF | KRTVAAPSVFIFPPSDEQLKSGTASVV |
| | PEPVTVSWNSGALTSGVHTFPAVLQSSG | CLLNNFYPREAKVQWKVDNALQSGN |
| | LYSLSSVVTVPSSSLGTQTYICNVNHKPS | SQESVTEQDSKDSTYSLSSTLTLSKA |
| | NTKVDKRVEPKSCGS | DYEKHKVYACEVTHQGLSSPVTKSFN |
| | | RGEC |
| NVS1d | SEQ ID NO: 175 | SEQ ID NO: 176 |
| | EVQLVESGGGLVQPGGSLRLSCTASGFS | EIVMTQSPSTLSASVGDRVIITCQASEII |
| | LTDYYYMTWVRQAPG KGLEWVGF IDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | GGGGVYHREAQSGKYKLTYAEAKAVCEF | FNRGECGSGGGGVYHREAQSGKYKL |
| | EGGHLATYKQLEAARKIGFHVCAAGWMA | TYAEAKAVCEFEGGHLATYKQLEAAR |
| | KGRVGYPIVKPGPNCGFGKTGIIDYGIRLN | KIGFHVCAAGWMAKGRVGYPIVKPGP |
| | RSERWDAYCYNPHA | NCGFGKTGIIDYGIRLNRSERWDAYC |
| | | YNPHA |
| NVS1e | SEQ ID NO: 177 | SEQ ID NO: 178 |
| | EVQLVESGGGLVQPGGSLRLSCTASGFS | EIVMTQSPSTLSASVGDRVIITCQASEII |
| | LTDYYYMTWVRQAPG KG LEWVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | | FNRGECGSGGGGVYHREAQSGKYKL |
| | | TYAEAKAVCEFEGGHLATYKQLEAAR |
| | | KIGFHVCAAGWMAKGRVGYPIVKPGP |
| | | NCGFGKTGIIDYGIRLNRSERWDAYC |
| | | YNPHAGSGGGGVYHREAQSGKYKLT |
| | | YAEAKAVCEFEGGHLATYKQLEAARKI |
| | | GFHVCAAGWMAKGRVGYPIVKPGPN |
| | | CGFGKTGIIDYGIRLNRSERWDAYCY |
| | | NPHA |
| NVS1f | SEQ ID NO: 179 | SEQ ID NO: 180 |
| | EVQLVESGGGLVQPGGSLRLSCTASGFS | VYHREARSGKYKLTYAEAKAVCEFEG |
| | LTDYYYMTWVRQAPGKGLEWVGFIDPDD | GHLATYKQLEAARKIGFHVCAAGWMA |
| | DPYYATWAKGRFTISRDNSKNTLYLQMN | KGRVGYPIVKPGPNCGFGKTGIIDYGI |
| | SLRAEDTAVYYCAGGDHNSGWGLDIWG | RLNRSERWDAYCYNPHAKGGGSEIV |
| | QGTLVTVSSASTKGPSVFPLAPSSKSTSG | MTQSPSTLSASVGDRVIITCQASEIIHS |
| | GTAALGCLVKDYFPEPVTVSWNSGALTS | WLAWYQQKPGKAPKLLIYLASTLASG |
| | GVHTFPAVLQSSGLYSLSSVVTVPSSSLG | VPSRFSGSGSGAEFTLTISSLQPDDFA |
| | TQTYICNVNHKPSNTKVDKRVEPKSCGS | TYYCQNVYLASTNGANFGQGTKLTVL |
| | GGGGVYHREARSGKYKLTYAEAKAVCEF | KRTVAAPSVFIFPPSDEQLKSGTASVV |
| | EGGHLATYKQLEAARKIGFHVCAAGWMA | CLLNNFYPREAKVQWKVDNALQSGN |
| | KGRVGYPIVKPGPNCGFGKTGIIDYGIRLN | SQESVTEQDSKDSTYSLSSTLTLSKA |
| | RSERWDAYCYNPHA | DYEKHKVYACEVTHQGLSSPVTKSFN |
| | | RGEC |
| NVS1g | SEQ ID NO: 181 | SEQ ID NO: 182 |
| | EVQLVESGGGLVQPGGSLRLSCTASGFS | EIVMTQSPSTLSASVGDRVIITCQASEII |
| | LTDYYYMTWVRQAPGKGLEWVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | GGGGVYHREARSGKYKLTYAEAKAVCEF | FNRGEC |
| | EGGHLATYKQLEAARKIGFHVCAAGWMA | |
| | KGRVGYPIVKPGPNCGFGKTGIIDYGIRLN | |
| | RSERWDAYCYNPHAGGGGGGSGVYHRE | |
| | ARSGKYKLTYAEAKAVCEFEGGHLATYK | |
| | QLEAARKIGFHVCAAGWMAKGRVGYPIV | |
| | KPGPNCGFGKTGIIDYGIRLNRSERWDAY | |
| | CYNPHAGSGGGGVYHREARSGKYKLTYA | |
| | EAKAVCEFEGGHLATYKQLEAARKIGFHV | |
| | CAAGWMAKGRVGYPIVKPGPNCGFGKT | |
| | GIIDYGIRLNRSERWDAYCYNPHAGSGGG | |
| | GVYHREARSGKYKLTYAEAKAVCEFEGG | |
| | HLATYKQLEAARKIGFHVCAAGWMAKGR | |
| | VGYPIVKPGPNCGFGKTGIIDYGIRLNRSE | |
| | RWDAYCYNPHA | |
| NVS1h | SEQ ID NO: 183 | SEQ ID NO: 184 |
| | EVQLVESGGGLVQPGGSLRLSCTASGFS | EIVMTQSPSTLSASVGDRVIITCQASEII |
| | LTDYYYMTWVRQAPGKGLEWVGFIDPDD | HSWLAWYQQKPGKAPKLLIYLASTLA |
| | DPYYATWAKGRFTISRDNSKNTLYLQMN | SGVPSRFSGSGSGAEFTLTISSLQPD |
| | SLRAEDTAVYYCAGGDHNSGWGLDIWG | DFATYYCQNVYLASTNGANFGQGTKL |
| | QGTLVTVSSASTKGPSVFPLAPSSKSTSG | TVLKRTVAAPSVFIFPPSDEQLKSGTA |
| | GTAALGCLVKDYFPEPVTVSWNSGALTS | SVVCLLNNFYPREAKVQWKVDNALQS |
| | GVHTFPAVLQSSGLYSLSSVVTVPSSSLG | GNSQESVTEQDSKDSTYSLSSTLTLS |
| | TQTYICNVNHKPSNTKVDKRVEPKSCGS | KADYEKHKVYACEVTHQGLSSPVTKS |
| | GGGGVYHREARSGKYKLTYAEAKAVCEF | FNRGEC |
| | EGGHLATYKQLEAARKIGFHVCAAGWMA | |
| | KGRVGYPIVKPGPNCGFGKTGIIDYGIRLN | |
| | RSERWDAYCYNPHAGSGGGGVYHREAR | |
| | SGKYKLTYAEAKAVCEFEGGHLATYKQLE | |
| | AARKIGFHVCAAGWMAKGRVGYPIVKPG | |
| | PNCGFGKTGIIDYGIRLNRSERWDAYCYN | |
| | PHA | |
| NVS1j | SEQ ID NO: 9 | SEQ ID NO: 185 |
| | EVQLVESGGGLVQPGGSLRLSCTASGFS | EIVMTQSPSTLSASVGDRVIITCQASEII |
| | LTDYYYMTWVRQAPGKGLEWVGFIDPDD | HSWLAWYQQKPGKAPKLLIYLASTLA |
| | DPYYATWAKGRFTISRDNSKNTLYLQMN | SGVPSRFSGSGSGAEFTLTISSLQP |
| | SLRAEDTAVYYCAGGDHNSGWGLDIWG | DDFATYYCQNVYLASTNGANFGQGT |
| | QGTLVTVSSASTKGPSVFPLAPSSKSTSG | KLTVLKRTVAAPSVFIFPPSDEQLKSG |
| | GTAALGCLVKDYFPEPVTVSWNSGALTS | TASVVCLLNNFYPREAKVQWKVDNAL |
| | GVHTFPAVLQSSGLYSLSSVVTVPSSSLG | QSGNSQESVTEQDSKDSTYSLSSTLT |
| | TQTYICNVNHKPSNTKVDKRVEPKSCGS | LSKADYEKHKVYACEVTHQGLSSPVT |
| | | KSFNRGECGSGGGGVYHREAQSGKY |
| | | KLTYAEAKAVCEFEGGHLATYKQLEA |
| | | ARKIGFHVCAAGWMAKGRVGYPIVKP |
| | | GPNCGFGKTGIIDYGIRLNRSERWDA |
| | | YCYNPHA |

### Example 2: Rabbit Intra-Articular PK Determination

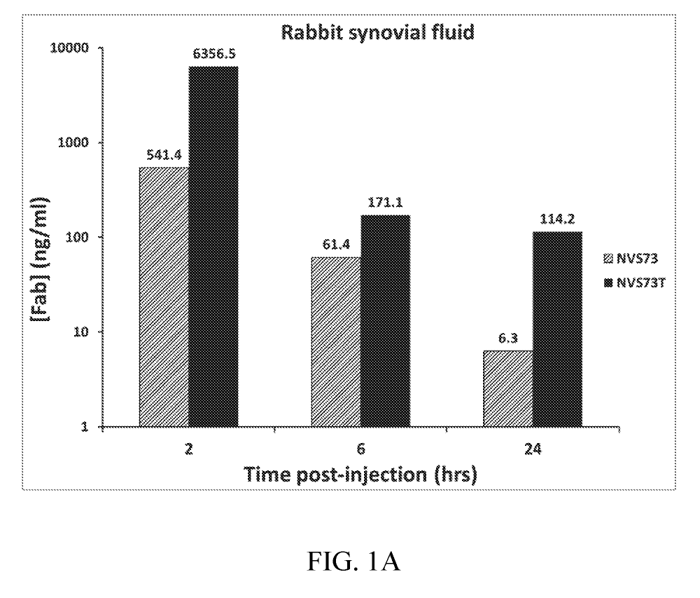

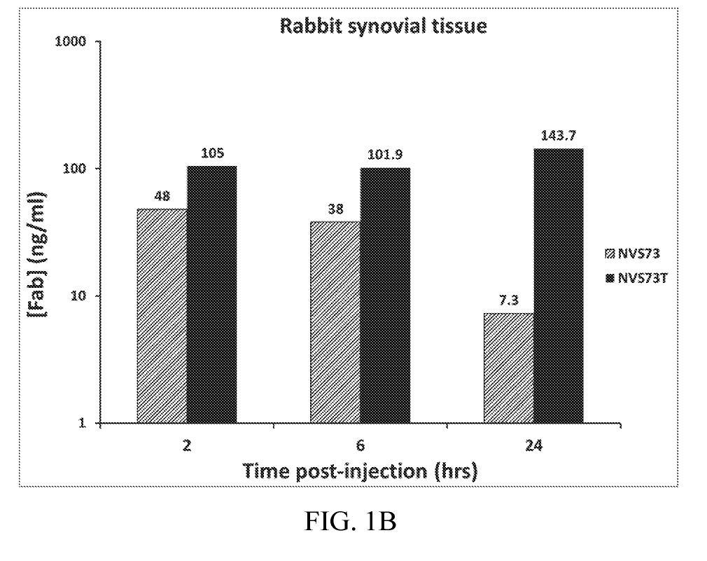

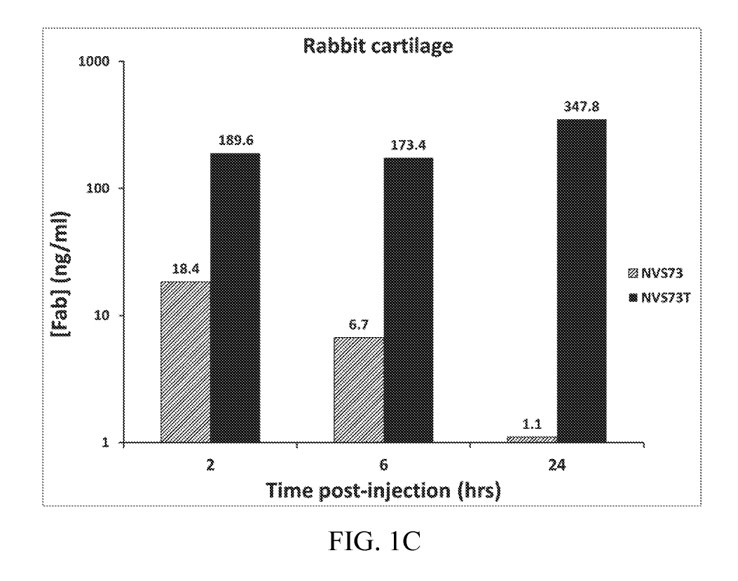

Intra-articular concentrations of a Fab linked to an HA-binding peptide tag (NVS73T) in rabbit joint was compared to its untagged version (NVS73) using standard methods as described below and shown in FIG. 1.

### Rabbit PK Study

Animals:

Female New Zealand White rabbits weighing between 1.8 and 2.2 kg were used throughout. The rabbits were held in group housing and placed in individual cages on the morning of the experiment. This study was performed in accordance with the animal experimentation guidelines and laws laid down by the Swiss Federal and Cantonal Authorities, and specifically as described in Basel-Stadt Experimental License No. 1438.

### Pharmacokinetic Experiment:

The rabbits were anaesthetised with a mixed s.c. injection of acepromazine, xylazine and ketamine (1 mg/kg, 2 mg/kg and 60 mg/kg, respectively). Intra-articular injections into the left knee joint were performed using a 25 G needle and syringe. The rabbits received either, 100 ug anti-TNFα Fab (n=3), NVS73 or 100 ug hyaluronic acid binding peptide tagged anti-TNFα Fab (n=3), NVS73T. A further 2 rabbits, without i.a. injection, were included as matrix controls (baseline measurements) for the mass spectrometric method.

At times 2, 6 and 24 h after the intra-articular injections, the rabbits were sedated with a s.c. injection of acepromazine (1 mg/kg). The rabbits were then killed by an i.v. overdose of sodium pentobarbital (500 mg/kg), followed by an intra-cardiac injection of saturated KCl solution using a 20 G needle. Blood samples were taken directly from the heart using the same needle. The blood was aliquoted into serum preparation tubes (Sarstedt) and placed on ice. Blood samples were also taken, via the lateral ear vein after sedation, from the remaining 4 rabbits at time 2 h, and the remaining 2 rabbits at time 6 h.

The left knee joints were lavaged to obtain synovial fluid samples after injection with 200 ul of 3.8% sodium citrate solution via a 23 G needle and 1 ml syringe. The synovial washout fluid was aliquoted into Eppendorf tubes and placed on ice.

The skin over the knees was dampened with 70% alcohol and opened by cutting vertically over the center of the joint. The knee joints were opened via a transverse cut above the patella, through the muscle and tendon. The joint was opened by cutting diagonally down on the medial and lateral sides, and the patella reflected outwards and downwards to gain access to the synovial tissue at the front of the joint. The synovial tissue was removed from its attachment at the front of the tibia and dissected free of the patella tendon using a scalpel blade. The tissue was cut into four pieces and placed into Eppendorf tubes on ice.

The patella was trimmed of excess tissue and snap frozen for histological analysis.

Samples of cartilage were removed from the patella groove of the femur using a scalpel blade, transferred into Eppendorf tubes and placed on ice.

Serum, synovial fluid, synovial tissue and cartilage samples were stored at −80° C. prior to mass spectrometric analysis.

The harvested tissues and fluids were further homogenized mechanically using a TissueLyzer (QIAGEN®). Antibody levels in the vitreous were measured by ELISA or mass spectrometry.

### Mass Spectrometry Method

### Reduction, Alkylation and Digestion:

Samples in each well were thawed at room temperature for 10 minutes. 150 uL of 8M Urea (FisherScientific®, Cat No. U15-500) in 50 mM Tris-HCl (Fisher Scientific®, BP153-500) was added to each sample well, followed by addition of 4 uL of 2M DTT (SigmaAldrich®, Cat. No. D9779) to a final concentration of 40 mM DTT. The plate was heated at 58 deg C. for 45 minutes to denature the proteins. Subsequently, cool the plate to room temperature, then add 8 uL of 1M Iodoacetamide (SigmaAldrich®, Cat. No. 11149) for a final concentration of 40 mM and incubate at room temperature for 45 minutes in the dark. Dilute final concentration of urea to below 2M by adding 1.3 mL of 50 mM ammonium bicarbonate (Fisher Scientific®, Cat. No. BP2413-500). Add 10 uL of 0.1 ug/uL trypsin (Promega®, Cat. No. V5111) and incubate at 37° C. overnight.

### SPE Cleanup and Filtration:

After digestion, add formic acid (Fluke, Cat. No. 56302-50ML-F) to each sample to a final concentration of 1% (v/v) to quench trypsin digestion. Oasis® MCX plate (Waters, Cat. No. 186000259) is used to clean up the digested sample. The collected sample solution from cleanup was dried down completely using SpeedVac (ThermoFisher Savant). Once the sample is dried, 60 uL of buffer (0.1% formic acid, 1% ACN (Sigma Aldrich, Cat. No. 34998-4L) and 20 pg/uL heavy labeled internal standard (custom made by ThermoFisher) solution is added to each well, and the plate was shaken for 20 minutes. The reconstituted peptide solution was filtered using AcroPrep™ advanced 96-well filter plates for ultrafiltration (Pall Life Sciences, Cat. No. 8164) filter with 10 KDa MWCO.

### LC-MS/MS Analysis:

5 uL of each filtered samples was loaded to a 300 umλ150 mm Symmetry® C18 column (Waters®, Cat. No. 186003498). Separation was achieved by applying a 5 min gradient from 5% B (acetonitrile in 0.1% formic acid) to 20% B with a flow rate of 5 uL/min. Two peptides (HC_T3: GPSVFPLAPSSK and DDA2: TGIIDYGIR), and two transitions for each peptide (HC_T3: 594.19/699.82 and 594.19/847; DDA2: 504.58/623.68 and 504.58/736.84) were monitored for each sample using Waters Xevo TQS mass spectrometer (Waters). Drug molecules containing these peptides were quantified using MS signals resulted from these transitions.

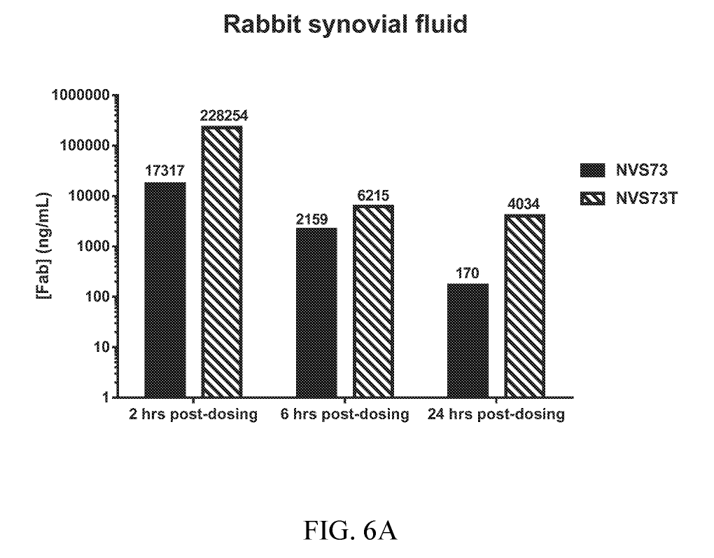

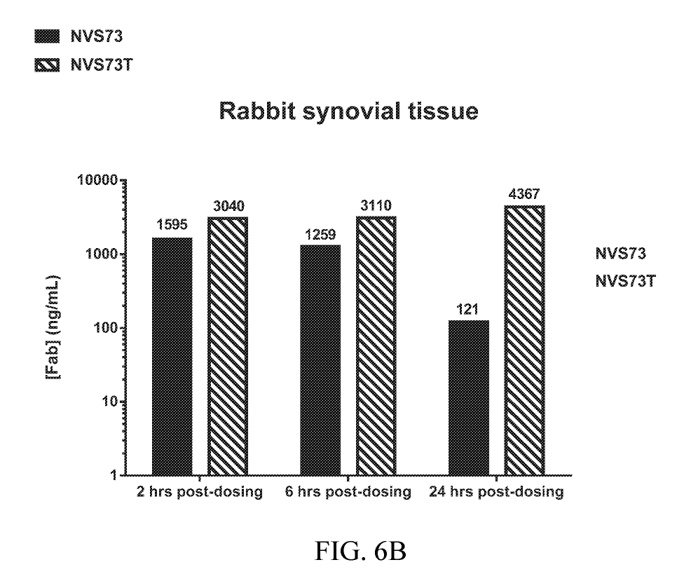

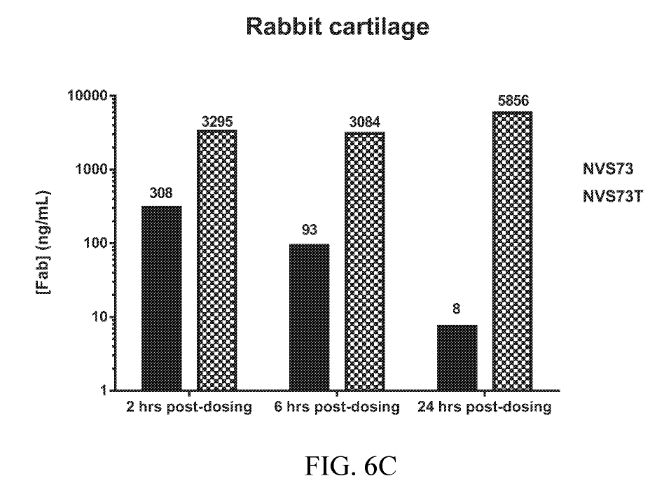

FIGS. 1A-C were plotted based on data calculated from standard curves that were plotted in ng of drug vs mass spectrometric signal. Since the joint contains both tissues (synovial tissue and cartilage) and fluids (synovial fluids), new calculations were performed in which FIGS. 6A-C were generated from the same original data used for FIGS. 1A-C but using standard curves that were plotted in ng/ml of drug vs mass spectrometric signal.

### Example 3: Rat Traditional Intra-Articular PK/PD Determination

### Animals:

Female Lewis rats weighing between 175 and 195 g were used throughout. The rats were held in groups of 5 animals in IVC (individually ventilated cage) racks. This study was performed in accordance with the animal experimentation guidelines and laws laid down by the Swiss Federal and Cantonal Authorities, and specifically as described in Basel-Stadt Experimental License No. 1438.

### Pharmacokinetic/Pharmacodynamic Experiment:

The rats were anaesthetised with 3.5-5% isoflurane in air in an anaesthetic induction chamber. Intra-articular injections into the left knee joints were performed using a 30 G needle and 1 ml syringe under isoflurane anaesthesia. Groups of 5 rats received either, 100 ug anti-TNFα Fab, NVS73) or 52 ug hyaluronic acid tagged anti-TNFα Fab, NVS73T at times −24 h, −6 h or −2 h.

A further group of 4 rats, without i.a. injection, were included as matrix controls (baseline measurements) for the mass spectrometry method.

At time 0 h after the intra-articular Fab injections, the right and left knee diameters of the rats were measured using digital calipers, followed by the intra-articular injection of recombinant human TNFα (30 ug) into each left knee under isoflurane anaesthesia. After 6 hours, the diameters of the right and left rat knees were again measured by digital calipers and the animals bled under isoflurane anaesthesia. The blood was collected into serum preparation tubes (Sarstedt) and placed on ice. The rats were then killed via CO₂ inhalation for collection of synovial fluid washout, synovial tissue and cartilage.

The left knee joints were lavaged after injection of 100 ul of 3.8% sodium citrate solution via a 25 G needle and 1 ml syringe. The synovial washout fluid was placed into Eppendorf tubes on ice.

The skin over the knees was dampened with 70% alcohol and opened by cutting vertically over the center of the joint. The knee joints were opened via a transverse cut above the patella, through the muscle and tendon. The joint was opened by cutting diagonally down on the medial and lateral sides, and the patella reflected outwards and downwards to gain access to the synovial tissue at the front of the joint. The synovial tissue was removed from its attachment at the front of the tibia and dissected free of the patella tendon using a scalpel blade. The tissue was placed in Eppendorf tubes on ice.

Samples of cartilage were removed from the patella groove of the femur using a scalpel blade, transferred into Eppendorf tubes and placed on ice.

Serum, synovial fluid, synovial tissue and cartilage samples were stored at −80° C. prior to mass spectrometric analysis.

### Data and Statistical Analysis:

Knee swelling was calculated as a ratio of the left knee diameter (TNFα injected knee)/right knee diameter (non-injected knee) for each rat. The mean±sem was calculated for each time and treatment group. Data were analysed by ANOVA, followed by post hoc tests for multiple comparisons.

### Gyrolab Method

### Sample Preparation

Samples were thawed at room temperature for 10 minutes. 5 uL of sample is then diluted 1:2 in Rexxip H Buffer (Gyros AB®, Inc. Cat P0004823) in a 96-well PCR plate (Thermo Scientific® AB-800, 0.2 mL Skirted 96-well PCR plate). Samples were sealed (Gyros AB®, Inc. microplate foil Cat P0003313) mixed gently by pipetting a few times so as to minimize formation of bubbles. Ensuring that no bubbles are found in the bottom of the wells, the samples were placed in the Gyrolab™ xP workstation. A 3-step C-A-D method is executed on the Gyrolab™ xP workstation; capture antibody is flowed through the system first, followed by the analyte (samples), and then the detector antibody. The Gyrolab™ xP workstation performs washes of PBS 0.01% Tween20 (Calbiochem®, Inc. Cat 655206) in between each step. The standard curve for free drug measurement was prepared neat in naïve rabbit/rat joint tissue/fluid/cartilage (matrix) at 24,000 ng/mL and then serially diluted 1:5 in neat naïve rabbit/rat matrix. The standard curve is then diluted 1:2 in Rexxip Hmax, such that final matrix concentration is at 50% and standard series begins at 12,000 ng/mL and extends to 0.768 ng/mL

### Detection of Fabs

Total and free purified drug constructs were analyzed in the Gyrolab™ xP workstation using a Bioaffy200 CD (Gyros AB, Inc. Cat P0004253). Free drug is measured by applying 100 ug/mL biotin-labeled TNF (Novartis) to a column containing streptavidin coated particles. Vitreous samples are applied to the activated columns and detected by capillary action with 25 nM alexafluor 647 labeled goat anti-Human IgG-heavy and light chain antibody (Bethyl Laboratories®, Cat A80-319A). Note that alexafluor 647 labeling was performed using Life Technologies labeling kit (Cat A-20186). The capture reagent was prepared in PBS 0.01% Tween20 and the detector reagent in Rexxip F (Gyros AB®, Inc. P0004825). Total drug is measured by applying 100 ug/mL biotin-labeled goat anti-Human IgG-heavy and light chain antibody (Bethyl Laboratories®, Cat A80-319B). Vitreous samples are applied to the activated columns and detected by capillary action with 10 nM alexafluor-647 labeled goat anti-Human IgG-heavy and light chain antibody (Bethyl Laboratories®, Cat A80-319A).

### Rat PD

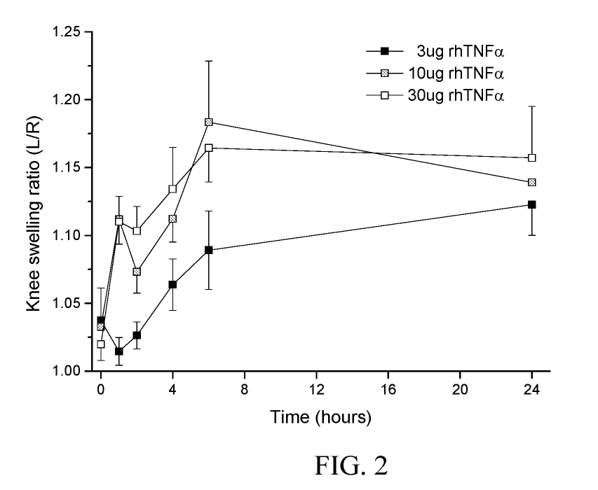

Knee swelling pharmacodynamic model: Injection of 3, 10 or 30 ug of recombinant human TNFα into the knee joints of rats produced a time dependent increase in joint swelling which was maximal at 6 hours to 24 hours (see FIG. 2). The dose of 30 ug was chosen for the subsequent pharmacodynamic experiment, since this produced the maximal swelling response.

### Effect of Fabs on Knee Swelling:

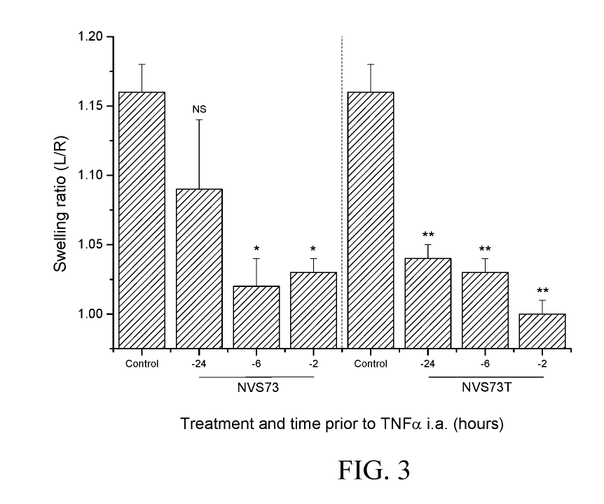

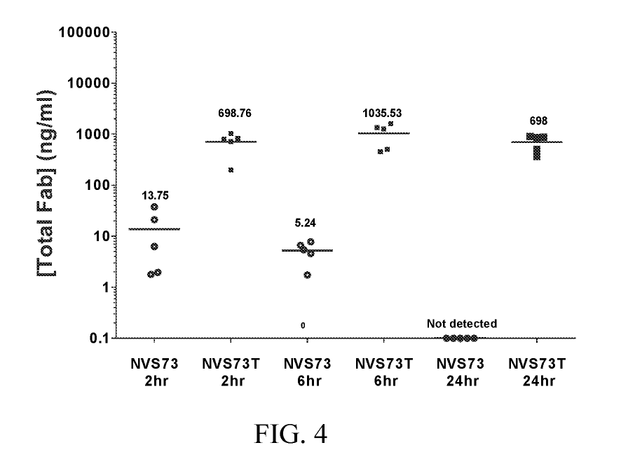

The effect of an i.a. injection of either an anti-TNFα Fab (NVS73) or a hyaluronic acid binding peptide tagged anti-TNFα Fab (NVS73T), 24, 6 or 2 hours prior to the recombinant human TNFα i.a. injection is shown in FIG. 3. The injection of the anti-TNFα Fab at times −6 and −2 h showed inhibition of the knee swelling response, but no inhibition was seen when the injection was given 24 hours prior to the swelling stimulus. By contrast, the hyaluronic acid tagged anti-TNFα Fab demonstrated a marked and significant inhibition of the swelling response at all pre-stimulus injection time points. This shows that the hyaluronic acid binding peptide tagged anti-TNFα Fab (NVS73T) has a longer residence time in the joint, and even when injected 24 hours prior to the proinflammatory TNFα stimulus it is able to inhibit the swelling response.

### Example 4: Rat PK/PD Study with Antibodies

### Animals:

Female Lewis rats weighing between 185 and 200 g were used throughout. The rats were held in groups of 5 animals in IVC (individually ventilated cage) racks. This study was performed in accordance with the animal experimentation guidelines and laws laid down by the Swiss Federal and Cantonal Authorities, and specifically as described in Basel-Stadt Experimental License No. 1438.

### Pharmacokinetic/Pharmacodynamic:

The rats were anaesthetised with 3.5-5% isoflurane in air in an anaesthetic induction chamber. Intravenous injections were performed using a 25 G needle and 1 ml syringe under isoflurane anaesthesia. Groups of 5 rats received either, 80 μg or 190 μg adalimumab (Humira®), 80 μg mAb1 (CH3—hyaluronic acid tagged adalimumab (Humira®)) or 190 μg mAb2 (Kchain—hyaluronic acid tagged adalimumab (Humira®)) at times −24 h or −6 h.

A further group of 5 rats, receiving an i.v. injection of PBS, were included as matrix controls (baseline measurements) for the mass spectrometry method.

At time 0 h after the i.v. injections, the right and left knee diameters of the rats were measured using digital calipers, followed by the intra-articular injection of recombinant human TNFα (30 μg) into each left knee under isoflurane anaesthesia. After 24 hours, the diameters of the right and left rat knees were again measured by digital calipers and the animals bled under isoflurane anaesthesia. The blood was collected into serum preparation tubes (Sarstedt) and placed on ice. The rats were then killed via CO₂ inhalation for collection of synovial fluid washout, synovial tissue and cartilage.

The left knee joints were lavaged after injection of 100 μl of 3.8% sodium citrate solution via a 25 G needle and 1 ml syringe. The synovial washout fluid was placed into Eppendorf tubes on ice.

The skin over the knees was dampened with 70% alcohol and opened by cutting vertically over the center of the joint. The knee joints were opened via a transverse cut above the patella, through the muscle and tendon. The joint was opened by cutting diagonally down on the medial and lateral sides, and the patella reflected outwards and downwards to gain access to the synovial tissue at the front of the joint. The synovial tissue was removed from its attachment at the front of the tibia and dissected free of the patella tendon using a scalpel blade. The tissue was placed in Eppendorf tubes on ice.

Samples of cartilage were removed from the patella groove of the femur using a scalpel blade, transferred into Eppendorf tubes and placed on ice.

Serum, synovial fluid, synovial tissue and cartilage samples were stored at −80° C. prior to mass spectrometric analysis.

### Data and Statistical Analysis:

Knee swelling was calculated as a ratio of the left knee diameter (TNFα injected knee)/right knee diameter (non-injected knee) for each rat. The mean±sem was calculated for each time and treatment group. Area under the curve (AUC) calculations were performed using an Excel spreadsheet. Data were analysed by ANOVA, followed by post hoc tests for multiple comparisons.

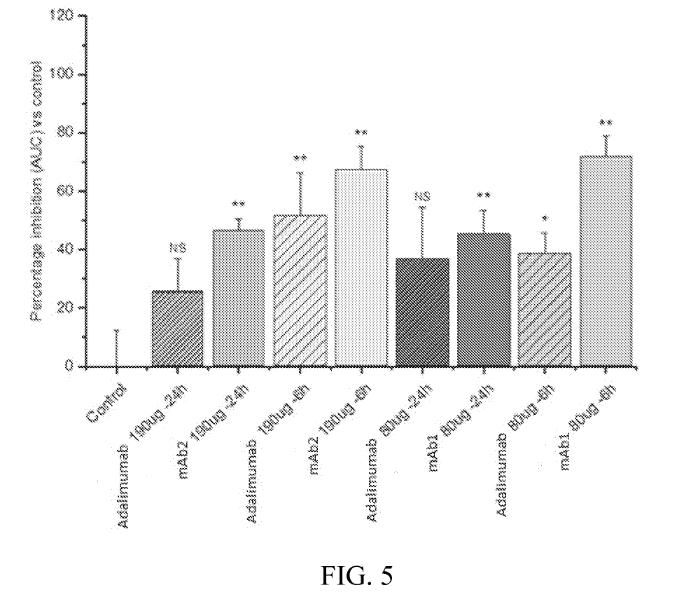

Effect of i.v. antibodies on knee swelling: The effect of an i.v. injection of either adalimumab or two versions of hyaluronic acid tagged adalimumab, 24, or 6 hours prior to the recombinant human TNFα i.a. injection are shown in FIG. 5. The injection of adalimumab at 80 μg or 190 μg at time −6 h showed inhibition of the knee swelling response, but no inhibition was seen at either dose when the i.v. injection was given 24 hours prior to the swelling stimulus. By contrast, both 80 μg mAb1 and 190 μg mAb2 demonstrated a marked and significant inhibition of the swelling response at both pre-stimulus injection time points. This suggests that the hyaluronic acid tagged versions of adalimumab (Humira®) can enter the joint from the circulation and also have a longer residence time in the joint, as they still significantly inhibit knee swelling even when injected systemically 24 hours prior to the proinflammatory TNFα stimulus.

### REFERENCE LIST

Campochiaro, P. A., Channa, R., Berger, B. B., Heier, J. S., Brown, D. M., Fiedler, U., Hepp, J., and Stumpp, M. T. (2012). Treatment of Diabetic Macular Edema With a Designed Ankyrin Repeat Protein That Binds Vascular Endothelial Growth Factor: A Phase 1/2 Study. Am J Ophthalmol.
Day, S., Acquah, K., Mruthyunjaya, P., Grossman, D. S., Lee, P. P., and Sloan, F. A. (2011). Ocular Complications After Anti-Vascular Endothelial Growth Factor Therapy in Medicare Patients With Age-Related Macular Degeneration. Am J Ophthalmol.
Ferrara, N., Damico, L., Shams, N., Lowman, H., and Kim, R. (2006). Development of ranibizumab, an anti-vascular endothelial growth factor antigen binding fragment, as therapy for neovascular age-related macular degeneration. Retina 26, 859-870.
Ferrara, N., Hillan, K. J., Gerber, H. P., and Novotny, W. (2004). Discovery and development of bevacizumab, an anti-VEGF antibody for treating cancer. Nat Rev Drug Discov 3, 391-400.
Levin, A. M. and Weiss, G. A. (2006). Optimizing the affinity and specificity of proteins with molecular display. Mol Biosyst. 2, 49-57.
Lipovsek, D. (2011). Adnectins: engineered target-binding protein therapeutics. Protein Eng Des Sel 24, 3-9.
Muether, P. S., Hermann, M. M., Viebahn, U., Kirchhof, B., and Fauser, S. (2012). Vascular Endothelial Growth Factor in Patients with Exudative Age-related Macular Degeneration Treated with Ranibizumab. Ophthalmology 119, 2082-2086.
Ng, E. W., Shima, D. T., Calias, P., Cunningham, E. T., Jr., Guyer, D. R., and Adamis, A. P. (2006). Pegaptanib, a targeted anti-VEGF aptamer for ocular vascular disease. Nat Rev Drug Discov 5, 123-132.
Patel, R. D., Momi, R. S., and Hariprasad, S. M. (2011). Review of ranibizumab trials for neovascular age-related macular degeneration. Semin. Ophthalmol 26, 372-379.
Pluckthun, A. (2012). Ribosome display: a perspective. Methods Mol Biol 805, 3-28.
Rosenfeld, P. J., Brown, D. M., Heier, J. S., Boyer, D. S., Kaiser, P. K., Chung, C. Y., and Kim, R. Y. (2006). Ranibizumab for neovascular age-related macular degeneration. N. Engl. J. Med. 355, 1419-1431.
Stewart, M. W., Grippon, S., and Kirkpatrick, P. (2012). Aflibercept. Nat Rev Drug Discov 11, 269-270.
Wittrup, K. D. (2001). Protein engineering by cell-surface display. Curr. Opin. Biotechnol. 12, 395-399.
Zhang, M., Zhang, J., Yan, M., Li, H., Yang, C., and Yu, D. (2008). Recombinant anti-vascular endothelial growth factor fusion protein efficiently suppresses choridal neovasularization in monkeys. Mol. Vis. 14, 37-49.
Laurent and Fraser, Functions of the proteoglycans, Ciba Foundation Symposium 124, 1986, p 9-29
J. Necas, L. Bartosikova, P. Brauner, and J. Kolar. Veterinarni Medicina, 53, 2008 (8): 397-411
Mao H, Hart S A, Schink A, Pollok B A. J Am Chem Soc. 2004 Mar. 10; 126(9):2670-1
Yazaki P J, Wu A M, Tsai S W, et al. Tumor targeting of radiometal labeled anti-CEA recombinant T84.66 diabody and t84.66 minibody: comparison to radioiodinated fragments. Bioconjug Chem 2001; 12:220-8.)
Aiello, L. P. (2005). Angiogenic pathways in diabetic retinopathy. N Engl J Med 353, 839-841.
Anand-Apte, B., Ebrahem, Q., Cutler, A., Farage, E., Sugimoto, M., Hollyfield, J., and Folkman, J. (2010). Betacellulin induces increased retinal vascular permeability in mice. PLoS. ONE. 5, e13444.
Boyer, David S. A Phase 2b Study of Fovista™, a Platelet Derived Growth Factor (PDGF) inhibitor in combination with a Vascular Endothelial Growth Factor (VEGF) inhibitor for Neovascular Age-Related Macular Degeneration (AMD). ARVO 2013. 2013.
Ref Type: Abstract
Hara, C., Kasai, A., Gomi, F., Satooka, T., Sakimoto, S., Nakai, K., Yoshioka, Y., Yamamuro, A., Maeda, S., and Nishida, K. (2013). Laser-induced choroidal neovascularization in mice attenuated by deficiency in the apelin-APJ system. Invest Ophthalmol. Vis. Sci. 54, 4321-4329.
Kaiser, P. K. (2013). Emerging therapies for neovascular age-related macular degeneration: drugs in the pipeline. Ophthalmology 120, S11-S15.
Kaiser, Peter K., Boyer, David S., and Campochiaro, Peter A. Integrin Peptide Therapy: The First Wet AMD Experience. ARVO 2013. 2013.
Ref Type: Abstract
Nussenblatt, R. B., Liu, B., Wei, L., and Sen, H. N. (2013). The immunological basis of degenerative diseases of the eye. Int. Rev. Immunol. 32, 97-112.
Oliner, J. D., Bready, J., Nguyen, L., Estrada, J., Hurh, E., Ma, H., Pretorius, J., Fanslow, W., Nork, T. M., Leedle, R. A., Kaufman, S., and Coxon, A. (2012). AMG 386, a selective angiopoietin 1/2-neutralizing peptibody, inhibits angiogenesis in models of ocular neovascular diseases. Invest Ophthalmol. Vis. Sci. 53, 2170-2180.
Patel, R. D., Momi, R. S., and Hariprasad, S. M. (2011). Review of ranibizumab trials for neovascular age-related macular degeneration. Semin. Ophthalmol 26, 372-379.
Patel, S. (2009a). Combination therapy for age-related macular degeneration. Retina 29, S45-S48.
Patel, S. (2009b). Combination therapy for age-related macular degeneration. Retina 29, S45-S48.
Rosenfeld, P. J., Brown, D. M., Heier, J. S., Boyer, D. S., Kaiser, P. K., Chung, C. Y., and Kim, R. Y. (2006). Ranibizumab for neovascular age-related macular degeneration. N. Engl. J. Med. 355, 1419-1431.
Watanabe, D., Suzuma, K., Matsui, S., Kurimoto, M., Kiryu, J., Kita, M., Suzuma, I., Ohashi, H., Ojima, T., Murakami, T., Kobayashi, T., Masuda, S., Nagao, M., Yoshimura, N., and Takagi, H. (2005). Erythropoietin as a Retinal Angiogenic Factor in Proliferative Diabetic Retinopathy. N Engl J Med 353, 782-792.

1. A peptide tagged molecule comprising a peptide tag that binds hyaluronan (HA), wherein said peptide tag comprises a sequence selected from the group consisting of:
a) SEQ ID NO:32, SEQ ID NO: 33, SEQ ID NO: 34, SEQ ID NO: 35, SEQ ID NO: 36, SEQ ID NO:204, SEQ ID NO: 205, SEQ ID NO:206, SEQ ID NO:207, and SEQ ID NO: 220; or
b) 95 consecutive amino acids of the sequence of SEQ ID NO:32, SEQ ID NO: 33, SEQ ID NO: 34, SEQ ID NO: 35, SEQ ID NO: 36, SEQ ID NO:204, SEQ ID NO: 205, SEQ ID NO:206, SEQ ID NO:207, and SEQ ID NO: 220;
and wherein said peptide tag is linked to a protein.

2. The peptide tagged molecule of claim 1, wherein said protein is a protein that binds TNFα.

3. The peptide tagged molecule of claim 2, wherein said protein that binds TNFα comprises heavy chain CDR1, CDR2, and CDR3 sequences of SEQ ID NOS: 108, 109, and 110, respectively and light chain CDR1, CDR2, and CDR3 sequences of SEQ ID NOs 117, 118 and 119, respectively.

4. The peptide tagged molecule of claim 2, wherein said protein binds TNFα comprises heavy chain CDR1, CDR2, and CDR3 sequences of SEQ ID NOS: 208, 209, and 210, respectively and light chain CDR1, CDR2, and CDR3 sequences of SEQ ID NOs 213, 214 and 215, respectively.

5. The peptide tagged molecule of claim 1, wherein the peptide tag comprises an amino acid sequence that has at least 95% sequence identity to an amino acid sequence of any one of SEQ ID NO:32, SEQ ID NO:33, SEQ ID NO:34, SEQ ID NO:35, SEQ ID NO:36, SEQ ID NO:204, SEQ ID NO: 205, SEQ ID NO:206, SEQ ID NO:207, or SEQ ID NO: 220.

6. The peptide tagged molecule as claimed in claim 3, wherein the molecule is an isolated antibody or antigen binding fragment.

7. The peptide tagged molecule of claim 2, wherein the molecule comprises an amino acid sequence that has at least 95% sequence identity to an amino acid sequence of any one of SEQ ID NO:113, SEQ ID NO:122, SEQ ID NO: 212, SEQ ID NO: 217, SEQ ID NO: 218 or SEQ ID NO: 219.

8. The peptide tagged molecule of claim 1 wherein said protein is further linked to one or more peptide tags selected from the group consisting of SEQ ID NO:32, SEQ ID NO:33, SEQ ID NO:34, SEQ ID NO:35, SEQ ID NO:36, SEQ ID NO:204, SEQ ID NO: 205, SEQ ID NO:206, SEQ ID NO:207, and SEQ ID NO: 220.

9. The peptide tagged molecule of claim 1, wherein the protein comprises an amino acid sequence of any one of SEQ ID NO:113, SEQ ID NO:115, SEQ ID NO:122, SEQ ID NO: 212, SEQ ID NO: 217, SEQ ID NO: 218 or SEQ ID NO: 219.

10. The peptide tagged molecule of claim 1, wherein said peptide tag is linked at the N terminus and/or the C terminus to said protein.

11. The peptide tagged molecule of claim 1, wherein said peptide tag is linked directly to said protein or nucleic acid.

12. The peptide tagged molecule of claim 1, wherein said peptide tag is linked indirectly to said protein or nucleic acid via a linker.

13. The peptide tagged molecule as claimed in claim 11, wherein the molecule is an isolated antibody or antigen binding fragment thereof, and comprises a variable heavy chain domain and a variable light chain domain having the sequences of: SEQ ID NO: 111 and SEQ ID NO: 120, respectively.

14. The peptide tagged molecule as claimed in claim 11, wherein the molecule is an isolated antibody or antigen binding fragment thereof, and comprises a variable heavy chain domain and a variable light chain domain having the sequences of: SEQ ID NO: 211 and SEQ ID NO: 216, respectively.

15. The peptide tagged molecule as claimed in claim 11, wherein the molecule is an isolated antibody or antigen binding fragment thereof, and comprises a heavy chain and a light chain sequence of SEQ ID NO: 113 and SEQ ID NO: 122, respectively.

16. The peptide tagged molecule as claimed in claim 12, comprising the sequences of SEQ ID NOs: 115 and 122.

17. The peptide tagged molecule as claimed in claim 12, comprising the sequences of SEQ ID NOs: 212 and 217.

18. The peptide tagged molecule as claimed in claim 12, comprising the sequences of SEQ ID NOs: 218 and 219.

19. The peptide tagged molecule of claim 1, wherein said peptide tagged molecule has an increased half-life, increased mean residence time, or decreased clearance in synovial fluid relative to said protein not linked to said peptide tag.

20. A composition comprising a peptide tagged molecule as claimed in claim 1 and a pharmaceutically acceptable excipient, diluent or carrier.

21. The composition as claimed in claim 20 formulated for intra-articular delivery.

22. A nucleic acid encoding a peptide tag of claim 1.

23. A nucleic acid encoding a peptide tagged molecule as claimed in claim 2.

24. An expression vector comprising the nucleic acid as claimed in claim 22.

25. A host cell comprising the expression vector as claimed in claim 24.

26. The peptide tagged molecule of claim 1, for use as a medicament.

27. The peptide tagged molecule of claim 1, for use in treating a condition or disorder associated with synovial joint disease in a subject.

28. A method of treating arthritis, comprising administering the peptide tagged molecule of claim 1.

29. A method for treating a condition or disorder selected from the group consisting of rheumatoid arthritis, systemic lupus erythematosus, gout, pseudo-gout, ankylosing spondylitis, psoriatic arthritis, gonorrhea, tuberculosis, osteomyelitis, and osteoarthritis, comprising administering the peptide tagged molecule of claim 1.

30. A composition comprising the peptide tagged molecule of claim 1 and synovial fluid.

31. A method of treating a condition or disorder of the joint in a subject, the method comprising administering to the subject a composition as claimed in claim 20.

32. The method of claim 31, wherein said joint is known to have or is suspected of having said condition or disorder.

33. A method of treating arthritis in a subject comprising administering to a joint of said subject the peptide tagged molecule of claim 1.

34. The method of claim 33, wherein said joint is known to have or is suspected of having arthritis.

35. A method of treating joint injury in a subject comprising administering to a joint of said subject the peptide tagged molecule of claim 1.

36. The method of claim 35, wherein said joint is known to have or is suspected of having a joint injury.

37. The method of claim 35, wherein said joint is an acromioclavicular joint.

38. The method of claim 35, wherein said joint is an elbow joint.

39. The method of claim 35, wherein said joint is a pivot joint selected from the group consisting of atlanto-axial joint, proximal radioulnar joint, and distal radioulnar joint.

40. The method of claim 35, wherein said joint is a condyloid joint.

41. The method of claim 35, wherein said joint is a saddle joint selected from the group consisting of carpometacarpal or trapeziometacarpal joint of thumb.

42. The method of claim 35, wherein said joint is a ball and socket joint selected from the group consisting of shoulder and hip joints.

43. The method of claim 35, wherein said joint is the knee joint.

44. The method of claim 35, wherein said joint is a hinge joint.

45. The method of claim 35, wherein said joint is an interphalangeal joint.

46. A method of increasing the intra-articular half-life of a protein comprising linking the protein to a peptide tag that binds hyaluronan (HA), wherein said peptide tag comprises a sequence selected from the group consisting of:
a) SEQ ID NO: 33, SEQ ID NO: 34, SEQ ID NO: 35, SEQ ID NO: 36, SEQ ID NO:204, SEQ ID NO: 205, SEQ ID NO:206, SEQ ID NO:207, and SEQ ID NO: 220; or
b) 95 consecutive amino acids of the sequence of SEQ ID NO: 33, SEQ ID NO: 34, SEQ ID NO: 35, SEQ ID NO: 36, SEQ ID NO:204, SEQ ID NO: 205, SEQ ID NO:206, SEQ ID NO:207, and SEQ ID NO: 220.


SEQUENCE LISTING (221 sequences)

SEQ ID NO: 1 - protein, 6 aa, Homo sapiens
DYYYMT

SEQ ID NO: 2 - protein, 16 aa, Homo sapiens
FIDPDDDPYYATWAKG

SEQ ID NO: 3 - protein, 11 aa, Homo sapiens
GDHNSGWGLDI

SEQ ID NO: 4 - protein, 8 aa, Homo sapiens
GFSLTDYY

SEQ ID NO: 5 - protein, 5 aa, Homo sapiens
DPDDD

SEQ ID NO: 6 - protein, 11 aa, Homo sapiens
GDHNSGWGLDI

SEQ ID NO: 7 - protein, 120 aa, Homo sapiens
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 8 - DNA, 360 bp, Homo sapiens
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggcgatcacaatagcggctggggcctggatatctggggtcagggcaccctggtcaccgtgtctagc

SEQ ID NO: 9 - protein, 223 aa, Homo sapiens
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 10 - DNA, 669 bp, Homo sapiens
gaggtgcaattggttgaatctgggggcggactggtgcagcccggtggatctttgcgcctgtcctgtacagcttctggcttctccttgaccgactactattacatgacttgggttcgccaagccccaggcaaagggcttgaatgggtggggttcattgaccccgacgatgatccttactacgccacatgggcaaagggccggtttactatcagccgggataattccaaaaacacattgtatttgcaaatgaactcactgagagcagaagatacggctgtgtactattgcgcaggcggcgatcataactccggctggggcctggacatctgggggcaggggaccctggtgacagtcagctcagcctcaacgaaggggcccagcgtgtttcctttggccccaagcagcaagtccacgtccggtgggactgcagctcttggttgtctggtcaaggattatttcccagaacccgtgaccgtgtcttggaacagtggtgcattgacatcaggagtgcatacattcccagctgtgctgcagagctctggcctgtatagcctttcctctgttgtcacggtgcccagctccagcctggggacgcagacctatatttgtaacgtgaaccataaaccctccaacaccaaggttgataaaagagtggagcccaagtcttgt

SEQ ID NO: 11 - protein, 11 aa, Homo sapiens
QASEIIHSWLA

SEQ ID NO: 12 - protein, 7 aa, Homo sapiens
LASTLAS

SEQ ID NO: 13 - protein, 12 aa, Homo sapiens
QNVYLASTNGAN

SEQ ID NO: 14 - protein, 7 aa, Homo sapiens
SEIIHSW

SEQ ID NO: 15 - protein, 3 aa, Homo sapiens
LAS

SEQ ID NO: 16 - protein, 9 aa, Homo sapiens
VYLASTNGA

SEQ ID NO: 17 - protein, 111 aa, Homo sapiens
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLK

SEQ ID NO: 18 - DNA, 333 bp, Homo sapiens
gagatcgtgatgactcagtcacctagcaccctgagcgctagtgtgggcgatagagtgattatcacctgtcaggctagtgaaattattcactcctggctggcctggtatcagcagaagcccggtaaagcccctaagctgctgatctacctggcctctaccctggctagtggcgtgccctctaggtttagcggtagcggtagtggcgccgagttcaccctgactatctctagcctgcagcccgacgacttcgctacctactactgtcagaacgtctacctggctagtactaacggcgctaacttcggtcagggcactaagctgaccgtgctgaag

SEQ ID NO: 19 - protein, 218 aa, Homo sapiens
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - DNA, 654 bp, Homo sapiens
gagatcgtgatgactcagtcacctagcaccctgagcgctagtgtgggcgatagagtgattatcacctgtcaggctagtgaaattattcactcctggctggcctggtatcagcagaagcccggtaaagcccctaagctgctgatctacctggcctctaccctggctagtggcgtgccctctaggtttagcggtagcggtagtggcgccgagttcaccctgactatctctagcctgcagcccgacgacttcgctacctactactgtcagaacgtctacctggctagtactaacggcgctaacttcggtcagggcactaagctgaccgtgctgaagcggaccgtggccgctcctagtgtgtttatcttcccacctagcgacgagcagctgaagtcaggcaccgctagtgtcgtgtgcctgctgaacaacttctaccctagagaagctaaggtgcagtggaaagtggataacgccctgcagtcaggtaatagtcaggaatcagtcaccgagcaggactctaaggatagcacctatagcctgtctagcacactgaccctgtctaaggccgactacgagaagcacaaggtctacgcctgcgaagtgactcaccagggactgtctagccccgtgactaagtcctttaatagaggcgagtgc

SEQ ID NO: 21 - protein, 325 aa, Artificial Sequence
  Description of Artificial Sequence: Synthetic fusion protein sequence
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 22 - DNA, 975 bp, Artificial Sequence
  Description of Artificial Sequence: Synthetic DNA for fusion protein sequence
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggcgatcacaatagcggctggggcctggatatctggggtcagggcaccctggtcaccgtgtctagcgcctctactaagggacctagcgtgttccccctggcccctagctctaagtctactagcggcggcaccgccgctctgggctgcctggtcaaggactacttccccgagcccgtgaccgtcagctggaatagcggcgctctgactagcggagtgcacaccttccccgccgtgctgcagtctagcggcctgtatagcctgtctagcgtcgtgaccgtgcctagctctagcctgggcactcagacctatatctgtaacgtgaaccacaagccctctaacactaaggtggacaagcgggtggaacctaagtcctgcggtagcggcggaggcggagtctatcacagagaggctagatcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgct

SEQ ID NO: 23 - protein, 325 aa, Artificial Sequence
  Description of Artificial Sequence: Synthetic fusion protein sequence
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 24 - DNA, 975 bp, Artificial Sequence
  Description of Artificial Sequence: Synthetic DNA for fusion protein sequence
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggtgatcacaatagcggctggggcctggatatctggggtcaaggcaccctggtcaccgtgtctagcgcctctactaagggcccctcagtgttccccctggcccctagctctaagtctactagcggcggcaccgccgctctgggctgcctggtcaaggactacttccccgagcccgtgaccgtcagctggaatagcggcgctctgactagcggagtgcacaccttccccgccgtgctgcagtctagcggcctgtatagcctgtctagcgtcgtgaccgtgcctagctctagcctgggcactcagacctatatctgtaacgtgaaccacaagccctctaacactaaggtggacaagcgggtggaacctaagtcctgcggtagcggcggaggcggagtctatcacagagaggctcagtcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 25 - protein, 325 aa, Artificial Sequence
  Description of Artificial Sequence: Synthetic fusion protein sequence
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAASGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 26 - DNA, 975 bp, Artificial Sequence
  Description of Artificial Sequence: Synthetic DNA for fusion protein sequence
gaggtgcagctggtggaatcaggcggcggactggtgcagcctggcggtagcctgagactgagctgcaccgctagtggctttagcctgaccgactactactatatgacctgggtcagacaggcccctggtaaaggcctggagtgggtcggctttatcgaccccgacgacgacccctactacgctacctgggctaagggccggttcactatctctagggataactctaagaacaccctgtacctgcagatgaatagcctgagagccgaggacaccgccgtctactactgcgccggcggtgatcacaatagcggctggggcctggatatctggggtcaaggcaccctggtcaccgtgtctagcgcctctactaagggcccctcagtgttccccctggcccctagctctaagtctactagcggcggcaccgccgctctgggctgcctggtcaaggactacttccccgagcccgtgaccgtcagctggaatagcggcgctctgactagcggagtgcacaccttccccgccgtgctgcagtctagcggcctgtatagcctgtctagcgtcgtgaccgtgcctagctctagcctgggcactcagacctatatctgtaacgtgaaccacaagccctctaacactaaggtggacaagcgggtggaacctaagtcctgcggtagcggcggaggcggagtctatcacagagaggctgctagcggtaaatacaagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 27 - protein, 327 aa, Artificial Sequence
  Description of Artificial Sequence: Synthetic fusion protein sequence
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGACGVYHREAQSGKYKLTYAEAKAVCEFEGGHLATYKQLECARKIGFHVCAAGWMAKGRVGYPIVKPGPNCGFGKTGIIDYGIRLNRSERWDAYCYNPHA

SEQ ID NO: 28 - DNA, 981 bp, Artificial Sequence
  Description of Artificial Sequence: Synthetic DNA for fusion protein sequence
gaagtgcagctggtggaaagcggcggaggcctggtgcagcctggcggatctctgagactgagctgtaccgccagcggcttcagcctgaccgactactactacatgacctgggtccgacaggcccctggcaagggactggaatgggtcggattcatcgaccccgacgacgacccctactacgccacatgggccaagggccggttcaccatcagccgggacaacagcaagaacaccctgtacctgcagatgaacagcctgcgggccgaggacaccgccgtgtactattgtgccggcggagatcacaacagcggctggggcctggatatctggggacagggaacactggtcaccgtgtctagcgccagcaccaagggccctagcgtgttccctctggcccctagcagcaagagcacatctggcggaacagccgccctgggctgcctggtcaaggactactttcccgagcccgtgaccgtgtcctggaactctggcgctctgacaagcggcgtgcacacctttccagccgtgctgcagagcagcggcctgtactctctgagcagcgtggtcacagtgcccagctctagcctgggaacccagacctacatctgcaacgtgaaccacaagcccagcaacaccaaggtggacaagcgggtggaacccaagagctgcggatccggcggaggcgcctgtggcgtgtatcacagggaggcccagagcggcaagtacaagctcacctacgccgaggccaaggccgtgtgcgaattcgagggcggccacctggccacctacaagcagctggagtgcgccaggaagatcggcttccacgtgtgtgccgccggctggatggccaaaggcagagtgggctaccccatcgtgaaacccggccccaactgcggcttcggcaagacaggcatcatcgactacggcatcaggctgaacaggagcgagaggtgggacgcctactgctacaacccccacgcc

SEQ ID NO: 29 - protein, 325 aa, Artificial Sequence
  Description of Artificial Sequence: Synthetic fusion protein sequence
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLCTYKQLEAARKIGFHVCAAGWMAKGRVGYPIVKPGPNCGFGKTGIIDYGIRLNRSERWDAYCCNPHA

SEQ ID NO: 30 - DNA, 975 bp, Artificial Sequence
  Description of Artificial Sequence: Synthetic DNA for fusion protein sequence
gaagtgcagctggtggaaagcggcggaggcctggtgcagcctggcggatctctgagactgagctgtaccgccagcggcttcagcctgaccgactactactacatgacctgggtccgacaggcccctggcaagggactggaatgggtcggattcatcgaccccgacgacgacccctactacgccacatgggccaagggccggttcaccatcagccgggacaacagcaagaacaccctgtacctgcagatgaacagcctgcgggccgaggacaccgccgtgtactattgtgccggcggagatcacaacagcggctggggcctggatatctggggacagggaacactggtcaccgtgtctagcgccagcaccaagggccctagcgtgttccctctggcccctagcagcaagagcacatctggcggaacagccgccctgggctgcctggtcaaggactactttcccgagcccgtgaccgtgtcctggaactctggcgctctgacaagcggcgtgcacacctttccagccgtgctgcagagcagcggcctgtactctctgagcagcgtggtcacagtgcccagctctagcctgggaacccagacctacatctgcaacgtgaaccacaagcccagcaacaccaaggtggacaagcgggtggaacccaagagctgcggatccggcggcggcggagtgtatcacagagaggcccagagcggcaagtacaagctgacctacgccgaggccaaggccgtgtgtgagttcgagggcggccacctgtgcacctacaagcagctggaggccgccaggaagatcggcttccacgtgtgtgccgccggctggatggctaaaggcagggtgggctaccccattgtgaagcccggccccaattgcggcttcggcaagaccggcatcatcgactacggcatcaggctgaacaggagcgagaggtgggacgcctactgctgcaacccccacgcc

SEQ ID NO: 31 - protein, 5 aa, Artificial Sequence
  Description of Artificial Sequence: Synthetic linker
GSGGG

SEQ ID NO: 32 - protein, 98 aa, Artificial Sequence
  Description of Artificial Sequence: Synthetic protein tag
GVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAK

SEQ ID NO: 33 - protein, 97 aa, Artificial Sequence
  Description of Artificial Sequence: Synthetic protein tag
GVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 34 - protein, 97 aa, Artificial Sequence
  Description of Artificial Sequence: Synthetic protein tag
GVYHREAASGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 35 - protein, 99 aa, Artificial Sequence
  Description of Artificial Sequence: Synthetic protein tag
ACGVYHREAQSGKYKLTYAEAKAVCEFEGGHLATYKQLECARKIGFHVCAAGWMAKGRVGYPIVKPGPNCGFGKTGIIDYGIRLNRSERWDAYCYNPHA

SEQ ID NO: 36 - protein, 97 aa, Artificial Sequence
  Description of Artificial Sequence: Synthetic protein tag
GVYHREAQSGKYKLTYAEAKAVCEFEGGHLCTYKQLEAARKIGFHVCAAGWMAKGRVGYPIVKPGPNCGFGKTGIIDYGIRLNRSERWDAYCCNPHA

SEQ ID NO: 37 - protein, 5 aa, Homo sapiens
SYAIS

SEQ ID NO: 38 - protein, 17 aa, Homo sapiens
GIGPFFGTANYAQKFQG

SEQ ID NO: 39 - protein, 7 aa, Homo sapiens
DTPYFDY

SEQ ID NO: 40 - protein, 116 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSS

SEQ ID NO: 41 - DNA, 348 bp, Homo sapiens
gaggtgcaattggttcagtctggcgcggaagtgaaaaaaccgggcagcagcgtgaaagtgagctgcaaagcctccggaggcactttttcttcttatgccatttcttgggtgcgccaagcccctgggcagggtctcgagtggatgggcggtatcggtccgttttttggcactgcgaattacgcgcagaagtttcagggccgggtgaccattaccgcggatgaaagcaccagcaccgcgtatatggaactgagcagcctgcgtagcgaagatacggccgtgtattattgcgcgcgtgatactccttattttgattattggggccaaggcaccctggtgacggttagctca

SEQ ID NO: 42 - protein, 219 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 43 - DNA, 657 bp, Homo sapiens
gaggtgcaattggtccaaagcggcgctgaggtcaagaagcctggcagcagcgtgaaggtctcctgcaaggccagcggcggcacattctccagctatgctatcagctgggtcagacaagcccccggccaaggactggaatggatgggaggaatcggccctttcttcggaaccgccaactacgcccagaagtttcagggaagggtgaccatcaccgccgatgagagcacatccacagcctatatggagctctccagcctgagatccgaagacaccgccgtctactactgcgctagggacaccccctacttcgactattggggccagggcacactcgtgaccgtgagctcagccagcaccaaaggccctagcgtcttccccctggctccttccagcaagagcacaagcggaggaacagctgctctcggctgcctggtcaaggactacttccccgagcctgtcacagtgtcctggaatagcggagccctgaccagcggcgtgcatacattccccgctgtgctccagagctccggcctctacagcctcagctccgtggtcaccgtccctagctcctccctgggcacacagacctacatctgcaacgtcaaccacaagccctccaacaccaaggtggacaagagggtggagcccaaaagctgt

SEQ ID NO: 44 - protein, 321 aa, Artificial Sequence
  Description of Artificial Sequence: Synthetic fusion protein sequence
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 45 - DNA, 963 bp, Artificial Sequence
  Description of Artificial Sequence: Synthetic DNA for fusion protein sequence
gaggtgcaattggtccaaagcggcgctgaggtcaagaagcctggcagcagcgtgaaggtctcctgcaaggccagcggcggcacattctccagctatgctatcagctgggtcagacaagcccccggccaaggactggaatggatgggaggaatcggccctttcttcggaaccgccaactacgcccagaagtttcagggaagggtgaccatcaccgccgatgagagcacatccacagcctatatggagctctccagcctgagatccgaagacaccgccgtctactactgcgctagggacaccccctacttcgactattggggccagggcacactcgtgaccgtgagctcagccagcaccaaaggccctagcgtcttccccctggctccttccagcaagagcacaagcggaggaacagctgctctcggctgcctggtcaaggactacttccccgagcctgtcacagtgtcctggaatagcggagccctgaccagcggcgtgcatacattccccgctgtgctccagagctccggcctctacagcctcagctccgtggtcaccgtccctagctcctccctgggcacacagacctacatctgcaacgtcaaccacaagccctccaacaccaaggtggacaagagggtggagcccaaaagctgtggatccggaggaggcggcgtgtatcatagagaggcccagtccggcaagtacaagctgacctacgccgaagccaaggccgtgtgtgagttcgagggcggacacctggctacctacaaacagctcgaagccgctaggaagatcggattccacgtgtgcgccgccggatggatggccaaaggcagagtgggctaccccattgtcaagcccggacccaactgcggattcggcaagaccggcatcatcgactacggcatcaggctcaacaggtccgagagatgggacgcttactgctacaatccccacgcc

SEQ ID NO: 46 - protein, 11 aa, Homo sapiens
SGDSIPNYYVY

SEQ ID NO: 47 - protein, 7 aa, Homo sapiens
DDSNRPS

SEQ ID NO: 48 - protein, 11 aa, Homo sapiens
QSFDSSLNAEV

SEQ ID NO: 49 - protein, 108 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVL

SEQ ID NO: 50 - DNA, 324 bp, Homo sapiens
tcctatgaactcacacagcccctgagcgtgagcgtggccctgggccagaccgcccggatcacctgctccggcgacagcatccccaactactacgtgtactggtaccagcagaagcccggccaggcccccgtgctggtgatctacgacgacagcaaccggcccagcggcatccccgagcggttcagcggcagcaacagcggcaacaccgccaccctgaccatttccagagcacaggcaggcgacgaggccgactactactgccagagcttcgacagcagcctgaacgccgaggtgttcggcggagggaccaagttaaccgtccta

SEQ ID NO: 51 - protein, 214 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 52 - DNA, 642 bp, Homo sapiens
agctacgagctgacccagcccctgagcgtgagcgtggccctgggccagaccgccaggatcacctgcagcggcgacagcatccccaactactacgtgtactggtatcagcagaagcccggccaggcccccgtgctggtgatctacgacgacagcaacaggcccagcggcatccccgagaggttcagcggcagcaacagcggcaacaccgccaccctgaccatcagcagagcccaggccggcgacgaggccgactactactgccagagcttcgacagctcactgaacgccgaggtgttcggcggagggaccaagctgaccgtgctgggccagcctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

SEQ ID NO: 53 - protein, 5 aa, Homo sapiens
SYAIS

SEQ ID NO: 54 - protein, 17 aa, Homo sapiens
RIIPIFGTANYAQKFQG

SEQ ID NO: 55 - protein, 8 aa, Homo sapiens
HGGYSFDS

SEQ ID NO: 56 - protein, 7 aa, Homo sapiens
GGTFNSY

SEQ ID NO: 57 - protein, 6 aa, Homo sapiens
IPIFGT

SEQ ID NO: 58 - protein, 8 aa, Homo sapiens
HGGYSFDS

SEQ ID NO: 59 - protein, 117 aa, Homo sapiens
EVQLVQSGAEVKKPGSSVKVSCKASGGTFNSYAISWVRQAPGQGLEWMGRIIPIFGTANYAQKFQGRVTITADESTSTAYMELSSLRSEDTAVYYCARHGGYSFDSWGQGTLVTVSS

SEQ ID NO: 60 - DNA, 351 bp, Homo sapiens
gaggtgcagctggtgcagagcggagccgaagtgaagaaacccggcagcagcgtgaaggtgtcctgcaaggccagcggcggcaccttcaacagctacgccatcagctgggtgcgccaggctcctggacagggcctggaatggatgggccggatcatccccatcttcggcaccgccaactacgcccagaaattccagggcagagtgaccatcaccgccgacgagagcaccagcaccgcctacatggaactgagcagcctgagaagcgaggacaccgccgtgtactactgtgcccggcacggcggctacagcttcgatagctggggccagggcaccctggtgaccgtgagctca

SEQ ID NO: 61 - protein, 220 aa, Homo sapiens
EVQLVQSGAEVKKPGSSVKVSCKASGGTFSSYAISWVRQAPGQGLEWMGRIIPIFGTANYAQKFQGRVTITADESTSTAYMELSSLRSEDTAVYYCARHGGYSFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 62 - DNA, 660 bp, Homo sapiens
gaggtgcagctggtgcagagcggagccgaagtgaagaaacccggcagcagcgtgaaggtgtcctgcaaggccagcggcggcaccttcaacagctacgccatcagctgggtgcgccaggctcctggacagggcctggaatggatgggccggatcatccccatcttcggcaccgccaactacgcccagaaattccagggcagagtgaccatcaccgccgacgagagcaccagcaccgcctacatggaactgagcagcctgagaagcgaggacaccgccgtgtactactgtgcccggcacggcggctacagcttcgatagctggggccagggcaccctggtgaccgtgagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgt

SEQ ID NO: 63 - protein, 322 aa, Artificial Sequence
  Description of Artificial Sequence: Synthetic fusion protein sequence
EVQLVQSGAEVKKPGSSVKVSCKASGGTFSSYAISWVRQAPGQGLEWMGRIIPIFGTANYAQKFQGRVTITADESTSTAYMELSSLRSEDTAVYYCARHGGYSFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 64 - DNA, 966 bp, Artificial Sequence
  Description of Artificial Sequence: Synthetic DNA for fusion protein sequence
gaggtgcaattggtgcagagcggagctgaggtgaagaagcccggcagctccgtcaaggtgagctgcaaagcctccggaggcaccttttcctcctacgctatctcctgggtgaggcaagcccccggacaaggactggagtggatgggcaggatcatccccatcttcggaaccgccaactacgcccagaaattccagggcagggtgaccatcaccgccgacgaaagcaccagcaccgcctacatggagctctccagcctgaggagcgaggacaccgctgtgtactactgcgccagacacggcggctactatttcgacagctggggccagggcacactggtgaccgtgagctcagcaagcaccaaaggaccctccgtctttcctctggcccccagcagcaagtccacaagcggaggaaccgctgccctgggatgtctcgtgaaggactacttccctgagcccgtgacagtgtcctggaatagcggcgccctgacaagcggcgtgcacacatttcccgccgtcctgcaaagctccggcctctatagcctgagctccgtcgtgacagtcccctccagctccctgggaacccagacctacatctgcaacgtcaaccacaagcccagcaacacaaaggtggacaagagggtcgagcctaagagctgtggatccggcggcggaggagtgtaccatagggaggcccagagcggaaagtacaagctgacctatgccgaggctaaggccgtctgcgaattcgagggcggccatctggccacctacaagcaactggaggccgctaggaagatcggcttccacgtctgcgccgctggatggatggccaagggcagagtgggctatcccatcgtgaagcccggccccaactgcggcttcggaaagacaggcatcatcgactacggcatcaggctcaacaggagcgagaggtgggacgcttactgctacaacccccatgcc

SEQ ID NO: 65 - protein, 11 aa, Homo sapiens
SGDNLGSKYVD

SEQ ID NO: 66 - protein, 7 aa, Homo sapiens
SDNNRPS

SEQ ID NO: 67 - protein, 10 aa, Homo sapiens
QTYTSGNNYL

SEQ ID NO: 68 - protein, 7 aa, Homo sapiens
DNLGSKY

SEQ ID NO: 69 - protein, 3 aa, Homo sapiens
SDN

SEQ ID NO: 70 - protein, 8 aa, Homo sapiens
YTSGNNYL

SEQ ID NO: 71 - protein, 108 aa, Homo sapiens
SYELTQPPSVSVAPGQTARISCSGDNLGSKYVDWYQQKPGQAPVLVIYSDNNRPSGIPERFSGSNSGNTATLTISGTQAEDEADYYCQTYTSGNNYLVFGGGTKLTVL

SEQ ID NO: 72 - DNA, 324 bp, Homo sapiens
agctacgagctgactcagcccccttctgtgtctgtggcccctggccagaccgccagaatcagctgcagcggcgacaacctgggcagcaaatacgtggactggtatcagcagaagcccggccaggctcccgtgctggtgatctacagcgacaacaaccggcccagcggcatccctgagcggttcagcggcagcaacagcggcaataccgccaccctgaccatcagcggcacccaggccgaggacgaggccgactactactgccagacctacaccagcggcaacaactacctggtgttcggaggcggaacaaagttaaccgtccta

SEQ ID NO: 73 - protein, 214 aa, Homo sapiens
SYELTQPPSVSVAPGQTARISCSGDNLGSKYVDWYQQKPGQAPVLVIYSDNNRPSGIPERFSGSNSGNTATLTISGTQAEDEADYYCQTYTSGNNYLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 74 - DNA, 642 bp, Homo sapiens
agctacgagctgactcagcccccttctgtgtctgtggcccctggccagaccgccagaatcagctgcagcggcgacaacctgggcagcaaatacgtggactggtatcagcagaagcccggccaggctcccgtgctggtgatctacagcgacaacaaccggcccagcggcatccctgagcggttcagcggcagcaacagcggcaataccgccaccctgaccatcagcggcacccaggccgaggacgaggccgactactactgccagacctacaccagcggcaacaactacctggtgttcggaggcggaacaaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 75 - protein, 5 aa, Homo sapiens
SYWIG

SEQ ID NO: 76 - protein, 17 aa, Homo sapiens
WIDPYRSEIRYSPSFQG

SEQ ID NO: 77 - protein, 8 aa, Homo sapiens
VSSEPFDS

SEQ ID NO: 78 - protein, 7 aa, Homo sapiens
GYSFTSY

SEQ ID NO: 79 - protein, 6 aa, Homo sapiens
DPYRSE

SEQ ID NO: 80 - protein, 8 aa, Homo sapiens
VSSEPFDS

SEQ ID NO: 81 - protein, 117 aa, Homo sapiens
EVQLVQSGAEVKKPGESLKISCKGSGYSFTSYWIGWVRQMPGKGLEWMGWIDPYRSEIRYSPSFQGQVTISADKSISTAYLQWSSLKASDTAMYYCARVSSEPFDSWGQGTLVTVSS

SEQ ID NO: 82 - DNA, 351 bp, Homo sapiens
gaggtccaattggtccaatccggagccgaagtcaagaaacccggcgagtccctcaaaatcagctgcaagggctccggctactccttcaccagctactggatcggatgggtgaggcagatgcccggcaaaggcctcgagtggatgggctggatcgacccctataggtccgagattaggtacagcccctccttccagggccaggtcaccatctccgccgacaagagcatcagcaccgcctacctccaatggtcctccctcaaggcctccgataccgccatgtattactgcgccagggtcagcagcgagccctttgacagctggggccagggaaccctcgtgaccgtcagctca

SEQ ID NO: 83 - protein, 220 aa, Homo sapiens
EVQLVQSGAEVKKPGESLKISCKGSGYSFTSYWIGWVRQMPGKGLEWMGWIDPYRSEIRYSPSFQGQVTISADKSISTAYLQWSSLKASDTAMYYCARVSSEPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 84 - DNA, 660 bp, Homo sapiens
gaggtccaattggtccaatccggagccgaagtcaagaaacccggcgagtccctcaaaatcagctgcaagggctccggctactccttcaccagctactggatcggatgggtgaggcagatgcccggcaaaggcctcgagtggatgggctggatcgacccctataggtccgagattaggtacagcccctccttccagggccaggtcaccatctccgccgacaagagcatcagcaccgcctacctccaatggtcctccctcaaggcctccgataccgccatgtattactgcgccagggtcagcagcgagccctttgacagctggggccagggaaccctcgtgaccgtcagctcagccagcaccaaaggacctagcgtgttccccctcgctccctcctccaagagcacatccggcggaaccgctgctctgggatgtctcgtcaaggactacttccccgagcccgtgaccgtgagctggaatagcggcgccctgacctccggagtccacacattccccgctgtcctgcagagcagcggcctgtatagcctgtcctccgtcgtgaccgtccctagcagctccctgggaacccagacctacatctgcaacgtcaaccacaagcctagcaacaccaaggtggacaagagggtggagcccaaatcctgc

SEQ ID NO: 85 - protein, 322 aa, Artificial Sequence
  Description of Artificial Sequence: Synthetic fusion protein sequence
EVQLVQSGAEVKKPGESLKISCKGSGYSFTSYWIGWVRQMPGKGLEWMGWIDPYRSEIRYSPSFQGQVTISADKSISTAYLQWSSLKASDTAMYYCARVSSEPFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 86 - DNA, 966 bp, Artificial Sequence
  Description of Artificial Sequence: Synthetic DNA for fusion protein sequence
gaggtccaattggtccaatccggagccgaagtcaagaaacccggcgagtccctcaaaatcagctgcaagggctccggctactccttcaccagctactggatcggatgggtgaggcagatgcccggcaaaggcctcgagtggatgggctggatcgacccctataggtccgagattaggtacagcccctccttccagggccaggtcaccatctccgccgacaagagcatcagcaccgcctacctccaatggtcctccctcaaggcctccgataccgccatgtattactgcgccagggtcagcagcgagccctttgacagctggggccagggaaccctcgtgaccgtcagctcagccagcaccaaaggacctagcgtgttccccctcgctccctcctccaagagcacatccggcggaaccgctgctctgggatgtctcgtcaaggactacttccccgagcccgtgaccgtgagctggaatagcggcgccctgacctccggagtccacacattccccgctgtcctgcagagcagcggcctgtatagcctgtcctccgtcgtgaccgtccctagcagctccctgggaacccagacctacatctgcaacgtcaaccacaagcctagcaacaccaaggtggacaagagggtggagcccaaatcctgcggatccggaggaggcggcgtgtatcacagagaggcccagagcggcaagtacaagctcacatacgctgaggccaaagccgtgtgcgaattcgagggcggacatctggccacatataagcagctggaggccgccaggaagatcggcttccacgtgtgcgctgccggctggatggccaaaggcagagtgggctaccctatcgtcaagcccggccccaactgcggctttggcaagaccggcatcatcgactacggcatcaggctcaacaggtccgaaaggtgggatgcctactgctacaatccccacgcc

SEQ ID NO: 87 - protein, 11 aa, Homo sapiens
SGDKLGDHYAY

SEQ ID NO: 88 - protein, 7 aa, Homo sapiens
DDSKRPS

SEQ ID NO: 89 - protein, 10 aa, Homo sapiens
ATWTFEGDYV

SEQ ID NO: 90 - protein, 7 aa, Homo sapiens
DKLGDHY

SEQ ID NO: 91 - protein, 3 aa, Homo sapiens
DDS

SEQ ID NO: 92 - protein, 7 aa, Homo sapiens
WTFEGDY

SEQ ID NO: 93 - protein, 107 aa, Homo sapiens
SYVLTQPPSVSVAPGKTARITCSGDKLGDHYAYWYQQKPGQAPVLVIYDDSKRPSGIPERFSGSNSGNTATLTISRVEAGDEADYYCATWTFEGDYVFGGGTKLTVL

SEQ ID NO: 94 - DNA, 321 bp, Homo sapiens
tcctacgtcctgacacaacctcccagcgtgagcgtcgctcctggcaagacagccagaatcacctgcagcggcgacaagctgggcgaccactacgcctactggtatcagcagaaacccggccaagctcccgtgctggtgatctatgacgacagcaagagaccctccggcatccctgagagattcagcggaagcaactccggcaacaccgccaccctgaccatcagcagggtcgaagccggcgatgaggccgactactactgcgccacctggacctttgagggcgactacgtgttcggaggcggcaccaagttaaccgtccta

SEQ ID NO: 95 - protein, 213 aa, Homo sapiens
SYVLTQPPSVSVAPGKTARITCSGDKLGDHYAYWYQQKPGQAPVLVIYDDSKRPSGIPERFSGSNSGNTATLTISRVEAGDEADYYCATWTFEGDYVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 96 - DNA, 639 bp, Homo sapiens
tcctacgtcctgacacaacctcccagcgtgagcgtcgctcctggcaagacagccagaatcacctgcagcggcgacaagctgggcgaccactacgcctactggtatcagcagaaacccggccaagctcccgtgctggtgatctatgacgacagcaagagaccctccggcatccctgagagattcagcggaagcaactccggcaacaccgccaccctgaccatcagcagggtcgaagccggcgatgaggccgactactactgcgccacctggacctttgagggcgactacgtgttcggaggcggcaccaagttaaccgtcctaggacagcctaaggccgctccctccgtgacactgtttccccctagcagcgaggagctgcaggccaacaaggccaccctcgtgtgcctcatctccgacttctaccctggcgccgtcacagtcgcctggaaagccgacagctcccccgtcaaagctggcgtggagaccaccacccccagcaagcagagcaacaacaagtacgccgcctcctcctatctgagcctgacccccgagcagtggaagagccacaggagctactcctgccaggtgacacacgagggcagcaccgtcgagaagaccgtcgctcccaccgagtgcagc

SEQ ID NO: 97 - protein, 206 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 98 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 99 - protein, 1658 aa, Homo sapiens
QEQTYVISAPKIFRVGASENIVIQVYGYTEAFDATISIKSYPDKKFSYSSGHVHLSSENKFQNSAILTIQPKQLPGGQNPVSYVYLEVVSKHFSKSKRMPITYDNGFLFIHTDKPVYTPDQSVKVRVYSLNDDLKPAKRETVLTFIDPEGSEVDMVEEIDHIGIISFPDFKIPSNPRYGMWTIKAKYKEDFSTTGTAYFEVKEYVLPHFSVSIEPEYNFIGYKNFKNFEITIKARYFYNKVVTEADVYITFGIREDLKDDQKEMMQTAMQNTMLINGIAQVTFDSETAVKELSYYSLEDLNNKYLYIAVTVIESTGGFSEEAEIPGIKYVLSPYKLNLVATPLFLKPGIPYPIKVQVKDSLDQLVGGVPVTLNAQTIDVNQETSDLDPSKSVTRVDDGVASFVLNLPSGVTVLEFNVKTDAPDLPEENQAREGYRAIAYSSLSQSYLYIDWTDNHKALLVGEHLNIIVTPKSPYIDKITHYNYLILSKGKIIHFGTREKFSDASYQSINIPVTQNMVPSSRLLVYYIVTGEQTAELVSDSVWLNIEEKCGNQLQVHLSPDADAYSPGQTVSLNMATGMDSWVALAAVDSAVYGVQRGAKKPLERVFQFLEKSDLGCGAGGGLNNANVFHLAGLTFLTNANADDSQENDEPCKEILRPRRTLQKKIEEIAAKYKHSVVKKCCYDGACVNNDETCEQRAARISLGPRCIKAFTECCVVASQLRANISHKDMQLGRLHMKTLLPVSKPEIRSYFPESWLWEVHLVPRRKQLQFALPDSLTTWEIQGVGISNTGICVADTVKAKVFKDVFLEMNIPYSVVRGEQIQLKGTVYNYRTSGMQFCVKMSAVEGICTSESPVIDHQGTKSSKCVRQKVEGSSSHLVTFTVLPLEIGLHNINFSLETWFGKEILVKTLRVVPEGVKRESYSGVTLDPRGIYGTISRRKEFPYRIPLDLVPKTEIKRILSVKGLLVGEILSAVLSQEGINILTHLPKGSAEAELMSVVPVFYVFHYLETGNHWNIFHSDPLIEKQKLKKKLKEGMLSIMSYRNADYSYSVWKGGSASTWLTAFALRVLGQVNKYVEQNQNSICNSLLWLVENYQLDNGSFKENSQYQPIKLQGTLPVEARENSLYLTAFTVIGIRKAFDICPLVKIDTALIKADNFLLENTLPAQSTFTLAISAYALSLGDKTHPQFRSIVSALKREALVKGNPPIYRFWKDNLQHKDSSVPNTGTARMVETTAYALLTSLNLKDINYVNPVIKWLSEEQRYGGGFYSTQDTINAIEGLTEYSLLVKQLRLSMDIDVSYKHKGALHNYKMTDKNFLGRPVEVLLNDDLIVSTGFGSGLATVHVTTVVHKTSTSEEVCSFYLKIDTQDIEASHYRGYGNSDYKRIVACASYKPSREESSSGSSHAVMDISLPTGISANEEDLKALVEGVDQLFTDYQIKDGHVILQLNSIPSSDFLCVRFRIFELFEVGFLSPATFTVYEYHRPDKQCTMFYSTSNIKIQKVCEGAACKCVEADCGQMQEELDLTISAETRKQTACKPEIAYAYKVSITSITVENVFVKYKATLLDIYKTGEAVAEKDSEITFIKKVTCTNAELVKGRQYLIMGKEALQIKYNFSFRYIYPLDSLTWIEYWPRDTTCSSCQAFLANLDEFAEDIFLNGC

SEQ ID NO: 100 - protein, 442 aa, Homo sapiens
DPVLCFTQYEESSGKCKGLLGGGVSVEDCCLNTAFAYQKRSGGLCQPCRSPRWSLWSTWAPCSVTCSEGSQLRYRRCVGWNGQCSGKVAPGTLEWQLQACEDQQCCPEMGGWSGWGPWEPCSVTCSKGTRTRRRACNHPAPKCGGHCPGQAQESEACDTQQVCPTHGAWATWGPWTPCSASCHGGPHEPKETRSRKCSAPEPSQKPPGKPCPGLAYEQRRCTGLPPCPVAGGWGPWGPVSPCPVTCGLGQTMEQRTCNHPVPQHGGPFCAGDATRTHICNTAVPCPVDGEWDSWGEWSPCIRRNMKSISCQEIPGQQSRGRTCRGRKFDGHRCAGQQQDIRHCYSIQHCPLKGSWSEWSTWGLCMPPCGPNPTRARQRLCTPLLPKYPPTVSMVEGQGEKNVTFWGRPLPRCEELQGQKLVVEEKRPCLHVPACKDPEEEEL

SEQ ID NO: 101 - protein, 157 aa, Homo sapiens [sequence omitted]

SEQ ID NO: 102 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 103 - DNA, 294 bp, Artificial Sequence
  Description of Artificial Sequence: Synthetic DNA for protein tag sequence
ggagtctatcacagagaggctagatcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgctaag

SEQ ID NO: 104 - DNA, 291 bp, Artificial Sequence
  Description of Artificial Sequence: Synthetic DNA for protein tag sequence
ggagtctatcacagagaggctcagtcaggcaagtataagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 105 - DNA, 291 bp, Artificial Sequence
  Description of Artificial Sequence: Synthetic DNA for protein tag sequence
ggagtctatcacagagaggctgctagcggtaaatacaagctgacctacgccgaggctaaggccgtgtgcgagttcgagggcggtcacctggctacctataagcagctggaagccgctagaaagatcggctttcacgtgtgcgccgctggctggatggctaagggtagagtgggctaccctatcgtgaagcctggccctaactgcggcttcggtaaaaccggaattatcgactacgggattaggctgaatagatcagagcgctgggacgcctactgctataaccctcacgcc

SEQ ID NO: 106 - DNA, 300 bp, Artificial Sequence
  Description of Artificial Sequence: Synthetic DNA for protein tag sequence
ggcgcctgtggcgtgtatcacagggaggcccagagcggcaagtacaagctcacctacgccgaggccaaggccgtgtgcgaattcgagggcggccacctggccacctacaagcagctggagtgcgccaggaagatcggcttccacgtgtgtgccgccggctggatggccaaaggcagagtgggctaccccatcgtgaaacccggccccaactgcggcttcggcaagacaggcatcatcgactacggcatcaggctgaacaggagcgagaggtgggacgcctactgctacaacccccacgcc

SEQ ID NO: 107 - DNA, 291 bp, Artificial Sequence
  Description of Artificial Sequence: Synthetic DNA for protein tag sequence
ggagtgtatcacagagaggcccagagcggcaagtacaagctgacctacgccgaggccaaggccgtgtgtgagttcgagggcggccacctgtgcacctacaagcagctggaggccgccaggaagatcggcttccacgtgtgtgccgccggctggatggctaaaggcagggtgggctaccccattgtgaagcccggccccaattgcggcttcggcaagaccggcatcatcgactacggcatcaggctgaacaggagcgagaggtgggacgcctactgctgcaacccccacgcc

SEQ ID NO: 108 - protein, 11 aa, Homo sapiens
GFTISRSYWIC

SEQ ID NO: 109 - protein, 18 aa, Homo sapiens
CIYGDNDITPLYANWAKG

SEQ ID NO: 110 - protein, 10 aa, Homo sapiens
LGYADYAYDL

SEQ ID NO: 111 - protein, 121 aa, Homo sapiens
EVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSS

SEQ ID NO: 112 - DNA, 363 bp, Homo sapiens
gaggtccagctggtggagagcggaggaggaagcgtccagcctggaggcagcctgagactgagctgcaccgccagcggcttcaccatcagcaggagctactggatctgctgggtgaggcaggctcctggcaagggactcgagtgggtgggctgcatctacggcgacaacgacatcacccccctctacgccaactgggctaagggcaggttcaccattagcagggacaccagcaagaacaccgtgtacctccagatgaacagcctgagggccgaggataccgccacctactattgcgccaggctgggctacgccgattacgcctatgacctctggggccagggcaccacagtgaccgtcagctca

SEQ ID NO: 113 - protein, 224 aa, Homo sapiens
EVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 114 - DNA, 672 bp, Homo sapiens
gaggtccagctggtggagagcggaggaggaagcgtccagcctggaggcagcctgagactgagctgcaccgccagcggcttcaccatcagcaggagctactggatctgctgggtgaggcaggctcctggcaagggactcgagtgggtgggctgcatctacggcgacaacgacatcacccccctctacgccaactgggctaagggcaggttcaccattagcagggacaccagcaagaacaccgtgtacctccagatgaacagcctgagggccgaggataccgccacctactattgcgccaggctgggctacgccgattacgcctatgacctctggggccagggcaccacagtgaccgtcagctcagcctccaccaagggaccttccgtgttccccctggcccctagctccaagtccaccagcggaggaacagccgctctgggctgtctggtgaaggactacttccccgagcctgtgaccgtgtcctggaattccggcgccctcacaagcggagtgcataccttccccgccgtgctgcaaagctccggactgtactccctctccagcgtggtgacagtgccttccagcagcctcggcacccagacctacatctgcaacgtgaaccacaagccctccaataccaaggtggacaagagggtcgagcctaaaagctgt

SEQ ID NO: 115 - protein, 326 aa, Artificial Sequence
  Description of Artificial Sequence: Synthetic fusion protein sequence
EVQLVESGGGSVQPGGSLRLSCTASGFTISRSYWICWVRQAPGKGLEWVGCIYGDNDITPLYANWAKGRFTISRDTSKNTVYLQMNSLRAEDTATYYCARLGYADYAYDLWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 116 - DNA, 978 bp, Artificial Sequence
  Description of Artificial Sequence: Synthetic DNA for fusion protein sequence
gaggtccagctggtggagagcggaggaggaagcgtccagcctggaggcagcctgagactgagctgcaccgccagcggcttcaccatcagcaggagctactggatctgctgggtgaggcaggctcctggcaagggactcgagtgggtgggctgcatctacggcgacaacgacatcacccccctctacgccaactgggctaagggcaggttcaccattagcagggacaccagcaagaacaccgtgtacctccagatgaacagcctgagggccgaggataccgccacctactattgcgccaggctgggctacgccgattacgcctatgacctctggggccagggcaccacagtgaccgtcagctcagcctccaccaagggaccttccgtgttccccctggcccctagctccaagtccaccagcggaggaacagccgctctgggctgtctggtgaaggactacttccccgagcctgtgaccgtgtcctggaattccggcgccctcacaagcggagtgcataccttccccgccgtgctgcaaagctccggactgtactccctctccagcgtggtgacagtgccttccagcagcctcggcacccagacctacatctgcaacgtgaaccacaagccctccaataccaaggtggacaagagggtcgagcctaaaagctgtggatccggaggaggcggcgtgtatcatagagaggcccagtccggcaagtacaagctgacctacgccgaagccaaggccgtgtgtgagttcgagggcggacacctggctacctacaaacagctcgaagccgctaggaagatcggattccacgtgtgcgccgccggatggatggccaaaggcagagtgggctaccccattgtcaagcccggacccaactgcggattcggcaagaccggcatcatcgactacggcatcaggctcaacaggtccgagagatgggacgcttactgctacaatccccacgcc

SEQ ID NO: 117 - protein, 13 aa, Homo sapiens
QSSQSVYGNIWMA

SEQ ID NO: 118 - protein, 7 aa, Homo sapiens
QASKLAS

SEQ ID NO: 119 - protein, 11 aa, Homo sapiens
QGNFNTGDRYA

SEQ ID NO: 120 - protein, 113 aa, Homo sapiens
EIVMTQSPSTLSASVGDRVIITCQSSQSVYGNIWMAWYQQKPGRAPKLLIYQASKLASGVPSRFSGSGSGAEFTLTISSLQPDDFATYYCQGNFNTGDRYAFGQGTKLTVLKR

SEQ ID NO: 121 - DNA, 339 bp, Homo sapiens
gagatcgtcatgacccagagccccagcacactcagcgcctccgtgggagacagggtgatcatcacctgccagtcctcccagtccgtgtacggcaacatctggatggcctggtaccagcagaagcccggcagagcccccaagctgctgatctaccaggccagcaagctcgcctccggagtgcccagcagattttccggctccggatccggagccgagttcacactgaccatcagcagcctgcagcccgatgacttcgccacctactattgccagggcaacttcaacaccggcgacaggtacgcctttggccagggcaccaagctgaccgtcctcaagcgt

SEQ ID NO: 122 - protein, 219 aa, Homo sapiens
EIVMTQSPSTLSASVGDRVIITCQSSQSVYGNIWMAWYQQKPGRAPKLLIYQASKLASGVPSRFSGSGSGAEFTLTISSLQPDDFATYYCQGNFNTGDRYAFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - DNA, 657 bp, Homo sapiens
gagatcgtcatgacccagagccccagcacactcagcgcctccgtgggagacagggtgatcatcacctgccagtcctcccagtccgtgtacggcaacatctggatggcctggtaccagcagaagcccggcagagcccccaagctgctgatctaccaggccagcaagctcgcctccggagtgcccagcagattttccggctccggatccggagccgagttcacactgaccatcagcagcctgcagcccgatgacttcgccacctactattgccagggcaacttcaacaccggcgacaggtacgcctttggccagggcaccaagctgaccgtcctcaagcgtacggtggctgctcccagcgtcttcatcttcccccccagcgatgagcagctcaagagcggcacagcctccgtggtgtgcctcctgaacaacttctaccctagggaggccaaggtgcaatggaaggtggacaacgccctgcagagcggcaacagccaggagtccgtgaccgagcaggactccaaggacagcacctacagcctgagcagcacactcaccctgagcaaagccgactacgagaagcacaaggtctacgcctgcgaggtgacccatcagggcctgtccagccccgtgaccaagagcttcaacagaggcgagtgc

SEQ ID NO: 124 - protein, 4 aa, Artificial Sequence
  Description of Artificial Sequence: Synthetic linker
GSGG

SEQ ID NO: 125-126 - skipped (2 entries)

SEQ ID NO: 127 - protein, 103 aa, Artificial Sequence
  Description of Artificial Sequence: Synthetic peptide tag
GSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAK

SEQ ID NO: 128 - protein, 31 aa, Artificial Sequence
  Description of Artificial Sequence: Synthetic peptide tag
GSGGGKQKIKHVVKLKGSGGGKLKSQLVKRK

SEQ ID NO: 129 - protein, 46 aa, Artificial Sequence
  Description of Artificial Sequence: Synthetic peptide tag
GSGGGKNGRYSISRGSGGGRDGTRYVQKGEYRGSGGGRRRCGQKKK

SEQ ID NO: 130 - protein, 109 aa, Artificial Sequence
  Description of Artificial Sequence: Synthetic peptide tag
GSGGGVFPYHPRGGRYKLTFAEAQRACAEQDGILASAEQLHAAWRDGLDWCNAGWLRDGSVQYPVNRPREPCGGLGGTGSAGGGGDANGGLRNYGYRHNAEERYDAFCF

SEQ ID NO: 131 - protein, 101 aa, Artificial Sequence
  Description of Artificial Sequence: Synthetic peptide tag
GSGGEVFYVGPARRLTLAGARAQCRRQGAALASVGQLHLAWHEGLDQCDPGWLADGSVRYPIQTPRRRCGGPAPGVRTVYRFANRTGFPSPAERFDAYCFR

SEQ ID NO: 132 - protein, 73 aa, Artificial Sequence
  Description of Artificial Sequence: Synthetic peptide tag
GSGGLKQKIKHVVKLKDENSQLKSEVSKLRSQLVKRKQNGSGGAHWQFNALTVRGGGSSTMMSRSHKTRSHHV

SEQ ID NO: 133 - protein, 103 aa, Artificial Sequence
  Description of Artificial Sequence: Synthetic peptide tag
GSGGGVFHLRSPLGQYKLTFDKAREACANEAATMATYNQLSYAQKAKYHLCSAGWLETGRVAYPTAFASQNCGSGVVGIVDYGPRPNKREMWDVFCYRMKDVN

SEQ ID NO: 134 - protein, 48 aa, Artificial Sequence
  Description of Artificial Sequence: Synthetic peptide tag
GSGGGHQNLKQKIKHVVKLKDENSQLKSEVSKLRSQLAKKKQSETKLQ

SEQ ID NO: 135 - protein, 106 aa, Artificial Sequence
  Description of Artificial Sequence: Synthetic peptide tag
GSGGGGVYHREARSGKYKLTYAEAKAVCEFEGGHLATYKQLEAARKIGFHVCSAGWLETGRVAYPTAFASQNCGSGVVGIVDYGIRLQRSERWDAYCYNPHAKAHP

SEQ ID NO: 136 - protein, 29 aa, Artificial Sequence
  Description of Artificial Sequence: Synthetic peptide tag
GSGGGKVGKSPPVRGSGGGHREARSGKYK

SEQ ID NO: 137 - protein, 26 aa, Artificial Sequence
  Description of Artificial Sequence: Synthetic peptide tag
GSKQKIKHVVKLKGGGSREARSGKYK

SEQ ID NO: 138 - protein, 40 aa, Artificial Sequence
  Description of Artificial Sequence: Synthetic peptide tag
GSGGGKGGNGEPRGDTYRAYGSGGGKGGPQVTRGDVFTMP

SEQ ID NO: 139 - protein, 24 aa, Artificial Sequence
  Description of Artificial Sequence: Synthetic peptide tag
GSGGGRRANAALKAGELYKSILYG

SEQ ID NO: 140 - protein, 24 aa, Artificial Sequence
  Description of Artificial Sequence: Synthetic peptide tag
GSGGGRRANAALKAGELYKSILYG

SEQ ID NO: 141 - skipped

SEQ ID NO: 142 - protein, 214 aa, Homo sapiens
DIQVTQSPSSLSASVGDRVTITCITSTDIDDDMNWYQQKPGKVPKLLISGGNTLRPGVPSRFSGSGSGTDFTLTISSLQPEDVATYYCLQSDSLPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 216 aa, Homo sapiens
QLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 144 - protein, 214 aa, Homo sapiens; identical to SEQ ID NO: 142

SEQ ID NO: 145 - protein, 318 aa, Homo sapiens
QLVQSGPELKKPGASVKVSCKASGYTFTNYGMNWVRQAPGQGLEWMGWINTYTGETTYADDFKGRFVFSLDTSVSTAYLQISSLKAEDTAVYYCEREGGVNNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 146 - protein, 469 aa, Homo sapiens
GGGGGPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPRIPKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 147 - protein, 571 aa, Homo sapiens
GGGGGPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPRIPKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCRVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 148 - protein, 166 aa, Homo sapiens; identical to SEQ ID NO: 98

SEQ ID NO: 149 - protein, 276 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDRGSGGGGVYHREAQSGKYYLTYAEAKAVCEFEGGHLATYKQLEAARKIGFHVCAAGWMAKGRVGYPIVKPGPNCGFGKTGIIDYGIRLNRSERWDAYCYNPHAGSHHHHHH

SEQ ID NO: 150 - DNA, 30 bp, Artificial Sequence
  source: /note=“Description of Artificial Sequence: Synthetic oligonucleotide”
  source: /note=“Description of Combined DNA/RNA Molecule: Synthetic oligonucleotide”
  misc_feature (9)..(10): /note=“Nucleotides at these positions are separated by HEG”
  misc_feature (21)..(22): /note=“Nucleotides at these positions are separated by HEG”
caggcuacgcgtagagcaucatgatccugt

SEQ ID NO: 151 - protein, 120 aa, Artificial Sequence
  Description of Artificial Sequence: Synthetic conjugate
LPETGGGGGGSGGGGVYHREAQSGKYYLTYAEAKAVCEFEGGHLATYKQLEAARKIGFHVCAAGWMAKGRVGYPIVKPGPNCGFGKTGIIDYGIRLNRSERWDAYCYNPHAGGSHHHHHH

SEQ ID NO: 152 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 153 - protein, 272 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGHHHHHH

SEQ ID NO: 154 - protein, 432 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - skipped

SEQ ID NO: 156 - protein, 533 aa, Homo sapiens
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGGGVYHREAISGKYYLTYAEAKAVCEFEGGHLATYKQLEAAQQIGFHVCAAGWMAKGRVGYPIVKPGPNCGFGKTGIIDYGIRLQRSERWDAYCYNPHA

SEQ ID NO: 157 - protein, 453 aa, Homo sapiens
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 214 aa, Homo sapiens
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 555 aa, Homo sapiens
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 160 - protein, 214 aa, Homo sapiens; identical to SEQ ID NO: 158

SEQ ID NO: 161 - protein, 450 aa, Homo sapiens
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 162 - protein, 218 aa, Homo sapiens; identical to SEQ ID NO: 19

SEQ ID NO: 163 - protein, 450 aa, Homo sapiens; identical to SEQ ID NO: 161

SEQ ID NO: 164 - protein, 320 aa, Homo sapiens
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 165 - protein, 259 aa, Homo sapiens
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGGSGGGGSGGGGSSGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSHHHHHH

SEQ ID NO: 166 - protein, 361 aa, Homo sapiens
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGGSGGGGSGGGGSSGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAHHHHHH

SEQ ID NO: 167 - protein, 142 aa, Homo sapiens
SDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAAGSLPETGGGSGHHHHHH

SEQ ID NO: 168 - protein, 237 aa, Homo sapiens
SDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAAGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGHHHHHH

SEQ ID NO: 169 - protein, 164 aa, Homo sapiens
SDLGKKLLEAARAGQDDEVRILMANGADVNAFDWMGWTPLHLAAHEGHLEIVEVLLKNGADVNATDVSGYTPLHLAAADGHLEIVEVLLKYGADVNTKDNTGWTPLHLSADLGRLEIVEVLLKYGADVNAQDKFGKTAFDISIDNGNEDLAEILQKAAHHHHHH

SEQ ID NO: 170 - protein, 270 aa, Homo sapiens
SDLGKKLLEAARAGQDDEVRILMANGADVNAFDWMGWTPLHLAAHEGHLEIVEVLLKNGADVNATDVSGYTPLHLAAADGHLEIVEVLLKYGADVNTKDNTGWTPLHLSADLGRLEIVEVLLKYGADVNAQDKFGKTAFDISIDNGNEDLAEILQKAAGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGHHHHHH

SEQ ID NO: 171 - protein, 325 aa, Homo sapiens; identical to SEQ ID NO: 23

SEQ ID NO: 172 - protein, 223 aa, Homo sapiens
GGGGGEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 173 - protein, 326 aa, Homo sapiens
VYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGS

SEQ ID NO: 174 - protein, 319 aa, Homo sapiens
VYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAKGGGSEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - protein, 325 aa, Homo sapiens; identical to SEQ ID NO: 23

SEQ ID NO: 176 - protein, 320 aa, Homo sapiens; identical to SEQ ID NO: 164

SEQ ID NO: 177 - protein, 225 aa, Homo sapiens
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGS

SEQ ID NO: 178 - protein, 422 aa, Homo sapiens
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 179 - protein, 325 aa, Homo sapiens; identical to SEQ ID NO: 21

SEQ ID NO: 180 - protein, 319 aa, Homo sapiens; identical to SEQ ID NO: 174

SEQ ID NO: 181 - protein, 633 aa, Homo sapiens
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGGGGGGSGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 182 - protein, 218 aa, Homo sapiens; identical to SEQ ID NO: 19

SEQ ID NO: 183 - protein, 427 aa, Homo sapiens
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHAGSGGGGVYHREARSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 184 - protein, 218 aa, Homo sapiens; identical to SEQ ID NO: 19

SEQ ID NO: 185 - protein, 320 aa, Homo sapiens; identical to SEQ ID NO: 164

SEQ ID NO: 186 - skipped

SEQ ID NO: 187 - protein, 5 aa, Artificial Sequence
  Description of Artificial Sequence: Synthetic optional cloning sequence
GGGGG

SEQ ID NO: 188 - protein, 6 aa, Artificial Sequence
  Description of Artificial Sequence: Synthetic optional purification sequence
HHHHHH

SEQ ID NO: 189 - protein, 10 aa, Homo sapiens
GFTFSVYGMN

SEQ ID NO: 190 - protein, 17 aa, Homo sapiens
IIWYDGDNQYYADSVKG

SEQ ID NO: 191 - protein, 9 aa, Homo sapiens
DLRTGPFDY

SEQ ID NO: 192 - protein, 118 aa, Homo sapiens [sequence omitted]

SEQ ID NO: 193 - DNA, 354 bp, Homo sapiens
caggtgcagctggtggaatctggcggcggagtggtgcagcctggcagaagcctgagactgagctgtgccgccagcggcttcaccttcagcgtgtacggcatgaactgggtgcgccaggcccctggcaaaggcctggaatgggtggccatcatttggtacgacggcgacaaccagtactacgccgacagcgtgaagggccggttcaccatcagccgggacaacagcaagaacaccctgtacctgcagatgaacggcctgcgggccgaggataccgccgtgtactactgcgccagggacctgagaacaggccccttcgattattggggccagggcaccctcgtgaccgtgtctagc

SEQ ID NO: 194 - protein, 221 aa, Homo sapiens
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 195 - DNA, 663 bp, Homo sapiens
caggtgcagctggtggaatctggcggcggagtggtgcagcctggcagaagcctgagactgagctgtgccgccagcggcttcaccttcagcgtgtacggcatgaactgggtgcgccaggcccctggcaaaggcctggaatgggtggccatcatttggtacgacggcgacaaccagtactacgccgacagcgtgaagggccggttcaccatcagccgggacaacagcaagaacaccctgtacctgcagatgaacggcctgcgggccgaggataccgccgtgtactactgcgccagggacctgagaacaggccccttcgattattggggccagggcaccctcgtgaccgtgtctagcgcctctacaaagggccccagcgtgttccctctggcccctagcagcaagtctaccagcggaggaacagccgccctgggctgcctcgtgaaggactactttcccgagcccgtgacagtgtcctggaactctggcgccctgacaagcggcgtgcacacctttccagccgtgctgcagagcagcggcctgtactctctgagcagcgtcgtgactgtgcccagcagctctctgggcacccagacctacatctgcaacgtgaaccacaagcccagcaacaccaaggtggacaagcgggtggaacccaagagctgt

SEQ ID NO: 196 - protein, 323 aa, Artificial Sequence
  Description of Artificial Sequence: Synthetic fusion protein sequence
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGSGGGGVYHREAQSGKYKLTYAEAKAVCEFEGGHLATYKQLEAARKIGFHVCAAGWMAKGRVGYPIVKPGPNCGFGKTGIIDYGIRLNRSERWDAYCYNPHA

SEQ ID NO: 197 - DNA, 663 bp, Artificial Sequence; identical to SEQ ID NO: 195
  Description of Artificial Sequence: Synthetic DNA for fusion protein sequence

SEQ ID NO: 198 - protein, 11 aa, Homo sapiens
RASQSIGSSLH

SEQ ID NO: 199 - protein, 7 aa, Homo sapiens
YASQSFS

SEQ ID NO: 200 - protein, 9 aa, Homo sapiens
HQSSSLPFT

SEQ ID NO: 201 - protein, 108 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKR

SEQ ID NO: 202 - protein, 214 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - DNA, 642 bp, Homo sapiens
gagatcgtgctgacccagagccccgactttcagagcgtgacccccaaagaaaaagtgaccatcacctgtcgggccagccagagcatcggctctagcctgcactggtatcagcagaagcccgaccagtcccccaagctgctgattaagtacgccagccagtccttcagcggcgtgcccagcagattttctggcagcggctccggcaccgacttcaccctgaccatcaacagcctggaagccgaggacgccgctgcctactactgtcaccagagcagcagcctgcccttcacctttggccctggcaccaaggtggacatcaagcggacagtggccgctccctccgtgttcatcttcccacctagcgacgagcagctgaagtctggcacagccagcgtcgtgtgcctgctgaacaacttctacccccgcgaggccaaggtgcagtggaaagtggacaacgccctgcagagcggcaacagccaggaaagcgtgaccgagcaggacagcaaggactccacctacagcctgagcagcaccctgacactgagcaaggccgactacgagaagcacaaggtgtacgcctgcgaagtgacccaccagggcctgtctagccccgtgaccaagagcttcaaccggggcgagtgc

SEQ ID NO: 204 - protein, 97 aa, Artificial Sequence
  Description of Artificial Sequence: Synthetic peptide tag
GVYHREAISGKYYLTYAEAKAVCEFEGGHLATYKQLLAAQKIGFHVCAAGWMAKGRVGYPIVKPGPNCGFGKTGIIDYGIRLNRSERWDAYCYNPHA

SEQ ID NO: 205 - protein, 97 aa, Artificial Sequence
  Description of Artificial Sequence: Synthetic peptide tag
GVYHREAISGKYYLTYAEAKAVCEFEGGHLATYKQLQAAQKIGFHVCAAGWMAKGRVGYPIVKPGPNCGFGKTGIIDYGIRLNRSERWDAYCYNPHA

SEQ ID NO: 206 - protein, 99 aa, Artificial Sequence
  Description of Artificial Sequence: Synthetic peptide tag
ACGVYHREAISGKYYLTYAEAKAVCEFEGGHLATYKQLLAAQKIGFHVCAAGWMAKGRVGYPIVKPGPNCGFGKTGIIDYGIRLNRSERWDAYCYNPHA

SEQ ID NO: 207 - protein, 99 aa, Artificial Sequence
  Description of Artificial Sequence: Synthetic peptide tag
ACGVYHREAISGKYYLTYAEAKAVCEFEGGHLATYKQLQAAQKIGFHVCAAGWMAKGRVGYPIVKPGPNCGFGKTGIIDYGIRLNRSERWDAYCYNPHA

SEQ ID NO: 208 - protein, 10 aa, Homo sapiens
GFTFDDYAMH

SEQ ID NO: 209 - protein, 17 aa, Homo sapiens
AITWNSGHIDYADSVEG

SEQ ID NO: 210 - protein, 12 aa, Homo sapiens
VSYLSTASSLDY

SEQ ID NO: 211 - protein, 121 aa, Homo sapiens [sequence omitted]

SEQ ID NO: 212 - protein, 573 aa, Homo sapiens
MKHLWFFLLLVAAPRWVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGSGGGACGVYHREAQSGKYYLTYAEAKAVCEFEGGHLATYKQLLCAQKIGFHVCAAGWMAKGRVGYPIVKPGPNCGFGKTGIIDYGIRLNRSERWDAYCYNPHA

SEQ ID NO: 213 - protein, 11 aa, Homo sapiens
RASQGIRNYLA

SEQ ID NO: 214 - protein, 7 aa, Homo sapiens
AASTLQS

SEQ ID NO: 215 - protein, 9 aa, Homo sapiens
QRYNRAPYT

SEQ ID NO: 216 - protein, 107 aa, Homo sapiens [sequence omitted]

SEQ ID NO: 217 - protein, 234 aa, Homo sapiens
MVLQTQVFISLLLWISGAYGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 218 - protein, 469 aa, Homo sapiens
MKHLWFFLLLVAAPRWVLSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 219 - protein, 338 aa, Homo sapiens
MVLQTQVFISLLLWISGAYGDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSGGGACGVYHREAQSGKYYLTYAEAKAVCEFEGGHLATYKQLLCAQKIGFHVCAAGWMAKGRVGYPIVKPGPNCGFGKTGIIDYGIRLNRSERWDAYCYNPHA

SEQ ID NO: 220 - protein, 99 aa, Artificial Sequence
  Description of Artificial Sequence: Synthetic peptide tag
ACGVYHREAQSGKYYLTYAEAKAVCEFEGGHLATYKQLLCAQKIGFHVCAAGWMAKGRVGYPIVKPGPNCGFGKTGIIDYGIRLNRSERWDAYCYNPHA

SEQ ID NO: 221 - DNA, 324 bp, Homo sapiens
gagatcgtgctgacccagagccccgactttcagagcgtgacccccaaagaaaaagtgaccatcacctgtcgggccagccagagcatcggctctagcctgcactggtatcagcagaagcccgaccagtcccccaagctgctgattaagtacgccagccagtccttcagcggcgtgcccagcagattttctggcagcggctccggcaccgacttcaccctgaccatcaacagcctggaagccgaggacgccgctgcctactactgtcaccagagcagcagcctgcccttcacctttggccctggcaccaaggtggacatcaagcgg